(19) United States
(12) Patent Application Publication
(10) Pub. No.: US 2025/0288589 A1
(43) Pub. Date: Sep. 18, 2025

(54) PYRIDOPYRIMIDINES AND METHODS OF THEIR USE

(71) Applicant: Kineta, Inc., Seattle, WA (US)

(72) Inventors: Gnanasambandam Kumaravel, Lexington, MA (US); Madeline Macdonnell, Brighton, MA (US); Hairuo Peng, Needham, MA (US); Kerem Ozboya, Cambridge, MA (US); Iwona Wrona, Sharon, MA (US); Bertrand Le Bourdonnec, Northborough, MA (US); Matthew Lucas, Lexington, MA (US); Vanessa Kuria, Somerville, MA (US); Byron DeLaBarre, Arlington, MA (US)

(21) Appl. No.: 18/717,167

(22) PCT Filed: Dec. 8, 2022
(86) PCT No.: PCT/US2022/052206

§ 371 Date: Jun. 6, 2024

Related U.S. Application Data
(60) Provisional application No. 63/287,336, filed on Dec. 8, 2021.

(51) Int. Cl. A61K 31/5377; C07D 239/48; C07D 401/12; C07D 401/14; C07D 413/14; C07D 471/04; C07D 473/00; C07D 473/28; C07D 487/04; C07D 491/052; C07D 519/00

(52) U.S. Cl. CPC A61K 31/5377; C07D 239/48; C07D 401/12; C07D 401/14; C07D 413/14; C07D 471/04; C07D 473/00; C07D 473/28; C07D 487/04; C07D 491/052; C07D 519/00

(57) ABSTRACT

Disclosed are compounds useful in the treatment of neurological disorders. The compounds described herein, alone or in combination with other pharmaceutically active agents, can be used for treating or preventing neurological diseases.

### FIELD OF THE INVENTION

The invention relates to bicyclic heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention features a compound of Formula I:

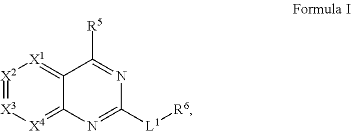

or a pharmaceutically acceptable salt thereof,
where
X¹ is N or CR¹;
X² is N or CR²;
X³ is N or CR³;
X⁴ is N or CR⁴;
where one and only one of X¹, X², X³, and X⁴ is N;
R⁵ is

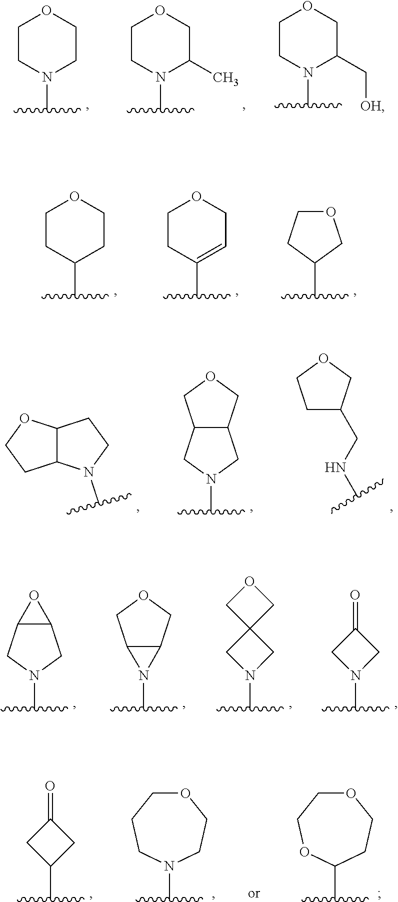

L¹ is

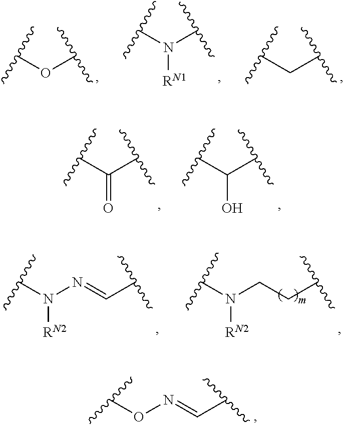

optionally substituted C₁₋₉ heteroarylene having at least one 5-membered ring, or optionally substituted C₂-C₉ heterocyclylene, and R⁶ is optionally substituted C₁-C₆ alkyl, optionally substituted C₆-C₁₀ aryl, optionally substituted C₃-C₁₀ carbocyclyl, optionally substituted C₂-C₉ heteroaryl, optionally substituted C₂-C₉ heterocyclyl, optionally substituted C₆-C₁₀ aryl C₁-C₆ alkyl, or optionally substituted C₂-C₉ heterocyclyl C₁-C₆ alkyl; or L¹ and R⁶ combine to form an optionally substituted C₂-C₉ oxyheteroaryl, optionally substituted pyrimidin-4-yl, optionally substituted indazol-1-yl, optionally substituted indazol-2-yl, optionally substituted indazol-3-yl, optionally substituted benzotriazol-1-yl, optionally substituted pyrazin-2-yl, or optionally substituted pyrid-2-yl, or a C₆-C₁₀ aryl optionally substituted with an optionally substituted C₂-C₉ heteroaryl;
R¹ is hydrogen, halogen, or optionally substituted C₁₋₆ alkyl;
R² is hydrogen or optionally substituted C₂-C₉ heterocyclyl;
R³ is hydrogen, halogen, optionally substituted C₁₋₆ alkyl, or

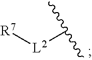

R⁴ is hydrogen, halogen, or optionally substituted C₁₋₆ alkyl;
L² is absent,

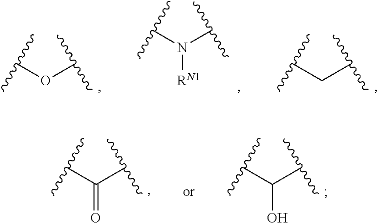

R⁷ is optionally substituted C₆₋₁₀ aryl, optionally substituted C₁-C₆ alkyl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3.

In some embodiments, R⁵ is

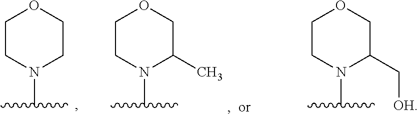

In some embodiments, R⁵ is

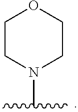 [SMILES: *N1CCOCC1]

In some embodiments, the compound has the structure of Formula Ia:

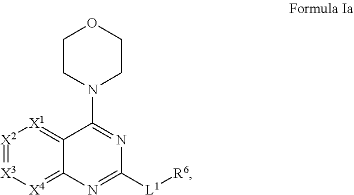

or a pharmaceutically acceptable salt thereof.

In some embodiments, X¹ is N. In some embodiments, X² is N. In some embodiments, X³ is N.

In some embodiments, X⁴ is N.

In some embodiments, the compound has the structure of Formula II:

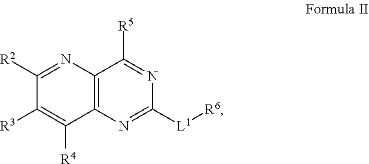

or a pharmaceutically acceptable salt thereof.

In some embodiments, R² is optionally substituted C₂-C₉ heterocyclyl. In some embodiments, R² is

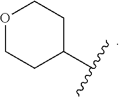 [SMILES: *C1CCOCC1]

In some embodiments, R² is halogen or optionally substituted C₁₋₆ alkyl.

In some embodiments, the compound has the structure of Formula IIa:

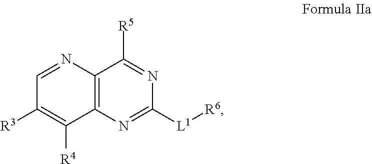

or a pharmaceutically acceptable salt thereof.

In some embodiments, R⁴ is halogen or optionally substituted C₁-C₆ alkyl.

In some embodiments, the compound has the structure of Formula IIb:

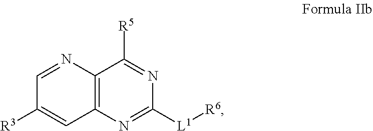

or a pharmaceutically acceptable salt thereof.

In some embodiments, the compound has the structure:

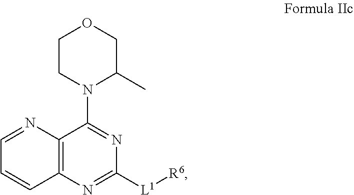

or a pharmaceutically acceptable salt thereof.

In some embodiments, the compound has the structure of Formula IId:

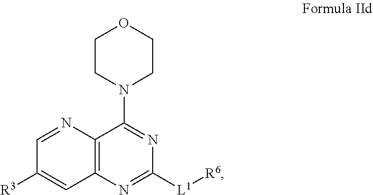

or a pharmaceutically acceptable salt thereof.

In some embodiments, R³ is hydrogen. In some embodiments, R³ is halogen, optionally substituted C₁-C₆ heteroalkyl, or optionally substituted C₁-C₆ alkyl.

In some embodiments, R³ is Br.

In some embodiments, R³ is

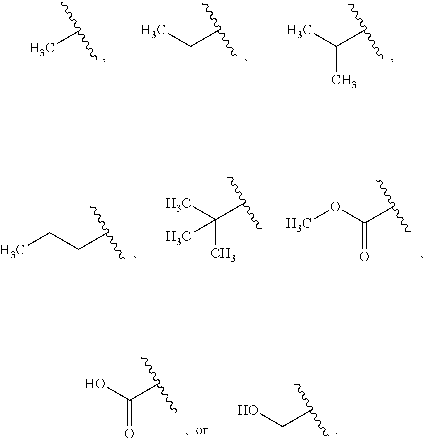

In some embodiments, R³ is

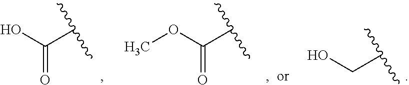

In some embodiments, R³ is

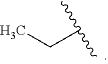 [SMILES: *CC]

In some embodiments, R³ is

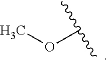 [SMILES: *OC]

In some embodiments, R³ is

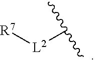

In some embodiments, L² is absent.

In some embodiments, L² is

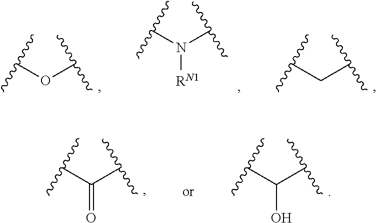

In some embodiments, R^{N1} is hydrogen or

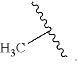

In some embodiments, R^{N1} is hydrogen.

In some embodiments, L² is

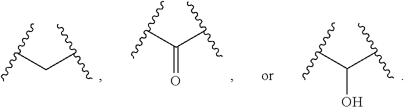

In some embodiments, R⁷ is optionally substituted C₆-C₁₀ aryl.

In some embodiments, R⁷ is optionally substituted C₃-C₁₀ carbocyclyl, optionally substituted C₂-C₉ heteroaryl, or optionally substituted C₂-C₉ heterocyclyl.

In some embodiments, R⁷ is optionally substituted C₃-C₁₀ carbocyclyl.

In some embodiments, R⁷ is

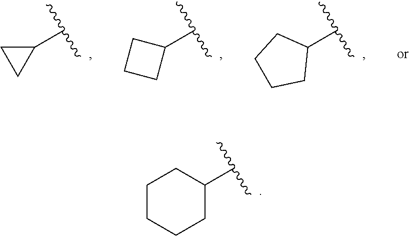

In some embodiments, R⁷ is

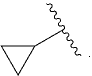 [SMILES: *C1CC1]

In some embodiments, R⁷ is optionally substituted C₁₋₉ heteroaryl or optionally substituted C₁₋₉ heterocyclyl.

In some embodiments, R⁷ is optionally substituted C₁₋₉ heteroaryl.

In some embodiments, R⁷ is

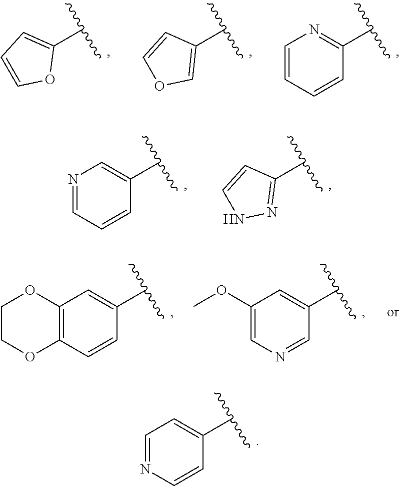

In some embodiments, R⁷ is

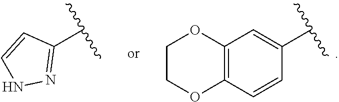 [SMILES: *c1cc[nH]n1 ; *c1ccc2c(c1)OCCO2]

In some embodiments, R⁷ is

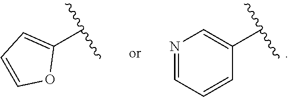 [SMILES: *c1ccco1 ; *c1cccnc1]

In some embodiments, R⁷ is

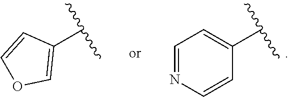 [SMILES: *c1ccoc1 ; *c1ccncc1]

In some embodiments, R⁷ is or optionally substituted C₁₋₉ heterocyclyl.

In some embodiments, R⁷ is

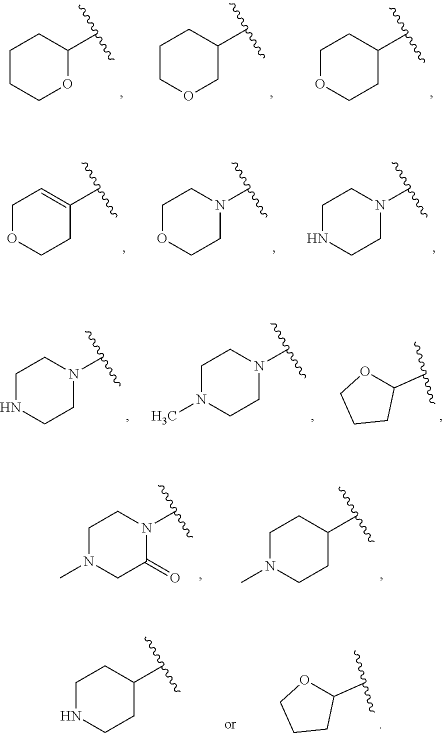

In some embodiments, R⁷ is

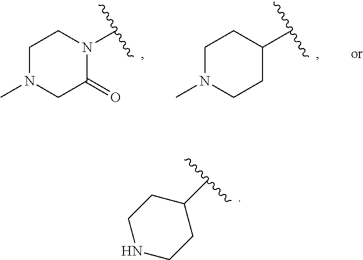

In some embodiments, R⁷ is

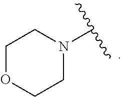 [SMILES: *N1CCOCC1]

In some embodiments, R⁷ is

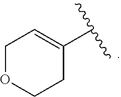 [SMILES: *C1=CCOCC1]

In some embodiments, R⁷ is

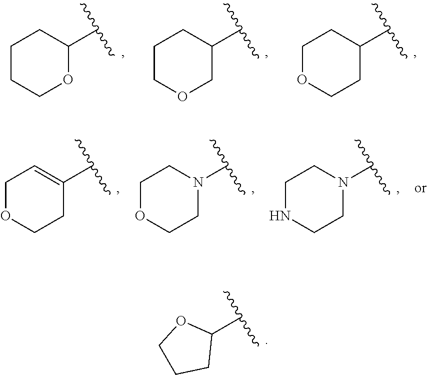

In some embodiments, R⁷ is

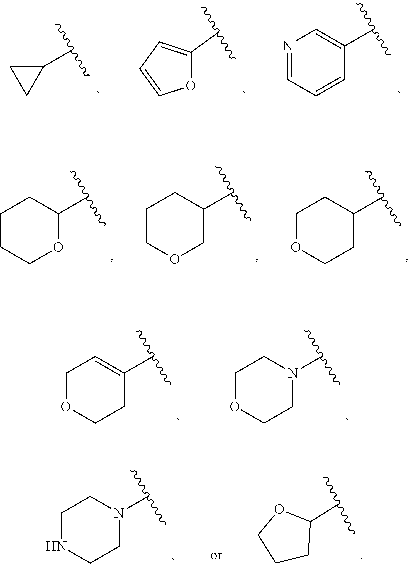

In some embodiments, R⁷ is optionally substituted C₁-C₆ alkyl. In some embodiments, R⁷ is

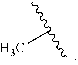

In some embodiments, the compound has the structure of Formula IIe:

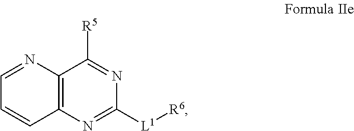

or a pharmaceutically acceptable salt thereof.

In some embodiments, the compound has the structure of Formula IIf:

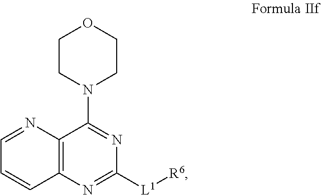

or a pharmaceutically acceptable salt thereof.

In some embodiments, the compound has the structure of Formula IIg:

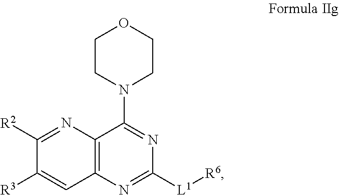

or a pharmaceutically acceptable salt thereof.

In some embodiments, the compound has the structure of Formula IIh:

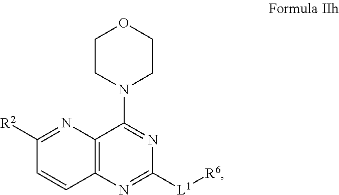

or a pharmaceutically acceptable salt thereof.

In some embodiments, R² is optionally substituted C₁₋₉ heterocyclyl.

In some embodiments, R² is

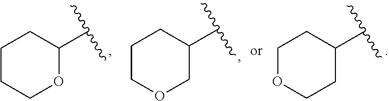

In some embodiments, R² is

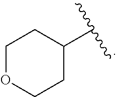 [SMILES: *C1CCOCC1]

In some embodiments, the compound has the structure of Formula III:

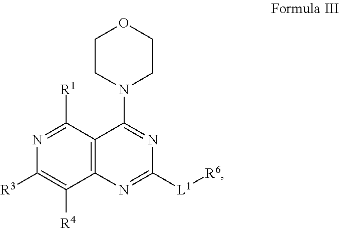

or a pharmaceutically acceptable salt thereof.

In some embodiments, the compound has the structure of Formula IIIa:

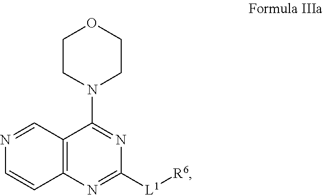

or a pharmaceutically acceptable salt thereof.

In some embodiments, the compound has the structure of Formula IV:

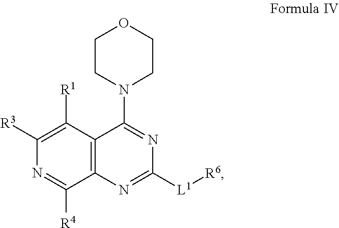

or a pharmaceutically acceptable salt thereof.

In some embodiments, the compound has the structure of Formula IVa:

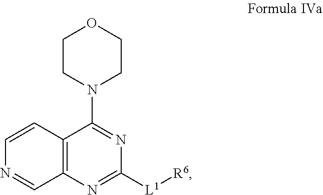

or a pharmaceutically acceptable salt thereof.

In some embodiments, the compound has the structure of Formula V:

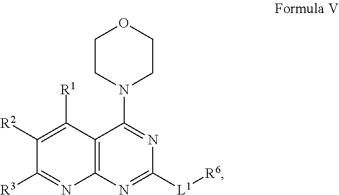

or a pharmaceutically acceptable salt thereof.

In some embodiments, the compound has the structure of Formula Va:

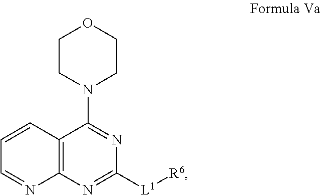

or a pharmaceutically acceptable salt thereof.

In some embodiments, L¹ is

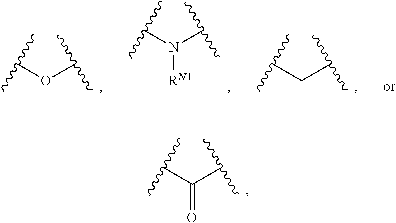

In some embodiments, L¹ is

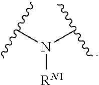

In some embodiments, R^{N1} is hydrogen or

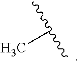

In some embodiments, R^{N1} is hydrogen.

In some embodiments, L¹ is

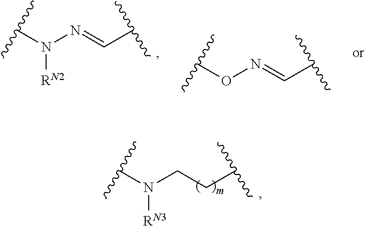

In some embodiments, L¹ is

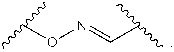 [SMILES: */C=N/O*]

In some embodiments, L¹ is

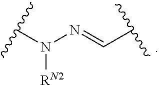

In some embodiments, R^{N2} is hydrogen or

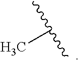

In some embodiments, R^{N2} is hydrogen.

In some embodiments, L¹ is

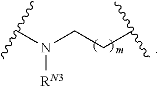

In some embodiments, m is 1.

In some embodiments, R^{N3} is hydrogen or

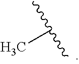

In some embodiments, R^{N3} is hydrogen.

In some embodiments, L¹ is optionally substituted C₁₋₉ heteroarylene having at least one 5-membered ring or non-aromatic optionally substituted C₁₋₉ heterocyclylene.

In some embodiments, L¹ is optionally substituted C₁₋₉ heteroarylene having at least one 5-membered ring.

In some embodiments, L¹ is optionally substituted pyrazole-diyl.

In some embodiments, L¹ is optionally substituted monocyclic 5-membered C₁₋₉ heteroarylene.

In some embodiments, L¹ is optionally substituted pyrazole-diyl or optionally substituted triazole-diyl.

In some embodiments, L¹ is

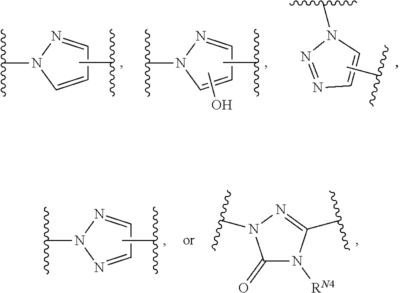

where R^{N4} is hydrogen or optionally substituted C₁₋₆ alkyl.

In some embodiments, L¹ is

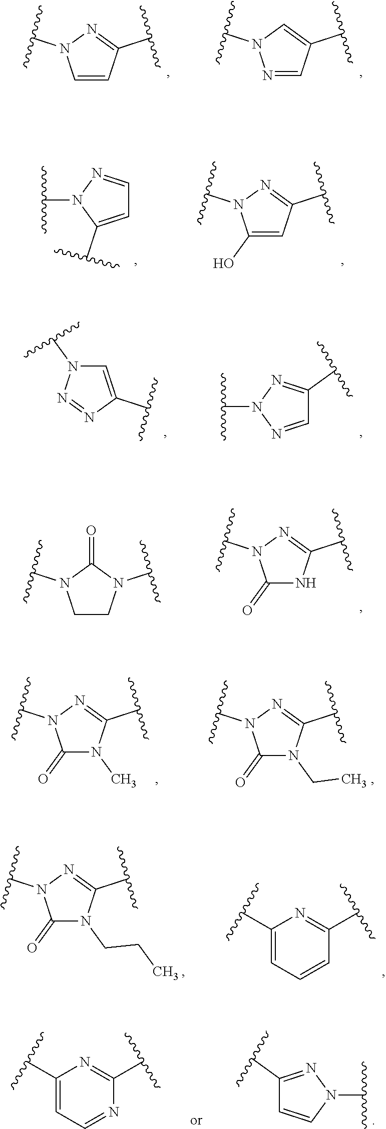

In some embodiments, L¹ is

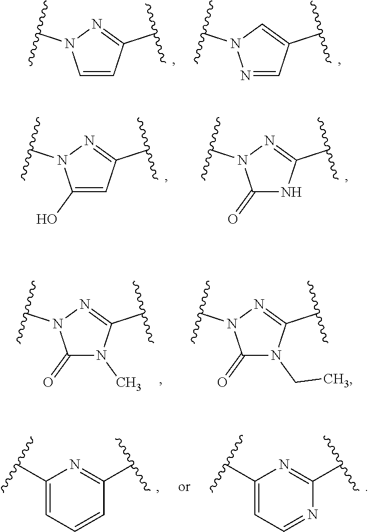

In some embodiments, L¹ is

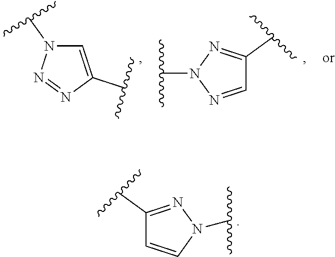

In some embodiments, L¹ is optionally substituted non-aromatic C₁₋₉ heterocyclylene.

In some embodiments, L¹ is optionally substituted non-aromatic C₁₋₅ heterocyclylene.

In some embodiments, L¹ is optionally substituted non-aromatic C₁₋₄ heterocyclylene.

In some embodiments, L¹ is optionally substituted 5-membered non-aromatic C₁₋₅ heterocyclylene.

In some embodiments, L¹ is

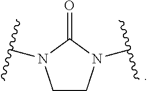 [SMILES: *N1CCN(*)C1=O]

In some embodiments, R⁶ is optionally substituted C₁₋₆ alkyl.

In some embodiments, R⁶ is

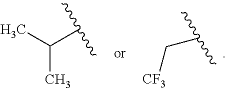

In some embodiments, R⁶ is optionally substituted C₆₋₁₀ aryl, optionally substituted C₃₋₁₀ carbocyclyl, optionally substituted C₁₋₉ heteroaryl, optionally substituted C₁₋₉ heterocyclyl, optionally substituted C₆-C₁₀ aryl C₁-C₆ alkyl, or optionally substituted C₂-C₉ heterocyclyl C₁-C₆ alkyl.

In some embodiments, R⁶ is optionally substituted C₆₋₁₀ aryl.

In some embodiments, R⁶ is

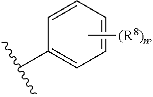

where n is 0, 1, 2, 3, 4, or 5; and each R⁸ is, independently, independently, halogen, optionally substituted C₁₋₆ heteroalkyl, optionally substituted C₂-C₉ heteroaryl, hydroxyl, or optionally substituted C₁₋₆ alkyl.

In some embodiments, each R⁸ is, independently, F, hydroxyl, pyrazol-4-yl,

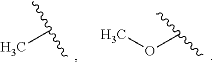

In some embodiments, each R⁸ is hydroxyl, pyrazol-4-yl, or

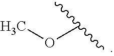 [SMILES: *OC]

In some embodiments, n is 0 or 1.

In some embodiments, R⁶ is

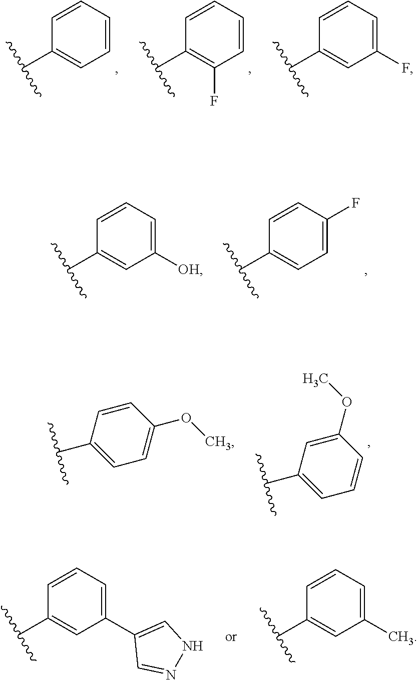

In some embodiments, R⁶ is

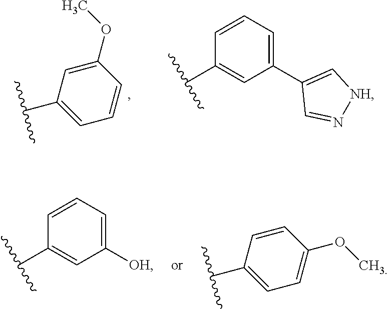

In some embodiments, R⁶ is optionally substituted C₃₋₁₀ carbocyclyl.

In some embodiments, R⁶ is

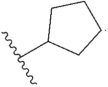 [SMILES: *C1CCCC1]

In some embodiments, R⁶ is optionally substituted C₁₋₉ heteroaryl.

In some embodiments, R⁶ is optionally substituted monocyclic C₁₋₉ heteroaryl.

In some embodiments, R⁶ is

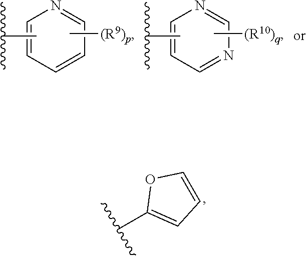

or where p is 0, 1, 2, or 3; q is 0, 1, or 2; each R⁹ is, independently, halogen or optionally substituted C₁₋₆ alkyl; and each R¹⁰ is, independently, halogen or optionally substituted C₁₋₆ alkyl.

In some embodiments, p is 0.

In some embodiments, q is 0.

In some embodiments, R⁶ is

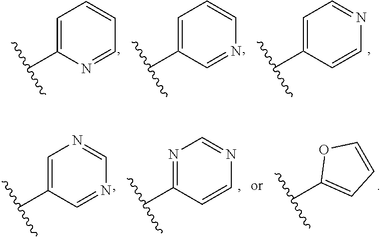

In some embodiments, R⁶ is optionally substituted polycyclic C₁₋₉ heteroaryl.

In some embodiments, R⁶ is

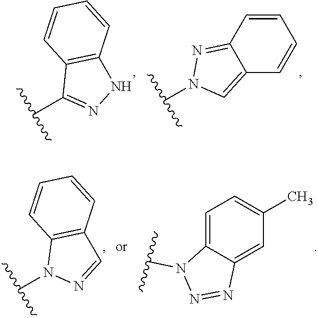 [SMILES: *c1n[nH]c2ccccc12 ; *n1cc2ccccc2n1 ; *n1ncc2ccccc21 ; *n1nnc2cc(C)ccc21]

In some embodiments, R⁶ is

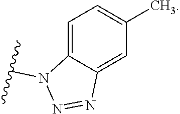 [SMILES: *n1nnc2cc(C)ccc21]

In some embodiments, R⁶ is optionally substituted C₁₋₉ heterocyclyl.

In some embodiments, R⁶ is optionally substituted monocyclic C₁₋₉ heterocyclyl.

In some embodiments, R⁶ is

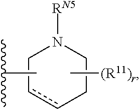

where [symbol]
represents a single bond or a double bond; r is 0, 1, 2, 3, 4, 5, or 6; each R¹¹ is, independently, halogen or optionally substituted C₁₋₆ alkyl; and R^{N5} is hydrogen, optionally substituted C₁₋₆ alkyl, or optionally substituted C₁₋₆ heteroalkyl.

In some embodiments, r is 0, 1, or 2.

In some embodiments, R^{N5} is

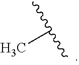

In some embodiments, R⁶ is

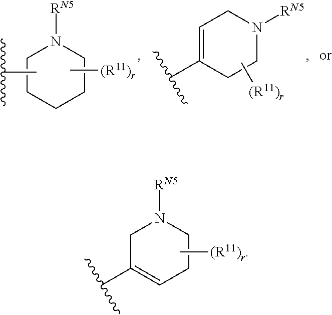

In some embodiments, R⁶ is

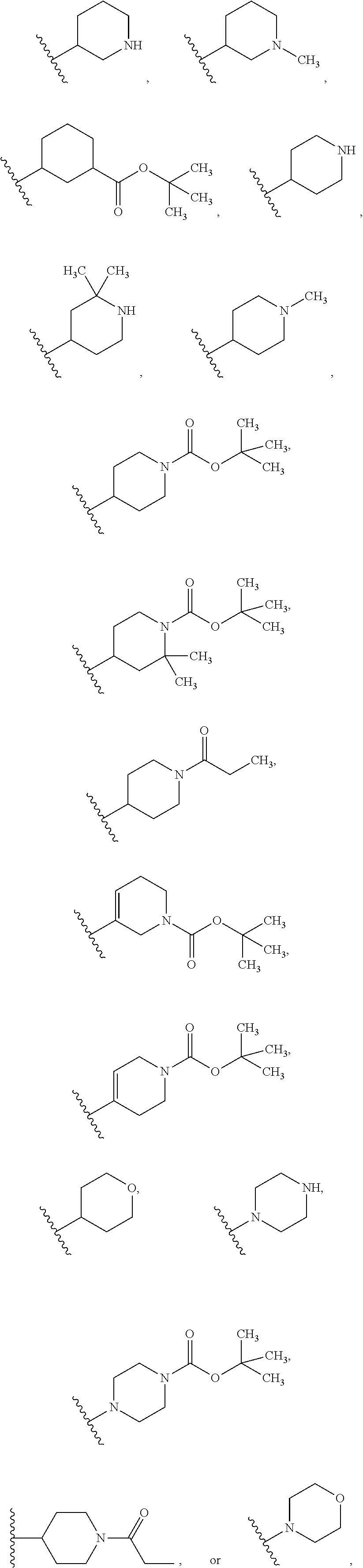

In some embodiments, R⁶ is optionally substituted C₂-C₉ heterocyclyl C₁-C₆ alkyl.

In some embodiments, R⁶ is

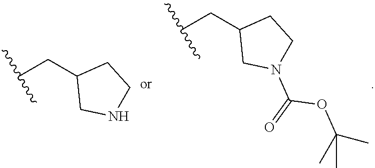 [SMILES: *CC1CCNC1 ; *CC1CCN(C(=O)OC(C)(C)C)C1]

In some embodiments, R⁶ is optionally substituted C₆-C₁₀ aryl C₁-C₆ alkyl.

In some embodiments, R⁶ is

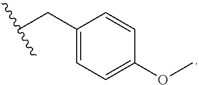 [SMILES: *Cc1ccc(OC)cc1]

In some embodiments, -L¹-R⁶ is

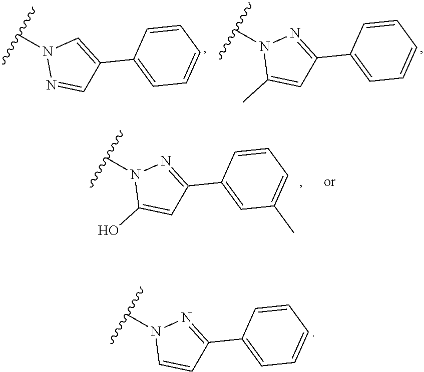

In some embodiments, L¹-R⁶ is

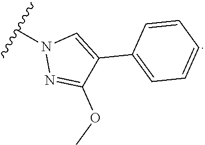 [SMILES: *n1cc(-c2ccccc2)c(OC)n1]

In some embodiments, L¹ and R⁶ combine to form a C₆-C₁₀ aryl optionally substituted with an optionally substituted C₂-C₉ heteroaryl. In some embodiments, L¹ and R⁶ combine to form a C₆-C₁₀ aryl optionally substituted with an optionally substituted pyrazol-3-yl. In some embodiments, L¹ and R⁶ combine to form

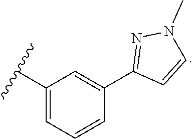 [SMILES: *c1cccc(-c2ccn(C)n2)c1]

In some embodiments, L¹ and R⁶ combine to form optionally substituted pyrimidin-4-yl, optionally substituted pyrid-2-yl, optionally substituted indazol-1-yl, optionally substituted inazol-2-yl, optionally substituted indazol-3-yl, optionally substituted benzotriazole-1-yl, or optionally substituted pyrazin-2-yl

In some embodiments, the optionally substituted pyrimidin-4-yl is a pyrimidin-4-yl substituted at position 2.

In some embodiments, L¹ and R⁶ combine to form optionally substituted pyrid-2-yl. In some embodiments, the optionally substituted pyrid-2-yl is a pyrid-2-yl substituted at position 5.

In some embodiments, L¹ and R⁶ combine to form

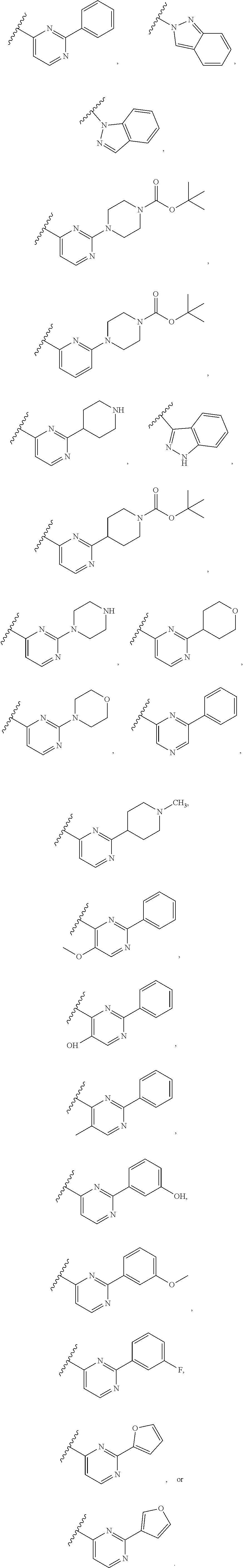

In some embodiments, L¹ and R⁶ combine to form

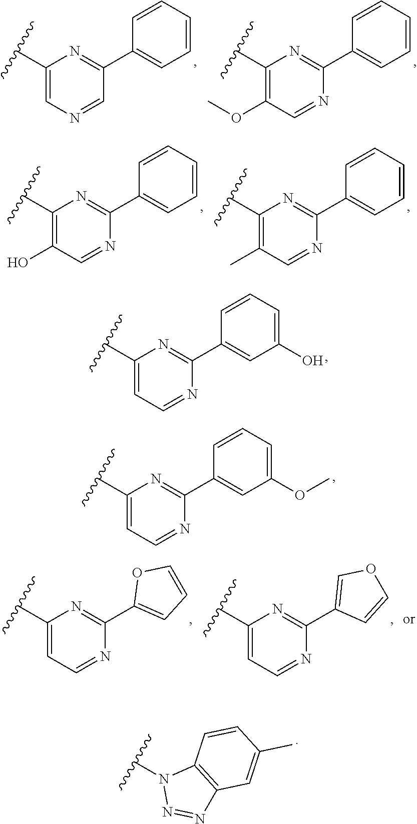

In some embodiments, L¹ and R⁶ combine to form

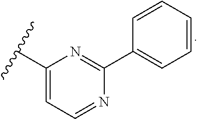 [SMILES: *c1ccnc(-c2ccccc2)n1]

In some embodiments, L¹ is pyrazol-1-yl substituted with phenyl and R³ is optionally substituted piperidin-4-yl. In some embodiments, the compound has the structure:

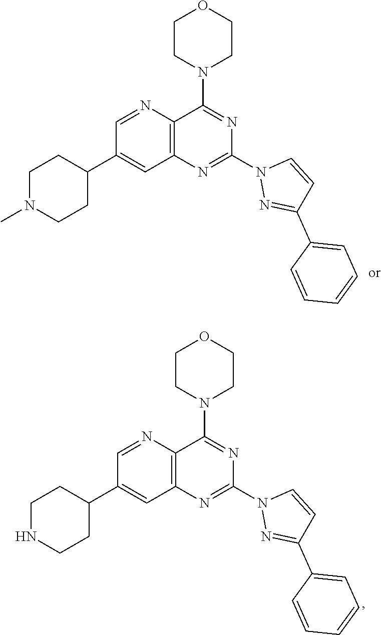 [SMILES: CN1CCC(c2cnc3c(N4CCOCC4)nc(-n4ccc(-c5ccccc5)n4)nc3c2)CC1 ; c1ccc(-c2ccn(-c3nc(N4CCOCC4)c4ncc(C5CCNCC5)cc4n3)n2)cc1]

or a pharmaceutically acceptable salt thereof.

In some embodiments, L¹ is pyrazol-1-yl optionally substituted at position 4 with phenyl, and R³ is optionally substituted morpholin-4-yl. In some embodiments, the compound has the structure:

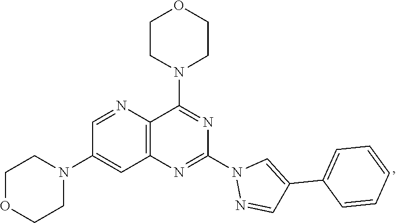 [SMILES: c1ccc(-c2cnn(-c3nc(N4CCOCC4)c4ncc(N5CCOCC5)cc4n3)c2)cc1]

or a pharmaceutically acceptable salt thereof.

In some embodiments, L1 is pyrazol-1-yl substituted at position 4 with optionally substituted phenyl, and R³ is hydrogen or C₁-C₆ alkyl substituted with hydroxyl. In some embodiments, the compound has the structure:

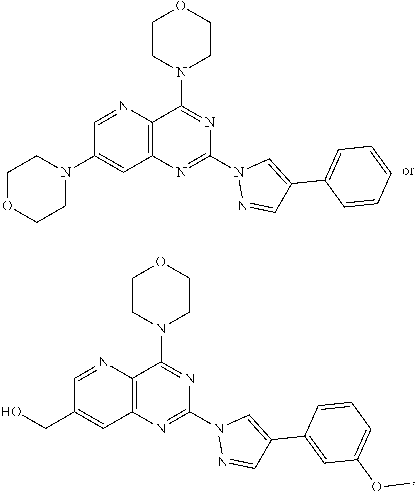 [SMILES: c1ccc(-c2cnn(-c3nc(N4CCOCC4)c4ncc(N5CCOCC5)cc4n3)c2)cc1 ; COc1cccc(-c2cnn(-c3nc(N4CCOCC4)c4ncc(CO)cc4n3)c2)c1]

or a pharmaceutically acceptable salt thereof.

In some embodiments, L¹-R⁶ is

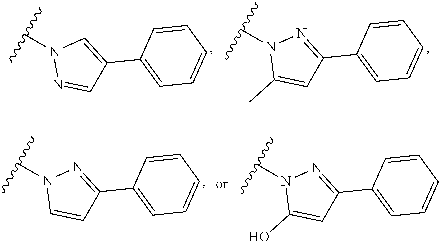

R⁷ is hydrogen,

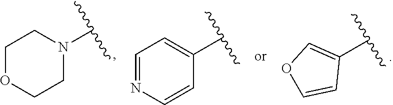 [SMILES: *N1CCOCC1 ; *c1ccncc1 ; *c1ccoc1]

In some embodiments, L¹-R⁶ is

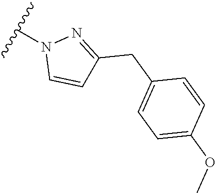 [SMILES: *n1ccc(Cc2ccc(OC)cc2)n1]

### and R³ is

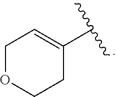 [SMILES: *C1=CCOCC1]

In some embodiments, L¹ and R⁶ combine to form

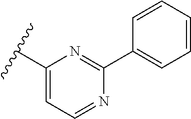 [SMILES: *c1ccnc(-c2ccccc2)n1]

and R⁷ is pyrid-4-yl or morpholin-4-yl.

In some embodiments, the compound is any one of compounds 16, 17, 27-31, 34-37, 39, 40, 43-49, 54, 56, 65, 67, 68, 70, 74-76, 93-108, 114, 116, 117, or 135-137 in Table 1, or a pharmaceutically acceptable salt thereof. In some embodiments, the compound is any one of compounds 1-15, 18-26, 32, 33, 38, 41, 42, 50-53, 55, 57-64, 66, 69, 71-73, 77-82, 109-113, 115, or 122-132 in Table 1, or a pharmaceutically acceptable salt thereof.

In an aspect, the disclosure features compounds 1-137 in Table 1 and pharmaceutically acceptable salts thereof.

In an aspect, the invention features a pharmaceutical composition including any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compound described herein or a pharmaceutical composition containing one or more compounds described herein.

In some embodiments, the method includes administering to the subject in need thereof an effective amount of the compound of Formula VI:

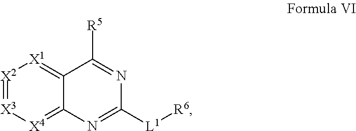

or a pharmaceutically acceptable salt thereof,
where
X¹ is N or CR¹;
X² is N or CR²;
X³ is N or CR³;
X⁴ is N or CR⁴;
R⁵ is

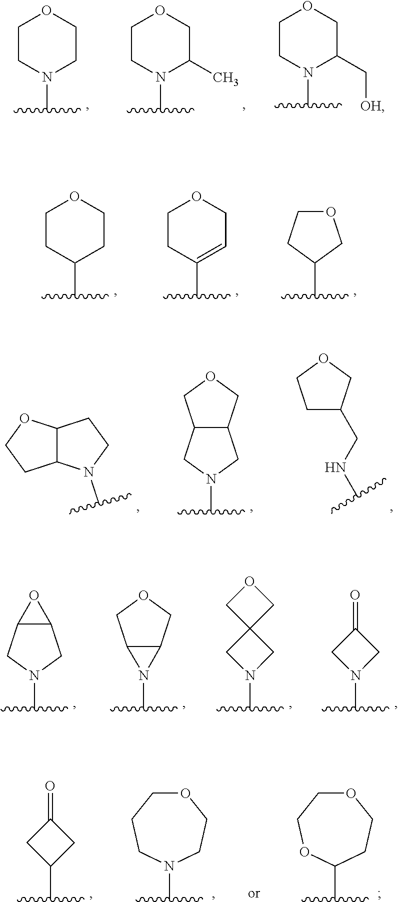

L¹ is absent,

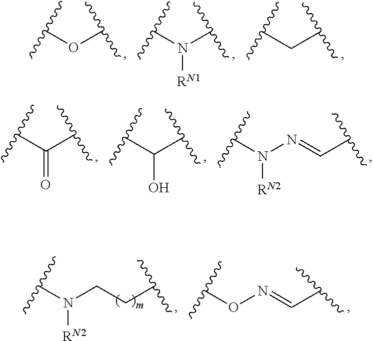

optionally substituted C₁₋₉ heteroarylene, or optionally substituted C₁₋₉ heterocyclylene;
R⁶ is halogen, optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl,
R¹ is hydrogen, halogen, optionally substituted C₁₋₆ alkyl, or

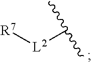

R² is hydrogen, halogen, optionally substituted C₁₋₆ alkyl, or

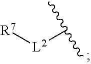

R³ is hydrogen, halogen, optionally substituted C₁₋₆ alkyl, or

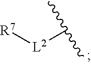

R⁴ is hydrogen, halogen, optionally substituted C₁₋₆ alkyl, or

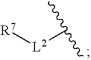

L² is absent,

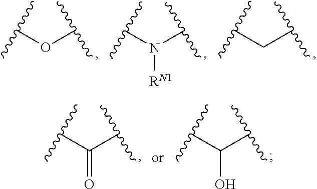

R⁷ is optionally substituted C₆₋₁₀ aryl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3;
where one and only one of X¹, X², X³, and X⁴ is N.

In some embodiments, the compound has the structure of any one of compounds 1-134 in Table 1.

In another aspect, the invention features a method of inhibiting PIKfyve in a cell expressing PIKfyve protein, the method including contacting the cell with any of the foregoing compounds, or a pharmaceutically acceptable salt thereof.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361 S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

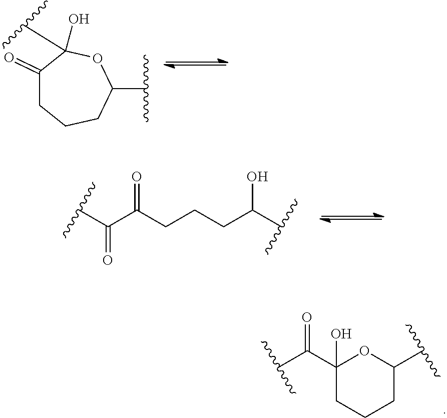 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, where X is optionally substituted” (e.g., “alkyl, where said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, where each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), where each of these recited R^{N1} groups can be optionally substituted; or two R^{N1} combine to form an alkylene or heteroalkylene, and where each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₆₋₁₀ aryl C₁-C₆ alkyl, C₆₋₁₀ aryl C₁-C₁₀ alkyl, or C₆₋₁₀ aryl C₁-C₂₀ alkyl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The terms “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated, non-aromatic carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring and containing one, two, three, or four ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₂-C₉ heteroaryl C₁-C₆ alkyl, C₂-C₉ heteroaryl C₁-C₁₀ alkyl, or C₂-C₉ heteroaryl C₁-C₂₀ alkyl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, where no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₂-C₉ heterocyclyl C₁-C₆ alkyl, C₂-C₉ heterocyclyl C₁-C₁₀ alkyl, or C₂-C₉ heterocyclyl C₁-C₂₀ alkyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, orthiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, where such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “including” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361 S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361 S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

Exemplary PIKfyve inhibitors described herein include a compound of Formula I:

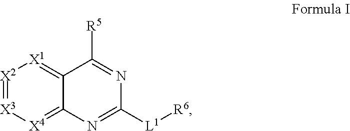

or a pharmaceutically acceptable salt thereof,
where
X¹ is N or CR¹;
X² is N or CR²;
X³ is N or CR³;
X⁴ is N or CR⁴;
R⁵ is

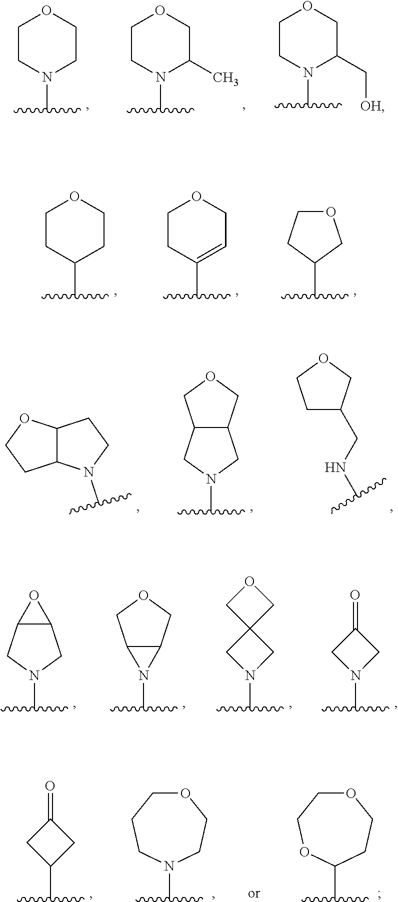

L¹ is

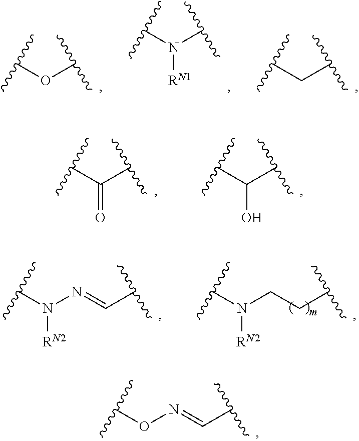

optionally substituted C₁₋₉ heteroarylene having at least one 5-membered ring, or optionally substituted C₂-C₉ heterocyclylene and R⁶ is optionally substituted C₁-C₆ alkyl, optionally substituted C₆-C₁₀ aryl, optionally substituted C₃-C₁₀ carbocyclyl, optionally substituted C₂-C₉ heteroaryl, optionally substituted C₂-C₉ heterocyclyl, optionally substituted C₆-C₁₀ aryl C₁-C₆ alkyl, or optionally substituted C₂-C₉ heterocyclyl C₁-C₆ alkyl; or L¹ and R⁶ combine to form an optionally substituted C₂-C₉ oxyheteroaryl, optionally substituted pyrimidin-4-yl, optionally substituted indazol-1-yl, optionally substituted indazol-2-yl, optionally substituted indazol-3-yl, optionally substituted benzotriazol-1-yl, optionally substituted pyrazin-2-yl, optionally substituted pyrid-2-yl, or a C₆-C₁₀ aryl optionally substituted with an optionally substituted C₂-C₉ heteroaryl;
R¹ is hydrogen, halogen, or optionally substituted C₁₋₆ alkyl;
R² is hydrogen or optionally substituted C₂-C₉ heterocyclyl;
R³ is hydrogen, halogen, optionally substituted C₁-C₆ alkyl, optionally substituted C₁-C₆ heteroalkyl, or

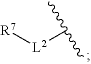

R⁴ is hydrogen, halogen, or optionally substituted C₁₋₆ alkyl;
L² is absent,

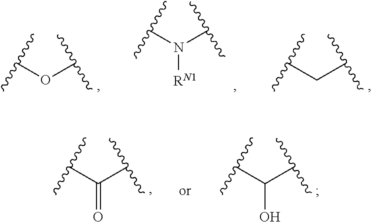

R⁷ is optionally substituted C₆₋₁₀ aryl, optionally substituted C₁-C₆ alkyl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3;
where one and only one of X¹, X², X³, and X⁴ is N.

In some embodiments, L¹ is

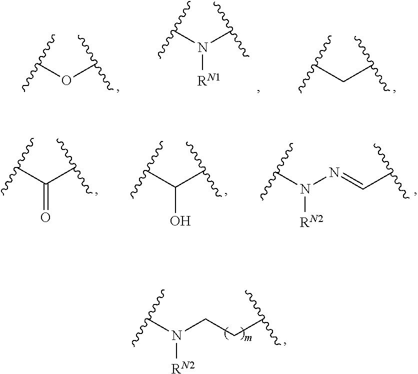

optionally substituted C₁₋₉ heteroarylene having at least one 5-membered ring, or optionally substituted non-aromatic C₁₋₉ heterocyclylene; and R⁶ is optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl; or L¹ and R⁶ combine to form an optionally substituted C₂₋₉ oxyheteroaryl, optionally substituted pyrimidin-4-yl, or optionally substituted pyrid-2-yl.

PIKfyve inhibitors described herein include a compound of formula Ia:

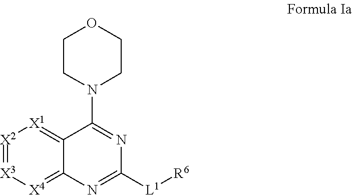

or a pharmaceutically acceptable salt thereof, where all variables are as described herein.

PIKfyve inhibitors described herein include a compound of formula II:

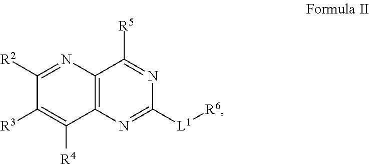

or a pharmaceutically acceptable salt thereof, where all variables are as described herein.

PIKfyve inhibitors described herein include a compound of formula IIa:

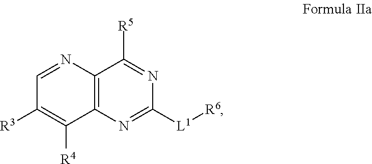

or a pharmaceutically acceptable salt thereof, where all variables are as described herein.

PIKfyve inhibitors described herein include a compound of formula IIb:

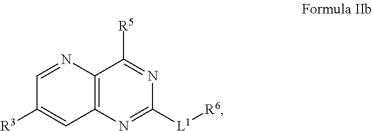

or a pharmaceutically acceptable salt thereof, where all variables are as described herein.

PIKfyve inhibitors described herein include a compound of formula IIc:

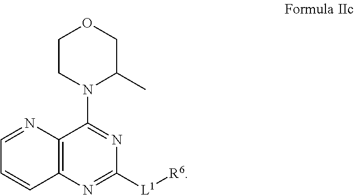

or a pharmaceutically acceptable salt thereof, where all variables are as described herein.

PIKfyve inhibitors described herein include a compound of formula IId:

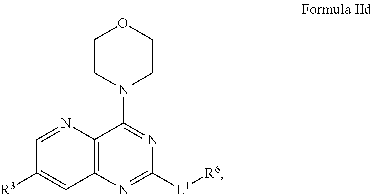

or a pharmaceutically acceptable salt thereof, where all variables are as described herein.

PIKfyve inhibitors described herein include a compound of formula IIe:

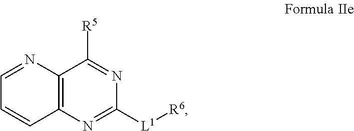

or a pharmaceutically acceptable salt thereof, where all variables are as described herein. PIKfyve inhibitors described herein include a compound of formula IIf:

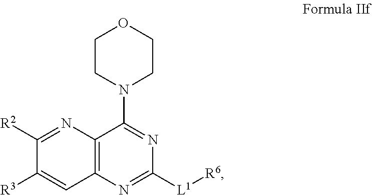

or a pharmaceutically acceptable salt thereof, where all variables are as described herein.

PIKfyve inhibitors described herein include a compound of formula IIg:

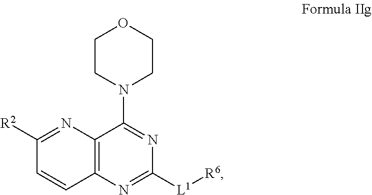

or a pharmaceutically acceptable salt thereof, where all variables are as described herein.

PIKfyve inhibitors described herein include a compound of formula III:

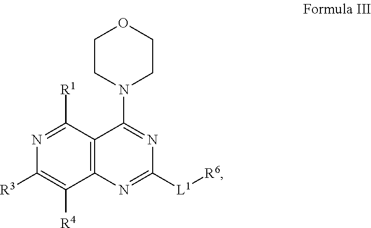

or a pharmaceutically acceptable salt thereof, where all variables are as described herein.

PIKfyve inhibitors described herein include a compound of formula IIIa:

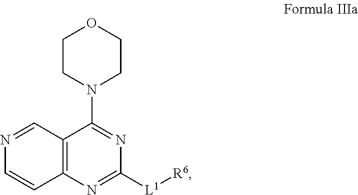

or a pharmaceutically acceptable salt thereof, where all variables are as described herein.

PIKfyve inhibitors described herein include a compound of formula IV:

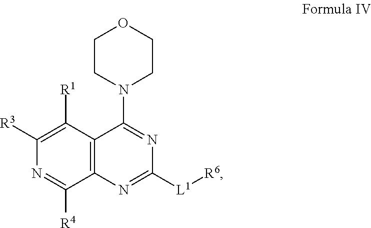

or a pharmaceutically acceptable salt thereof, where all variables are as described herein.

PIKfyve inhibitors described herein include a compound of formula IVa:

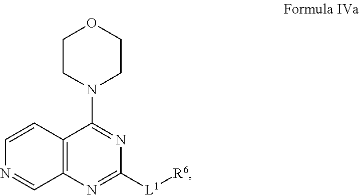

or a pharmaceutically acceptable salt thereof, where all variables are as described herein.

PIKfyve inhibitors described herein include a compound of formula V:

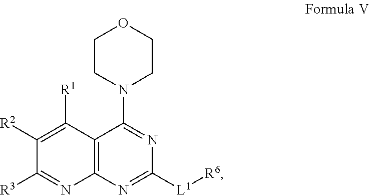

or a pharmaceutically acceptable salt thereof, where all variables are as described herein.

PIKfyve inhibitors described herein include a compound of formula Va:

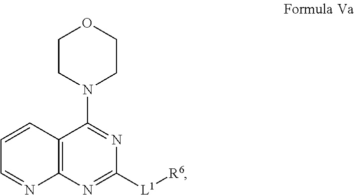

or a pharmaceutically acceptable salt thereof, where all variables are as described herein.

PIKfyve inhibitors described herein include a compound of formula VI:

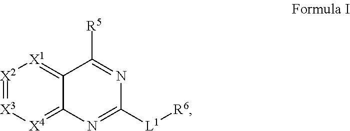

or a pharmaceutically acceptable salt thereof,
where
X¹ is N or CR¹;
X² is N or CR²;
X³ is N or CR³;
X⁴ is N or CR⁴;
R⁵ is

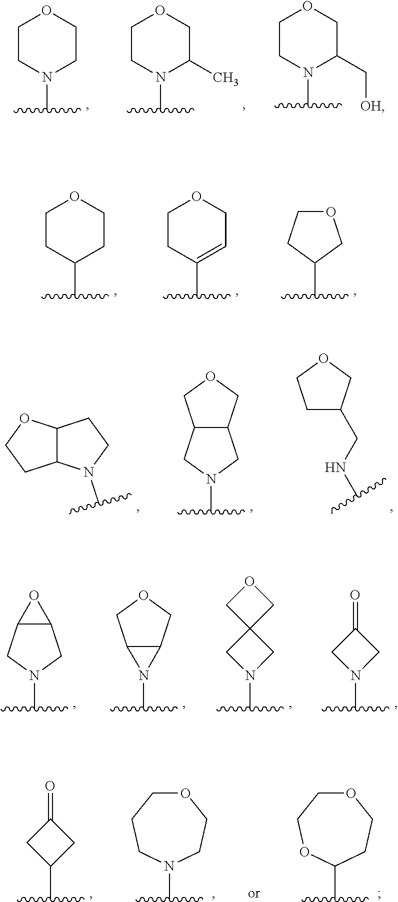

L¹ is absent,

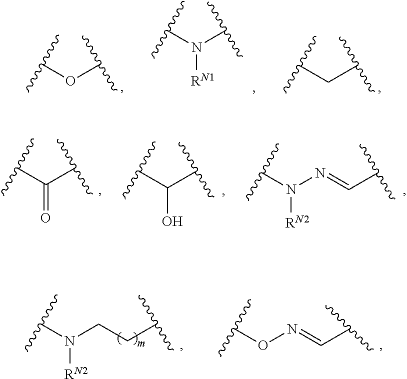

optionally substituted C₁₋₉ heteroarylene, or optionally substituted C₁₋₉ heterocyclylene;
R⁶ is halogen, optionally substituted C₁₋₆ alkyl, optionally substituted C₆₋₁₀ aryl, optionally substituted C₃₋₁₀ carbocyclyl, optionally substituted C₁₋₉ heteroaryl, optionally substituted C₁₋₉ heterocyclyl, or optionally substituted —C₁₋₆ alkylene-C₁₋₉ heterocyclyl,
R¹ is hydrogen, halogen, optionally substituted C₁₋₆ alkyl, or

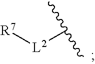

R² is hydrogen, halogen, optionally substituted C₁₋₆ alkyl, or

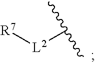

R³ is hydrogen, halogen, optionally substituted C₁₋₆ alkyl, or

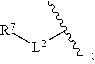

R⁴ is hydrogen, halogen, optionally substituted C₁₋₆ alkyl, or

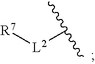

L² is absent,

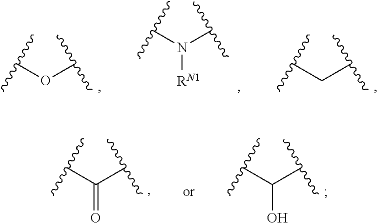

R⁷ is optionally substituted C₆₋₁₀ aryl, optionally substituted C₃₋₁₀ carbocyclyl, optionally substituted C₁₋₉ heteroaryl, or optionally substituted C₁₋₉ heterocyclyl;
each of R^{N1}, R^{N2} and R^{N3} is, independently, hydrogen or optionally substituted C₁₋₆ alkyl; and
m is 0, 1, 2, or 3;
where one and only one of X¹, X², X³, and X⁴ is N.

Exemplary PIKfyve inhibitors described herein include the compounds in Table 1.

TABLE 1
Compounds of the Invention

| # | Structure |
|---|---|
| 1 | 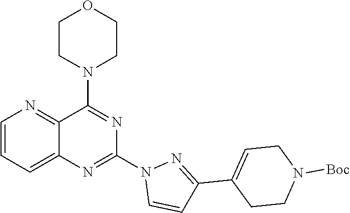 [SMILES: CC(C)(C)OC(=O)N1CC=C(c2ccn(-c3nc(N4CCOCC4)c4ncccc4n3)n2)CC1] |
| 2 | 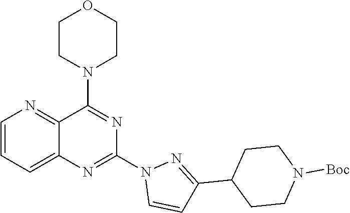 [SMILES: CC(C)(C)OC(=O)N1CCC(c2ccn(-c3nc(N4CCOCC4)c4ncccc4n3)n2)CC1] |
| 3 | 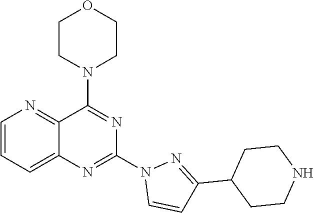 [SMILES: c1cnc2c(N3CCOCC3)nc(-n3ccc(C4CCNCC4)n3)nc2c1] |
| 4 | 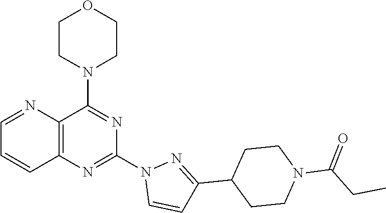 [SMILES: CCC(=O)N1CCC(c2ccn(-c3nc(N4CCOCC4)c4ncccc4n3)n2)CC1] |
| 5 | 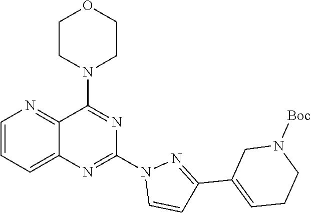 [SMILES: CC(C)(C)OC(=O)N1CCC=C(c2ccn(-c3nc(N4CCOCC4)c4ncccc4n3)n2)C1] |
| 6 | 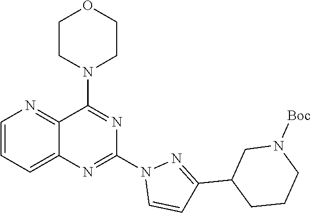 [SMILES: CC(C)(C)OC(=O)N1CCCC(c2ccn(-c3nc(N4CCOCC4)c4ncccc4n3)n2)C1] |
| 7 | 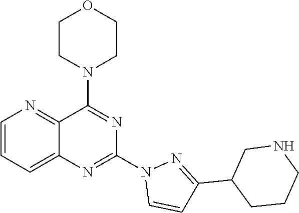 [SMILES: c1cnc2c(N3CCOCC3)nc(-n3ccc(C4CCCNC4)n3)nc2c1] |
| 8 | 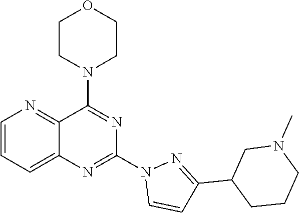 [SMILES: CN1CCCC(c2ccn(-c3nc(N4CCOCC4)c4ncccc4n3)n2)C1] |
| 9 | 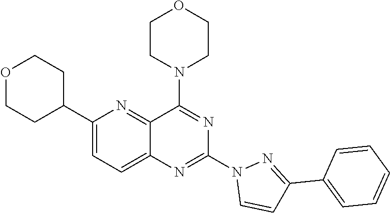 [SMILES: c1ccc(-c2ccn(-c3nc(N4CCOCC4)c4nc(C5CCOCC5)ccc4n3)n2)cc1] |
| 10 | 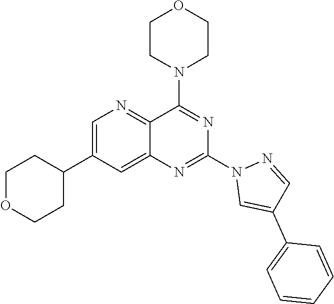 [SMILES: c1ccc(-c2cnn(-c3nc(N4CCOCC4)c4ncc(C5CCOCC5)cc4n3)c2)cc1] |
| 11 | 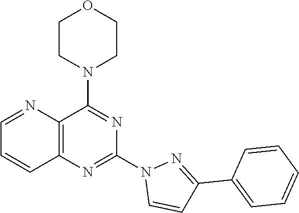 [SMILES: c1ccc(-c2ccn(-c3nc(N4CCOCC4)c4ncccc4n3)n2)cc1] |
| 12 | 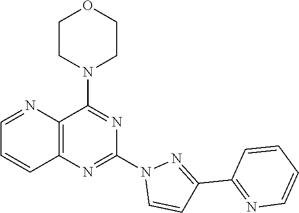 [SMILES: c1ccc(-c2ccn(-c3nc(N4CCOCC4)c4ncccc4n3)n2)nc1] |
| 13 | 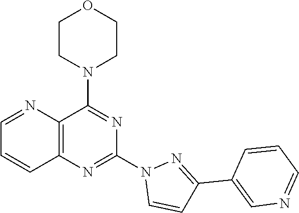 [SMILES: c1cncc(-c2ccn(-c3nc(N4CCOCC4)c4ncccc4n3)n2)c1] |
| 14 | 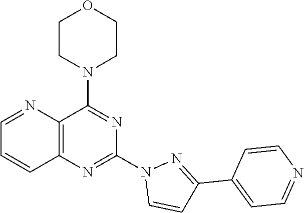 [SMILES: c1cnc2c(N3CCOCC3)nc(-n3ccc(-c4ccncc4)n3)nc2c1] |
| 15 | 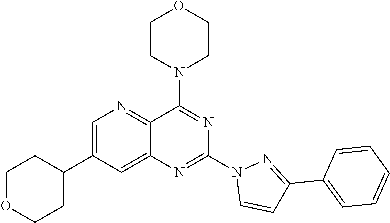 [SMILES: c1ccc(-c2ccn(-c3nc(N4CCOCC4)c4ncc(C5CCOCC5)cc4n3)n2)cc1] |
| 16 | 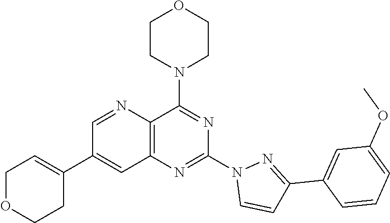 [SMILES: COc1cccc(-c2ccn(-c3nc(N4CCOCC4)c4ncc(C5=CCOCC5)cc4n3)n2)c1] |
| 17 | 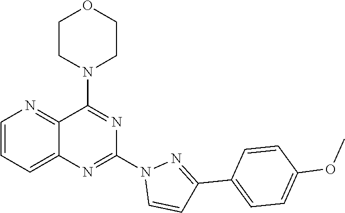 [SMILES: COc1ccc(-c2ccn(-c3nc(N4CCOCC4)c4ncccc4n3)n2)cc1] |
| 18 | 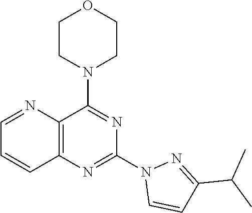 [SMILES: CC(C)c1ccn(-c2nc(N3CCOCC3)c3ncccc3n2)n1] |
| 19 | 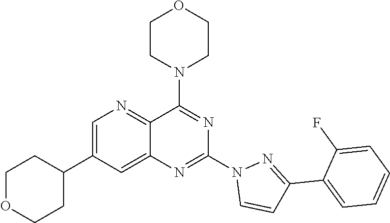 [SMILES: Fc1ccccc1-c1ccn(-c2nc(N3CCOCC3)c3ncc(C4CCOCC4)cc3n2)n1] |
| 20 | 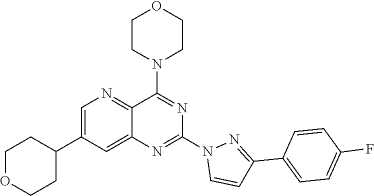 [SMILES: Fc1ccc(-c2ccn(-c3nc(N4CCOCC4)c4ncc(C5CCOCC5)cc4n3)n2)cc1] |
| 21 | 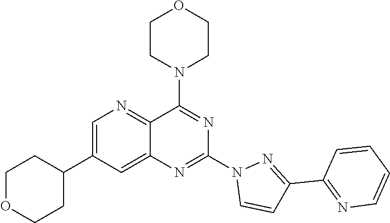 [SMILES: c1ccc(-c2ccn(-c3nc(N4CCOCC4)c4ncc(C5CCOCC5)cc4n3)n2)nc1] |
| 22 | 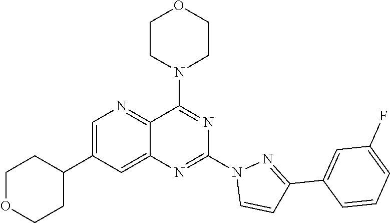 [SMILES: Fc1cccc(-c2ccn(-c3nc(N4CCOCC4)c4ncc(C5CCOCC5)cc4n3)n2)c1] |
| 23 | 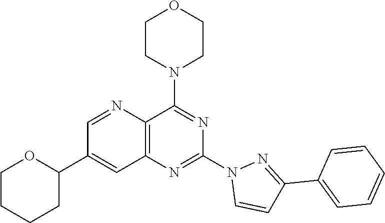 [SMILES: c1ccc(-c2ccn(-c3nc(N4CCOCC4)c4ncc(C5CCCCO5)cc4n3)n2)cc1] |
| 24 | 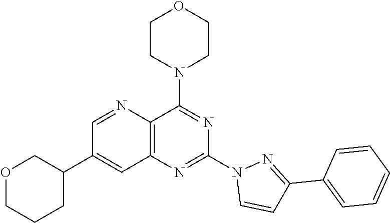 [SMILES: c1ccc(-c2ccn(-c3nc(N4CCOCC4)c4ncc(C5CCCOC5)cc4n3)n2)cc1] |
| 25 | 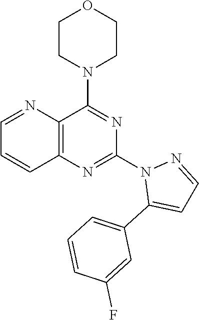 [SMILES: Fc1cccc(-c2ccnn2-c2nc(N3CCOCC3)c3ncccc3n2)c1] |
| 26 | 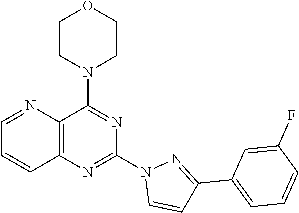 [SMILES: Fc1cccc(-c2ccn(-c3nc(N4CCOCC4)c4ncccc4n3)n2)c1] |
| 27 | 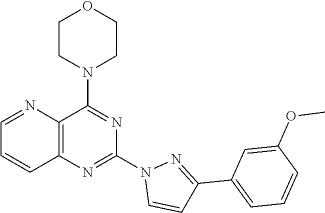 [SMILES: COc1cccc(-c2ccn(-c3nc(N4CCOCC4)c4ncccc4n3)n2)c1] |
| 28 | 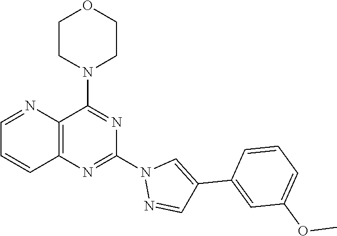 [SMILES: COc1cccc(-c2cnn(-c3nc(N4CCOCC4)c4ncccc4n3)c2)c1] |
| 29 | 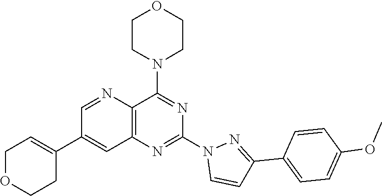 [SMILES: COc1ccc(-c2ccn(-c3nc(N4CCOCC4)c4ncc(C5=CCOCC5)cc4n3)n2)cc1] |
| 30 | 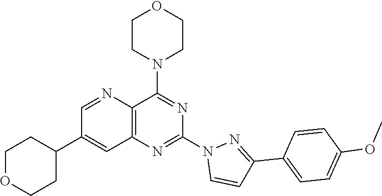 [SMILES: COc1ccc(-c2ccn(-c3nc(N4CCOCC4)c4ncc(C5CCOCC5)cc4n3)n2)cc1] |
| 31 | 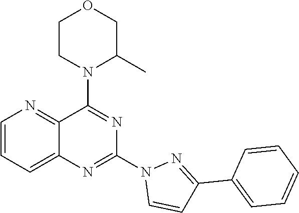 [SMILES: CC1COCCN1c1nc(-n2ccc(-c3ccccc3)n2)nc2cccnc12] |
| 32 | 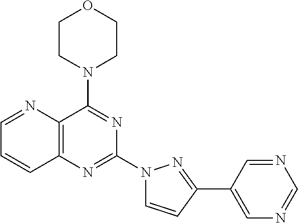 [SMILES: c1cnc2c(N3CCOCC3)nc(-n3ccc(-c4cncnc4)n3)nc2c1] |
| 33 | 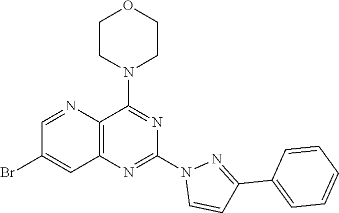 [SMILES: Brc1cnc2c(N3CCOCC3)nc(-n3ccc(-c4ccccc4)n3)nc2c1] |
| 34 | 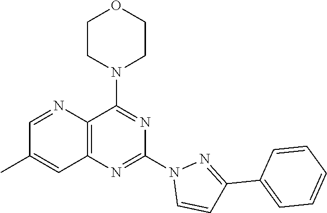 [SMILES: Cc1cnc2c(N3CCOCC3)nc(-n3ccc(-c4ccccc4)n3)nc2c1] |
| 35 | 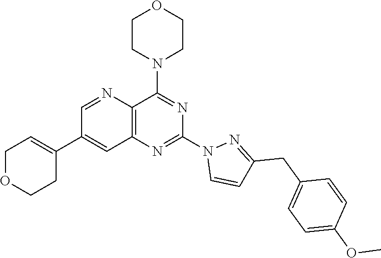 [SMILES: COc1ccc(Cc2ccn(-c3nc(N4CCOCC4)c4ncc(C5=CCOCC5)cc4n3)n2)cc1] |
| 36 | 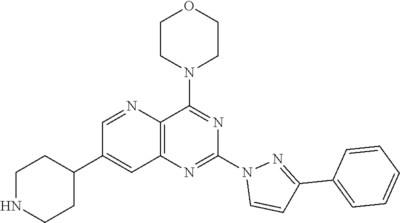 [SMILES: c1ccc(-c2ccn(-c3nc(N4CCOCC4)c4ncc(C5CCNCC5)cc4n3)n2)cc1] |
| 37 | 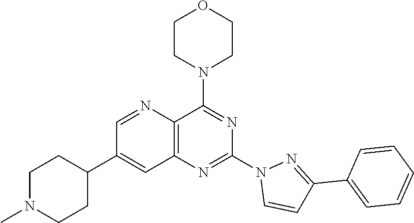 [SMILES: CN1CCC(c2cnc3c(N4CCOCC4)nc(-n4ccc(-c5ccccc5)n4)nc3c2)CC1] |
| 38 | 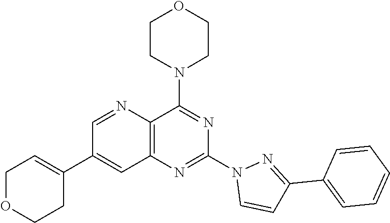 [SMILES: C1=C(c2cnc3c(N4CCOCC4)nc(-n4ccc(-c5ccccc5)n4)nc3c2)CCOC1] |
| 39 | 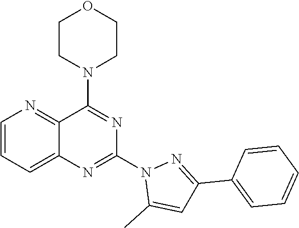 [SMILES: Cc1cc(-c2ccccc2)nn1-c1nc(N2CCOCC2)c2ncccc2n1] |
| 40 | 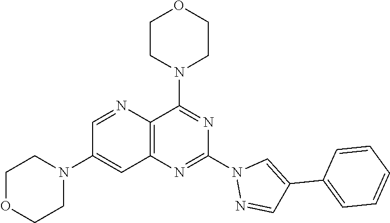 [SMILES: c1ccc(-c2cnn(-c3nc(N4CCOCC4)c4ncc(N5CCOCC5)cc4n3)c2)cc1] |
| 41 | 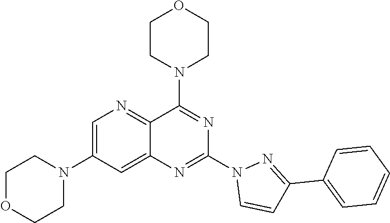 [SMILES: c1ccc(-c2ccn(-c3nc(N4CCOCC4)c4ncc(N5CCOCC5)cc4n3)n2)cc1] |
| 42 | 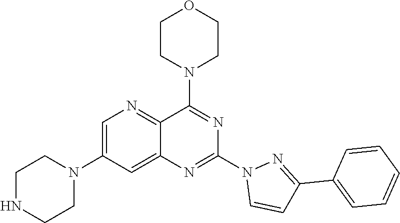 [SMILES: c1ccc(-c2ccn(-c3nc(N4CCOCC4)c4ncc(N5CCNCC5)cc4n3)n2)cc1] |
| 43 | 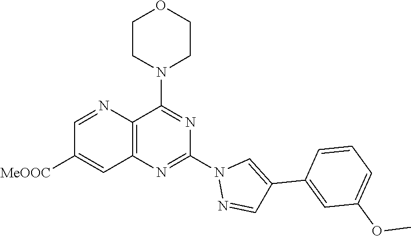 [SMILES: COC(=O)c1cnc2c(N3CCOCC3)nc(-n3cc(-c4cccc(OC)c4)cn3)nc2c1] |
| 44 | 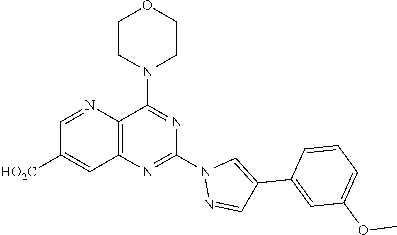 [SMILES: COc1cccc(-c2cnn(-c3nc(N4CCOCC4)c4ncc(C(=O)O)cc4n3)c2)c1] |
| 45 | 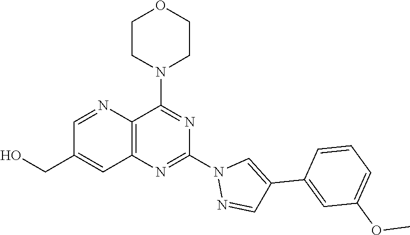 [SMILES: COc1cccc(-c2cnn(-c3nc(N4CCOCC4)c4ncc(CO)cc4n3)c2)c1] |
| 46 | 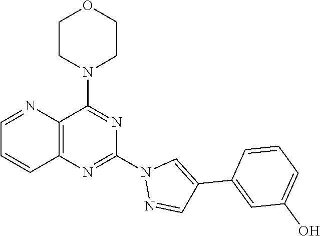 [SMILES: Oc1cccc(-c2cnn(-c3nc(N4CCOCC4)c4ncccc4n3)c2)c1] |
| 47 | 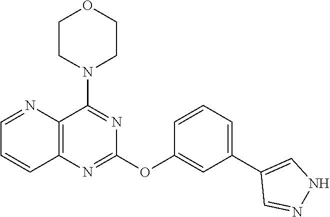 [SMILES: c1cc(Oc2nc(N3CCOCC3)c3ncccc3n2)cc(-c2cn[nH]c2)c1] |
| 48 | 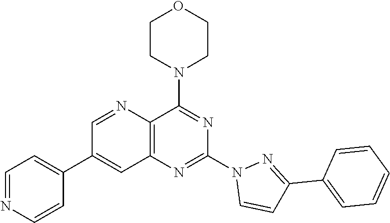 [SMILES: c1ccc(-c2ccn(-c3nc(N4CCOCC4)c4ncc(-c5ccncc5)cc4n3)n2)cc1] |
| 49 | 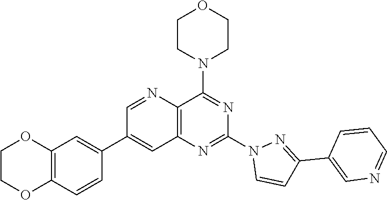 [SMILES: c1cncc(-c2ccn(-c3nc(N4CCOCC4)c4ncc(-c5ccc6c(c5)OCCO6)cc4n3)n2)c1] |
| 50 | 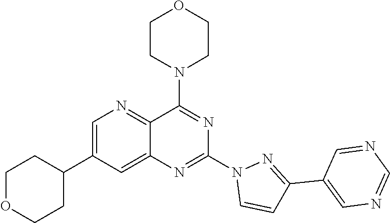 [SMILES: c1ncc(-c2ccn(-c3nc(N4CCOCC4)c4ncc(C5CCOCC5)cc4n3)n2)cn1] |
| 51 | 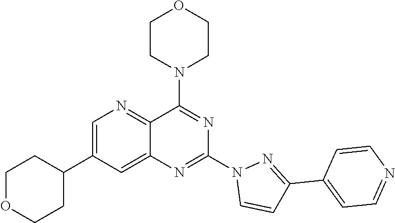 [SMILES: c1cc(-c2ccn(-c3nc(N4CCOCC4)c4ncc(C5CCOCC5)cc4n3)n2)ccn1] |
| 52 | 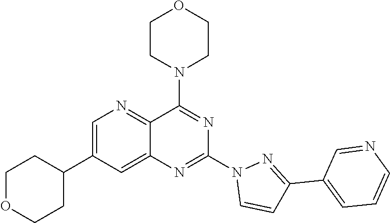 [SMILES: c1cncc(-c2ccn(-c3nc(N4CCOCC4)c4ncc(C5CCOCC5)cc4n3)n2)c1] |
| 53 | 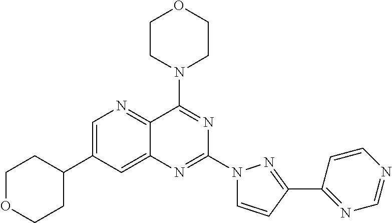 [SMILES: c1cc(-c2ccn(-c3nc(N4CCOCC4)c4ncc(C5CCOCC5)cc4n3)n2)ncn1] |
| 54 | 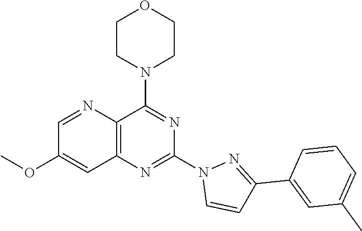 |
| 55 | 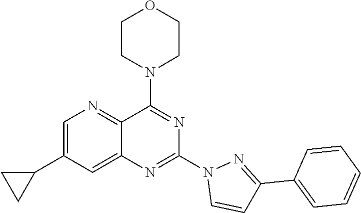 [SMILES: c1ccc(-c2ccn(-c3nc(N4CCOCC4)c4ncc(C5CC5)cc4n3)n2)cc1] |
| 56 | 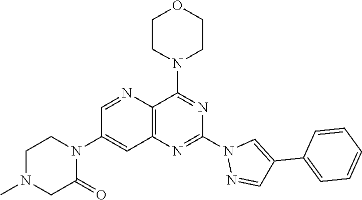 [SMILES: CN1CCN(c2cnc3c(N4CCOCC4)nc(-n4cc(-c5ccccc5)cn4)nc3c2)C(=O)C1] |
| 57 | 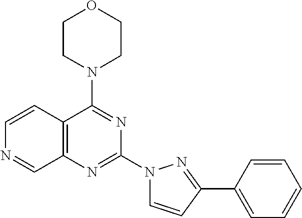 [SMILES: c1ccc(-c2ccn(-c3nc(N4CCOCC4)c4ccncc4n3)n2)cc1] |
| 58 | 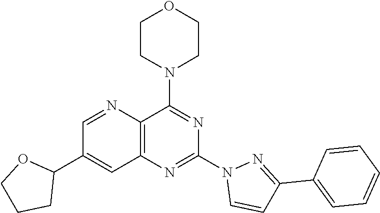 [SMILES: c1ccc(-c2ccn(-c3nc(N4CCOCC4)c4ncc(C5CCCO5)cc4n3)n2)cc1] |
| 59 | 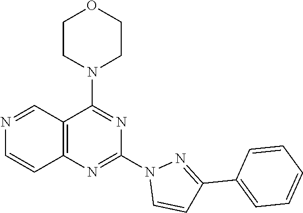 [SMILES: c1ccc(-c2ccn(-c3nc(N4CCOCC4)c4cnccc4n3)n2)cc1] |
| 60 | 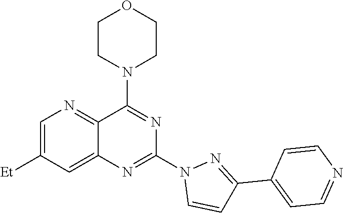 [SMILES: CCc1cnc2c(N3CCOCC3)nc(-n3ccc(-c4ccncc4)n3)nc2c1] |
| 61 | 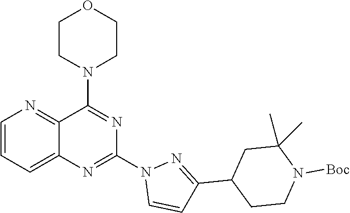 [SMILES: CC(C)(C)OC(=O)N1CCC(c2ccn(-c3nc(N4CCOCC4)c4ncccc4n3)n2)CC1(C)C] |
| 62 | 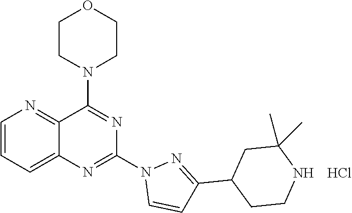 [SMILES: CC1(C)CC(c2ccn(-c3nc(N4CCOCC4)c4ncccc4n3)n2)CCN1 ; Cl] |
| 63 | 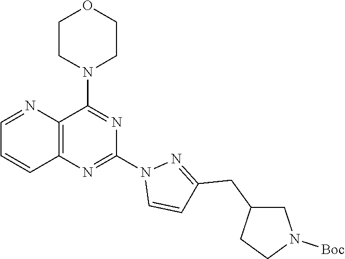 [SMILES: CC(C)(C)OC(=O)N1CCC(Cc2ccn(-c3nc(N4CCOCC4)c4ncccc4n3)n2)C1] |
| 64 | 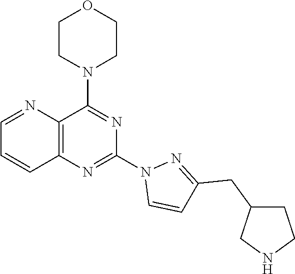 [SMILES: c1cnc2c(N3CCOCC3)nc(-n3ccc(CC4CCNC4)n3)nc2c1] |
| 65 | 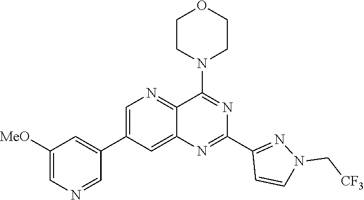 [SMILES: COc1cncc(-c2cnc3c(N4CCOCC4)nc(-c4ccn(CC(F)(F)F)n4)nc3c2)c1] |
| 66 | 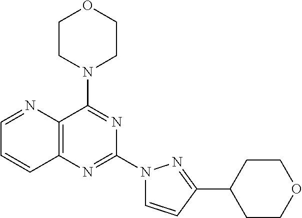 [SMILES: c1cnc2c(N3CCOCC3)nc(-n3ccc(C4CCOCC4)n3)nc2c1] |
| 67 | 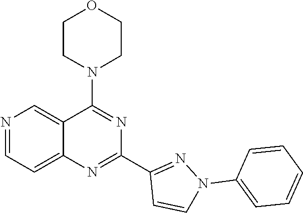 [SMILES: c1ccc(-n2ccc(-c3nc(N4CCOCC4)c4cnccc4n3)n2)cc1] |
| 68 | 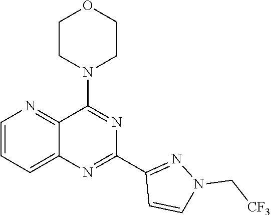 [SMILES: FC(F)(F)Cn1ccc(-c2nc(N3CCOCC3)c3ncccc3n2)n1] |
| 69 | 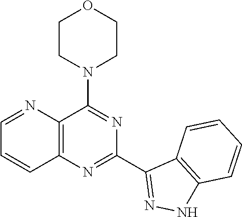 [SMILES: c1cnc2c(N3CCOCC3)nc(-c3n[nH]c4ccccc34)nc2c1] |
| 70 | 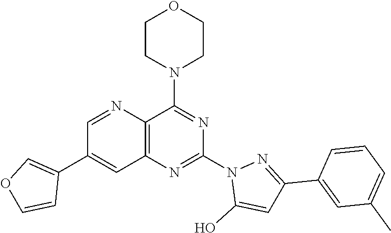 [SMILES: Cc1cccc(-c2cc(O)n(-c3nc(N4CCOCC4)c4ncc(-c5ccoc5)cc4n3)n2)c1] |
| 71 | 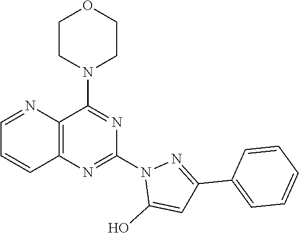 [SMILES: Oc1cc(-c2ccccc2)nn1-c1nc(N2CCOCC2)c2ncccc2n1] |
| 72 | 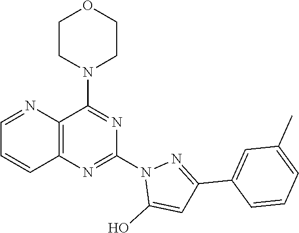 [SMILES: Cc1cccc(-c2cc(O)n(-c3nc(N4CCOCC4)c4ncccc4n3)n2)c1] |
| 73 | 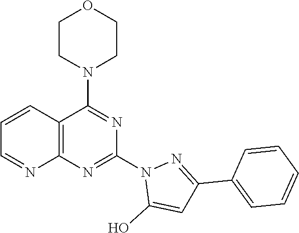 [SMILES: Oc1cc(-c2ccccc2)nn1-c1nc(N2CCOCC2)c2cccnc2n1] |
| 74 | 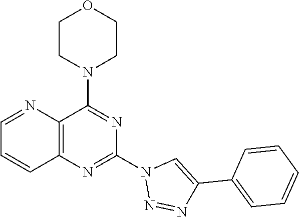 [SMILES: c1ccc(-c2cn(-c3nc(N4CCOCC4)c4ncccc4n3)nn2)cc1] |
| 75 | 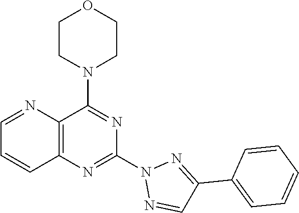 [SMILES: c1ccc(-c2cnn(-c3nc(N4CCOCC4)c4ncccc4n3)n2)cc1] |
| 76 | 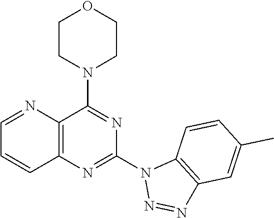 [SMILES: Cc1ccc2c(c1)nnn2-c1nc(N2CCOCC2)c2ncccc2n1] |
| 77 | 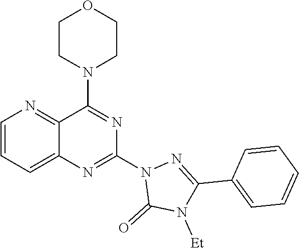 [SMILES: CCn1c(-c2ccccc2)nn(-c2nc(N3CCOCC3)c3ncccc3n2)c1=O] |
| 78 | 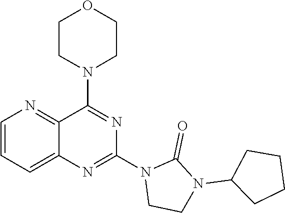 [SMILES: O=C1N(c2nc(N3CCOCC3)c3ncccc3n2)CCN1C1CCCC1] |
| 79 | 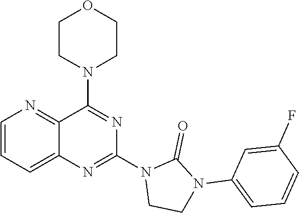 [SMILES: O=C1N(c2cccc(F)c2)CCN1c1nc(N2CCOCC2)c2ncccc2n1] |
| 80 | 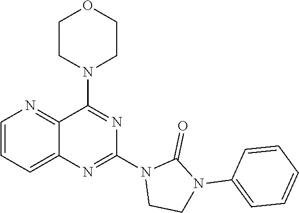 [SMILES: O=C1N(c2ccccc2)CCN1c1nc(N2CCOCC2)c2ncccc2n1] |
| 81 | 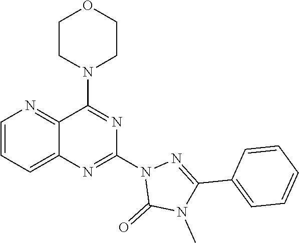 [SMILES: Cn1c(-c2ccccc2)nn(-c2nc(N3CCOCC3)c3ncccc3n2)c1=O] |
| 82 | 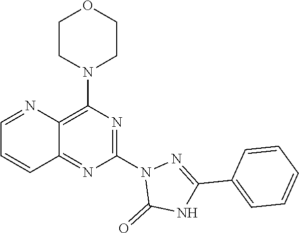 [SMILES: O=c1[nH]c(-c2ccccc2)nn1-c1nc(N2CCOCC2)c2ncccc2n1] |
| 83 | 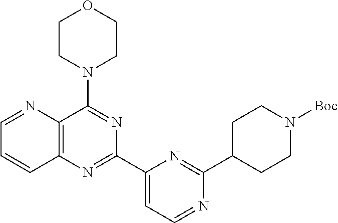 [SMILES: CC(C)(C)OC(=O)N1CCC(c2nccc(-c3nc(N4CCOCC4)c4ncccc4n3)n2)CC1] |
| 84 | 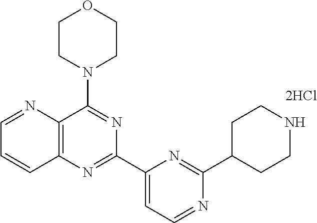 [SMILES: c1cnc2c(N3CCOCC3)nc(-c3ccnc(C4CCNCC4)n3)nc2c1] |
| 85 | 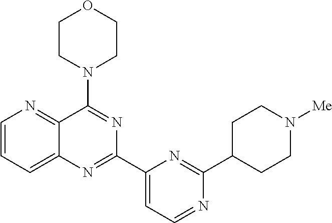 [SMILES: CN1CCC(c2nccc(-c3nc(N4CCOCC4)c4ncccc4n3)n2)CC1] |
| 86 | 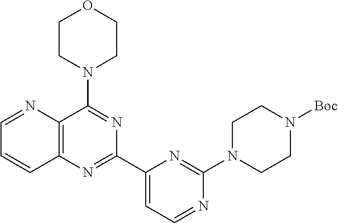 [SMILES: CC(C)(C)OC(=O)N1CCN(c2nccc(-c3nc(N4CCOCC4)c4ncccc4n3)n2)CC1] |
| 87 | 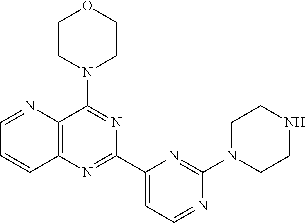 [SMILES: c1cnc2c(N3CCOCC3)nc(-c3ccnc(N4CCNCC4)n3)nc2c1] |
| 88 | 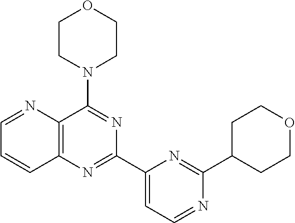 [SMILES: c1cnc2c(N3CCOCC3)nc(-c3ccnc(C4CCOCC4)n3)nc2c1] |
| 89 | 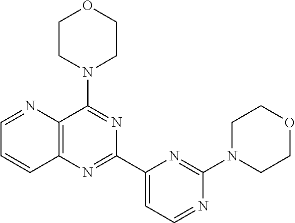 [SMILES: c1cnc2c(N3CCOCC3)nc(-c3ccnc(N4CCOCC4)n3)nc2c1] |
| 90 | 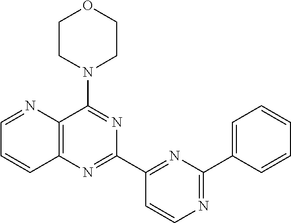 [SMILES: c1ccc(-c2nccc(-c3nc(N4CCOCC4)c4ncccc4n3)n2)cc1] |
| 91 | 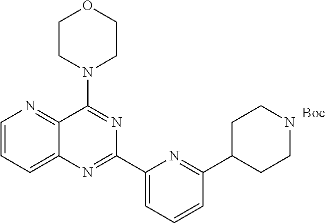 [SMILES: c1cc(-c2nc(N3CCOCC3)c3ncccc3n2)nc(C2CCNCC2)c1 ; "Boc"] |
| 92 | 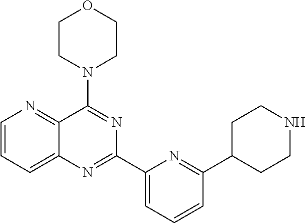 [SMILES: c1cc(-c2nc(N3CCOCC3)c3ncccc3n2)nc(C2CCNCC2)c1] |
| 93 | 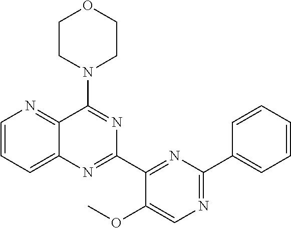 [SMILES: COc1cnc(-c2ccccc2)nc1-c1nc(N2CCOCC2)c2ncccc2n1] |
| 94 | 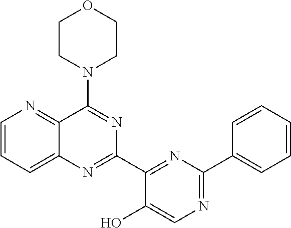 [SMILES: Oc1cnc(-c2ccccc2)nc1-c1nc(N2CCOCC2)c2ncccc2n1] |
| 95 | 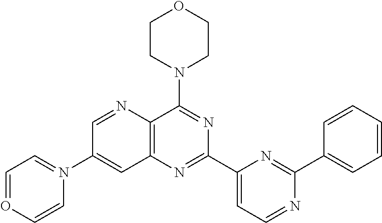 |
| 96 | 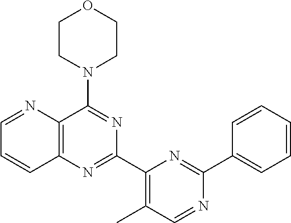 [SMILES: Cc1cnc(-c2ccccc2)nc1-c1nc(N2CCOCC2)c2ncccc2n1] |
| 97 | 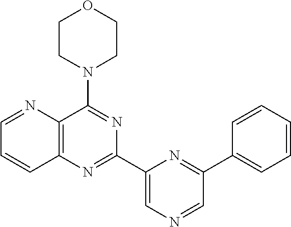 [SMILES: c1ccc(-c2cncc(-c3nc(N4CCOCC4)c4ncccc4n3)n2)cc1] |
| 98 | 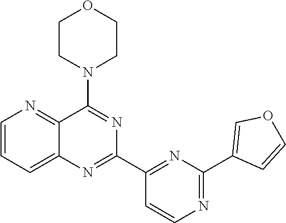 [SMILES: c1cnc2c(N3CCOCC3)nc(-c3ccnc(-c4ccoc4)n3)nc2c1] |
| 99 | 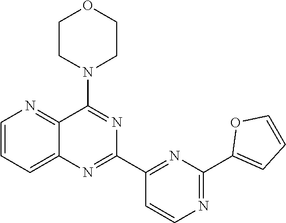 [SMILES: c1coc(-c2nccc(-c3nc(N4CCOCC4)c4ncccc4n3)n2)c1] |
| 100 | 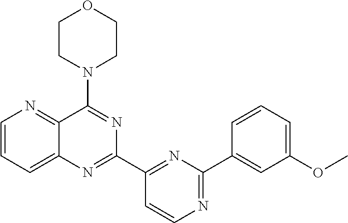 [SMILES: COc1cccc(-c2nccc(-c3nc(N4CCOCC4)c4ncccc4n3)n2)c1] |
| 101 | 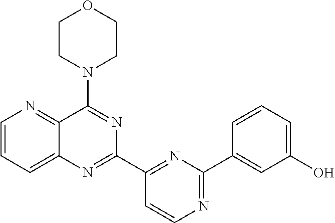 [SMILES: Oc1cccc(-c2nccc(-c3nc(N4CCOCC4)c4ncccc4n3)n2)c1] |
| 102 | 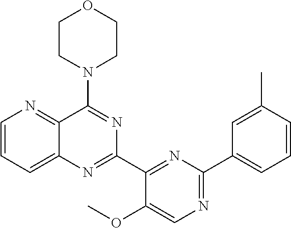 [SMILES: COc1cnc(-c2cccc(C)c2)nc1-c1nc(N2CCOCC2)c2ncccc2n1] |
| 103 | 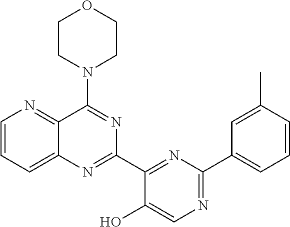 [SMILES: Cc1cccc(-c2ncc(O)c(-c3nc(N4CCOCC4)c4ncccc4n3)n2)c1] |
| 104 | 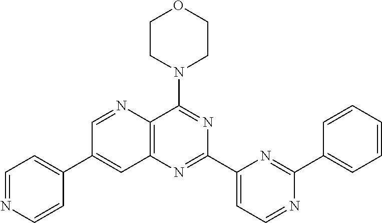 [SMILES: c1ccc(-c2nccc(-c3nc(N4CCOCC4)c4ncc(-c5ccncc5)cc4n3)n2)cc1] |
| 105 | 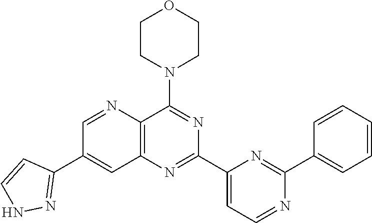 [SMILES: c1ccc(-c2nccc(-c3nc(N4CCOCC4)c4ncc(-c5cc[nH]n5)cc4n3)n2)cc1] |
| 106 | 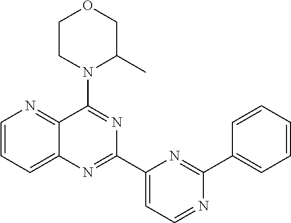 [SMILES: CC1COCCN1c1nc(-c2ccnc(-c3ccccc3)n2)nc2cccnc12] |
| 107 | 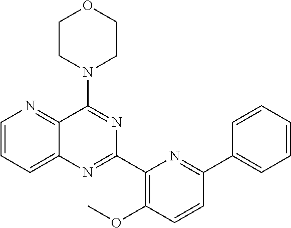 [SMILES: COc1ccc(-c2ccccc2)nc1-c1nc(N2CCOCC2)c2ncccc2n1] |
| 108 | 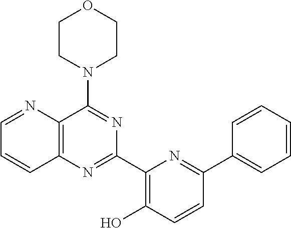 [SMILES: Oc1ccc(-c2ccccc2)nc1-c1nc(N2CCOCC2)c2ncccc2n1] |
| 109 | 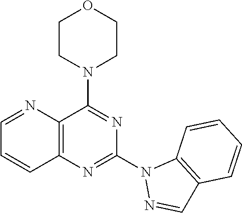 [SMILES: c1ccc2c(c1)cnn2-c1nc(N2CCOCC2)c2ncccc2n1] |
| 110 | 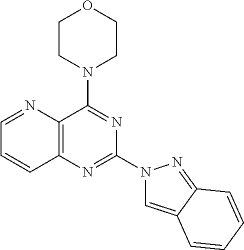 [SMILES: c1ccc2nn(-c3nc(N4CCOCC4)c4ncccc4n3)cc2c1] |
| 111 | 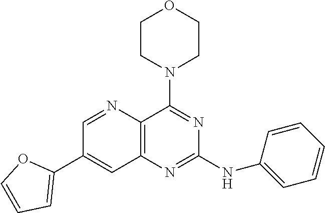 [SMILES: c1ccc(Nc2nc(N3CCOCC3)c3ncc(-c4ccco4)cc3n2)cc1] |
| 112 | 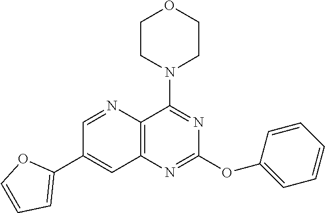 [SMILES: c1ccc(Oc2nc(N3CCOCC3)c3ncc(-c4ccco4)cc3n2)cc1] |
| 113 | 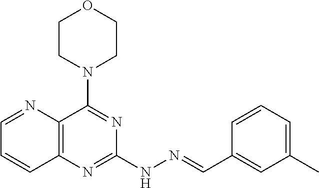 [SMILES: Cc1cccc(/C=N/Nc2nc(N3CCOCC3)c3ncccc3n2)c1] |
| 114 | 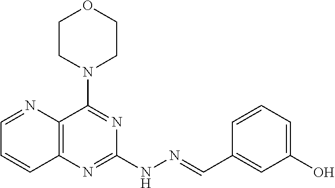 [SMILES: Oc1cccc(/C=N/Nc2nc(N3CCOCC3)c3ncccc3n2)c1] |
| 115 | 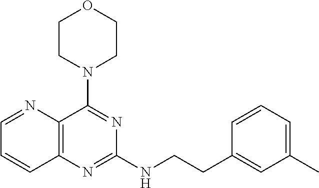 [SMILES: Cc1cccc(CCNc2nc(N3CCOCC3)c3ncccc3n2)c1] |
| 116 | 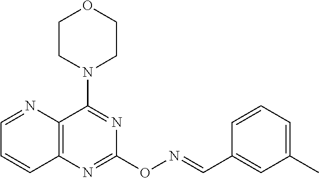 [SMILES: Cc1cccc(/C=N/Oc2nc(N3CCOCC3)c3ncccc3n2)c1] |
| 117 | 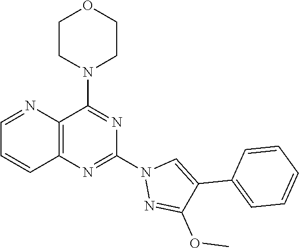 [SMILES: COc1nn(-c2nc(N3CCOCC3)c3ncccc3n2)cc1-c1ccccc1] |
| 122 | 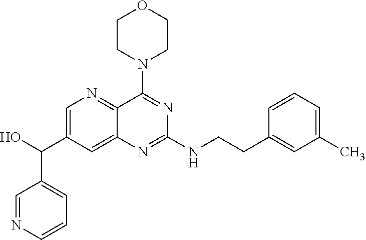 [SMILES: Cc1cccc(CCNc2nc(N3CCOCC3)c3ncc(C(O)c4cccnc4)cc3n2)c1] |
| 123 | 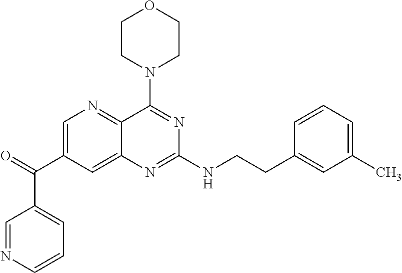 [SMILES: Cc1cccc(CCNc2nc(N3CCOCC3)c3ncc(C(=O)c4cccnc4)cc3n2)c1] |
| 124 | 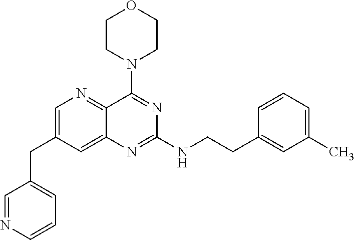 [SMILES: Cc1cccc(CCNc2nc(N3CCOCC3)c3ncc(Cc4cccnc4)cc3n2)c1] |
| 125 | 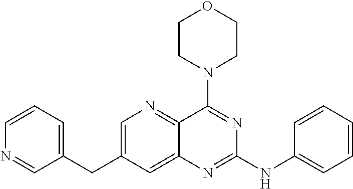 [SMILES: c1ccc(Nc2nc(N3CCOCC3)c3ncc(Cc4cccnc4)cc3n2)cc1] |
| 126 | 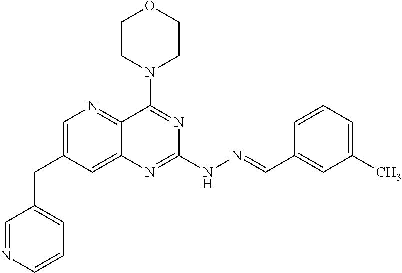 [SMILES: Cc1cccc(/C=N/Nc2nc(N3CCOCC3)c3ncc(Cc4cccnc4)cc3n2)c1] |
| 127 | 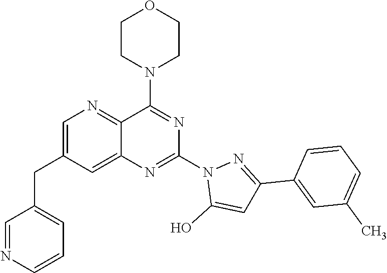 [SMILES: Cc1cccc(-c2cc(O)n(-c3nc(N4CCOCC4)c4ncc(Cc5cccnc5)cc4n3)n2)c1] |
| 128 | 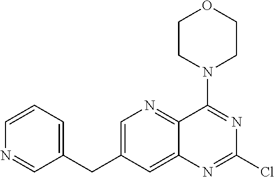 [SMILES: Clc1nc(N2CCOCC2)c2ncc(Cc3cccnc3)cc2n1] |
| 129 | 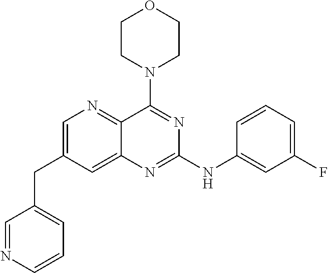 [SMILES: Fc1cccc(Nc2nc(N3CCOCC3)c3ncc(Cc4cccnc4)cc3n2)c1] |
| 130 | 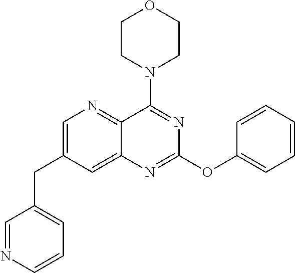 [SMILES: c1ccc(Oc2nc(N3CCOCC3)c3ncc(Cc4cccnc4)cc3n2)cc1] |
| 131 | 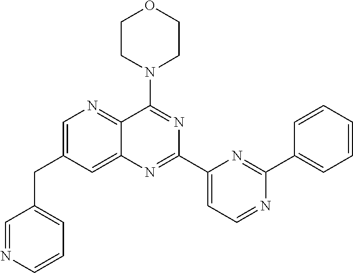 [SMILES: c1ccc(-c2nccc(-c3nc(N4CCOCC4)c4ncc(Cc5cccnc5)cc4n3)n2)cc1] |
| 132 | 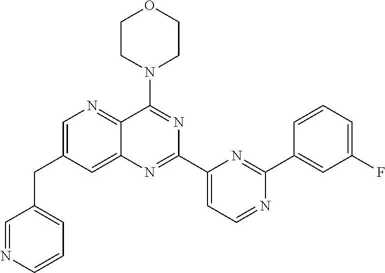 [SMILES: Fc1cccc(-c2nccc(-c3nc(N4CCOCC4)c4ncc(Cc5cccnc5)cc4n3)n2)c1] |
| 133 | 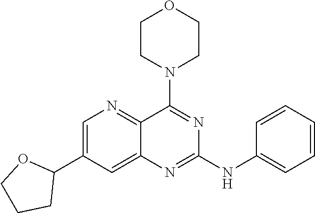 [SMILES: c1ccc(Nc2nc(N3CCOCC3)c3ncc(C4CCCO4)cc3n2)cc1] |
| 134 | 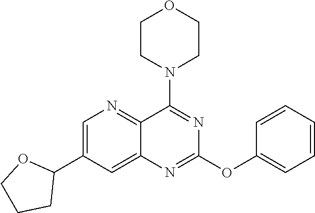 [SMILES: c1ccc(Oc2nc(N3CCOCC3)c3ncc(C4CCCO4)cc3n2)cc1] |
| 135 | 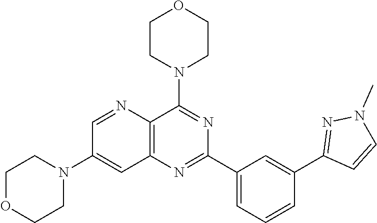 [SMILES: Cn1ccc(-c2cccc(-c3nc(N4CCOCC4)c4ncc(N5CCOCC5)cc4n3)c2)n1] |
| 136 | 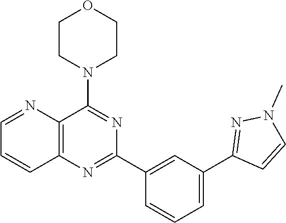 [SMILES: Cn1ccc(-c2cccc(-c3nc(N4CCOCC4)c4ncccc4n3)c2)n1] |
| 137 | 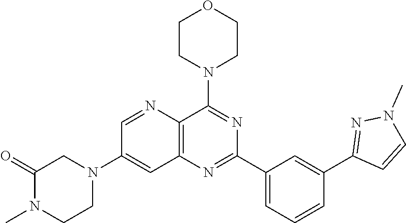 [SMILES: CN1CCN(c2cnc3c(N4CCOCC4)nc(-c4cccc(-c5ccn(C)n5)c4)nc3c2)CC1=O] |

an pharmaceutically acceptable salt thereof.

Preferably, the compound is of formula IIb:

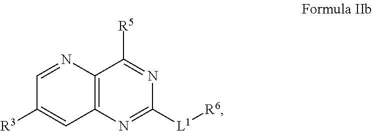

or a pharmaceutically acceptable salt thereof, where the variables are as described herein.

### Methods of Treatment

### Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition including a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions including a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### EXAMPLES

### Abbreviations

| | |
|---|---|
| ACN | Acetonitrile |
| 9-BBN | 9-Borabicyclo[3.3.1]nonane |
| CDI | 1,1′-Carbonyldiimidazole |
| CO | Carbon monoxide |
| DCM | Dichloromethane |
| DIPEA | N,N-Diisopropylethylamine |
| DMAc | Dimethylacetamide |
| DMEDA | 1,2-Dimethylethylenediamine |
| DMF | N,N-Dimethylformamide |
| DMF-DMA | N,N-Dimethylformamide dimethyl acetal |
| DMSO | Dimethylsulfoxide |
| EA | Ethyl acetate |
| FA | Formic acid |
| For NMR | S—singlet, d—doublet, dd—doublet-of-doublet, dt—doublet of triplet, q—quartet, |
| | dq—doublet of quartet, bs—broad singlet, dpent—doublet of pentet, t—triplet, |
| | pent—pentet; hept—heptet |
| h | Hour(s) |
| NaHMDS | Sodium hexamethyldisilazide |
| Pd(tBu3P)2 | Bis(tri-tert-butylphosphine)palladium(0) |
| Pd2(dba)3 | Tris(dibenzylideneacetone)dipalladium(0) |
| PdCl2(dppf) | [1,1′-Bis(diphenylphosphino)ferrocene]dichloropalladium(II) |
| PE:EA | Petroleum ether:Ethyl acetate |
| RT | Room temperature |
| tBuBrettPhos | [(2-Di-tert-butylphosphino-3,6-dimethoxy-2′,4′,6′-triisopropyl-1,1′-biphenyl)-2-(2′- |
| PD G3 | amino-1,1′-biphenyl)]palladium(II) methanesulfonate |
| TFA | Trifluoroacetic acid |
| THF | Tetrahydrafuran |
| TRIXIEPHOS | ditert-butyl-(1-naphthalen-1-ylnaphthalen-2-yl)phosphane |
| Xantphos | 4,5-Bis(diphenylphosphino)-9,9-dimethylxanthene |
| XPhos | 2-Dicyclohexylphosphino-2′,4′,6′-triisopropylbiphenyl |

### Preparation of Compounds—General Schemes

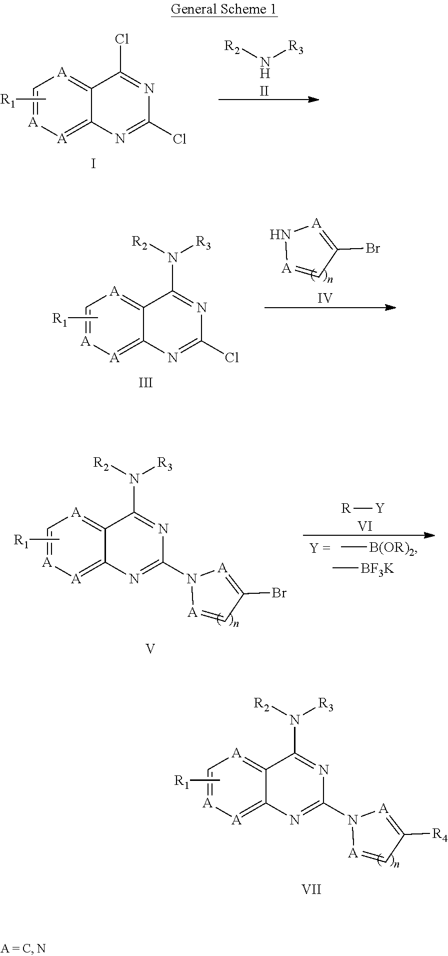

An appropriately substituted dichloropyrimidine I is coupled with appropriately substituted amine II under basic conditions (e.g. triethylamine) to afford appropriately substituted aryl chloride III. Aryl chloride III is reacted with appropriately substituted amine IV under basic conditions (e.g. cesium carbonate) to afford appropriately substituted aryl bromide V. Aryl bromide V is coupled with appropriately substituted boronate ester VI in the presence of a palladium catalyst (e.g. palladium tetrakis) to afford desired product VII.

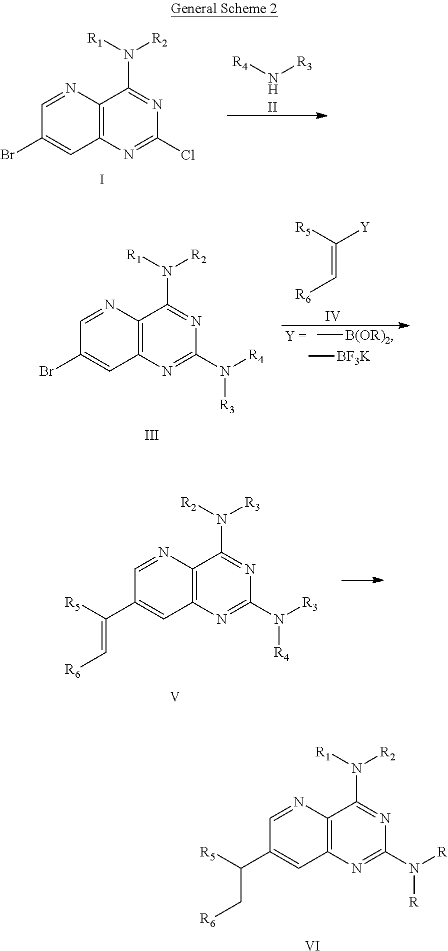

An appropriately substituted aryl chloride I is reacted with amine II under basic conditions (e.g. cesium carbonate) to afford appropriately substituted aryl bromide III. Aryl bromide III can be coupled with appropriately substituted boronate ester IV in the presence of a palladium catalyst (e.g. palladium tetrakis) to afford appropriately substituted pyridopyrimidine V, which can be hydrogenated in the presence of palladium on carbon to afford desired pyridopyrimidine VI.

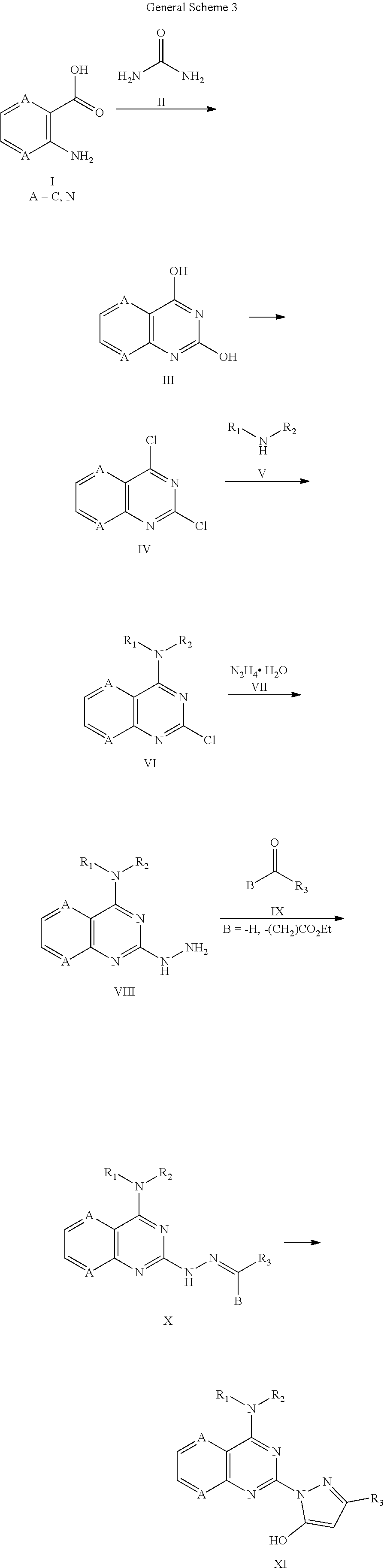

An appropriately substituted carboxylic acid I and urea II are reacted with heat to give appropriately substituted diol III, which is chlorinated with phosphorus oxychloride to give appropriately substituted aryl chloride IV. Aryl chloride IV is reacted with appropriately substituted amine V under basic conditions (e.g. triethylamine) to give appropriately substituted aryl chloride VI. Aryl chloride VI is reacted with hydrazine hydrate VII with heat to give appropriately substituted hydrazine VII. Hydrazine VII is reacted with appropriately substituted aldehyde or enone IX under acidic conditions (e.g. acetic acid) to give desired hydrazone X. When “B” is appropriately substituted enone IX, hydrazone X can be further cyclized to give desired hydroxypyrazole XI.

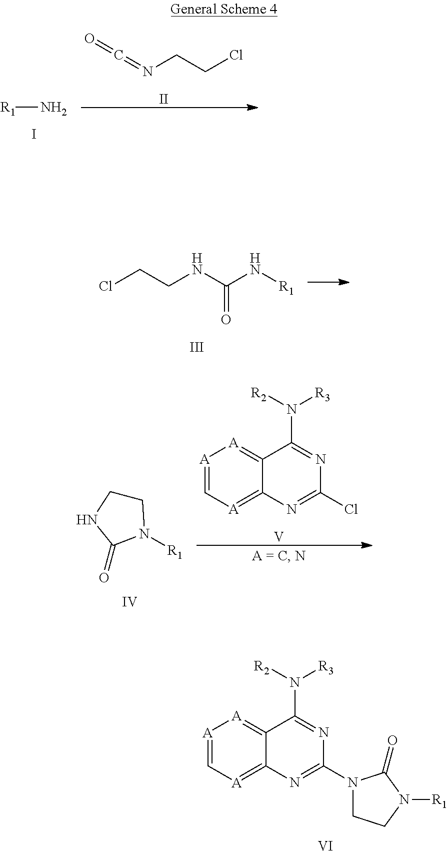

An appropriately substituted amine I is reacted with 1-chloro-2-isocyanatoethane II to give appropriately substituted urea III. Urea III is cyclized under basic conditions (e.g. sodium hydride) to give cyclic urea IV. Urea IV is reacted with appropriately substituted aryl chloride V to give desired product VI.

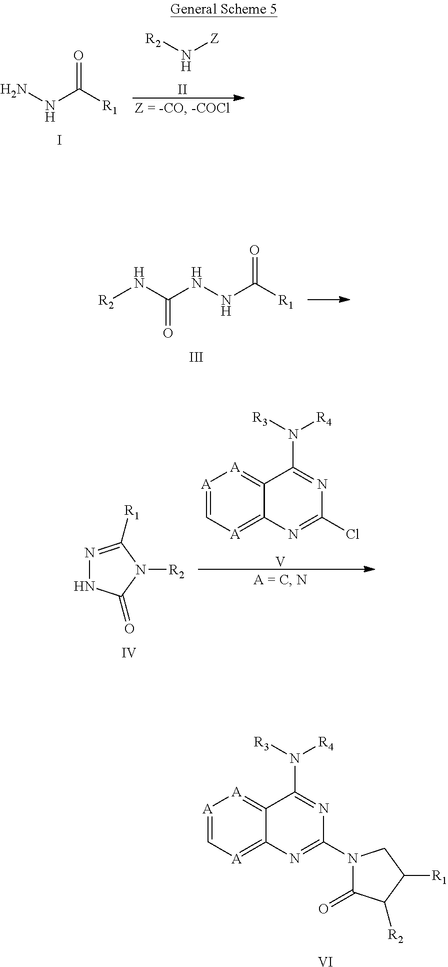

An appropriately substituted hydrazine I is reacted with appropriately substituted isocyanate or carbamoyl chloride II to give appropriately substituted carbohydrazide III, which is cyclized under basic conditions (e.g. sodium hydroxide) to give appropriately substituted triazolone IV. Triazolone IV is reacted with appropriately substituted aryl chloride V under basic conditions (e.g. cesium carbonate) to give desired product VI.

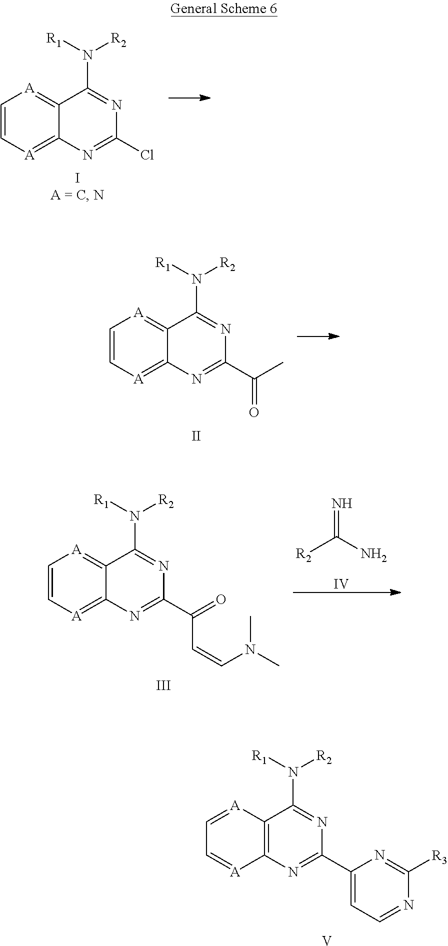

An appropriately substituted aryl chloride I is reacted with tributyl(1-ethoxyvinyl)stannane in the presence of a palladium catalyst (e.g. bis(triphenylphosphine)palladium(II) dichloride) to afford appropriately substituted acetylpyrimidine II. Acetylpyrimidine II is reacted with N,N-dimethylformamide dimethyl acetal to afford appropriately substituted enone III. Enone III is reacted with appropriately substituted amidine IV under basic conditions (e.g. sodium ethoxide) to afford desired pyrimidine V.

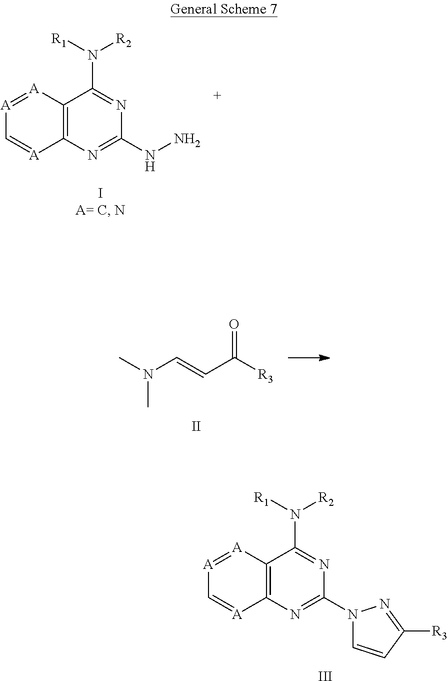

An appropriately substituted hydrazine I is reacted with an appropriately substituted enone II under acidic conditions (e.g. acetic acid) to afford desired pyrazole III.

### Synthesis of tert-butyl 4-[1-(4-morpholinopyrido[3,2-d]pyrimidin-2-yl)pyrazol-3-yl]-3,6-dihydro-2H-pyridine-1-carboxylate (Compound 1), tert-butyl 4-[1-(4-morpholinopyrido[3,2-d]pyrimidin-2-yl)pyrazol-3-yl]piperidine-1-carboxylate (Compound 2), 4-[2-[3-(4-piperidyl)pyrazol-1-yl]pyrido[3,2-d]pyrimidin-4-yl]morpholine (Compound 3) and 1-[4-[1-(4-morpholinopyrido[3,2-d]pyrimidin-2-yl)pyrazol-3-yl]-1-piperidyl]propan-1-one (Compound 4)

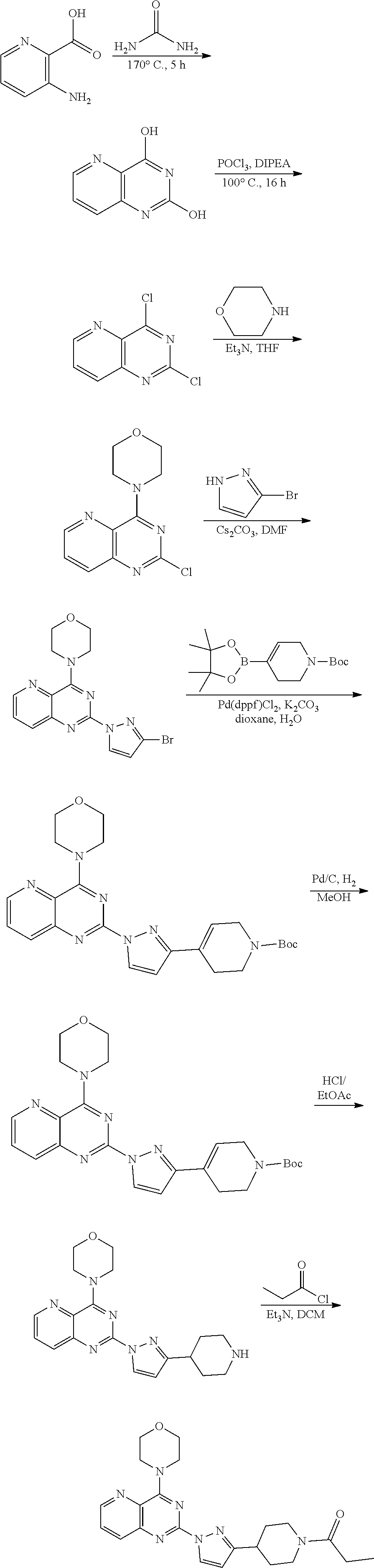

### Step 1: Synthesis of pyrido[3,2-d]pyrimidine-2,4-diol

A solution of 3-aminopicolinic acid (4.00 g, 29 mmol) and urea (2.60 g, 44 mmol) in ethanol (5.0 mL) was stirred at 1700° C. under nitrogen atmosphere for 6 h. The reaction mixture was concentrated under reduced pressure and deionized water (100 mL) added to the residue. The resultant solution was then acidified with 1.5 M hydrochloric acid solution until a precipitated was formed. The precipitate was collected by filtration, washed with water (2×50 mL) and methanol (2×50 mL) to obtain pyrido[3,2-d]pyrimidine-2,4-diol (3.00 g, 63%) as white solid. ¹H NMR (400 MHz, Dimethylsulfoxide-d₆) δ 11.30 (bs, 2H), 8.44 (d, J=3.0 Hz, 1H), 7.70-7.48 (m, 2H); LCMS (ESI) m/z: 164.1 [M+H]⁺.

### Step 2: Synthesis of 2,4-dichloropyrido[3,2-d]pyrimidine

To a mixture of pyrido[3,2-d]pyrimidine-2,4-diol (1.60 g, 10 mmol) and phosphorus oxychloride (30 mL) was added N,N-diisopropylethylamine (2 mL) and the reaction mixture was stirred at 130° C. for 10 h. The reaction mixture was then concentrated under reduced pressure, and the volatiles were azeotroped with toluene (2×100 mL). The obtained residue was dissolved in ethyl acetate, filtered over celite and the filtrate was concentrated under reduced pressure to obtain 2,4-dichloropyrido[3,2-d]pyrimidine (1.30 g, 65%) as white solid. LCMS (ESI) m/z: 200.0 [M+H]⁺.

### Step 3: Synthesis of 4-(2-chloropyrido[3,2-d]pyrimidin-4-yl)morpholine

To a solution of 2,4-dichloropyrido[3,2-d]pyrimidine (5 g, 25.00 mmol) in THF (100 mL) was added morpholine (2.29 g, 26.25 mmol) and Et₃N (2.66 g, 26.25 mmol) at 0° C. The mixture was warmed up and stirred at 20° C. for 3 h and concentrated. The residue was dissolved in 150 mL chloroform, washed three times with saturated aqueous sodium bicarbonate solution, dried over Na₂SO₄, filtered and concentrated to obtain 4-(2-chloropyrido[3,2-d]pyrimidin-4-yl)morpholine (5.6 g, 89%) as a pale yellow solid. ¹H NMR (400 MHz, CHLOROFORM-d) δ=8.67 (dd, J=4.2, 1.8 Hz, 1H), 8.01 (dd, J=8.6, 1.8 Hz, 1H), 7.60 (dd, J=8.6, 4.2 Hz, 1H), 4.57 (bs, 4H), 3.86 (t, J=4.8 Hz, 4H).

### Step 4: Synthesis of 4-[2-(3-bromopyrazol-1-yl)pyrido[3,2-d]pyrimidin-4-yl]morpholine

To a solution of 4-(2-chloropyrido[3,2-d]pyrimidin-4-yl)morpholine (2 g, 7.98 mmol) in DMF (40 mL) were added 3-bromo-1H-pyrazole (1.17 g, 7.98 mmol) and Cs₂CO₃ (5.20 g, 15.96 mmol). The mixture was stirred at 100° C. for 16 h. 50 mL of water was added to the reaction mixture and it was extracted with ethyl acetate (60 mL*2). The combined organic layers were washed with brine (30 mL), dried over Na₂SO₄ and concentrated to obtain 4-[2-(3-bromopyrazol-1-yl)pyrido[3,2-d]pyrimidin-4-yl]morpholine (2.5 g, 87%) as a yellow solid. ¹H NMR (400 MHz, CHLOROFORM-d) δ=8.64 (dd, J=3.9, 1.8 Hz, 1H), 8.46 (d, J=2.6 Hz, 1H), 8.23 (dd, J=8.6, 1.5 Hz, 1H), 7.60 (dd, J=8.6, 4.2 Hz, 1H), 6.47 (d, J=2.6 Hz, 1H), 4.60 (bs, 4H), 3.91 (t, J=4.8 Hz, 4H).

### Step 5: Synthesis of tert-butyl 4-[1-(4-morpholinopyrido[3,2-d]pyrimidin-2-yl)pyrazol-3-yl]-3,6-dihydro-2H-pyridine-1-carboxylate

To a solution of 4-[2-(3-bromopyrazol-1-yl)pyrido[3,2-d]pyrimidin-4-yl]morpholine (0.5 g, 1.38 mmol) in dioxane (5 mL) and H₂O (1 mL) were added tert-butyl 4-(4,4,5,5-tetramethyl-1,3,2-dioxaborolan-2-yl)-3,6-dihydro-2H-pyridine-1-carboxylate (642 mg, 2.08 mmol), K₂CO₃ (478 mg, 3.46 mmol) and Pd(dppf)Cl₂ (101 mg, 0.138 mmol). The mixture was stirred at 60° C. for 3 h under nitrogen and then 15 mL of water was added to the mixture. It was then extracted with ethyl acetate (30 mL*2), washed with brine (15 mL) and dried over Na₂SO₄. The combined organic layer was concentrated and the crude product was purified by flash column chromatography (ISCO 10 g silica, 10-30% ethyl acetate in petroleum ether, gradient over 20 min) to obtain tert-butyl 4-[1-(4-morpholinopyrido[3,2-d]pyrimidin-2-yl)pyrazol-3-yl]-3,6-dihydro-2H-pyridine-1-carboxylate (400 mg, 62%) as a pale yellow solid. ¹H NMR (400 MHz, CHLOROFORM-d) δ=8.63 (dd, J=4.0, 1.3 Hz, 1H), 8.54 (d, J=2.6 Hz, 1H), 8.21 (dd, J=8.5, 1.2 Hz, 1H), 7.60 (dd, J=8.4, 4.2 Hz, 1H), 6.55 (d, J=2.4 Hz, 1H), 6.35 (bs, 1H), 4.62 (bs, 4H), 4.16-4.07 (m, 2H), 3.93 (t, J=4.8 Hz, 4H), 3.64 (t, J=5.2 Hz, 2H), 2.80 (bs, 2H), 1.50 (s, 9H).

### Step 6: Synthesis of tert-butyl 4-[1-(4-morpholinopyrido[3,2-d]pyrimidin-2-yl)pyrazol-3-yl]piperidine-1-carboxylate

To a solution of tert-butyl 4-[1-(4-morpholinopyrido[3,2-d]pyrimidin-2-yl)pyrazol-3-yl]-3,6-dihydro-2H-pyridine-1-carboxylate (350 mg, 755 umol) in MeOH (10 mL) was added Pd/C (100 mg, 10% purity) under argon. The resultant mixture hydrogenated under H₂ balloon (˜15 psi) at 25° C. for 12 h. It was then filtered and the filtrate was concentrated in vacuo. The crude product was purified by flash column (ISCO 4 g silica, 20-50% ethyl acetate in petroleum ether, gradient over 20 min) to obtain tert-butyl 4-[1-(4-morpholinopyrido[3,2-d]pyrimidin-2-yl)pyrazol-3-yl]piperidine-1-carboxylate (250 mg) as a pale yellow solid. ¹H NMR (400 MHz, CHLOROFORM-d) δ=8.63 (dd, J=4.1, 1.7 Hz, 1H), 8.50 (d, J=2.4 Hz, 1H), 8.26 (dd, J=8.5, 1.7 Hz, 1H), 7.59 (dd, J=8.5, 4.1 Hz, 1H), 6.30 (d, J=2.6 Hz, 1H), 4.61 (bs, 4H), 4.33-4.09 (m, 2H), 3.97-3.90 (m, 4H), 3.15-3.03 (m, 1H), 2.84 (t, J=12.6 Hz, 2H), 2.07-1.93 (m, 2H), 1.75-1.60 (m, 2H), 1.49 (s, 9H).

### Step 7: Synthesis of 4-[2-[3-(4-piperidyl)pyrazol-1-yl]pyrido[3,2-d]pyrimidin-4-yl]morpholine·HCl

A mixture of tert-butyl 4-[1-(4-morpholinopyrido[3,2-d]pyrimidin-2-yl)pyrazol-3-yl]piperidine-1-carboxylate (130 mg, 0.279 umol) in 4M HCl/EtOAc (10 mL) was stirred at 25° C. for 1 h. The reaction mixture was concentrated to obtain 4-[2-[3-(4-piperidyl)pyrazol-1-yl]pyrido[3,2-d]pyrimidin-4-yl]morpholine·HCl (130 mg, crude) as pale yellow solid.

### Step 8: Synthesis of 1-[4-[1-(4-morpholinopyrido[3,2-d]pyrimidin-2-yl)pyrazol-3-yl]-1-piperidyl]propan-1-one

To a solution of 4-[2-[3-(4-piperidyl)pyrazol-1-yl]pyrido[3,2-d]pyrimidin-4-yl]morpholine·HCl (130 mg, 323 umol) in DCM (3 mL) were added Et₃N (98 mg, 970 umol) and propanoyl chloride (36 mg, 388 umol) at 0° C. The mixture was warmed up and stirred at 20° C. for 1 h and concentrated. The resultant crude product was purified by prep-HPLC (Waters Xbridge Prep OBD C18 150*40 10u column, 30-50% acetonitrile in an a 10 mM ammonium bicarbonate solution in water, 8 min gradient) to obtain 1-[4-[1-(4-morpholinopyrido[3,2-d]pyrimidin-2-yl)pyrazol-3-yl]-1-piperidyl]propan-1-one (71 mg, 52%) as a white solid. ¹H NMR (400 MHz, CHLOROFORM-d) δ=8.63 (dd, J=4.2, 1.8 Hz, 1H), 8.50 (d, J=2.6 Hz, 1H), 8.26 (dd, J=8.6, 1.8 Hz, 1H), 7.59 (dd, J=8.6, 4.2 Hz, 1H), 6.29 (d, J=2.6 Hz, 1H), 4.93-4.24 (m, 5H), 4.00-3.87 (m, 5H), 3.26-3.07 (m, 2H), 2.69 (t, J=11.7 Hz, 1H), 2.39 (q, J=7.5 Hz, 2H), 2.17-1.99 (m, 2H), 1.78-1.65 (m, 2H), 1.18 (t, J=7.5 Hz, 3H). LCMS (ESI) for C₂₂H₂₇N₇O₂ [M+H]⁺: 422.3.

### Synthesis of tert-butyl 3-(1-(4-morpholinopyrido[3,2-d]pyrimidin-2-yl)-1H-pyrazol-3-yl)-5,6-dihydropyridine-1(2H)-carboxylate (Compound 5), tert-butyl 3-(1-(4-morpholinopyrido[3,2-d]pyrimidin-2-yl)-1H-pyrazol-3-yl)piperidine-1-carboxylate (Compound 6), 4-(2-(3-(piperidin-3-yl)-1H-pyrazol-1-yl)pyrido[3,2-d]pyrimidin-4-yl)morpholine (Compound 7) and 4-(2-(3-(1-methylpiperidin-3-yl)-1H-pyrazol-1-yl)pyrido[3,2-d]pyrimidin-4-yl)morpholine (Compound 8)

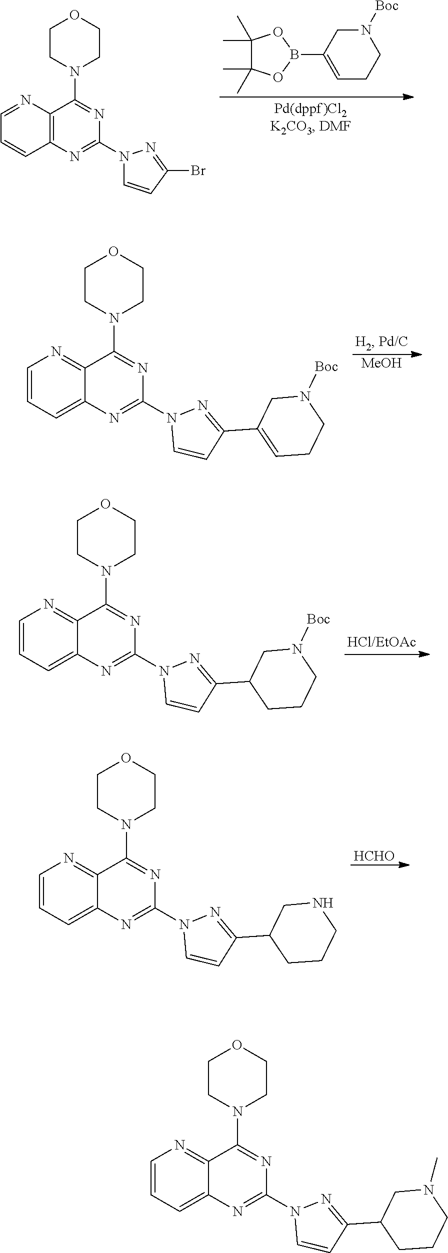

### Step 1: Synthesis of tert-butyl 3-(1-(4-morpholinopyrido[3,2-d]pyrimidin-2-yl)-1H-pyrazol-3-yl)-5,6-dihydropyridine-1(2H)-carboxylate

To a solution of 4-[2-(3-bromopyrazol-1-yl)pyrido[3,2-d]pyrimidin-4-yl]morpholine (400 mg, 1.11 mmol) in dioxane (6 mL) and H₂O (1.2 mL) were added tert-butyl 5-(4,4,5,5-tetramethyl-1,3,2-dioxaborolan-2-yl)-3,6-dihydro-2H-pyridine-1-carboxylate (411 mg, 1.33 mmol), K₂CO₃ (383 mg, 2.77 mmol), and Pd(dppf)Cl₂ (81 mg, 111 umol). The resultant mixture was stirred at 60° C. for 12 h under nitrogen. Then the reaction mixture was diluted with 2 mL H₂O and extracted with EtOAc (3 mL*3). The combined organic phase was dried over anhydrous Na₂SO₄ and concentrated under reduced pressure to give the crude product. It was purified by flash column (ISCO 40 g silica, 40-60% ethyl acetate in petroleum ether, gradient over 20 min) to obtain tert-butyl 5-[1-(4-morpholinopyrido[3,2-d]pyrimidin-2-yl)pyrazol-3-yl]-3,6-dihydro-2H-pyridine-1-carboxylate (480 mg, 94%) as a yellow solid. ¹H NMR (400 MHz, CHLOROFORM-d) δ 8.63 (dd, J=4.1, 1.7 Hz, 1H), 8.54 (d, J=2.6 Hz, 1H), 8.19 (dd, J=8.6, 1.5 Hz, 1H), 7.60 (dd, J=8.6, 4.2 Hz, 1H), 6.53 (bs, 2H), 4.63 (bs, 4H), 4.48 (s, 2H), 3.93 (t, J=4.8 Hz, 4H), 3.58 (t, J=5.4 Hz, 2H), 2.35 (bs, 2H), 1.51 (s, 9H). LCMS (ESI for C₂₄H₂₉N₇O₃ [M+H]⁺: 464.2.

### Step 2: Synthesis of tert-butyl 3-(1-(4-morpholinopyrido[3,2-d]pyrimidin-2-yl)-1H-pyrazol-3-yl)piperidine-1-carboxylate

To a solution of tert-butyl 5-[1-(4-morpholinopyrido[3,2-d]pyrimidin-2-yl)pyrazol-3-yl]-3,6-dihydro-2H-pyridine-1-carboxylate (340 mg, 734 umol) in MeOH (3 mL), was added PtO₂ (227 mg, 998 umol) and the resultant mixture was stirred at 25° C. for 1 h under hydrogen atmosphere. The mixture was filtered through celite and the filtrate was concentrated under vacuum. The crude product was purified by prep-HPLC (Waters Xbridge BEH C18 100*25 mm*5 um column; 42%-60% acetonitrile in an a 10 mM ammonium bicarbonate solution, 10 min gradient) to obtain tert-butyl 3-[1-(4-morpholinopyrido[3,2-d]pyrimidin-2-yl)pyrazol-3-yl]piperidine-1-carboxylate (300 mg) as white solid. 1H NMR (400 MHz, CHLOROFORM-d) δ=8.69-8.58 (m, 1H), 8.50 (d, J=2.4 Hz, 1H), 8.25 (d, J=8.4 Hz, 1H), 7.59 (dd, J=8.5, 4.1 Hz, 1H), 6.33 (d, J=2.6 Hz, 1H), 4.60 (bs, 3H), 4.36-4.03 (m, 2H), 4.01-3.86 (m, 4H), 3.15-3.05 (m, 1H), 3.02-2.92 (m, 1H), 2.83 (bs, 1H), 2.15 (bs, 1H), 1.73 (bs, 1H), 1.62 (bs, 3H), 1.46 (s, 9H). LCMS (ESI for C₂₄H₃₁N₇O₃ [M+H]⁺: 466.2.

### Step 3: Synthesis of 4-(2-(3-(piperidin-3-yl)-1H-pyrazol-1-yl)pyrido[3,2-d]pyrimidin-4-yl)morpholine

A solution of tert-butyl 3-[1-(4-morpholinopyrido[3,2-d]pyrimidin-2-yl)pyrazol-3-yl]piperidine-1-carboxylate (290 mg, 623 umol) in HCl/EtOAc (8 mL) was stirred at 25° C. for 1 h and concentrated. The mixture was basified by NH₃—H₂O to pH 9 at 0° C. and then it was concentrated again under vacuum. The resultant crude product was purified by prep-HPLC (Luna Omega 5u Polar C18 100 A column; 14-36% acetonitrile in an a 0.04% hydrochloric acid solution in water, 7 min gradient) to obtain 4-[2-[3-(3-piperidyl)pyrazol-1-yl]pyrido[3,2-d]pyrimidin-4-yl]morpholine (120 mg) as a white solid. ¹H NMR (400 MHz, DMSO-d₆) δ=9.07-8.64 (m, 2H), 8.29-8.17 (m, 1H), 7.82-7.77 (m, 1H), 6.53 (d, J=2.6 Hz, 1H), 4.57 (bs, 4H), 3.84 (t, J=4.5 Hz, 4H), 3.47-3.37 (m, 1H), 3.35-3.23 (m, 2H), 3.18-3.06 (m, 1H), 2.94 (bs, 1H), 2.18-2.06 (m, 1H), 1.96-1.84 (m, 2H), 1.78-1.67 (m, 1H). LCMS (ESI for C₁₉H₂₃N₇O [M+H]⁺: 366.2.

### Step 4: Synthesis of 4-(2-(3-(1-methylpiperidin-3-yl)-1H-pyrazol-1-yl)pyrido[3,2-d]pyrimidin-4-yl)morpholine

To a solution of 4-[2-[3-(3-piperidyl)pyrazol-1-yl]pyrido[3,2-d]pyrimidin-4-yl]morpholine (120 mg, 328 umol) in HCHO (2 mL), were added CH₃COOH (20 mg, 328 umol) and NaBH₃CN (21 mg, 328 umol, 1 eq) at 0° C., then the mixture was stirred at 20° C. for 12 h. The resultant reaction mixture was concentrated and the crude product was purified by prep-HPLC (Phenomenex gemini-NX C18 75*30 mm*3 um column; 15%-45% acetonitrile in an a 0.04% ammonium hydroxide and 10 mM ammonium bicarbonate solution, 10 min gradient) to obtain 4-[2-[3-(1-methyl-3-piperidyl)pyrazol-1-yl]pyrido[3,2-d]pyrimidin-4-yl]morpholine (29 mg, 23%) as a pale yellow solid. ¹H NMR (400 MHz, CHLOROFORM-d) δ 8.61 (dd, J=4.2, 1.7 Hz, 1H), 8.48 (d, J=2.9 Hz, 1H), 8.24 (dd, J=8.6, 1.7 Hz, 1H), 7.57 (dd, J=8.6, 4.2 Hz, 1H), 6.31 (d, J=2.4 Hz, 1H), 4.59 (bs, 4H), 4.03-3.66 (m, 4H), 3.35-3.16 (m, 2H), 2.89-2.80 (m, 1H), 2.20-2.05 (m, 3H), 2.00-1.99 (m, 2H), 1.98-1.95 (m, 1H), 1.86-1.71 (m, 2H), 1.56-1.37 (m, 1H). LCMS (ESI for C₂₀H₂₅N₇O [M+H]⁺: 380.2.

### Synthesis of 4-[2-(3-phenylpyrazol-1-yl)-6-tetrahydropyran-4-yl-pyrido[3,2-d]pyrimidin-4-yl]morpholine (Compound 9)

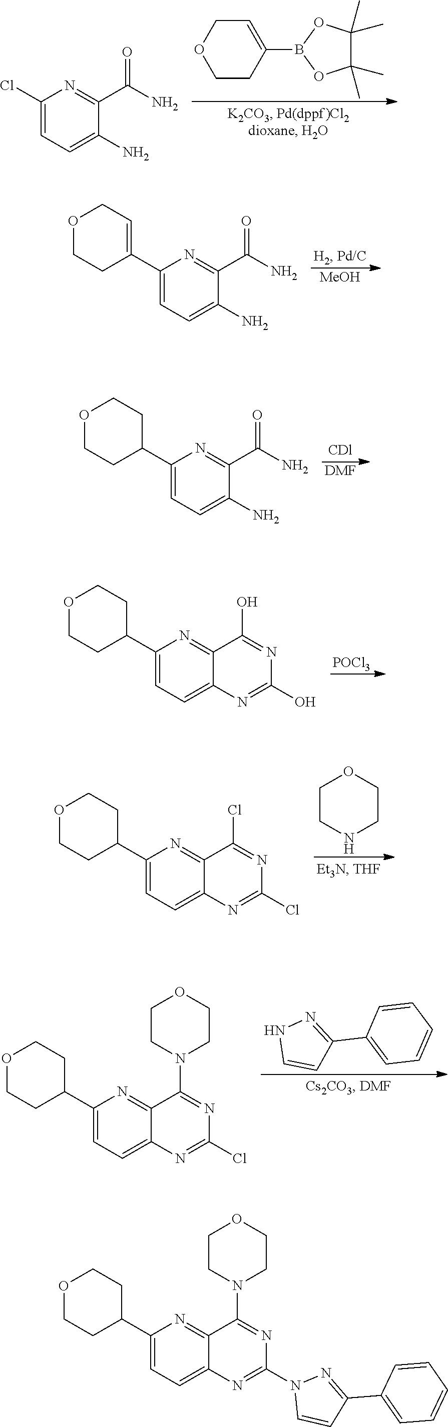

### Step 1: Synthesis of 3-amino-6-(3,6-dihydro-2H-pyran-4-yl)pyridine-2-carboxamide

To a solution of 3-amino-6-chloro-pyridine-2-carboxamide (3 g, 17.48 mmol) in dioxane (50 mL) and water (5 mL) were added 2-(3,6-dihydro-2H-pyran-4-yl)-4,4,5,5-tetramethyl-1,3,2-dioxaborolane (7.35 g, 34.97 mmol), K₂CO₃ (6.04 g, 43.71 mmol) and Pd(dppf)Cl₂ (640 mg, 874 umol). The resultant mixture was stirred at 80° C. for 16 h under nitrogen atmosphere. Water (20 mL) and EtOAc (50 mL) were added to the reaction mixture, filtered and filtrate was concentrated to obtain 3-amino-6-(3,6-dihydro-2H-pyran-4-yl)pyridine-2-carboxamide (2.2 g, 57%) as black solid. ¹H NMR (400 MHz, DMSO-d₆) δ=7.86 (bs, 1H), 7.49 (d, J=8.8 Hz, 1H), 7.33 (bs, 1H), 7.13 (d, J=8.8 Hz, 1H), 6.87 (bs, 2H), 6.51 (bs, 1H), 4.22 (d, J=2.4 Hz, 2H), 3.79 (t, J=5.4 Hz, 2H), 2.58-2.52 (m, 2H).

### Step 2: Synthesis of 3-amino-6-tetrahydropyran-4-yl-pyridine-2-carboxamide

To a solution of 3-amino-6-(3,6-dihydro-2H-pyran-4-yl)pyridine-2-carboxamide (2 g, 9.12 mmol) in MeOH (60 mL) was added Pd/C (1 g, 10% purity) under argon. The suspension was degassed under vacuum and purged with hydrogen several times. The mixture was stirred under hydrogen balloon (15 psi) at 20° C. for 16 h. The reaction mixture was filtered and the filtrate was concentrated in vacuo to obtain 3-amino-6-tetrahydropyran-4-yl-pyridine-2-carboxamide (1.6 g, 79%) as pale yellow solid. LCMS (ESI) m/z: 222.1 [M+H]+

### Step 3: Synthesis of 6-tetrahydropyran-4-ylpyrido[3,2-d]pyrimidine-2,4-diol

To a solution of 3-amino-6-tetrahydropyran-4-yl-pyridine-2-carboxamide (0.8 g, 3.62 mmol) in DMF (10 mL) was added CDI (879 mg, 5.42 mmol). The mixture was stirred at 90° C. for 16 h and cooled. The reaction mixture was filtered and the filtrate was concentrated in vacuo to obtain 6-tetrahydropyran-4-ylpyrido[3,2-d]pyrimidine-2,4-diol (0.3 g, 34%) as pale brown solid. LCMS (ESI) m/z: 248.1 [M+H]+

### Step 4: Synthesis of 2,4-dichloro-6-tetrahydropyran-4-yl-pyrido[3,2-d]pyrimidine

A mixture of 6-tetrahydropyran-4-ylpyrido[3,2-d]pyrimidine-2,4-diol (0.3 g, 1.21 mmol) in POCl₃ (4 mL) was stirred at 120° C. for 6 h. It was concentrated and 10 mL ice water was added. After stirring at 20° C. for 0.5 h, the mixture was basified by 2N NaOH (4 mL) and the reaction mixture was extracted with DCM (20 mL*2). The combined organic layers were washed with brine (15 mL), dried over Na₂SO₄ and concentrated to obtain 2,4-dichloro-6-tetrahydropyran-4-yl-pyrido[3,2-d]pyrimidine (200 mg) as brown solid. LCMS (ESI) m/z: 284.0 [M+H]+

### Step 5: Synthesis of 4-(2-chloro-6-tetrahydropyran-4-yl-pyrido[3,2-d]pyrimidin-4-yl)morpholine

To a solution of 2,4-dichloro-6-tetrahydropyran-4-yl-pyrido[3,2-d]pyrimidine (190 mg, 669 umol) in THF (8 mL) were added morpholine (61 mg, 702 umol) and Et₃N (71 mg, 702 umol) at 0° C. The resultant mixture was warmed up and stirred at 20° C. for 1 h. It was concentrated and the residue was dissolved in 30 mL chloroform, washed with a saturated aqueous solution of sodium bicarbonate (10 mL), dried over Na₂SO₄ and filtered. The resultant solution was concentrated to obtain 4-(2-chloro-6-tetrahydropyran-4-yl-pyrido[3,2-d]pyrimidin-4-yl)morpholine (0.2 g, crude) as pale brown solid.

### Step 6: Synthesis of 4-[2-(3-phenylpyrazol-1-yl)-6-tetrahydropyran-4-yl-pyrido[3,2-d]pyrimidin-4-yl]morpholine

To a solution of 4-(2-chloro-6-tetrahydropyran-4-yl-pyrido[3,2-d]pyrimidin-4-yl)morpholine (190 mg, 568 umol) in DMF (5 mL) were added 3-phenyl-1H-pyrazole (90 mg, 624 umol) and Cs₂CO₃ (370 mg, 1.14 mmol). The mixture was stirred at 100° C. for 16 h and then 15 mL of water was added to the reaction mixture. It was extracted with ethyl acetate (30 mL*2), washed with brine (15 mL), dried over Na₂SO₄ and concentrated. The crude product was purified by prep-HPLC (Nano-micro Kromasil C18 100*40 3u column; 1-42% acetonitrile in an a 0.04% hydrochloric acid solution in water, 8 min gradient) to obtain 4-[2-(3-phenylpyrazol-1-yl)-6-tetrahydropyran-4-yl-pyrido[3,2-d]pyrimidin-4-yl]morpholine (143 mg, 51%) as pale yellow solid. ¹H NMR (400 MHz, CHLOROFORM-d) δ=9.76 (d, J=8.8 Hz, 1H), 8.72 (bs, 1H), 8.19 (d, J=7.6 Hz, 2H), 7.69 (d, J=8.8 Hz, 1H), 7.51-7.33 (m, 3H), 7.00-6.93 (m, 1H), 5.28 (bs, 2H), 4.43 (bs, 2H), 4.14 (d, J=10.7 Hz, 2H), 4.03 (bs, 4H), 3.60 (dt, J=11.1, 3.4 Hz, 2H), 3.19-3.06 (m, 1H), 2.01-1.78 (m, 4H). LCMS (ESI) for C₂₅H₂₆N₆O₂ [M+H]⁺: 443.2.

### Synthesis of 4-[2-(4-phenylpyrazol-1-yl)-7-tetrahydropyran-4-yl-pyrido[3,2-d]pyrimidin-4-yl]morpholine (Compound 10)

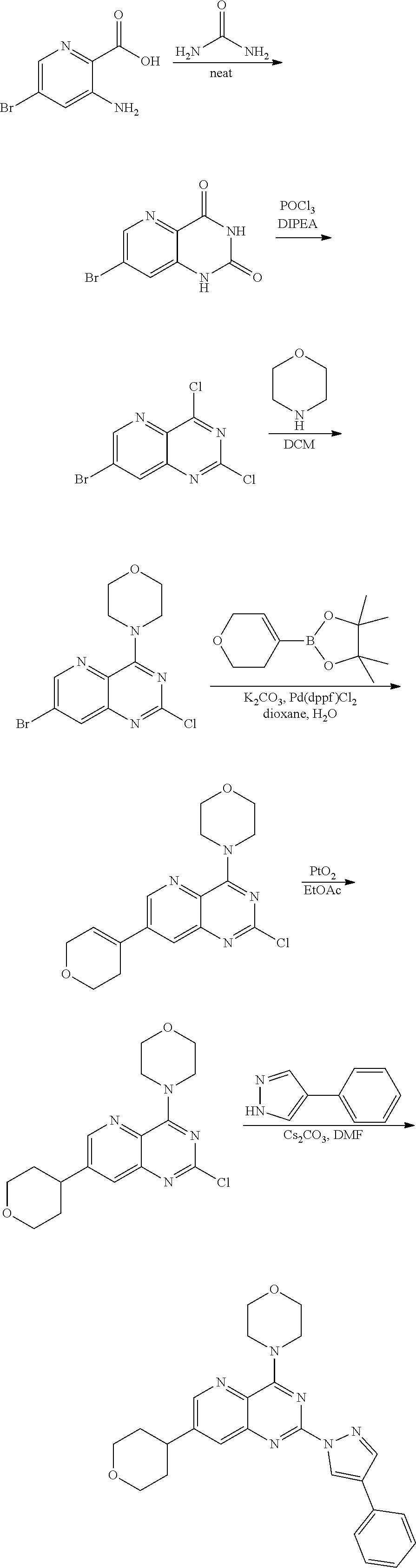

### Step 1: Synthesis of 7-bromo-1H-pyrido[3,2-d]pyrimidine-2,4-dione

A mixture of 3-amino-5-bromo-pyridine-2-carboxylic acid (5 g, 23.04 mmol) and urea (2.77 g, 46.08 mmol) was heated with stirring in a flask at 200° C. for 2 h. The mixture was cooled, water (100 mL) and MeOH (10 mL) were added to the flask and stirred. The resultant precipitate was filtered and dried obtain 7-bromo-1H-pyrido[3,2-d]pyrimidine-2,4-dione (4.6 g, 82%) as brown solid. ¹H NMR (400 MHz, DMSO-d₆) δ=11.55 (bs, 1H), 11.26 (bs, 1H), 8.52 (d, J=2.2 Hz, 1H), 7.73 (d, J=2.2 Hz, 1H).

### Step 2: Synthesis of 7-bromo-2,4-dichloro-pyrido[3,2-d]pyrimidine

To a mixture of 7-bromo-1H-pyrido[3,2-d]pyrimidine-2,4-dione (3 g, 12.40 mmol) in POCl₃ (25 mL) was added DIPEA (3.20 g, 24.79 mmol). The mixture was stirred at 120° C. for 1 h and concentrated. To the residue 30 mL ice water was added and stirred at 20° C. for 0.5 h and it was basified with 2N NaOH (30 mL). The resultant mixture was extracted with DCM (50 mL*2), washed with brine (30 mL), dried over Na₂SO₄ and concentrated to obtain 7-bromo-2,4-dichloro-pyrido[3,2-d]pyrimidine (3 g) as brown solid. ¹H NMR (400 MHz, CHLOROFORM-d) δ=9.11 (d, J=2.1 Hz, 1H), 8.49 (d, J=2.1 Hz, 1H)

### Step 3: Synthesis of 4-(7-bromo-2-chloro-pyrido[3,2-d]pyrimidin-4-yl)morpholine

To a solution of 7-bromo-2,4-dichloro-pyrido[3,2-d]pyrimidine (2.7 g, 9.68 mmol) in THF (50 mL) were added Et₃N (1.08 g, 10.65 mmol) and morpholine (928 mg, 10.65 mmol) at 0° C. The mixture was warmed up and stirred at 20° C. for 1 h and concentrated. The residue was dissolved in 100 mL chloroform, washed with saturated aqueous solution of sodium bicarbonate, dried over Na₂SO₄ and concentrated. The crude product was purified by flash column (ISCO 50 g silica, 0-50% ethyl acetate in petroleum ether, gradient over 20 min) to obtain 4-(7-bromo-2-chloro-pyrido[3,2-d]pyrimidin-4-yl)morpholine (2.5 g) as yellow solid.

### Step 4: Synthesis of 4-[2-chloro-7-(3,6-dihydro-2H-pyran-4-yl)pyrido[3,2-d]pyrimidin-4-yl]morpholine

To a solution of 4-(7-bromo-2-chloro-pyrido[3,2-d]pyrimidin-4-yl)morpholine (2.4 g, 7.28 mmol) in dioxane (30 mL) and H₂O (6 mL) were added 2-(3,6-dihydro-2H-pyran-4-yl)-4,4,5,5-tetramethyl-1,3,2-dioxaborolane (1.53 g, 7.28 mmol), K₂CO₃ (2.52 g, 18.20 mmol) and Pd(dppf)Cl₂ (266 mg, 364 umol) and the resultant stirred at 80° C. for 2 h under argon atmosphere. 30 mL of water was added to the reaction mixture and extracted with ethyl acetate (50 mL*2). The combined organic layers were washed with brine (30 mL), dried over Na₂SO₄ and concentrated. The crude product was purified by flash column (ISCO 20 g silica, 20-50% ethyl acetate in petroleum ether, gradient over 30 min) to obtain 4-[2-chloro-7-(3,6-dihydro-2H-pyran-4-yl)pyrido[3,2-d]pyrimidin-4-yl]morpholine (0.9 g, 2.70 mmol, 37%) as pale yellow solid.

### Step 5: Synthesis of 4-(2-chloro-7-tetrahydropyran-4-yl-pyrido[3,2-d]pyrimidin-4-yl)morpholine

To a solution of 4-[2-chloro-7-(3,6-dihydro-2H-pyran-4-yl)pyrido[3,2-d]pyrimidin-4-yl]morpholine (850 mg, 2.55 mmol) in EtOAc (20 mL) and DCM (20 mL) was added PtO₂ (580 mg, 2.55 mmol) under argon. The suspension was degassed under vacuum and purged with H₂ several times and further stirred under hydrogen balloon (15 psi) at 20° C. for 20 min. The mixture was then filtered and the filtrate was concentrated to obtain 4-(2-chloro-7-tetrahydropyran-4-yl-pyrido[3,2-d]pyrimidin-4-yl)morpholine (890 mg, crude) as pale yellow solid.

### Step 6: Synthesis of 4-[2-(4-phenylpyrazol-1-yl)-7-tetrahydropyran-4-yl-pyrido[3,2-d]pyrimidin-4-yl]morpholine

To a solution of 4-(2-chloro-7-tetrahydropyran-4-yl-pyrido[3,2-d]pyrimidin-4-yl)morpholine (0.1 g, 299 umol) in DMF (2 mL) were added 4-phenyl-1H-pyrazole (47 mg, 329 umol) and Cs₂CO₃ (195 mg, 597 umol). The resultant mixture was stirred at 100° C. for 16 h followed by the addition of 15 mL of water. The mixture was then extracted with ethyl acetate (30 mL*2), washed with brine (15 mL), dried over Na₂SO₄ and concentrated. The resultant crude product was purified by prep-HPLC (Phenomenex gemini-NX 150*30 5u column; 30-60% acetonitrile in an a 10 mM ammonium bicarbonate solution in water, 8 min gradient) to obtain 4-[2-(4-phenylpyrazol-1-yl)-7-tetrahydropyran-4-yl-pyrido[3,2-d]pyrimidin-4-yl]morpholine (65 mg, 49%) as pale yellow solid. ¹H NMR (400 MHz, CHLOROFORM-d) δ=8.79 (s, 1H), 8.56 (d, J=2.3 Hz, 1H), 8.12 (s, 1H), 8.06 (d, J=2.1 Hz, 1H), 7.67-7.60 (m, 2H), 7.43 (t, J=7.7 Hz, 2H), 7.34-7.27 (m, 1H), 4.65 (bs, 4H), 4.23-4.09 (m, 2H), 4.02-3.88 (m, 4H), 3.67-3.54 (m, 2H), 3.07-2.90 (m, 1H), 2.00-1.84 (m, 4H). LCMS (ESI) for C₂₅H₂₆N₆O₂ [M+H]⁺:443.3.

The following compounds were synthesized according to the protocol described above:

| Name | Structure | NMR, MS | # |
|---|---|---|---|
| 4-(2-(3-phenyl- 1H-pyrazol-1- yl)pyrido[3,2- d]pyrimidin-4- yl)morpholine | 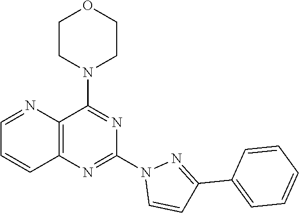 [SMILES: c1ccc(-c2ccn(-c3nc(N4CCOCC4)c4ncccc4n3)n2)cc1] | ¹H NMR (400 MHz, DMSO-d₆) δ 8.80 (d, J = 2.7 Hz, 1H), 8.76 (dd, J = 4.1, 1.7 Hz, 1H), 8.21 (dd, J = 8.5, 1.7 Hz, 1H), 8.01-7.95 (m, 2H), 7.83 (dd, J = 8.5, 4.1 Hz, 1H), 7.49 (t, J = 7.5 Hz, 2H), 7.40 (t, J = 7.3 Hz, 1H), 7.09 (d, J = 2.7 Hz, 1H), 4.58 (s, 4H), 3.83 (t, J = 8Hz, 4H). LCMS (ESI) m/z: 359.3 [M + H]⁺. | 11 |
| 4-(2-(3-(pyridin- 2-yl)-1H- pyrazol-1- yl)pyrido[3,2- d]pyrimidin-4- yl)morpholine | 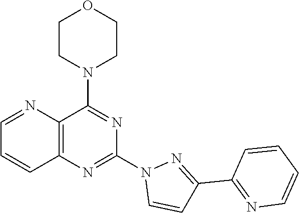 [SMILES: c1ccc(-c2ccn(-c3nc(N4CCOCC4)c4ncccc4n3)n2)nc1] | ¹H NMR (400 MHz, DMSO-d₆) δ 8.82 (d, J = 2.7 Hz, 1H), 8.77 (dd, J = 4.1, 1.7 Hz, 1H), 8.70-8.62 (m, 1H), 8.23 (dd, J = 8.5, 1.7 Hz, 1H), 8.15 (d, J = 7.9 Hz, 1H), 7.93 (td, J = 7.7, 1.8 Hz, 1H), 7.84 (dd, J = 8.5, 4.1 Hz, 1H), 7.41 (ddd, J = 7.5, 4.8, 1.1 Hz, 1H), 7.12 (d, J = 2.7 Hz, 1H), 4.60 (s, 4H), 3.84 (t, J = 8.0Hz, 4H). LCMS (ESI) m/z: 360.1 [M + H]⁺. | 12 |
| 4-(2-(3-(pyridin- 3-yl)-1H- pyrazol-1- yl)pyrido[3,2- d]pyrimidin-4- yl)morpholine | 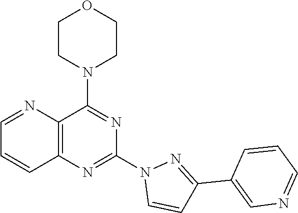 [SMILES: c1cncc(-c2ccn(-c3nc(N4CCOCC4)c4ncccc4n3)n2)c1] | ¹H NMR (400 MHz, DMSO-d₆) δ 9.24-9.13 (m, 1H), 8.84 (d, J = 2.7 Hz, 1H), 8.76 (dd, J = 4.1, 1.7 Hz, 1H), 8.60 (dd, J = 4.8, 1.6 Hz, 1H), 8.35 (dt, J = 8.0, 1.6Hz, 1H), 8.21 (dd, J = 8.5, 1.7 Hz, 1H), 7.83 (dd, J = 8.5, 4.1 Hz, 1H), 7.52 (ddd, J = 8.0, 4.8, 0.8 Hz, 1H), 7.20 (d, J = 2.7 Hz, 1H), 4.60 (bs, 4H), 3.92- 3.77 (m, 4H). LCMS (ESI) m/z: 360.1 [M + H]⁺. | 13 |
| 4-(2-(3-(pyridin- 4-yl)-1H- pyrazol-1- yl)pyrido[3,2- d]pyrimidin-4- yl)morpholine | 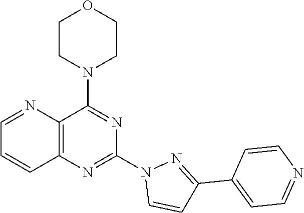 [SMILES: c1cnc2c(N3CCOCC3)nc(-n3ccc(-c4ccncc4)n3)nc2c1] | ¹H NMR (400 MHz, DMSO-d₆) δ 8.87 (d, J = 2.7 Hz, 1H), 8.78 (dd, J = 4.1, 1.7 Hz, 1H), 8.68 (dd, J = 4.5, 1.6 Hz, 2H), 8.23 (dd, J = 8.5, 1.7 Hz, 1H), 7.94 (dd, J = 4.5, 1.6 Hz, 2H), 7.84 (dd, J = 8.5, 4.2 Hz, 1H), 7.25 (d, J = 2.7 Hz, 1H), 4.59 (bs, 4H), 3.90-3.80 (m, 4H). LCMS (ESI) m/z: 360.1 [M + H]⁺. | 14 |
| 4-[2-(3- phenylpyrazol- 1-yl)-7- tetrahydro- pyran-4-yl- pyrido[3,2- d]pyrimidin-4- yl]morpholine | 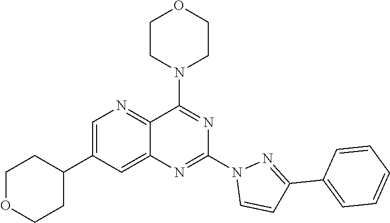 [SMILES: c1ccc(-c2ccn(-c3nc(N4CCOCC4)c4ncc(C5CCOCC5)cc4n3)n2)cc1] | ¹H NMR (400MHz, CHLOROFORM-d) δ = 9.57 (s, 1H), 8.76 (bs, 1H), 8.64 (d, J = 1.6 Hz, 1H), 8.08 (bs d, J = 6.8 Hz, 2H), 7.34-7.24 (m, 3H), 6.97 (bs, 1H), 5.43-4.22 (m, 4H), 4.20-4.11 (m, 2H), 3.98 (bs, 4H), 3.60 (dt, J = 11.4, 2.3Hz, 2H), 3.23-3.06 (m, 1H), 2.10-1.87 (m, 4H). LCMS (ESI) for C25H26N6O2 [M + H]+: 443.2. | 15 |
| 4-(7-(3,6- dihydro-2H- pyran-4-yl)-2- (3-(3- methoxyphenyl)- 1H-pyrazol-1- yl)pyrido[3,2- d]pyrimidin-4- yl)morpholine | 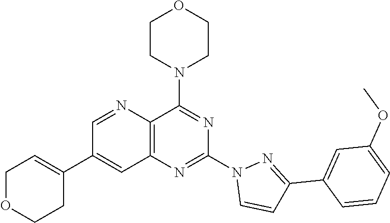 [SMILES: COc1cccc(-c2ccn(-c3nc(N4CCOCC4)c4ncc(C5=CCOCC5)cc4n3)n2)c1] | ¹H NMR (400 MHz, DMSO-d₆) δ 8.97 (d, J = 2.3 Hz, 1H), 8.78 (d, J = 2.7 Hz, 1H), 8.10 (d, J = 2.2 Hz, 1H), 7.56 (d, J = 7.8 Hz, 1H), 7.51 (d, J = 2.3Hz, 1H), 7.40 (t, J = 8.0 Hz, 1H), 7.10 (d, J = 2.7 Hz, 1H), 6.97 (dd, J = 7.9, 2.2Hz, 1H), 6.78 (s, 1H), 4.57 (bs, 4H), 4.32 (d, J = 2.6 Hz, 2H), 3.89 (t, J = 5.4 Hz, 2H), 3.87-3.78 (m, 7H), 2.61 (s, 2H); LCMS (ESI) m/z: 471.1 [M + H]⁺. | 16 |
| 4-(2-(3-(4- methoxyphenyl)- 1H-pyrazol-1- yl)pyrido[3,2- d]pyrimidin-4- yl)morpholine | 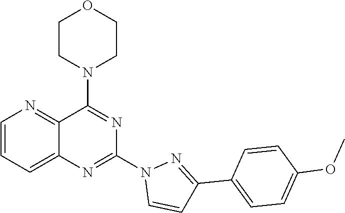 [SMILES: COc1ccc(-c2ccn(-c3nc(N4CCOCC4)c4ncccc4n3)n2)cc1] | ¹H NMR (400 MHz, CDCl₃) δ 8.63 (dd, J = 4.0, 1.6Hz, 1H), 8.60 (d, J = 4Hz, 1H), 8.25 (dd, J = 8.5, 1.7 Hz, 1H), 8.00-7.90 (m, 2H), 7.60 (dd, J = 8.5, 4.1 Hz, 1H), 6.97 (d, J = 8.8 Hz, 2H), 6.74 (d, J = 2.7 Hz, 1H), 4.64 (bs, 4H), 3.94 (t, J = 8Hz, 4H), 3.86 (s, 3H); LCMS (ESI) m/z: 389.1 [M + H]⁺. | 17 |
| 4-(2-(3- isopropyl-1H- pyrazol-1- yl)pyrido[3,2- d]pyrimidin-4- yl)morpholine | 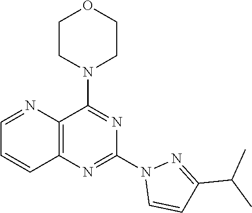 [SMILES: CC(C)c1ccn(-c2nc(N3CCOCC3)c3ncccc3n2)n1] | ¹H NMR (400 MHz, DMSO-d₆) δ 8.72 (dd, J = 4.1, 1.6 Hz, 1H), 8.60 (d, J = 2.6 Hz, 1H), 8.15 (dd, J = 8.5, 1.6 Hz, 1H), 7.80 (dd, J = 8.5, 4.1 Hz, 1H), 6.43 (d, J = 2.6 Hz, 1H), 4.50 (bs, 4H), 3.81 (t, J = 4Hz, 4H), 3.04-2.99 (m, 1H), 1.26 (d, J = 6.9 Hz, 6H). LCMS (ESI) m/z: 325.2 [M + H]⁺. | 18 |
| 4-[2-[3-(2- fluorophenyl) pyrazol-1-yl]-7- tetrahydropyran- 4-yl- pyrido[3,2- d]pyrimidin-4- yl]morpholine | 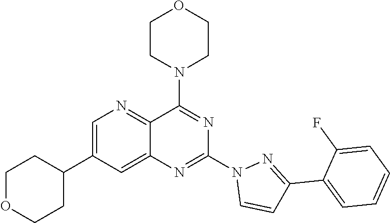 [SMILES: Fc1ccccc1-c1ccn(-c2nc(N3CCOCC3)c3ncc(C4CCOCC4)cc3n2)n1] | ¹H NMR (400MHz, CHLOROFORM-d) δ = 8.65 (d, J = 2.6 Hz, 1H), 8.56 (d, J = 2.1 Hz, 1H), 8.33 (dt, J = 7.7, 1.4Hz, 1H), 8.08 (d, J = 2.1 Hz, 1H), 7.38- 7.30 (m, 1H), 7.26-7.21 (m, 1H), 7.15 (dd, J = 11.1, 8.4Hz, 1H), 7.00-6.93 (m, 1H), 4.65 (bs, 4H), 4.23- 4.08 (m, 2H), 3.93 (t, J = 4.4Hz, 4H), 3.60 (dt, J = 11.2, 3.1Hz, 2H), 3.04-2.90 (m, 1H), 1.99-1.84 (m, 4H). LCMS (ESI) for C25H25FN6O2 [M + H]+: 461.3 | 19 |
| 4-[2-[3-(4- fluorophenyl) pyrazol-1-yl]-7- tetrahydropyran- 4-yl- pyrido[3,2- d]pyrimidin-4- yl]morpholine | 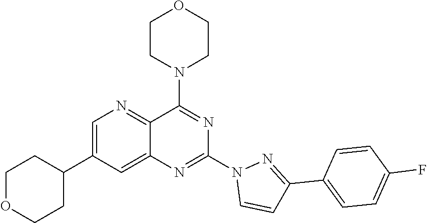 [SMILES: Fc1ccc(-c2ccn(-c3nc(N4CCOCC4)c4ncc(C5CCOCC5)cc4n3)n2)cc1] | ¹H NMR (400MHz, CHLOROFORM-d) δ = 8.63 (d, J = 2.8 Hz, 1H), 8.55 (d, J = 2.1 Hz, 1H), 8.07 (d, J = 2.1 Hz, 1H), 8.04-7.96 (m, 2H), 7.13 (t, J = 8.7 Hz, 2H), 6.76 (d, J = 2.8 Hz, 1H), 4.64 (bs, 4H), 4.15 (dd, J = 10.4, 2.8Hz, 2H), 3.94 (t, J = 4.8Hz, 4H), 3.60 (dt, J = 11.2, 3.2Hz, 2H), 3.05-2.92 (m, 1H), 1.98-1.82 (m, 4H). LCMS (ESI) for C25H25FN6O2 [M + H]+: 461.2. | 20 |
| 4-[2-[3-(2- pyridyl)pyrazol- 1-yl]-7- tetrahydropyran- 4-yl- pyrido[3,2- d]pyrimidin-4- yl]morpholine | 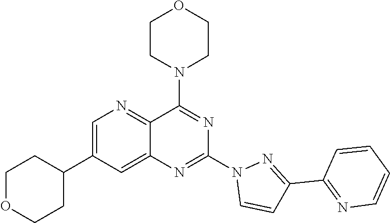 [SMILES: c1ccc(-c2ccn(-c3nc(N4CCOCC4)c4ncc(C5CCOCC5)cc4n3)n2)nc1] | ¹H NMR (400MHz, CHLOROFORM-d) δ = 8.71- 8.63 (m, 2H), 8.56 (d, J = 2.3 Hz, 1H), 8.38 (d, J = 8.0 Hz, 1H), 8.08 (d, J = 2.0 Hz, 1H), 7.79 (dt, J = 1.7, 7.7 Hz, 1H), 7.29 (d, J = 0.8 Hz, 1H), 7.20 (d, J = 2.6 Hz, 1H), 4.66 (bs, 4H), 4.16 (dd, J = 10.4, 2.8Hz, 2H), 4.03-3.89 (m, 4H), 3.61 (dt, J = 11.2, 3.1Hz, 2H), 3.08-2.93 (m, 1H), 2.03-1.81 (m, 4H). LCMS (ESI) for C24H25N7O2 [M + H]+: 444.3. | 21 |

### Synthesis of 4-[2-[3-(3-fluorophenyl)pyrazol-1-yl]-7-tetrahydropyran-4-yl-pyrido[3,2-d]pyrimidin-4-yl]morpholine (Compound 22)

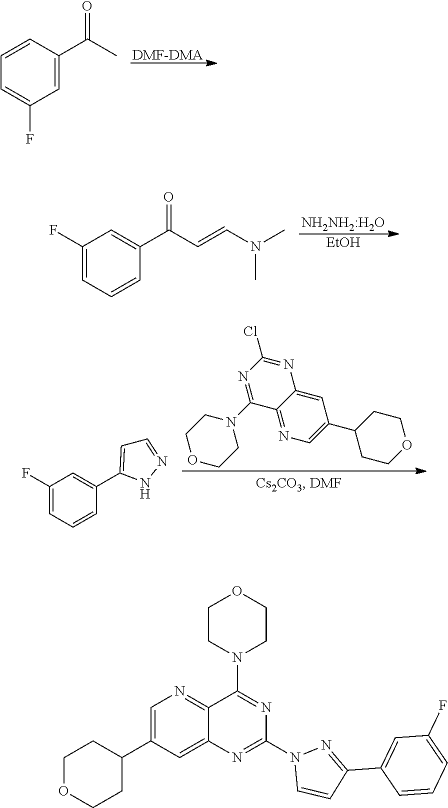

### Step 1: Synthesis of (E)-3-(dimethylamino)-1-(3-fluorophenyl)prop-2-en-1-one

A solution of 1-(3-fluorophenyl)ethanone (1 g, 7.24 mmol) in DMF-DMA (7 mL) was stirred at 100° C. for 12 h and concentrated. The resultant crude product was purified by flash column chromatography (ISCO 40 g silica, 0-40% ethyl acetate in petroleum ether, gradient over 20 min) to obtain (E)-3-(dimethylamino)-1-(3-fluorophenyl)prop-2-en-1-one (850 mg, 61%) as yellow solid. ¹H NMR (400 MHz, DMSO-d₆) δ=7.73 (d, J=11.6 Hz, 2H), 7.64 (d, J=10 Hz, 1H), 7.46 (dd, J=7.6, 1.6 Hz, 1H), 7.44-7.31 (m, 1H), 5.83 (d, J=12 Hz, 1H), 3.1 (s, 1H), 2.9 (s, 3H).

### Step 2: Synthesis of 5-(3-fluorophenyl)-1H-pyrazole

To a solution of (E)-3-(dimethylamino)-1-(3-fluorophenyl)prop-2-en-1-one (520 mg, 2.69 mmol) in EtOH (1 mL) was added hydrazine;hydrate (250 mg, 5.38 mmol) and the mixture was stirred at 15° C. for 10 h. 5 mL of water and 5 mL of ethyl acetate were added to the reaction mixture, the organic layer separated and aqueous layer was extracted with EtOAc (5 mL*3). The combined organic layers were washed with brine (5 mL*3), dried over Na₂SO₄, filtered and concentrated to obtain 5-(3-fluorophenyl)-1H-pyrazole (480 mg) as yellow solid. LCMS (ESI) m/z: 163.1 [M+H]⁺

### Step 3: Synthesis of 4-[2-[3-(3-fluorophenyl)pyrazol-1-yl]-7-tetrahydropyran-4-yl-pyrido[3,2-d]pyrimidin-4-yl]morpholine

A mixture of 4-(2-chloro-7-tetrahydropyran-4-yl-pyrido[3,2-d]pyrimidin-4-yl)morpholine (100 mg, 299 umol), 5-(3-fluorophenyl)-1H-pyrazole (53 mg, 329 umol) and Cs₂CO₃ (195 mg, 597 umol) in 1 mL of DMF was stirred at 100° C. for 10 h. The mixture was filtered and the filtrate was concentrated and subjected to prep-HPLC (Waters X bridge 150*30 mm*5 uM column; 30-60% acetonitrile in an a 10 mM ammonium bicarbonate solution in water, 8 min gradient) to afford 4-[2-[3-(3-fluorophenyl)pyrazol-1-yl]-7-tetrahydropyran-4-yl-pyrido[3,2-d]pyrimidin-4-yl]morpholine (54 mg, 39%) as white solid. 1H NMR (400 MHz, DMSO-d₆) δ=8.80 (d, J=2.4 Hz, 1H), 8.72 (d, J=2 Hz, 1H), 8.00 (s, 1H), 7.83 (d, J=7.6 Hz, 1H), 7.76 (d, J=10.4 Hz, 1H), 7.55 (q, 1H), 7.26-7.22 (m, 1H), 7.15 (d, J=2.8 Hz, 1H), 4.57 (bs, 4H), 4.02-3.99 (m, 2H), 3.84-3.82 (m, 4H), 3.83-3.50 (m, 2H), 3.48-3.06 (m, 1H), 1.85-1.77 (m, 4H). LCMS (ESI for C₂₅H₂₅FN₆O₂) [M+H]⁺: 461.2.

### Synthesis of 4-(2-(3-Phenyl-1H-pyrazol-1-yl)-7-(tetrahydro-2H-pyran-2-yl)pyrido[3,2-d]pyrimidin-4-yl)morpholine (Compound 23)

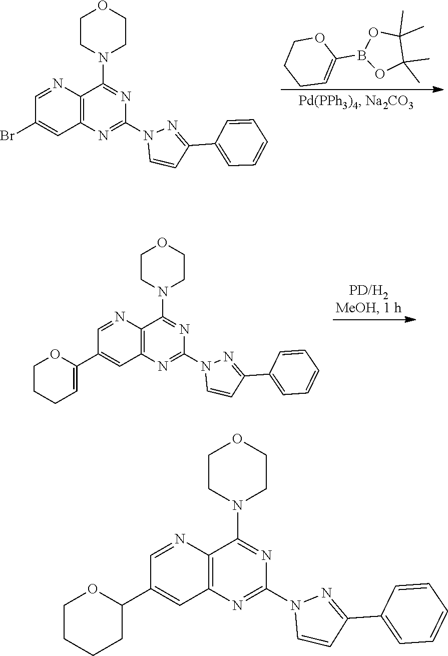 [SMILES: Brc1cnc2c(N3CCOCC3)nc(-n3ccc(-c4ccccc4)n3)nc2c1 ; CC1(C)OB(C2=CCCCO2)OC1(C)C ; C1=C(c2cnc3c(N4CCOCC4)nc(-n4ccc(-c5ccccc5)n4)nc3c2)OCCC1 ; c1ccc(-c2ccn(-c3nc(N4CCOCC4)c4ncc(C5CCCCO5)cc4n3)n2)cc1]

### Step 1: Synthesis of 4-(7-(3,4-Dihydro-2H-pyran-6-yl)-2-(3-phenyl-1H-pyrazol-1-yl)pyrido[3,2-d]pyrimidin-4-yl)morpholine

A solution of 4-(7-Bromo-2-(3-phenyl-1H-pyrazol-1-yl)pyrido[3,2-d]pyrimidin-4-yl)morpholine (100 mg, 0.23 mmol), 2-(3,4-dihydro-2H-pyran-6-yl)-4,4,5,5-tetramethyl-1,3,2-dioxaborolane (97 mg, 0.46 mmol), tetrakis(triphenylphosphine)palladium (23 mg, 0.023 mmol) and sodium carbonate (29 mg, 0.28 mmol) in water (0.5 mL) and dioxane (2.0 mL) was stirred at 80° C. for 4 h under argon atmosphere. Water (25 mL) was added to the mixture and then it was extracted with dichloromethane (25 ml*3). The combined organic layer was dried on Na₂SO₄, filtered and concentrated. The obtained residue was subjected to silica gel column chromatography (petroleum ether/ethyl acetate=4:1) to obtain 4-(7-(3,4-dihydro-2H-pyran-6-yl)-2-(3-phenyl-1H-pyrazol-1-yl)pyrido[3,2-d]pyrimidin-4-yl)morpholine (60 mg, 60%) as yellow solid. LCMS (ESI) m/z: 441.1 [M+H]⁺.

### Step 2: Synthesis of 4-(2-(3-Phenyl-1H-pyrazol-1-yl)-7-(tetrahydro-2H-pyran-2-yl)pyrido[3,2-d]pyrimidin-4-yl)morpholine

Palladium on carbon (4 mg, 10% loading) was added to a solution of 4-(7-(3,4-dihydro-2H-pyran-6-yl)-2-(3-phenyl-1H-pyrazol-1-yl)pyrido[3,2-d]pyrimidin-4-yl)morpholine (40 mg, 0.019 mmol) in methanol (5 ml) and the resultant mixture was stirred at 20° C. for 1 h under hydrogen atmosphere. The mixture was filtered, concentrated and subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 4-(2-(3-phenyl-1H-pyrazol-1-yl)-7-(tetrahydro-2H-pyran-2-yl)pyrido[3,2-d]pyrimidin-4-yl)morpholine (34.2 mg, 78%) as white solid. ¹H NMR (400 MHz, DMSO-d₆) δ 8.78 (d, J=2.0 Hz, 1H), 8.72 (d, J=1.2 Hz, 1H), 8.04 (d, J=1.2 Hz, 1H), 7.99-7.97 (m, 2H), 7.50-7.38 (m, 3H), 7.08 (d, J=1.6 Hz, 1H), 4.63-4.61 (m, 5H), 4.11 (d, J=8.4 Hz, 1H), 3.83-3.52 (m, 4H), 3.36-3.29 (m, 1H), 2.01-1.90 (m, 2H), 1.60-1.49 (m, 4H); LCMS (ESI) m/z: 443.3 [M+H]⁺.

### Synthesis of 4-(2-(3-Phenyl-1H-pyrazol-1-yl)-7-(tetrahydro-2H-pyran-3-yl)pyrido[3,2-d]pyrimidin-4-yl)morpholine (Compound 24)

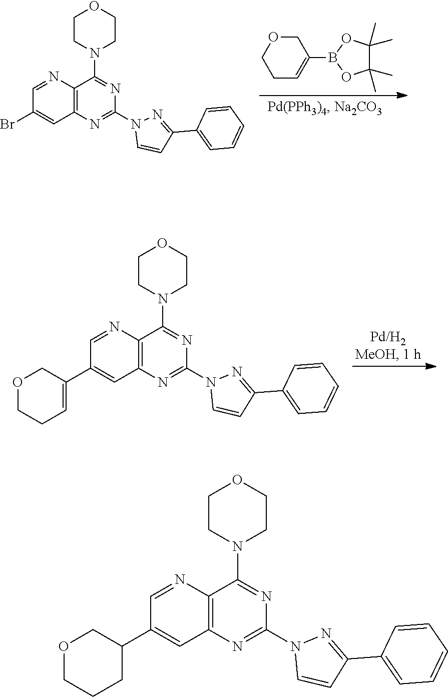 [SMILES: Brc1cnc2c(N3CCOCC3)nc(-n3ccc(-c4ccccc4)n3)nc2c1 ; CC1(C)OB(C2=CCCOC2)OC1(C)C ; C1=C(c2cnc3c(N4CCOCC4)nc(-n4ccc(-c5ccccc5)n4)nc3c2)COCC1 ; c1ccc(-c2ccn(-c3nc(N4CCOCC4)c4ncc(C5CCCOC5)cc4n3)n2)cc1]

### Step 1: Synthesis of 4-(7-(5,6-Dihydro-2H-pyran-3-yl)-2-(3-phenyl-1H-pyrazol-1-yl)pyrido[3,2-d]pyrimidin-4-yl)morpholine

A mixture of 4-(7-Bromo-2-(3-phenyl-1H-pyrazol-1-yl)pyrido[3,2-d]pyrimidin-4-yl)morpholine (60 mg, 0.14 mmol), 2-(3,4-dihydro-2H-pyran-6-yl)-4,4,5,5-tetramethyl-1,3,2-dioxaborolane (59 mg, 0.28 mmol), tetrakis(triphenylphosphine)palladium (16 mg, 0.014 mmol) and sodium carbonate (18 mg, 0.17 mmol) in water (0.5 mL) and dioxane (2.0 mL) was stirred at 80° C. for 4 under argon atmosphere. Water (25 mL) was added to the reaction mixture and then extracted with dichloromethane (25 ml*3). The combined organic layer was dried over Na₂SO₄, filtered and concentrated. The residue obtained was subjected to silica gel column chromatography (petroleum ether/ethyl acetate=4:1) to obtain 4-(7-(5,6-dihydro-2H-pyran-3-yl)-2-(3-phenyl-1H-pyrazol-1-yl)pyrido[3,2-d]pyrimidin-4-yl)morpholine (30 mg, 48%) as yellow solid. LCMS (ESI) m/z: 441.1 [M+H]⁺.

### Step 2: Synthesis of 4-(2-(3-Phenyl-1H-pyrazol-1-yl)-7-(tetrahydro-2H-pyran-3-yl)pyrido[3,2-d]pyrimidin-4-yl)morpholine

Palladium on carbon (4 mg) was added to a solution of 4-(7-(5,6-dihydro-2H-pyran-3-yl)-2-(3-phenyl-1H-pyrazol-1-yl)pyrido[3,2-d]pyrimidin-4-yl)morpholine (30 mg, 0.068 mmol) in methanol (5 ml) and the resultant mixture was stirred at 20° C. for 1 h under hydrogen atmosphere. It was then filtered and concentrated. The residue obtained was subjected to prep-HPLC(SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to afford 4-(2-(3-phenyl-1H-pyrazol-1-yl)-7-(tetrahydro-2H-pyran-3-yl)pyrido[3,2-d]pyrimidin-4-yl)morpholine (6.1 mg, 20%) as white solid. ¹H NMR (400 MHz, DMSO-d₆) δ 8.78 (d, J=2.4 Hz, 1H), 8.71 (d, J=2.0 Hz, 1H), 8.07 (d, J=2.0 Hz, 1H), 7.99-7.96 (m, 2H), 7.50 (t, J=4.8 Hz, 2H), 7.39 (t, J=5.4 Hz, 1H), 7.08 (d, J=2.8 Hz, 1H), 4.57 (bs, 4H), 3.97-3.94 (m, 2H), 3.91-3.81 (m, 4H), 3.56-3.47 (m, 2H), 3.08-3.06 (m, 1H), 2.07-1.72 (m, 2H), 1.71-1.69 (m, 2H); LCMS (ESI) m/z: 443.3 [M+H]⁺.

### Synthesis of 4-(2-(3-(3-fluorophenyl)-1H-pyrazol-1-yl)pyrido[3,2-d]pyrimidin-4-yl)morpholine (Compound 25) and 4-(2-(5-(3-fluorophenyl)-1H-pyrazol-1-yl)pyrido[3,2-d]pyrimidin-4-yl)morpholine (Compound 26)

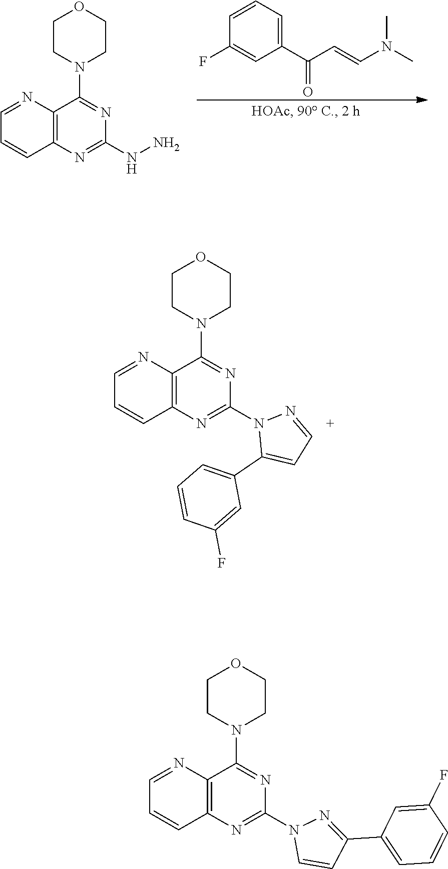 [SMILES: NNc1nc(N2CCOCC2)c2ncccc2n1 ; CN(C)/C=C/C(=O)c1cccc(F)c1 ; Fc1cccc(-c2ccnn2-c2nc(N3CCOCC3)c3ncccc3n2)c1 ; Fc1cccc(-c2ccn(-c3nc(N4CCOCC4)c4ncccc4n3)n2)c1]

A mixture of 4-(2-hydrazineylpyrido[3,2-d]pyrimidin-4-yl)morpholine (130 mg, 0.0.5 mmol) and (E)-3-(dimethylamino)-1-(3-fluorophenyl)prop-2-en-1-one (184 mg, 0.95 mmol) in acetic acid (3 mL) was stirred at 90° C. for 2 h. The reaction mixture was concentrated and the residue was purified by prep-HPLC (SunFire C18, 4.6*50 mm, 3.5 um column. The mobile phase was acetonitrile/10 mM ammonium bicarbonate aqueous solution) to obtain the target compounds:

Compound 25: 4-(2-(3-(3-fluorophenyl)-1H-pyrazol-1-yl)pyrido[3,2-d]pyrimidin-4-yl)morpholine (2.6 mg, 1%) was isolated as white solid. ¹H NMR (400 MHz, DMSO-d₆) δ 8.82 (d, J=2.7 Hz, 1H), 8.76 (dd, J=4.1, 1.7 Hz, 1H), 8.22 (dd, J=8.5, 1.7 Hz, 1H), 7.84 (dd, J=8.5, 4.3 Hz, 2H), 7.78 (d, J=9.7 Hz, 1H), 7.54 (dd, J=14.2, 8.0 Hz, 1H), 7.24 (t, J=8.4 Hz, 1H), 7.16 (d, J=2.7 Hz, 1H), 4.58 (bs, 4H), 3.87-3.81 (m, 4H). LCMS (ESI) m/z: 377.1 [M+H]⁺.

Compound 26: 4-(2-(5-(3-fluorophenyl)-1H-pyrazol-1-yl)pyrido[3,2-d]pyrimidin-4-yl)morpholine (12 mg, 6%) was isolated as white solid. ¹H NMR (400 MHz, DMSO-d₆) δ 8.78 (dd, J=4.1, 1.7 Hz, 1H), 8.11 (dd, J=8.5, 1.7 Hz, 1H), 7.86-7.78 (m, 2H), 7.41 (dd, J=15.3, 7.6 Hz, 1H), 7.21 (dd, J=7.1, 4.3 Hz, 2H), 7.11 (d, J=7.8 Hz, 1H), 6.70 (d, J=1.6 Hz, 1H), 3.58 (bs, 4H), 3.53 (s, 4H). LCMS (ESI) m/z: 377.1 [M+H]⁺.

### Synthesis of 4-(2-(3-(3-methoxyphenyl)-1H-pyrazol-1-yl)pyrido[3,2-d]pyrimidin-4-yl)morpholine (Compound 27)

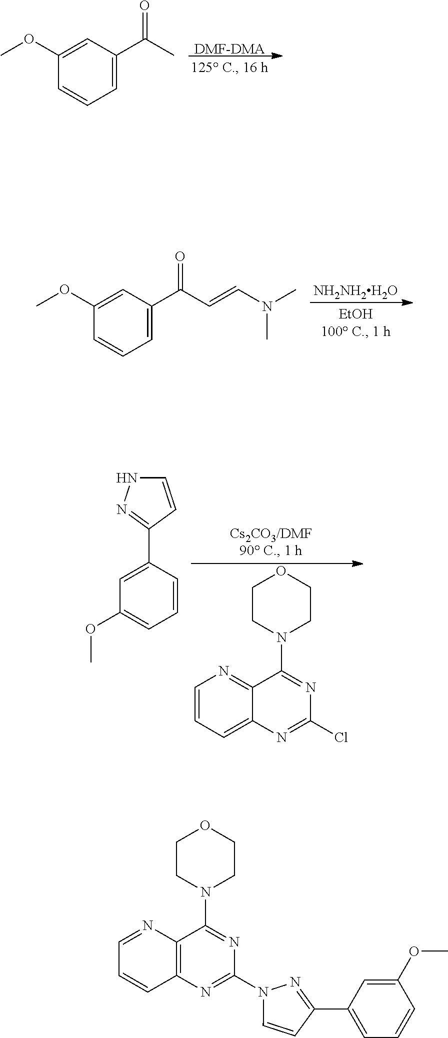

### Step 1: Synthesis of (E)-3-(dimethylamino)-1-(3-methoxyphenyl)prop-2-en-1-one

A mixture of 1-(3-methoxyphenyl)ethanone (10.0 g, 66.6 mmol) and DMF-DMA (20 mL) was stirred at 120° C. under nitrogen atmosphere for 16 h. The mixture was then poured into water and extracted with ethyl acetate (150 mL*2). The combined organic phase was concentrated to afford 3-(dimethylamino)-1-(3-methoxyphenyl)prop-2-en-1-one (18.0 g) as brown oil. LCMS (ESI) m/z: 206.2 [M+H]⁺.

### Step 2: Synthesis of 3-(3-methoxyphenyl)-1H-pyrazole

A mixture of 3-(dimethylamino)-1-(3-methoxyphenyl)prop-2-en-1-one (18.0 g), 85% hydrazine hydrate solution (8 mL) and ethanol (100 mL) was stirred at 100° C. for 1 h. The mixture was poured into water, extracted with ethyl acetate (200 mL*2) and the combined organics were concentrated. The residue was purified by silica gel column chromatography (30% petroleum ether in ethyl acetate) to afford 3-(3-methoxyphenyl)-1H-pyrazole (10.5 g) as off-white solid. LCMS (ESI) m/z: 175.2 [M+H]⁺.

### Step 3: Synthesis of 4-(2-(3-(3-methoxyphenyl)-1H-pyrazol-1-yl)pyrido[3,2-d]pyrimidin-4-yl)morpholine

A mixture of 4-(2-chloropyrido[3,2-d]pyrimidin-4-yl)morpholine (143 mg, 0.57 mmol), 3-(3-methoxyphenyl)-1H-pyrazole (100 mg, 0.57 mmol), cesium carbonate (370 mg, 1.14 mmol) and N,N-dimethylformamide (5 mL) was stirred at 90° C. for 2 h. The resultant mixture was filtered and the crude product from the filtrate was purified by prep-HPLC (Column Xbridge 21.2*250 mm C18, 10 um, Mobile Phase A: water (10 mmol/L ammonium bicarbonate) B: acetonitrile) to afford 4-(2-(3-(3-methoxyphenyl)-1H-pyrazol-1-yl)pyrido[3,2-d]pyrimidin-4-yl)morpholine (47.5 mg, 21%) as light yellow solid. ¹H NMR (400 MHz, CDCl₃) δ 8.68-8.59 (m, 2H), 8.24 (dd, J=8.5, 1.7 Hz, 1H), 7.66-7.58 (m, 2H), 7.58-7.53 (m, 1H), 7.34 (t, J=7.9 Hz, 1H), 6.92 (td, J=8.2, 2.6 Hz, 1H), 6.80 (d, J=2.7 Hz, 1H), 4.65 (bs, 4H), 3.98-3.93 (m, 4H), 3.91 (s, 3H). LCMS (ESI) m/z: 389.1 [M+H]⁺.

### Synthesis of 4-(2-(4-(3-methoxyphenyl)-1H-pyrazol-1-yl)pyrido[3,2-d]pyrimidin-4-yl)morpholine (Compound 28)

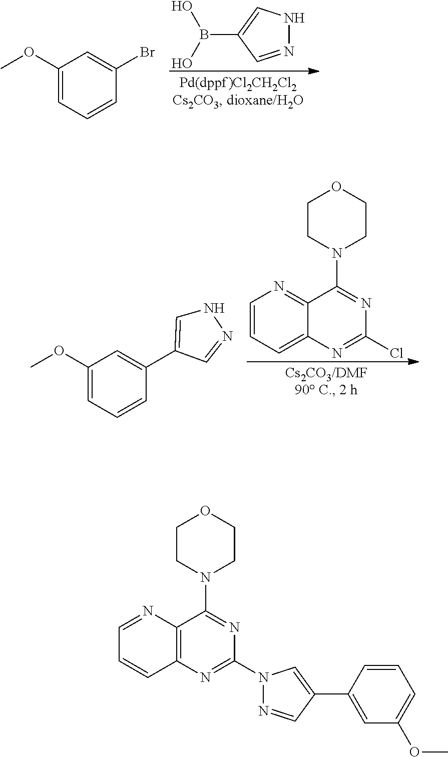

### Step 1: Synthesis of 4-(3-methoxyphenyl)-1H-pyrazole

A mixture of 1-bromo-3-methoxybenzene (930 mg, 5.0 mmol), 1H-pyrazol-4-ylboronic acid (560 mg, 5.0 mmol), 1,1′-bis(diphenylphosphino)ferrocene-palladium(II)dichloride dichloromethane complex (82 mg, 0.1 mmol), cesium carbonate (3.25 g, 10.0 mmol) in dioxane (20 mL) and water (4 mL) was stirred at 100° C. under nitrogen atmosphere for 2 h. The resultant mixture was poured into water, extracted with ethyl acetate (150 mL*2) and concentrated. The residue was purified by silica gel column chromatography (50% ethyl acetate in petroleum ether) to afford 4-(3-methoxyphenyl)-1H-pyrazole (350 mg, 40%) as brown oil. LCMS (ESI) m/z: 175.2 [M+H]⁺.

### Step 2: Synthesis of 4-(2-(4-(3-methoxyphenyl)-1H-pyrazol-1-yl)pyrido[3,2-d]pyrimidin-4-yl)morpholine

A mixture of 4-(2-chloropyrido[3,2-d]pyrimidin-4-yl)morpholine (160 mg, 0.64 mmol), 4-(3-methoxyphenyl)-1H-pyrazole (300 mg, 1.7 mmol) and cesium carbonate (416 mg, 1.28 mmol) in DMF (5 mL) was stirred at 90° C. for 2 h. The resultant product from the mixture was purified by prep-HPLC to afford 4-(2-(4-(3-methoxyphenyl)-1H-pyrazol-1-yl)pyrido[3,2-d]pyrimidin-4-yl)morpholine (32.4 mg, 13%) as yellow solid. ¹H NMR (400 MHz, CDCl₃) δ 8.78 (s, 1H), 8.65 (dd, J=4.1, 1.7 Hz, 1H), 8.23 (dd, J=8.5, 1.7 Hz, 1H), 8.10 (s, 1H), 7.62 (dd, J=8.5, 4.1 Hz, 1H), 7.34 (t, J=7.9 Hz, 1H), 7.22 (d, J=7.9 Hz, 1H), 7.17-7.12 (m, 1H), 6.85 (dd, J=7.9, 2.2 Hz, 1H), 4.65 (bs, 4H), 3.95 (t, J=4.0 Hz, 4H), 3.88 (s, 3H); LCMS (ESI) m/z: 389.1 [M+H]⁺.

### Synthesis of 4-(7-(3,6-dihydro-2H-pyran-4-yl)-2-(3-(4-methoxyphenyl)-1H-pyrazol-1-yl)pyrido[3,2-d]pyrimidin-4-yl)morpholine (Compound 29) and 4-(2-(3-(4-methoxyphenyl)-1H-pyrazol-1-yl)-7-(tetrahydro-2H-pyran-4-yl)pyrido[3,2-d]pyrimidin-4-yl)morpholine (Compound 30)

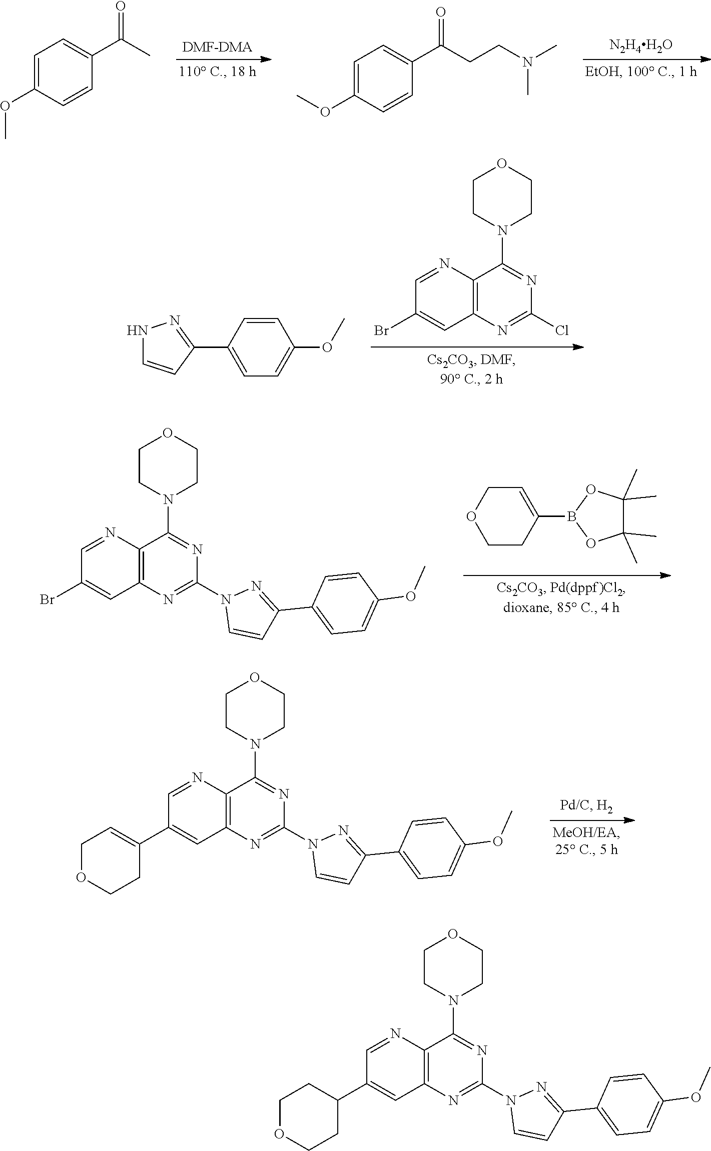

### Step 1: Synthesis of (E)-3-(dimethylamino)-1-(4-methoxyphenyl)prop-2-en-1-one

A mixture of 1-(4-methoxyphenyl)ethan-1-one (10 g, 66.6 mmol) and N,N-dimethylformamide dimethyl acetal (15.9 g, 133 mmol) was stirred at 110° C. for 18 h. The resultant reaction mixture was concentrated to obtain the target product (10 g, 73%) as yellow oil. LCMS (ESI) m/z: 206.1 [M+H]⁺.

### Step 2: Synthesis of 3-(4-methoxyphenyl)-1H-pyrazole

A mixture of (E)-3-(dimethylamino)-1-(4-methoxyphenyl)prop-2-en-1-one (5 g, 24.4 mmol) and hydrazine hydrate (4.9 g, 97.4 mmol) in ethanol (20 mL) was stirred at 100° C. for 30 min. The resultant reaction mixture was concentrated and purified by silica gel column chromatography (dichloromethane:methanol=30:1) to obtain the target product (3 g, 71%) as white solid. LCMS (ESI) m/z: 175.2 [M+H]⁺.

### Step 3: Synthesis of 4-(7-bromo-2-(3-(4-methoxyphenyl)-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (200 mg, 0.61 mmol), cesium carbonate (390 mg, 1.21 mmol) and 3-(4-methoxyphenyl)-1H-pyrazole (110 mg, 0.64 mmol) in N,N-dimethylformamide (5 mL) was stirred at 90° C. for 2 h. The reaction mixture was filtered and purified by silica gel column chromatography (petroleum ether:acetic ester=2:1) to obtain the target product (120 mg, 42%) as yellow solid. LCMS (ESI) m/z: 467.0/470.0 [M+H]⁺.

### Step 4: Synthesis of 4-(7-(3,6-dihydro-2H-pyran-4-yl)-2-(3-(4-methoxyphenyl)-1H-pyrazol-1-yl)pyrido[3,2-d]pyrimidin-4-yl)morpholine

To a solution of 4-(7-bromo-2-(3-(4-methoxyphenyl)-1H-pyrazol-1-yl)pyrido[3,2-d]pyrimidin-4-yl)morpholine (0.12 g, 0.26 mmol) in dioxane/water (3 mL/1 mL) were added 2-(3,6-dihydro-2H-pyran-4-yl)-4,4,5,5-tetramethyl-1,3,2-dioxaborolane (0.081 g, 0.39 mmol), cesium carbonate (0.21 g, 0.64 mmol) and [1,1′-bis(diphenylphosphino)ferrocene]dichloropalladium(II) (0.022 g, 0.03 mmol) at 25° C. and the resultant mixture was stirred at 85° C. for 4 h under argon atmosphere. The mixture was then filtered and the filtrate was subjected to prep-HPLC (Sun Fire C18, 4.6*50 mm, 3.5 um column Xbridge C18 3.5 μm 4.6×50 mm column. The mobile phase was acetonitrile/10 mM ammonium bicarbonate aqueous solution) to obtain 4-(7-(3,6-dihydro-2H-pyran-4-yl)-2-(3-(4-methoxyphenyl)-1H-pyrazol-1-yl)pyrido[3,2-d]pyrimidin-4-yl)morpholine (0.0309 g, 26%) as off-white solid. ¹H NMR (400 MHz, DMSO-d₆) δ 8.95 (d, J=2.2 Hz, 1H), 8.74 (d, J=2.7 Hz, 1H), 8.08 (d, J=2.1 Hz, 1H), 7.90 (d, J=8.8 Hz, 2H), 7.05 (d, J=8.7 Hz, 2H), 7.00 (d, J=2.6 Hz, 1H), 6.78 (s, 1H), 4.56 (bs, 4H), 4.31 (d, J=2.3 Hz, 2H), 3.89 (t, J=5.4 Hz, 2H), 3.87-3.79 (m, 7H), 2.61 (s, 2H); LCMS (ESI) m/z: 471.1 [M+H]⁺.

### Step 5: Synthesis of 4-(2-(3-(4-methoxyphenyl)-1H-pyrazol-1-yl)-7-(tetrahydro-2H-pyran-4-yl)pyrido[3,2-d]pyrimidin-4-yl)morpholine

A mixture of 4-(7-(3,6-dihydro-2H-pyran-4-yl)-2-(3-(4-methoxyphenyl)-1H-pyrazol-1-yl)pyrido[3,2-d]pyrimidin-4-yl)morpholine (20 mg, 0.043 mmol) and palladium (10% on activated carbon, 20 mg) in methanol/ethyl acetate (3 mL/3 mL) was stirred at 25° C. for 5 h under hydrogen atmosphere. The reaction mixture was filtered and concentrated to obtain the target product (10.4 mg, 52%) as off-white solid. ¹H NMR (400 MHz, DMSO-d₆) δ 8.74 (d, J=2.7 Hz, 1H), 8.71 (d, J=2.2 Hz, 1H), 7.99 (d, J=2.1 Hz, 1H), 7.93-7.88 (m, 2H), 7.07-7.02 (m, 2H), 6.99 (d, J=2.7 Hz, 1H), 4.57 (bs, 4H), 4.05-3.96 (m, 2H), 3.85-3.76 (m, 7H), 3.60-3.45 (m, 2H), 3.15-2.98 (m, 1H), 1.88-1.75 (m, 4H); LCMS (ESI) m/z: 473.1 [M+H]⁺.

### Synthesis of 3-methyl-4-(2-(3-phenyl-1H-pyrazol-1-yl)pyrido[3,2-d]pyrimidin-4-yl)morpholine (Compound 31)

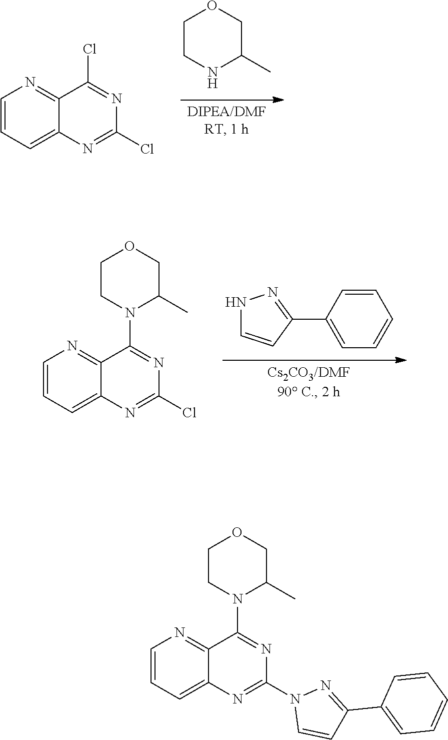

### Step 1: Synthesis of 4-(2-chloropyrido[3,2-d]pyrimidin-4-yl)-3-methylmorpholine

To a solution of 2,4-dichloropyrido[3,2-d]pyrimidine (1.5 g, 7.5 mmol), N,N-Diisopropylethylamine (1.94 g, 15.0 mmol) in N,N-dimethylformamide (15 mL) was added 3-methylmorpholine (758 mg, 7.5 mmol) at 28° C. After the addition, the mixture was stirred for 1 h and then poured into water (100 mL). The formed precipitate was collected by filtration and dried under vacuum to afford 4-(2-chloropyrido[3,2-d]pyrimidin-4-yl)-3-methylmorpholine (1.6 g, 80%) as yellow solid. LCMS (ESI) m/z: 265.1/267.1 [M+H]⁺.

### Step 2: Synthesis of 3-methyl-4-(2-(3-phenyl-1H-pyrazol-1-yl)pyrido[3,2-d]pyrimidin-4-yl)morpholine

A mixture of 4-(2-chloropyrido[3,2-d]pyrimidin-4-yl)-3-methylmorpholine (130 mg, 0.49 mmol), 3-phenyl-1H-pyrazole (71 mg, 0.49 mmol), cesium carbonate (325 mg, 1.0 mmol) and N,N-dimethylformamide (4 mL) was stirred at 90° C. for 2 h. The precipitate formed was removed by filtration and the filtrate was purified by prep-HPLC (Column Xbridge 21.2*250 mm C18, 10 um, Mobile Phase A: water (10 mmol/L ammonium bicarbonate) B: acetonitrile) to obtain 3-methyl-4-(2-(3-phenyl-1H-pyrazol-1-yl)pyrido[3,2-d]pyrimidin-4-yl)morpholine (115.8 mg, 63.2%) as white solid. ¹H NMR (500 MHz, CDCl₃) δ 8.66 (dd, J=3.2, 0.8 Hz, 1H), 8.65 (d, J=2.0 Hz, 1H), 8.28 (dd, J=8.5, 1.7 Hz, 1H), 8.05 (d, J=2.4 Hz, 2H), 7.63 (dd, J=8.5, 4.1 Hz, 1H), 7.46 (dd, J=10.4, 4.7 Hz, 2H), 7.40-7.31 (m, 1H), 6.83 (d, J=2.7 Hz, 1H), 6.50-4.50 (bs, 2H), 4.10 (d, J=9.7 Hz, 1H), 3.94 (dd, J=8.8, 2.8 Hz, 1H), 3.89 (d, J=9.6 Hz, 1H), 3.81 (td, J=11.8, 2.6 Hz, 1H), 3.71-3.67 (m, 1H), 1.54 (d, J=6.9 Hz, 3H); LCMS (ESI) m/z: 373.2 [M+H]⁺.

### Synthesis of 4-[2-(3-pyrimidin-5-ylpyrazol-1-yl)pyrido[3,2-d]pyrimidin-4-yl]morpholine (Compound 32)

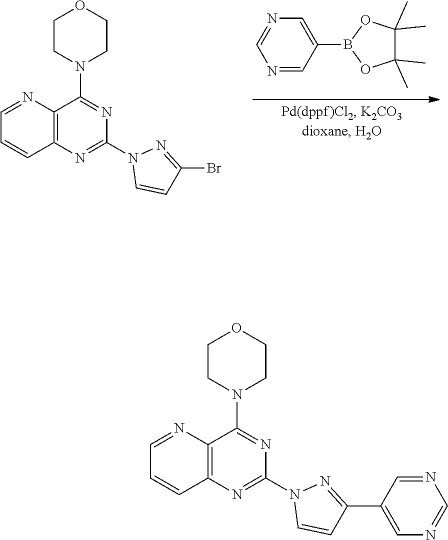 [SMILES: Brc1ccn(-c2nc(N3CCOCC3)c3ncccc3n2)n1 ; CC1(C)OB(c2cncnc2)OC1(C)C ; c1cnc2c(N3CCOCC3)nc(-n3ccc(-c4cncnc4)n3)nc2c1]

To a solution of 4-[2-(3-bromopyrazol-1-yl)pyrido[3,2-d]pyrimidin-4-yl]morpholine (200 mg, 554 umol) in dioxane (5 mL) and H₂O (1 mL) were added 5-(4,4,5,5-tetramethyl-1,3,2-dioxaborolan-2-yl)pyrimidine (171 mg, 831 umol), K₂CO₃ (191 mg, 1.38 mmol) and Pd(dppf)Cl₂ (41 mg, 55 umol) and the resultant mixture was stirred at 60° C. for 12 h under nitrogen. 15 mL of water was added to the mixture and extracted with ethyl acetate (30 mL*2). The combined organic layers were washed with brine (15 mL) and dried over Na₂SO₄. The organic layer was concentrated and the crude product was purified by prep-HPLC (Waters Xbridge Prep OBD C18 150*40 mm*10 um column; 30-65% acetonitrile in an a 10 mM ammonium bicarbonate solution in water, 8 min gradient) to obtain 4-[2-(3-pyrimidin-5-ylpyrazol-1-yl)pyrido[3,2-d]pyrimidin-4-yl]morpholine (14 mg, 38 umol, 7%) as a white solid. 1H NMR (400 MHz, CHLOROFORM-d) δ=9.34 (s, 2H), 9.22 (s, 1H), 8.76-8.67 (m, 2H), 8.27 (dd, J=8.6, 1.5 Hz, 1H), 7.66 (dd, J=8.5, 4.1 Hz, 1H), 6.88 (d, J=2.6 Hz, 1H), 4.9-4.45 (m, 4H), 4.00-3.93 (m, 4H) LCMS (ESI) for C₁₈H₁₆N₈O [M+H]⁺: 361.2

### Synthesis of 4-(7-bromo-2-(3-phenyl-1H-pyrazol-1-yl)pyrido[3,2-d]pyrimidin-4-yl)morpholine (Compound 33) and 4-(7-methyl-2-(3-phenyl-1H-pyrazol-1-yl)pyrido[3,2-d]pyrimidin-4-yl)morpholine (Compound 34)

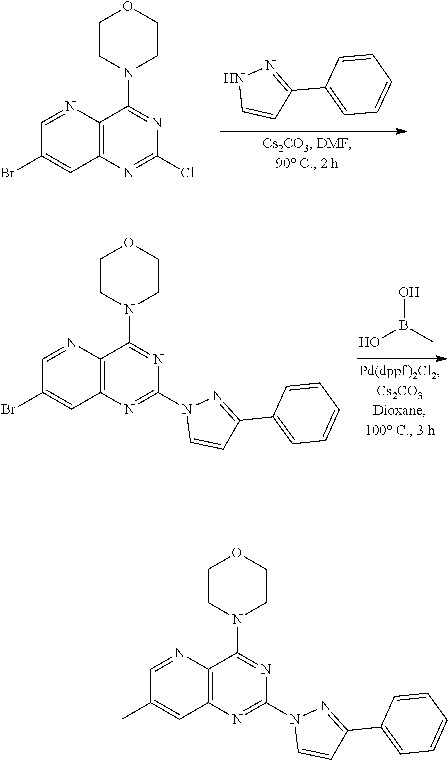

### Step 1: Synthesis of 4-(7-bromo-2-(3-phenyl-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (500 mg, 1.52 mmol), cesium carbonate (990 mg, 3.03 mmol) and 3-phenyl-1H-pyrazole (0.26 mg, 1.82 mmol) in DMF (10 mL) was stirred at 90° C. for 2 h. The resultant crude product was purified by silica gel column chromatography (petroleum ether:acetic ester=2:1) to obtain the target product (450 mg, 68%) as yellow solid. ¹H NMR (400 MHz, DMSO-d₆) δ 8.77 (dd, J=8.0, 2.4 Hz, 2H), 8.48 (d, J=2.0 Hz, 1H), 7.99-7.96 (m, 2H), 7.52-7.38 (m, 3H), 7.09 (d, J=2.7 Hz, 1H), 4.60 (bs, 4H), 3.83 (t, J=4.4 Hz, 4H); LCMS (ESI) m/z: 438.9 [M+H]⁺.

### Step 2: Synthesis of 4-(7-methyl-2-(3-phenyl-1H-pyrazol-1-yl)pyrido[3,2-d]pyrimidin-4-yl)morpholine

To a solution of 4-(7-bromo-2-(3-phenyl-1H-pyrazol-1-yl)pyrido[3,2-d]pyrimidin-4-yl)morpholine (0.1 g, 0.23 mmol) in dioxane/water (2 mL/0.5 mL) were added methylboronic acid (0.042 g, 0.69 mmol), cesium carbonate (0.26 g, 0.8 mmol) and 1,1′-bis(diphenylphosphino)ferrocene-palladium(II)dichloride dichloromethane complex (0.017 g, 0.023 mmol) at 25° C. The reaction mixture was stirred at 100° C. for 4 h under argon. It was then filtered and the crude product from the filtrate was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product as off-white solid. (39.1 mg, 46%). ¹H NMR (400 MHz, CDCl₃) δ 8.63 (d, J=2.7 Hz, 1H), 8.48 (d, J=1.9 Hz, 1H), 8.02 (d, J=6.9 Hz, 3H), 7.44 (t, J=7.5 Hz, 2H), 7.35 (t, J=7.3 Hz, 1H), 6.80 (d, J=2.7 Hz, 1H), 4.63 (s, 4H), 3.93 (t, J=4.0 Hz, 4H), 2.51 (s, 3H); LCMS (ESI) m/z: 373.2 [M+H]⁺.

### Synthesis of 4-(7-(3,6-dihydro-2H-pyran-4-yl)-2-(3-(4-methoxybenzyl)-1H-pyrazol-1-yl)pyrido[3,2-d]pyrimidin-4-yl)morpholine (Compound 35)

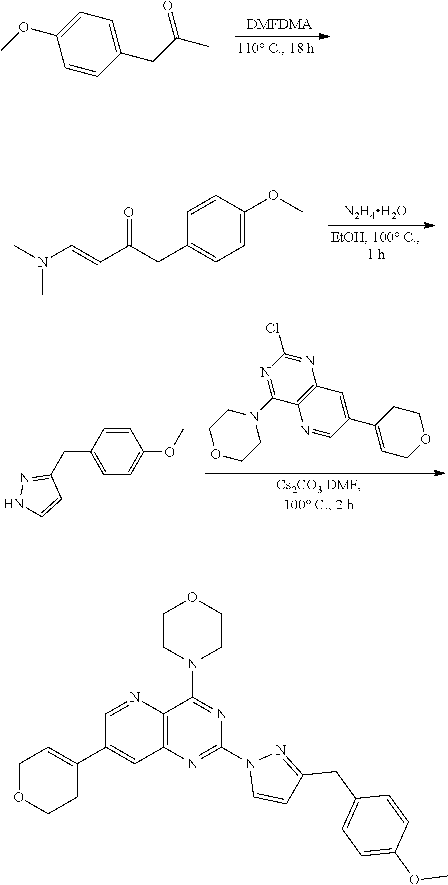

### Step 1: Synthesis of (E)-4-(dimethylamino)-1-(4-methoxyphenyl)but-3-en-2-one

A mixture of 1-(4-methoxyphenyl)propan-2-one (10 g, 60.9 mmol) and N,N-Dimethylformamide dimethyl acetal (14.5 g, 121.8 mmol) was stirred at 110° C. for 18 h. The resultant mixture was concentrated to obtain the desired product (10 g, 75%) as yellow oil.

### Step 2: Synthesis of 3-(4-methoxybenzyl)-1H-pyrazole

A mixture of (E)-4-(dimethylamino)-1-(4-methoxyphenyl)but-3-en-2-one (10 g, 68.4 mmol) and hydrazine hydrate (13.7 g, 273.6 mmol) in ethanol (50 mL) was stirred at 100° C. for 30 min. The resultant mixture was concentrated and the crude product obtained was purified using silica gel column chromatography (dichloromethane:methanol=30:1) to obtain the target compound (4 g, 60%) as white solid.

### Step 3: Synthesis of 4-(7-(3,6-dihydro-2H-pyran-4-yl)-2-(3-(4-methoxybenzyl)-1H-pyrazol-1-yl)pyrido[3,2-d]pyrimidin-4-yl)morpholine

A solution of 4-(2-chloro-7-(3,6-dihydro-2H-pyran-4-yl)pyrido[3,2-d]pyrimidin-4-yl)morpholine (0.07 g, 0.21 mmol), 3-(4-methoxybenzyl)-1H-pyrazole (0.044 g, 0.23 mmol) and cesium carbonate (0.137 g, 0.42 mmol) in DMF (2 mL) was stirred at 100° C. for 2 h. The reaction mixture was filtered and the filtrate was concentrated. The residue was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain 4-(7-(3,6-dihydro-2H-pyran-4-yl)-2-(3-(4-methoxybenzyl)-1H-pyrazol-1-yl)pyrido[3,2-d]pyrimidin-4-yl)morpholine as yellow solid. (0.0106 g, 10%). ¹H NMR (400 MHz, DMSO-d₆) δ 8.75 (s, 1H), 8.67 (d, J=2.2 Hz, 1H), 7.90 (d, J=2.1 Hz, 1H), 7.51 (d, J=8.7 Hz, 2H), 7.01 (d, J=8.8 Hz, 2H), 6.70 (dd, J=6.2, 1.8 Hz, 1H), 4.86 (dd, J=6.2, 3.2 Hz, 1H), 4.57 (bs, 4H), 4.06-3.91 (m, 2H), 3.87-3.73 (m, 8H), 2.41 (s, 3H), 2.31-2.21 (m, 1H), 1.96-1.82 (m, 1H). LCMS (ESI) m/z: 485.1 [M+H]⁺.

### Synthesis of 4-(2-(3-phenyl-1H-pyrazol-1-yl)-7-(piperidin-4-yl)pyrido[3,2-d]pyrimidin-4-yl)morpholine (Compound 36) and 4-(7-(1-methylpiperidin-4-yl)-2-(3-phenyl-1H-pyrazol-1-yl)pyrido[3,2-d]pyrimidin-4-yl)morpholine (Compound 37)

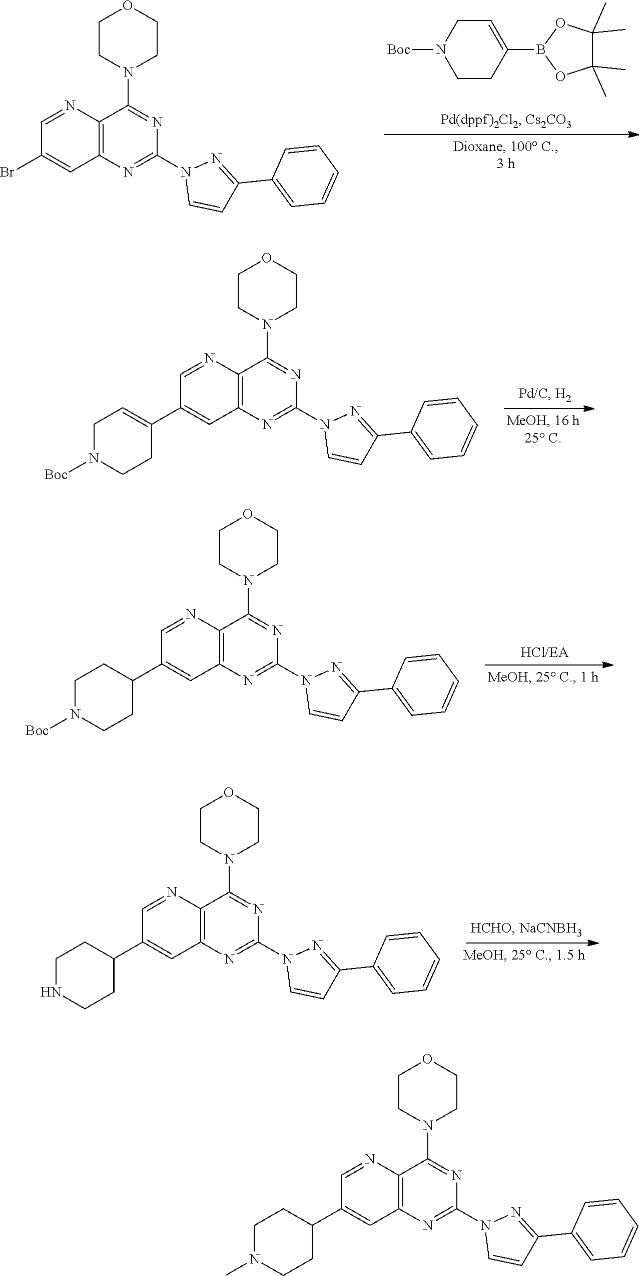 [SMILES: Brc1cnc2c(N3CCOCC3)nc(-n3ccc(-c4ccccc4)n3)nc2c1 ; CC(C)(C)OC(=O)N1CC=C(B2OC(C)(C)C(C)(C)O2)CC1 ; CC(C)(C)OC(=O)N1CC=C(c2cnc3c(N4CCOCC4)nc(-n4ccc(-c5ccccc5)n4)nc3c2)CC1 ; CC(C)(C)OC(=O)N1CCC(c2cnc3c(N4CCOCC4)nc(-n4ccc(-c5ccccc5)n4)nc3c2)CC1 ; c1ccc(-c2ccn(-c3nc(N4CCOCC4)c4ncc(C5CCNCC5)cc4n3)n2)cc1 ; CN1CCC(c2cnc3c(N4CCOCC4)nc(-n4ccc(-c5ccccc5)n4)nc3c2)CC1]

### Step 1: Synthesis of tert-butyl 4-(4-morpholino-2-(3-phenyl-1H-pyrazol-1-yl)pyrido[3,2-d]pyrimidin-7-yl)-3,6-dihydropyridine-1(2H)-carboxylate

To a solution of 4-(7-bromo-2-(3-phenyl-1H-pyrazol-1-yl)pyrido[3,2-d]pyrimidin-4-yl)morpholine (0.2 g, 0.46 mmol) in dioxane/water (4 mL/1 mL) were added tert-butyl 4-(4,4,5,5-tetramethyl-1,3,2-dioxaborolan-2-yl)-3,6-dihydropyridine-1(2H)-carboxylate (0.18 g, 0.59 mmol), cesium carbonate (0.52 g, 1.6 mmol) and bis(diphenylphosphino)ferrocene-palladium(II) dichloride dichloromethane complex (0.034 g, 0.046 mmol) at 25° C. and the resultant mixture was stirred at 100° C. for 2 h under argon atmosphere. It was then filtered and the filtrate was purified by silica gel column chromatography (petroleum ether:acetic ester=2:1) to obtain the target product as off-white solid (160 mg, 65%).

### Step 2: Synthesis of tert-butyl 4-(4-morpholino-2-(3-phenyl-1H-pyrazol-1-yl)pyrido[3,2-d]pyrimidin-7-yl)piperidine-1-carboxylate

A mixture of tert-butyl 4-(4-morpholino-2-(3-phenyl-1H-pyrazol-1-yl)pyrido[3,2-d]pyrimidin-7-yl)-3,6-dihydropyridine-1(2H)-carboxylate (100 mg, 0.19 mmol) and palladium on activated carbon (10% Pd, 40 mg) in methanol/ethyl acetate (10 mL/10 mL) was stirred at 25° C. for 18 h under hydrogen. The reaction mixture was filtered and concentrated to obtain the target product (100 mg, 100%) as white solid.

### Step 3: Synthesis of 4-(2-(3-phenyl-1H-pyrazol-1-yl)-7-(piperidin-4-yl)pyrido[3,2-d]pyrimidin-4-yl)morpholine

To a solution of tert-butyl 4-(4-morpholino-2-(3-phenyl-1H-pyrazol-1-yl)pyrido[3,2-d]pyrimidin-7-yl)piperidine-1-carboxylate (0.1 g, 0.18 mmol) in methanol (2 mL) was added hydrochloric acid/ethyl acetate (2 mL) and the reaction mixture was stirred at 25° C. for 1 h. It was then concentrated and the crude product obtained was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product (0.07 g, 86%) as off-white solid. ¹HNMR (400 MHz, DMSO-d₆) δ 8.78 (d, J=2.7 Hz, 1H), 8.69 (t, J=4.0 Hz, 1H), 7.99-7.96 (m, 2H), 7.94 (d, J=1.8 Hz, 1H), 7.49 (t, J=7.5 Hz, 2H), 7.41 (q, J=7.1 Hz, 1H), 7.07 (d, J=2.7 Hz, 1H), 4.59 (bs, 4H), 3.83 (t, J=4.0 Hz, 4H), 3.09 (d, J=12.3 Hz, 2H), 2.88 (t, J=11.9 Hz, 1H), 2.65 (t, J=11.1 Hz, 2H), 1.88-1.78 (m, 2H), 1.64 (dt, J=12.0, 8.4 Hz, 2H); LCMS (ESI) m/z: 441.8 [M+]⁺.

### Step 4: Synthesis of 4-(7-(1-methylpiperidin-4-yl)-2-(3-phenyl-1H-pyrazol-1-yl)pyrido[3,2-d]pyrimidin-4-yl)morpholine

A solution of 4-(2-(3-phenyl-1H-pyrazol-1-yl)-7-(piperidin-4-yl)pyrido[3,2-d]pyrimidin-4-yl)morpholine (55 mg, 0.12 mmol), 37% formaldehyde (5 drops) in methanol (3 mL) was stirred for 0.5 hour at 25° C. Then sodium cyanoborohydride (97 mg, 1.5 mmol) was added and the resultant mixture was stirred for 1 h at 25° C. The reaction mixture was then filtered, filtrate was concentrated and the residue was purified by prep-HPLC (SunFire C18, 4.6*50 mm, 3.5 um column Xbridge C18 3.5 μm 4.6×50 mm column. The mobile phase was acetonitrile/10 mM formic acid aqueous solution) to obtain the target product (33.1 mg, 58%) as off-white solid. ¹H NMR (400 MHz, DMSO-d₆) δ 8.78 (d, J=2.7 Hz, 1H), 8.71 (d, J=2.2 Hz, 1H), 8.16 (s, 1H), 7.98 (s, 2H), 7.97 (s, 1H), 7.49 (t, J=7.5 Hz, 2H), 7.40 (t, J=7.3 Hz, 1H), 7.08 (d, J=2.7 Hz, 1H), 4.57 (bs, 4H), 3.83 (t, J=4 Hz, 4H), 3.05 (d, J=11.3 Hz, 2H), 2.82 (s, 1H), 2.35 (s, 3H), 2.26 (t, J=11.6 Hz, 2H), 1.94 (d, J=10.8 Hz, 2H), 1.85 (t, J=10.6 Hz, 2H); LCMS (ESI) m/z: 456.2 [M+H]⁺.

### Synthesis of 4-(7-(3,6-dihydro-2H-pyran-4-yl)-2-(3-phenyl-1H-pyrazol-1-yl)pyrido[3,2-d]pyrimidin-4-yl)morpholine (Compound 38)

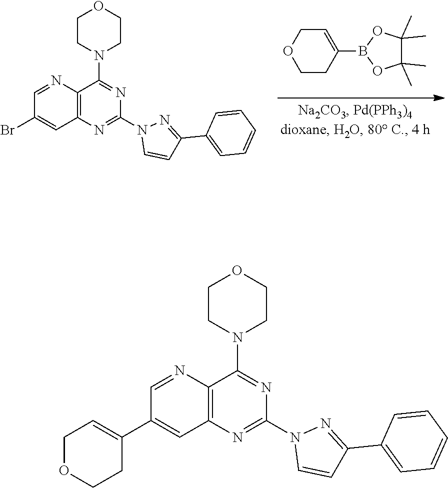 [SMILES: Brc1cnc2c(N3CCOCC3)nc(-n3ccc(-c4ccccc4)n3)nc2c1 ; CC1(C)OB(C2=CCOCC2)OC1(C)C ; C1=C(c2cnc3c(N4CCOCC4)nc(-n4ccc(-c5ccccc5)n4)nc3c2)CCOC1]

A solution of 4-(7-Bromo-2-(3-phenyl-1H-pyrazol-1-yl)pyrido[3,2-d]pyrimidin-4-yl)morpholine (60 mg, 0.14 mmol), 2-(3,6-Dihydro-2H-pyran-4-yl)-4,4,5,5-tetramethyl-1,3,2-dioxaborolane (59 mg, 0.28 mmol), tetrakis(triphenyl phosphine)palladium (16 mg, 0.014 mmol) and sodium carbonate (18 mg, 0.17 mmol) in water (0.5 mL) and dioxane (2.0 mL) was stirred at 80° C. for 4 h under argon atmosphere. Water (25 mL) was added and the resultant mixture extracted with dichloromethane (25 ml*3). The organic layer was dried over Na₂SO₄, filtered and concentrated under the reduced pressure. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 4-(7-(3,6-Dihydro-2H-pyran-4-yl)-2-(3-phenyl-1H-pyrazol-1-yl)pyrido[3,2-d]pyrimidin-4-yl)morpholine (30 mg, 48%) as white solid. 1H NMR (400 MHz, DMSO-d₆) δ 8.96 (d, J=2.0 Hz, 1H), 8.78 (d, J=2.4 Hz, 1H), 8.08 (d, J=1.6 Hz, 1H), 7.98-7.97 (m, 3H), 7.50-7.38 (m, 3H), 7.08 (d, J=2.8 Hz, 1H), 6.78 (s, 1H), 4.59 (bs, 4H), 4.31 (d, J=2.0 Hz, 2H), 3.90-3.83 (m, 6H), 2.60 (s, 2H); LCMS (ESI) m/z: 441.3 [M+H]⁺.

### Synthesis of 4-(2-(5-methyl-3-phenyl-1H-pyrazol-1-yl)pyrido[3,2-d]pyrimidin-4-yl)morpholine (Compound 39)

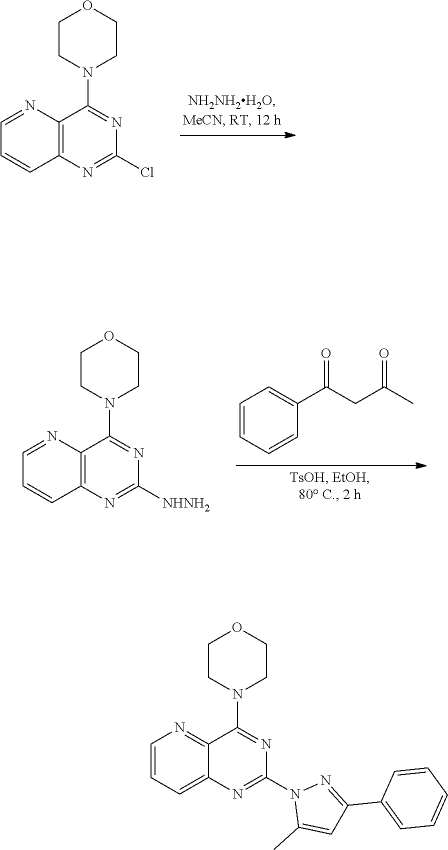 [SMILES: Clc1nc(N2CCOCC2)c2ncccc2n1 ; NNc1nc(N2CCOCC2)c2ncccc2n1 ; CC(=O)CC(=O)c1ccccc1 ; Cc1cc(-c2ccccc2)nn1-c1nc(N2CCOCC2)c2ncccc2n1]

### Step 1: Synthesis of 4-(2-hydrazineylpyrido[3,2-d]pyrimidin-4-yl)morpholine

4-(2-Chloropyrido[3,2-d]pyrimidin-4-yl)morpholine (200 mg, 0.8 mmol) was dissolved in acetonitrile (15 mL). Hydrazine hydrate (60%, 5 mL) was added and the mixture was stirred at room temperature overnight. The mixture was then concentrated, the resultant precipitate filtered, washed with methanol to obtain the target compound as yellow solid (140 mg, 71.06%).

### Step 2: Synthesis of 4-(2-(5-methyl-3-phenyl-1H-pyrazol-1-yl)pyrido[3,2-d]pyrimidin-4-yl)morpholine

A mixture of 4-(2-Hydrazineylpyrido[3,2-d]pyrimidin-4-yl)morpholine (140 mg, 0.57 mmol), 1-phenylbutane-1,3-dione (91 mg, 0.56 mmol) and p-toluene sulfonic acid monohydrate (40 mg, 0.21 mmol) in ethanol (10 mL) was stirred at 80° C. for 2 h under nitrogen atmosphere. The mixture was concentrated and the resultant crude product was purified by prep-HPLC (Column Xbridge 21.2*250 mm C18, 10 um, Mobile Phase A: water (10 mmol/L ammonium bicarbonate) B: acetonitrile) to afford 4-(2-(5-methyl-3-phenyl-1H-pyrazol-1-yl)pyrido[3,2-d]pyrimidin-4-yl)morpholine as white solid. (45.8 mg, 21.57%). ¹H NMR (400 MHz, CDCl₃) δ 8.60 (dd, J=4.1, 1.7 Hz, 1H), 8.26 (dd, J=8.5, 1.7 Hz, 1H), 7.58 (dd, J=8.5, 4.1 Hz, 1H), 7.35-7.29 (m, 5H), 6.28 (s, 1H), 3.52 (bs, 4H), 3.49 (s, 4H), 2.44 (s, 3H); LCMS (ESI) m/z: 373.2. [M+H]⁺.

### Synthesis of 4,4′-(2-(4-phenyl-1H-pyrazol-1-yl)pyrido[3,2-d]pyrimidine-4,7-diyl)dimorpholine (Compound 40)

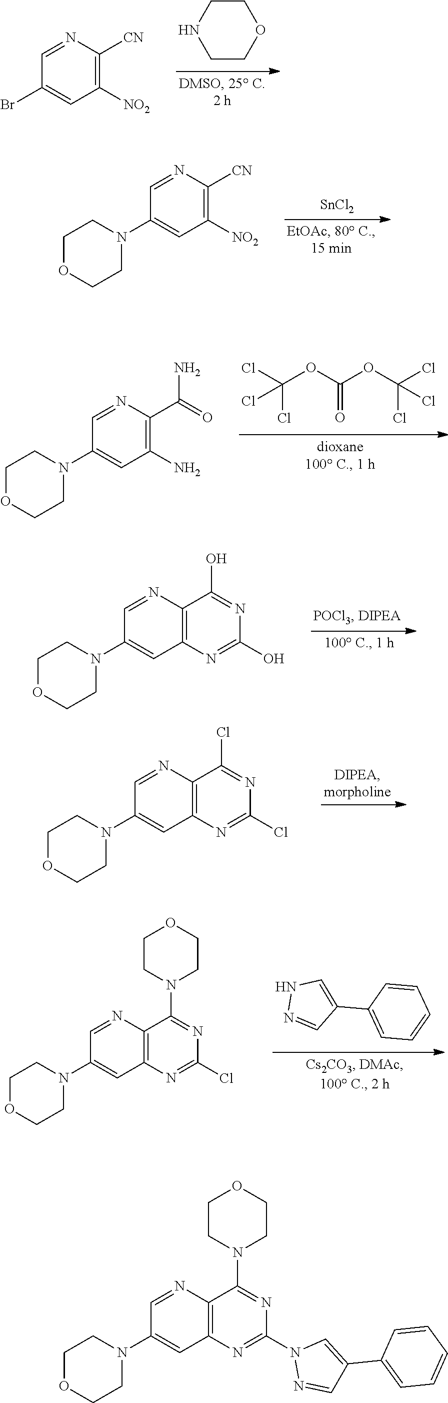

### Step 1: Synthesis of 5-morpholino-3-nitropicolinonitrile

To a stirred solution of 5-bromo-3-nitropicolinonitrile (10 g, 44 mmol) in dimethyl sulfoxide (60 mL) was added morpholine (7.6 mL, 88 mmol). The mixture was stirred at 25° C. for 2 h and then diluted with ethyl acetate (600 mL) and water (200 mL). The organic layer was separated, dried over magnesium sulfate, filtered and evaporated in vacuo. The resulting solid was washed with methanol (30 mL) to obtain 5-morpholino-3-nitropicolinonitrile as yellow solid (6 g, 58%). LCMS (ESI) m/z: 235.1 [M+H]⁺.

### Step 2: Synthesis of 3-amino-5-morpholinopicolinamide

To a stirred solution of 5-morpholino-3-nitropicolinonitrile (6 g, 25.6 mmol) in ethyl acetate (100 mL) was added stannous chloride. dihydrate (24.3 g, 128.1 mmol). The resultant mixture was heated at 80° C. for 15 min. The resultant precipitate was filtered and the solid was washed with 1.0M sodium hydroxide (300 mL) and brine (50 mL) and dried under vacuum to afford 3-amino-5-morpholinopicolinamide (2 g, 35%). LCMS (ESI) m/z: 223.2 [M+H]⁺.

### Step 3: Synthesis of 7-morpholinopyrido[3,2-d]pyrimidine-2,4-diol

Triphosgene (1.34 g, 4.5 mmol) was added to a solution of 3-amino-5-morpholinopicolinamide (2 g, 9 mmol) in dry dioxane (30 ml) under a nitrogen atmosphere. The resultant dark orange reaction mixture was stirred at 100° C. under a nitrogen atmosphere for 1 h. The mixture was cooled and the resultant precipitate was filtered and dried to obtain the target product (1.5 g, 67%) as red solid.

### Step 4: Synthesis of 4-(2,4-dichloropyrido[3,2-d]pyrimidin-7-yl)morpholine

To a mixture of 7-morpholinopyrido[3,2-d]pyrimidine-2,4-diol (1 g, 4 mmol) in phosphorus oxychloride (30 mL) was added N,N-diisopropylethylamine (2.0 mL) and the reaction mixture was stirred at 100° C. for 1 h. The volatiles were evaporated and azeotrophed with toluene (2×100 mL). The obtained residue was treated with ethyl acetate and filtered through a celite pad and filtrate was evaporated to obtain 7-4-(2,4-dichloropyrido[3,2-d]pyrimidin-7-yl)morpholine (0.4 g, 35%) as black solid. LCMS (ESI) m/z: 285.0/288.0 [M+H]⁺.

### Step 5: Synthesis of 4,4′-(2-chloropyrido[3,2-d]pyrimidine-4,7-diyl)dimorpholine

A solution of 4-(2,4-dichloropyrido[3,2-d]pyrimidin-7-yl)morpholine (0.4 g, 1.4 mmol) and morpholine (0.244 g, 2.8 mmol) in dichloromethane (10.0 mL) was stirred at 25° C. under nitrogen atmosphere for 2 h. The mixture was then concentrated and purified by flash column chromatography (ethyl acetate/petroleum ether 1:20) to obtain the target product (0.2 g, 43%) as off-white solid. LCMS (ESI) m/z: 336.1 [M+H]⁺.

### Step 6: Synthesis of 4,4′-(2-(4-phenyl-1H-pyrazol-1-yl)pyrido[3,2-d]pyrimidine-4,7-diyl)dimorpholine

To a solution of 4,4′-(2-chloropyrido[3,2-d]pyrimidine-4,7-diyl)dimorpholine (0.06 g, 0.18 mmol) in N,N-dimethylacetamide (3 mL) were added 4-phenyl-1H-pyrazole (0.36 g, 0.25 mmol) and cesium carbonate (0.116 g, 0.36 mmol) at 25° C. and the resultant mixture was stirred at 100° C. for 2 h under argon atmosphere. The mixture was then filtered and the filtrate was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product (0.06 g, 76%) as off-white solid. ¹H NMR (400 MHz, DMSO-d6) δ 9.05 (s, 1H), 8.66 (d, J=2.8 Hz, 1H), 8.26 (s, 1H), 7.84-7.74 (m, 2H), 7.42 (t, J=7.7 Hz, 2H), 7.32-7.23 (m, 2H), 4.53 (bs, 4H), 3.92-3.72 (m, 8H), 3.51-3.38 (m, 4H); LCMS (ESI) m/z: 444.2 [M+H]⁺.

The following compounds were synthesized according to the protocol described above:

| Name | Structure | NMR, MS | # |
|---|---|---|---|
| 4-[4-morpholino-2-(3- phenylpyrazol-1- yl)pyrido[3,2- d]pyrimidin-7- yl]morpholine | 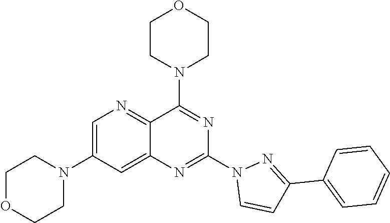 [SMILES: c1ccc(-c2ccn(-c3nc(N4CCOCC4)c4ncc(N5CCOCC5)cc4n3)n2)cc1] | ¹H NMR (400MHz, CHLOROFORM-d) δ = 8.63 (d, J = 2.6 Hz, 1H), 8.44 (d, J = 2.9 Hz, 1H), 8.09-7.97 (m, 2H), 7.47-7.40 (m, 3H), 7.39- 7.32 (m, 1H), 6.80 (d, J = 2.8 Hz, 1H), 4.58 (bs, 4H), 4.00-3.86 (m, 8H), 3.44-3.35 (m, 4H). LCMS (ESI) for C24H25N7O2 [M + H]⁺: 444.2. | 41 |
| 4-[2-(3-phenylpyrazol- 1-yl)-7-piperazin-1-yl- pyrido[3,2-d]pyrimidin- 4-yl]morpholine | 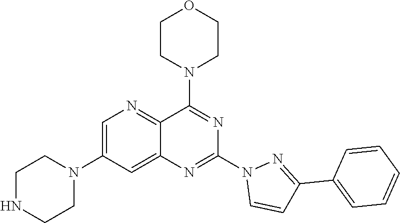 [SMILES: c1ccc(-c2ccn(-c3nc(N4CCOCC4)c4ncc(N5CCNCC5)cc4n3)n2)cc1] | ¹H NMR (400MHz, CHLOROFORM-d) δ = 8.63 (d, J = 2.6 Hz, 1H), 8.45 (d, J = 2.9 Hz, 1H), 8.07-7.99 (m, 2H), 7.49-7.31 (m, 4H), 6.80 (d, J = 2.8 Hz, 1H), 4.58 (bs, 4H), 3.99-3.88 (m, 4H), 3.46-3.35 (m, 4H), 3.14-3.04 (m, 4H). LCMS (ESI) for C24H26N8O [M + H]⁺: 443.3. | 42 |

### Synthesis of methyl 2-(4-(3-methoxyphenyl)-1H-pyrazol-1-yl)-4-morpholinopyrido[3,2-d]pyrimidine-7-carboxylate (Compound 43), 2-(4-(3-methoxyphenyl)-1H-pyrazol-1-yl)-4-morpholinopyrido[3,2-d]pyrimidine-7-carboxylic acid (Compound 44) and (2-(4-(3-methoxyphenyl)-1H-pyrazol-1-yl)-4-morpholinopyrido[3,2-d]pyrimidin-7-yl)methanol (Compound 45)

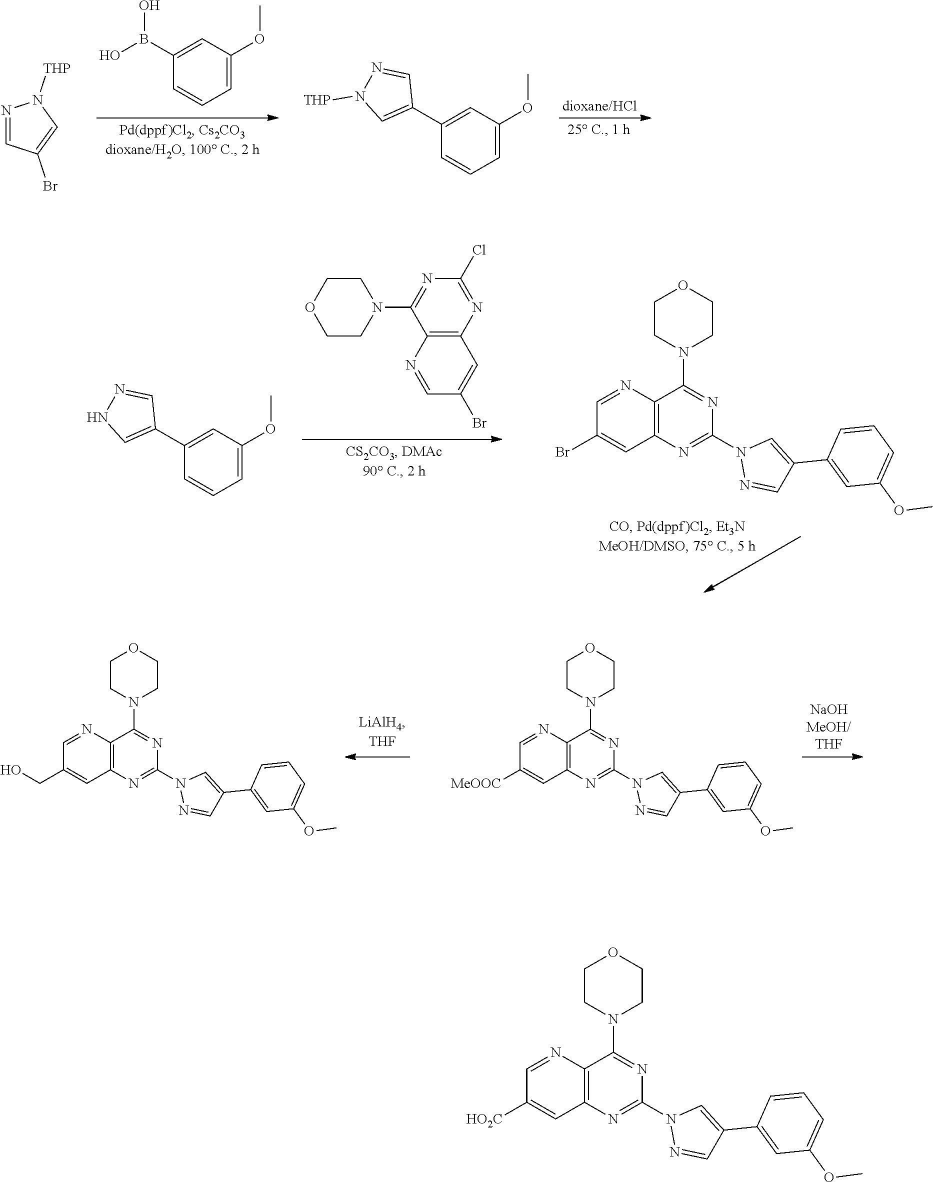

### Step 1: Synthesis of 4-(3-methoxyphenyl)-1-(tetrahydro-2H-pyran-2-yl)-1H-pyrazole

A mixture of 4-bromo-1-(tetrahydro-2H-pyran-2-yl)-1H-pyrazole (3 g, 13 mmol), (3-methoxyphenyl) boronic acid (2.76 g, 18.2 mmol), [1,1′-bis(diphenylphosphino)ferrocene]dichloropalladium(II) (0.95 g, 1.3 mmol) and cesium carbonate (8.46 g, 26 mmol) in dioxane/water (50/15 mL) was stirred at 100° C. for 2 h. The reaction mixture was diluted with ethyl acetate (200 mL) and water (100 mL), the phases separated and the aqueous phase was further extracted with ethyl acetate (100 mL*2). The combined organic layer was dried over magnesium sulfate, filtered and evaporated in vacuo. The resultant residue was purified by silica gel column (petroleum ether:ethyl acetate=5:1) to obtain the target product as yellow oil (2.6 g, 78%). LCMS (ESI) m/z: 259.1 [M+H]⁺.

### Step 2: Synthesis of 4-(3-methoxyphenyl)-1H-pyrazole hydrochloride

A mixture of 4-(3-methoxyphenyl)-1-(tetrahydro-2H-pyran-2-yl)-1H-pyrazole (2.6 g, 10.0 mmol) and HCl/dioxane (20 mL) was stirred at 25° C. for 1 h. The resultant mixture was filtered and dried to give the target product as white solid (1.4 g, 66%). LCMS (ESI) m/z: 175.2 [M+H]⁺.

### Step 3: Synthesis of 4-(7-bromo-2-(4-(3-methoxyphenyl)-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (200 mg, 0.6 mmol), cesium carbonate (490 mg, 1.5 mmol) and 4-(3-methoxyphenyl)-1H-pyrazole hydrochloride (170 mg, 0.8 mmol) in N,N-dimethylacetamide (4 mL) was stirred at 90° C. for 2 h. The reaction mixture was filtered and purified by silica gel column chromatography (petroleum ether:ethyl acetate=2:1) to obtain target product (100 mg, 35%) as yellow solid. LCMS (ESI) m/z: 467.1 [M+H]⁺.

### Step 4: Synthesis of methyl 2-(4-(3-methoxyphenyl)-1H-pyrazol-1-yl)-4-morpholinopyrido[3,2-d]pyrimidine-7-carboxylate

A mixture of 4-(7-bromo-2-(4-(3-methoxyphenyl)-1H-pyrazol-1-yl)pyrido[3,2-d]pyrimidin-4-yl)morpholine (0.25 g, 0.53 mmol), triethylamine (0.16 g, 1.6 mmol) and [1,1′-bis(diphenylphosphino)ferrocene]dichloropalladium(II) (0.037 g, 0.05 mmol) in dimethyl sulfoxide/Methanol (8 mL/8 mL) was stirred at 75° C. under carbon monoxide atmosphere for 5 h. The reaction mixture was diluted with water (25 mL) and extracted with ethyl acetate (25 mL*3), the combined organic layer was concentrated and purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product (0.1 g, 42%) as yellow oil. ¹H NMR (400 MHz, DMSO-d₆) δ 9.17-9.09 (m, 2H), 8.52 (d, J=2.1 Hz, 1H), 8.34 (s, 1H), 7.43-7.29 (m, 3H), 6.86 (d, J=8.7 Hz, 1H), 4.64 (bs, 4H), 3.98 (s, 3H), 3.88-3.81 (m, 7H); LCMS (ESI) m/z: 447.8 [M+H]⁺.

### Step 5: Synthesis of 2-(4-(3-methoxyphenyl)-1H-pyrazol-1-yl)-4-morpholinopyrido[3,2-d]pyrimidine-7-carboxylic acid

To a solution of methyl 2-(4-(3-methoxyphenyl)-1H-pyrazol-1-yl)-4-morpholinopyrido[3,2-d]pyrimidine-7-carboxylate (20 mg, 0.045 mmol) in Methanol (2 mL) and water (2 mL) was added sodium hydroxide (7 mg, 0.19 mmol) at 0° C. and stirred at 25° C. for 16 h. The reaction was concentrated and purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to afford the target compound 2-(4-(3-methoxyphenyl)-1H-pyrazol-1-yl)-4-morpholinopyrido[3,2-d]pyrimidine-7-carboxylic acid off-white solid (4.1 mg, 21%). ¹H NMR (400 MHz, DMSO-d₆) δ 9.10 (s, 1H), 9.07 (d, J=1.8 Hz, 1H), 8.32 (s, 1H), 8.30 (s, 1H), 7.40-7.30 (m, 3H), 6.85 (d, J=8.3 Hz, 1H), 4.59 (bs, 4H), 3.80-3.65 (m, 7H); LCMS (ESI) m/z: 432.8 [M+]⁺.

### Step 6: Synthesis of (2-(4-(3-methoxyphenyl)-1H-pyrazol-1-yl)-4-morpholinopyrido[3,2-d]pyrimidin-7-yl)methanol

To a solution of methyl 2-(4-(3-methoxyphenyl)-1H-pyrazol-1-yl)-4-morpholinopyrido[3,2-d]pyrimidine-7-carboxylate (100 mg, 0.22 mmol) in tetrahydrofuran (4 mL) was added solution of lithium aluminum hydride in tetrahydrofuran (0.34 ml, 0.34 mmol) at 0° C. and the mixture was warmed up and stirred at 25° C. for 1 h. The reaction mixture was then concentrated and purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to afford the desired compound (2-(4-(3-methoxyphenyl)-1H-pyrazol-1-yl)-4-morpholinopyrido[3,2-d]pyrimidin-7-yl)methanol as off-white solid (11 mg, 12%). ¹H NMR (400 MHz, DMSO) δ 9.10 (s, 1H), 8.69 (d, J=2.0 Hz, 1H), 8.31 (s, 1H), 8.02 (s, 1H), 7.42-7.28 (m, 3H), 6.89-6.82 (m, 1H), 5.61 (t, J=5.7 Hz, 1H), 4.75 (d, J=4.0 Hz, 2H), 4.60 (bs, 4H), 3.80-3.68 (m, 7H); LCMS (ESI) m/z: 419.8 [M+H]⁺.

### Synthesis of 3-(1-(4-morpholinopyrido[3,2-d]pyrimidin-2-yl)-1H-pyrazol-4-yl)phenol (Compound 46) and 4-(2-(3-(1H-pyrazol-4-yl)phenoxy)pyrido[3,2-d]pyrimidin-4-yl)morpholine (Compound 47)

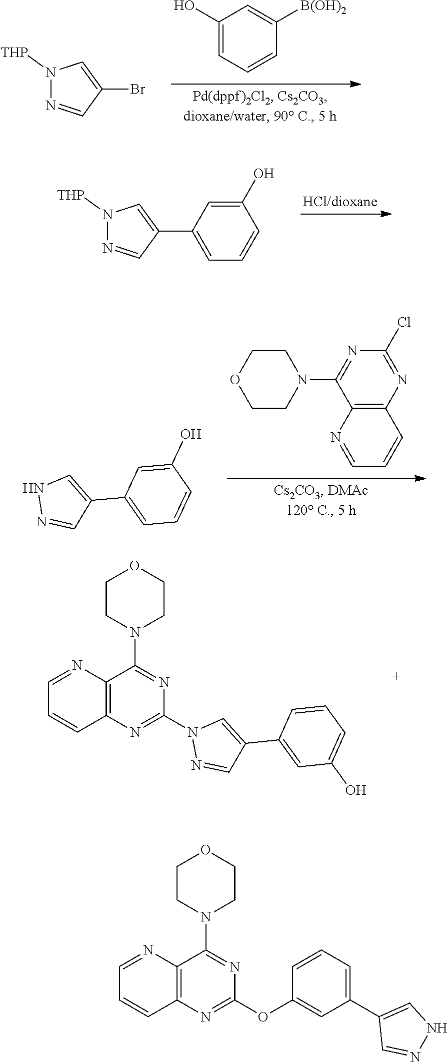

### Step 1: Synthesis of 3-(1-(tetrahydro-2H-pyran-2-yl)-1H-pyrazol-4-yl)phenol

A mixture of 4-bromo-1-(tetrahydro-2H-pyran-2-yl)-1H-pyrazole (690 mg, 3.0 mmol), (3-hydroxyphenyl) boronic acid (414 mg, 3.0 mmoll), [1,1′-bis(diphenylphosphino)ferrocene]dichloropalladium(II) (110 mg, 0.15 mmol) and cesium carbonate (2.93 g, 9.0 mmol) in dioxane (8 mL) and water (2 mL) was stirred at 90° C. under nitrogen atmosphere for 5 h. The reaction mixture was purified by using silica gel chromatography eluting with petroleum ether containing 20% ethyl acetate to obtain the target compound as brown solid. (600 mg, 81.97%)

### Step 2: Synthesis of 3-(1H-pyrazol-4-yl)phenol

A mixture of 3-(1-(tetrahydro-2H-pyran-2-yl)-1H-pyrazol-4-yl)phenol (600 mg, 2.46 mmol) in trifluoroacetic acid (2 mL) was stirred at room temperature for 16 h. The mixture was concentrated to obtain the target product as a brown solid (500 mg), which was directly used in the next step without further purification.

### Step 3: Synthesis of 3-(1-(4-morpholinopyrido[3,2-d]pyrimidin-2-yl)-1H-pyrazol-4-yl)phenol (Compound 46) and 4-(2-(3-(1H-pyrazol-4-yl)phenoxy)pyrido[3,2-d]pyrimidin-4-yl)morpholine (Compound 47)

A mixture of 3-(1H-pyrazol-4-yl)phenol (200 mg, 0.98 mmol), 4-(2-chloropyrido[3,2-d]pyrimidin-4-yl)morpholine (250 mg, 1.00 mmol) and cesium carbonate (1.63 g, 5.00 mmol) in N,N-dimethylformamide (5 mL) was stirred at 120° C. under nitrogen atmosphere for 16 h. The mixture was then filtered and purified by prep-HPLC (Column Xbridge 21.2*250 mm C18, 10 um, Mobile Phase A: water (10 mmol/L ammonium bicarbonate) B: acetonitrile) to obtain 3-(1-(4-morpholinopyrido[3,2-d]pyrimidin-2-yl)-1H-pyrazol-4-yl)phenol (26.1 mg, 7.12%) and 4-(2-(3-(1H-pyrazol-4-yl)phenoxy)pyrido[3,2-d]pyrimidin-4-yl)morpholine (14.6 mg, 3.98%) as white solids.

Compound 46: ¹H NMR (400 MHz, DMSO-d₆) δ 9.48 (s, 1H), 9.01 (s, 1H), 8.79-8.72 (m, 1H), 8.21 (s, 1H), 8.16 (d, J=8.5 Hz, 1H), 7.83 (dd, J=8.5, 4.1 Hz, 1H), 7.21 (d, J=4.5 Hz, 2H), 7.15 (s, 1H), 6.69 (s, 1H), 4.62 (s, 4H), 3.83 (d, J=4.3 Hz, 4H), LCMS (ESI) m/z: 375.0. [M+H]⁺.

Compound 47: ¹H NMR (400 MHz, DMSO-d₆) δ 12.80 (bs, 1H), 8.64 (dd, J=4.1, 1.7 Hz, 1H), 8.17 (bs, 2H), 7.89 (dd, J=8.5, 1.7 Hz, 1H), 7.69 (dd, J=8.5, 4.1 Hz, 1H), 7.48 (dd, J=4.0, 2.0 Hz, 2H), 7.39 (t, J=8.1 Hz, 1H), 7.05-6.99 (m, 1H), 4.40 (bs, 4H), 3.78-3.70 (m, 4H); LCMS (ESI) m/z: 375.0. [M+H]⁺.

### Synthesis of 4-(2-(3-phenyl-1H-pyrazol-1-yl)-7-(pyridin-4-yl)pyrido[3,2-d]pyrimidin-4-yl)morpholine (Compound 48)

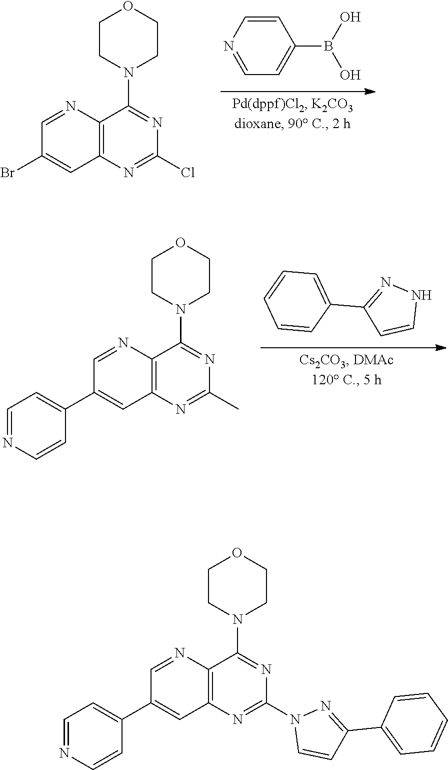 [SMILES: Clc1nc(N2CCOCC2)c2ncc(Br)cc2n1 ; OB(O)c1ccncc1 ; Cc1nc(N2CCOCC2)c2ncc(-c3ccncc3)cc2n1 ; c1ccc(-c2cc[nH]n2)cc1 ; c1ccc(-c2ccn(-c3nc(N4CCOCC4)c4ncc(-c5ccncc5)cc4n3)n2)cc1]

### Step 1: Synthesis of 4-(2-chloro-7-(pyridin-4-yl)pyrido[3,2-d]pyrimidin-4-yl)morpholine

A mixture of 4-(7-bromo-2-chloropyrido[3,2-d]pyrimidin-4-yl)morpholine (660 mg, 2.0 mmol), pyridin-4-ylboronic acid (246 mg, 2.0 mmol), potassium carbonate (414 mg, 3 mmol) and [1,1′-bis(diphenylphosphino)ferrocene]dichloropalladium (II) (73 mg, 0.1 mmol) in dioxane (8 mL) and water (2 mL) was stirred at 90° C. under nitrogen atmosphere for 2 h. After cooling to room temperature, the reaction mixture was filtered and the filtrate was concentrated under reduced pressure. The obtained residue was purified using column chromatography eluting with dichloromethane containing 20% methanol to obtain the target product as brown solid (500 mg, 76.27%). LCMS (ESI) m/z: 328.0 [M+H]⁺.

### Step 2: Synthesis of 4-(2-(3-phenyl-1H-pyrazol-1-yl)-7-(pyridin-4-yl)pyrido[3,2-d]pyrimidin-4-yl)morpholine

To a solution of 4-(2-chloro-7-(pyridin-4-yl)pyrido[3,2-d]pyrimidin-4-yl)morpholine (150 mg, 0.46 mmol) in N,N-dimethylacetamide (5 mL) were added 3-phenyl-1H-pyrazole (66 mg, 0.46 mmol) and cesium carbonate (750 mg, 2.30 mmol). The resultant mixture was stirred at 120° C. under nitrogen atmosphere for 5 h. It was filtered and the crude product from the filtrate was purified by prep-HPLC (Column Xbridge 21.2*250 mm C18, 10 um, Mobile Phase A: water (10 mmol/L ammonium bicarbonate) B: acetonitrile) to afford 4-(2-(3-phenyl-1H-pyrazol-1-yl)-7-(pyridin-4-yl)pyrido[3,2-d]pyrimidin-4-yl)morpholine as white solid (12.5 mg, 6.23%). ¹H NMR (400 MHz, DMSO-d₆) δ 9.20 (d, J=2.3 Hz, 1H), 8.83 (d, J=2.7 Hz, 1H), 8.78 (d, J=6.1 Hz, 2H), 8.66 (d, J=2.3 Hz, 1H), 8.04 (d, J=6.1 Hz, 2H), 7.99 (d, J=7.1 Hz, 2H), 7.50 (t, J=7.5 Hz, 2H), 7.41 (t, J=7.4 Hz, 1H), 7.11 (d, J=2.7 Hz, 1H), 4.65 (s, b4H), 3.88-3.83 (m, 4H); LCMS (ESI) m/z: 435.8 [M]⁺.

### Synthesis of 4-(7-(2,3-dihydrobenzo[b][1,4]dioxin-6-yl)-2-(3-(pyridin-3-yl)-1H-pyrazol-1-yl)pyrido[3,2-d]pyrimidin-4-yl)morpholine (Compound 49)

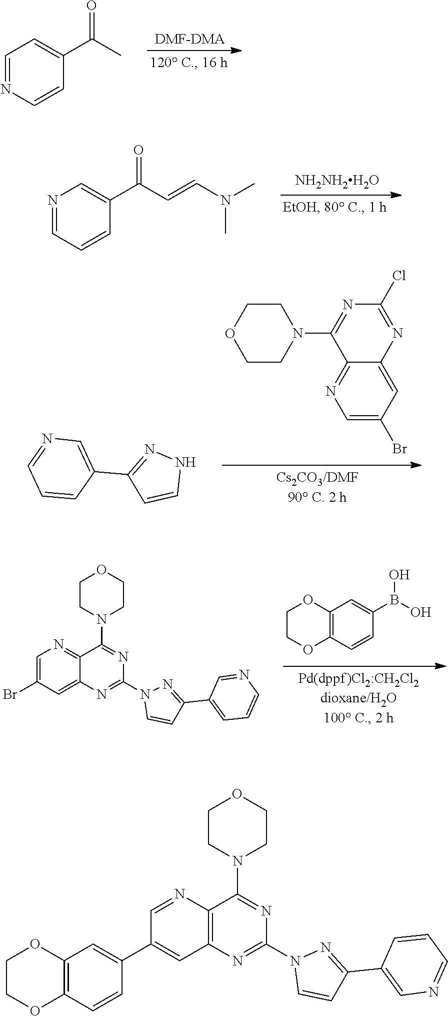

### Step 1: Synthesis of 3-(dimethylamino)-1-(pyridin-3-yl)prop-2-en-1-one

A mixture of 1-(pyridin-3-yl)ethanone (1.21 g, 10.0 mmol) and N,N-dimethylformamide dimethyl acetal (10 mL) was stirred at 120° C. for 16 h. The mixture was poured into water and extracted with ethyl acetate (100 mL*2). The combined organic phase was concentrated to afford (E)-3-(dimethylamino)-1-(pyridin-3-yl)prop-2-en-1-one (2.1 g) as brown oil, which was used in the next step without further purification. LCMS (ESI) m/z: 177.0 [M+H]⁺.

### Step 2: Synthesis of 3-(1H-pyrazol-3-yl)pyridine

A mixture of 3-(dimethylamino)-1-(pyridin-3-yl)prop-2-en-1-one (2.1 g), hydrazine hydrate (98%, 3 mL) and ethanol (30 mL) was stirred at 100° C. for 1 h. The mixture was poured into water and extracted with ethyl acetate (100 mL*2). The combined organic phase was concentrated and the residue was purified by silica gel column chromatography (ethyl acetate as eluent) to afford 3-(1H-pyrazol-3-yl)pyridine (540 mg, 3.7 mmol) as yellow oil. LCMS (ESI) m/z: 146.1 [M+H]⁺.

### Step 3: Synthesis of 4-(7-bromo-2-(3-(pyridin-3-yl)-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (328 mg, 1.0 mmol), 3-(1H-pyrazol-3-yl)pyridine (174 mg, 1.2 mmol) and cesium carbonate (650 mg, 2.0 mmol) in N,N-dimethylformamide (15 mL) was stirred at 100° C. for 2 h. The mixture was poured into water (100 mL) and the resultant precipitate was collected by filtration and washed with ethyl acetate (40 mL) to afford 4-(7-bromo-2-(3-(pyridin-3-yl)-1H-pyrazol-1-yl)pyrido[3,2-d]pyrimidin-4-yl)morpholine (260 mg) as off-white solid. LCMS (ESI) m/z: 437.6/439.6 [M+H]⁺.

### Step 4: Synthesis of 4-(7-(2,3-dihydrobenzo[b][1,4]dioxin-6-yl)-2-(3-(pyridin-3-yl)-1H-pyrazol-1-yl)pyrido[3,2-d]pyrimidin-4-yl)morpholine

A mixture of 4-(7-bromo-2-(3-(pyridin-3-yl)-1H-pyrazol-1-yl)pyrido[3,2-d]pyrimidin-4-yl)morpholine (200 mg), 2,3-dihydrobenzo[b][1,4]dioxin-6-ylboronic acid (180 mg, 1.0 mmol), 1,1′-bis(diphenylphosphino)ferrocene-palladium(II)dichloride dichloromethane complex (81 mg, 0.1 mmol) and cesium carbonate (650 mg, 2.0 mmol) in water (4 mL) and dioxane (20 mL) was stirred at 100° C. under nitrogen atmosphere for 2 h. The mixture was concentrated and the residue was purified successively by silica gel column chromatography (20% dichloromethane in methanol) and by prep-HPLC (Column Xbridge 21 0.2*250 mm C18, 10 um, Mobile Phase A: water (10 mmol/L ammonium bicarbonate) B: acetonitrile) to afford 4-(7-(2,3-dihydrobenzo[b][1,4]dioxin-6-yl)-2-(3-(pyridin-3-yl)-1H-pyrazol-1-yl)pyrido[3,2-d]pyrimidin-4-yl)morpholine (86.6 mg, 17%) as off-white solid. ¹H NMR (400 MHz, DMSO-d) δ 9.18 (s, 1H), 9.04 (d, J=2.1 Hz, 1H), 8.84 (d, J=2.6 Hz, 1H), 8.60 (d, J=3.7 Hz, 1H), 8.39-8.29 (m, 2H), 7.56-7.42 (m, 3H), 7.20 (d, J=2.6 Hz, 1H), 7.03 (d, J=8.4 Hz, 1H), 4.59 (bs, 4H), 4.32 (s, 4H), 3.85 ((, 4H); LCMS (ESI) m/z: 493.8 [M]⁺.

The following compounds were synthesized according to the protocol described above:

| Name | Structure | NMR, MS | # |
|---|---|---|---|
| 4-[2-(3-pyrimidin-5- ylpyrazol-1-yl)-7- tetrahydropyran-4-yl- pyrido[3,2-d]pyrimidin- 4-yl]morpholine | 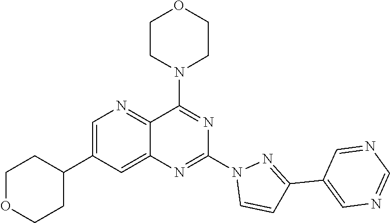 [SMILES: c1ncc(-c2ccn(-c3nc(N4CCOCC4)c4ncc(C5CCOCC5)cc4n3)n2)cn1] | ¹H NMR (400MHz, CHLOROFORM-d) δ = 9.34 (s, 2H), 9.22 (s, 1H), 8.71 (d, J = 2.8 Hz, 1H), 8.59 (d, J = 2.3 Hz, 1H), 8.08 (d, J = 2.0 Hz, 1H), 6.87 (d, J = 2.8 Hz, 1H), 4.65 (bs, 4H), 4.15 (dd, J = 10.4, 2.6Hz, 2H), 3.95 (t, J = 4.4Hz, 4H), 3.60 (dt, J = 11.2, 3.4Hz, 2H), 2.99 (hept, J = 5.2Hz, 1H), 1.99-1.83 (m, 4H). LCMS (ESI) for C23H24N8O2 [M + H]⁺: 445.3. | 50 |
| 4-[2-[3-(4- pyridyl)pyrazol-1-yl]-7- tetrahydropyran-4-yl- pyrido[3,2-d]pyrimidin- 4-yl]morpholine | 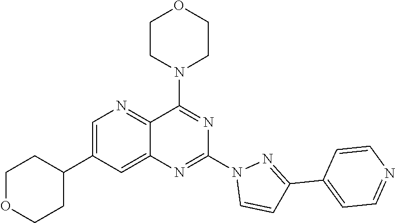 [SMILES: c1cc(-c2ccn(-c3nc(N4CCOCC4)c4ncc(C5CCOCC5)cc4n3)n2)ccn1] | ¹H NMR (400MHz, CHLOROFORM-d) δ = 8.74-8.65 (m, 3H), 8.58 (d, J = 2.3 Hz, 1H), 8.08 (d, J = 2.1 Hz, 1H), 7.95-7.86 (m, 2H), 6.89 (d, J = 2.8 Hz, 1H), 4.66 (br s, 4H), 4.16 (dd, J = 2.8, 10.5 Hz, 2H), 4.01-3.91 (m, 4H), 3.61 (dt, J = 3.2, 11.2 Hz, 2H), 3.05- 2.90 (m, 1H), 1.99-1.82 (m, 4H). LCMS (ESI) for C24H25N7O2 [M + H]⁺: 444.3. | 51 |
| 4-[2-[3-(3- pyridyl)pyrazol-1-yl]-7- tetrahydropyran-4-yl- pyrido[3,2-d]pyrimidin- 4-yl]morpholine | 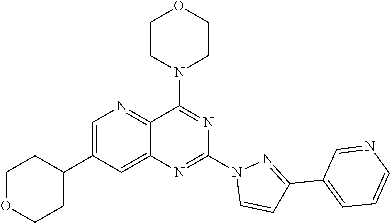 [SMILES: c1cncc(-c2ccn(-c3nc(N4CCOCC4)c4ncc(C5CCOCC5)cc4n3)n2)c1] | ¹H NMR (400MHz, CHLOROFORM-d) δ = 9.19 (d, J = 1.6 Hz, 1H), 8.67 (d, J = 2.8 Hz, 1H), 8.61 (dd, J = 4.8, 1.6Hz, 1H), 8.57 (d, J = 2.3 Hz, 1H), 8.37 (td, J = 8.0, 1.9Hz, 1H), 8.08 (d, J = 2.0 Hz, 1H), 7.43-7.32 (m, 1H), 6.86 (d, J = 2.8 Hz, 1H), 4.65 (bs, 4H), 4.15 (dd, J = 10.4, 2.8Hz, 2H), 3.94 (t, J = 4.4Hz, 4H), 3.60 (dt, J = 11.2, 3.3Hz, 2H), 2.98 (hept, J = 5.2Hz, 1H), 2.00-1.80 (m, 4H). LCMS (ESI) for C24H25N7O2 [M + H]⁺: 444.3. | 52 |
| 4-[2-(3-pyrimidin-4- ylpyrazol-1-yl)-7- tetrahydropyran-4-yl- pyrido[3,2-d]pyrimidin- 4-yl]morpholine | 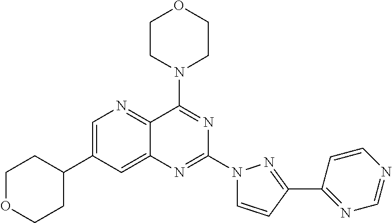 [SMILES: c1cc(-c2ccn(-c3nc(N4CCOCC4)c4ncc(C5CCOCC5)cc4n3)n2)ncn1] | ¹H NMR (400MHz, DMSO-d₆) δ = 9.27 (s, 1H), 8.97-8.92 (m, 2H), 8.88-8.75 (m, 1H), 8.13-8.12 (m, 1H), 8.03 (s, 1H), 7.23 (s, 1H), 4.57 (bs, 4H), 4.03-4.00 (m, 2H), 3.84 (m, 4H), 3.53-3.51 (m, 2H), 3.08 (m, 1H), 1.85-1.81 (m, 4H). LCMS (ESI) for C23H24N8O2 [M + H]⁺: 445.2. | 53 |

### Synthesis of 4-(7-methoxy-2-(3-m-tolyl-1H-pyrazol-1-yl)pyrido[3,2-d]pyrimidin-4-yl)morpholine (Compound 54)

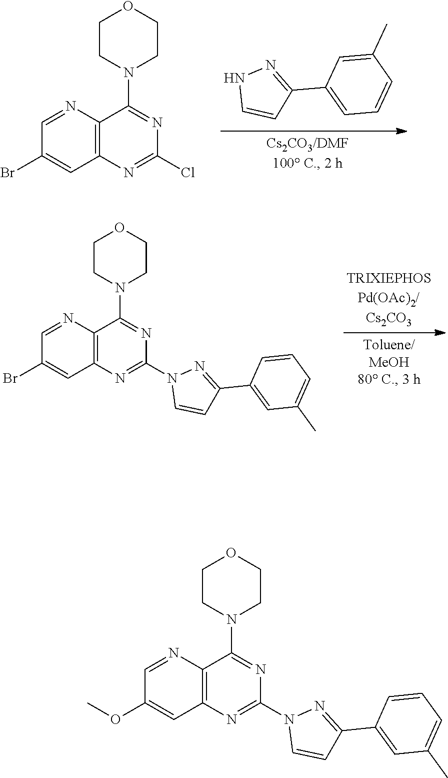

### Step 2: Synthesis of 4-(7-methoxy-2-(3-m-tolyl-1H-pyrazol-1-yl)pyrido[3,2-d]pyrimidin-4-yl)morpholine

A mixture of 4-(7-bromo-2-(3-m-tolyl-1H-pyrazol-1-yl)pyrido[3,2-d]pyrimidin-4-yl)morpholine (270 mg, 0.6 mmol), palladium (II) acetate (14 mg, 0.06 mmol), racemic-2-Di-t-butylphosphino-1,1′-binaphthyl (48 mg, 0.12 mmol) and cesium carbonate (390 mg, 1.2 mmol) in methanol (4 mL) and toluene (15 mL) was stirred at 80° C. for 3 h. The mixture filtered and the crude product from the filtrate was purified successively by silica gel column chromatography (20% methanol in dichloromethane) and prep-HPLC (Column Xbridge 21.2*250 mm C18, 10 um, Mobile Phase A: water (10 mmol/L ammonium bicarbonate) B: acetonitrile) to afford 4-(7-methoxy-2-(3-m-tolyl-1H-pyrazol-1-yl)pyrido[3,2-d]pyrimidin-4-yl)morpholine (42.4 mg, 16.7%) as white solid. ¹H NMR (400 MHz, CDCl₃) δ 8.61 (d, J=2.7 Hz, 1H), 8.35 (d, J=2.9 Hz, 1H), 7.91 (s, 1H), 7.77 (d, J=7.7 Hz, 1H), 7.56 (d, J=2.8 Hz, 1H), 7.32 (t, J=7.6 Hz, 1H), 7.17 (d, J=7.5 Hz, 1H), 6.80 (d, J=2.7 Hz, 1H), 4.58 (bs, 4H), 3.96 (s, 3H), 3.94-3.90 (m, 4H), 2.42 (s, 3H); LCMS (ESI) m/z: 402.8 [M+H]⁺.

### Synthesis of 4-[7-cyclopropyl-2-(3-phenylpyrazol-1-yl)pyrido[3,2-d]pyrimidin-4-yl]morpholine (Compound 55)

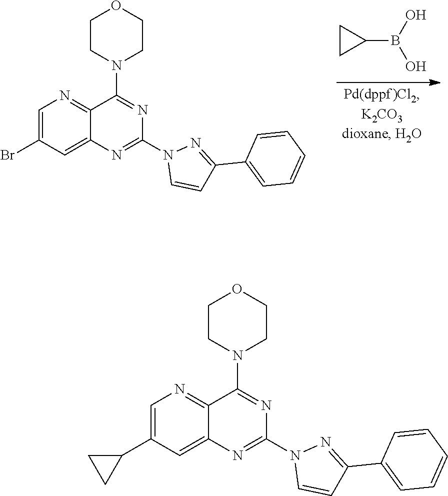 [SMILES: Brc1cnc2c(N3CCOCC3)nc(-n3ccc(-c4ccccc4)n3)nc2c1 ; OB(O)C1CC1 ; c1ccc(-c2ccn(-c3nc(N4CCOCC4)c4ncc(C5CC5)cc4n3)n2)cc1]

To a solution of 4-[7-bromo-2-(3-phenylpyrazol-1-yl)pyrido[3,2-d]pyrimidin-4-yl]morpholine (0.1 g, 229 umol) in dioxane (2 mL) and H₂O (0.4 mL) were added cyclopropyl boronic acid (39 mg, 457 umol), K₂CO₃ (79 mg, 572 umol) and Pd(dppf)Cl₂ (8 mg, 11 umol). The resultant mixture stirred at 80° C. for 16 h under nitrogen atmosphere. 10 mL of water was added to the mixture and it was extracted with ethyl acetate (20 mL*2). The combined organic layers were washed with brine (15 mL), dried over Na₂SO₄ and concentrated. The resultant residue was subjected to prep-HPLC (Welch Xtimate C18 150*25 5u column, 20-50% acetonitrile in an 0.04% hydrochloric acid solution in water, 8 min gradient) to afford 4-[7-cyclopropyl-2-(3-phenylpyrazol-1-yl)pyrido[3,2-d]pyrimidin-4-yl]morpholine (51 mg, 56%) as white solid. 1H NMR (400 MHz, DMSO-d₆) δ=8.88 (d, J=2.6 Hz, 1H), 8.64 (d, J=2.3 Hz, 1H), 8.08-8.00 (m, 2H), 7.92 (d, J=2.1 Hz, 1H), 7.55-7.40 (m, 3H), 7.19 (d, J=2.8 Hz, 1H), 4.63 (bs, 4H), 3.83 (t, J=4.4 Hz, 4H), 2.26-2.15 (m, 1H), 1.26-1.16 (m, 2H), 1.04-0.92 (m, 2H). LCMS (ESI) for C₂₃H₂₂N₆O [M+H]⁺: 399.2.

### Synthesis of 4-methyl-1-(4-morpholino-2-(4-phenyl-1H-pyrazol-1-yl)pyrido[3,2-d]pyrimidin-7-yl)piperazin-2-one (Compound 56)

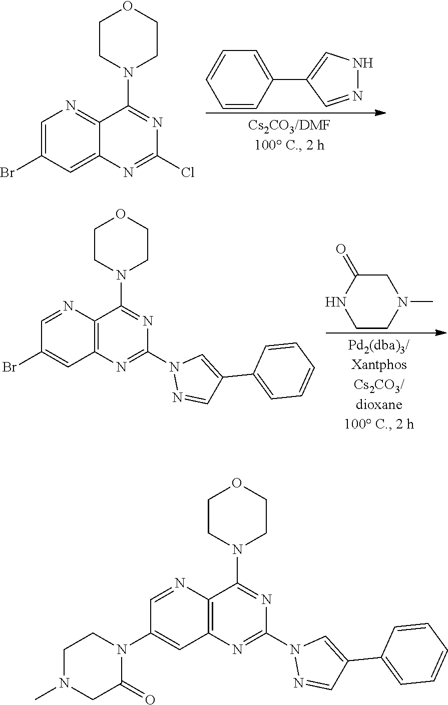

### Step 1: Synthesis of 4-(7-bromo-2-(4-phenyl-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (328 mg, 1.0 mmol), 4-phenyl-1H-pyrazole (237 mg, 1.5 mmol) and cesium carbonate (650 mg, 2.0 mmol) and N,N-dimethylformamide (15 mL) was stirred at 100° C. for 2 h. The mixture was poured into water (100 mL) and the resultant precipitate was collected by filtration and dried under vacuum to afford 4-(7-bromo-2-(4-phenyl-1H-pyrazol-1-yl)pyrido[3,2-d]pyrimidin-4-yl)morpholine (400 mg, 91%) as grey solid. LCMS (ESI) m/z: 438.6/439.6 [M+H]⁺.

### Step 2: Synthesis of 4-methyl-1-(4-morpholino-2-(4-phenyl-1H-pyrazol-1-yl)pyrido[3,2-d]pyrimidin-7-yl)piperazin-2-one

A mixture of 4-(7-bromo-2-(4-phenyl-1H-pyrazol-1-yl)pyrido[3,2-d]pyrimidin-4-yl)morpholine (200 mg, 0.45 mmol), 4-methylpiperazin-2-one (102 mg, 0.9 mmol), tris(dibenzylideneacetone)dipalladium(0) (41 mg, 0.045 mmol), 4,5-bis(diphenylphosphino)-9,9-dimethylxanthene (52 mg, 0.09 mmol) and cesium carbonate (292 mg, 0.9 mmol) in dioxane (10 mL) was stirred at 100° C. under nitrogen atmosphere for 2 h. The mixture was poured into water, extracted with dichloromethane (100 mL*2) and the combined organic phase was concentrated. The residue was purified successively by silica gel column chromatography (10% of methanol in dichloromethane) and prep-HPLC (Column Xbridge 21.2*250 mm C18, 10 um, Mobile Phase A: water (10 mmol/L ammonium bicarbonate) B: acetonitrile) to afford 4-methyl-1-(4-morpholino-2-(4-phenyl-1H-pyrazol-1-yl)pyrido[3,2-d]pyrimidin-7-yl)piperazin-2-one (66.0 mg, 31.1%) as white solid. ¹H NMR (400 MHz, CDCl₃) δ 8.94 (d, J=2.5 Hz, 1H), 8.77 (d, J=0.8 Hz, 1H), 8.11 (d, J=0.8 Hz, 1H), 7.99 (d, J=2.5 Hz, 1H), 7.67-7.58 (m, 2H), 7.42 (t, J=7.7 Hz, 2H), 7.33-7.28 (m, 1H), 4.64 (bs, 4H), 3.98-3.89 (m, 4H), 3 3.85 (t, J=4.0 Hz, 2H), 3.37 (s, 2H), 2.89 (t, J=4.0 Hz, 2H), 2.44 (s, 3H); LCMS (ESI) m/z: 470.9 [M]⁺.

### Synthesis of 4-(2-(3-phenyl-1H-pyrazol-1-yl)pyrido[3,4-d]pyrimidin-4-yl)morpholine (Compound 57)

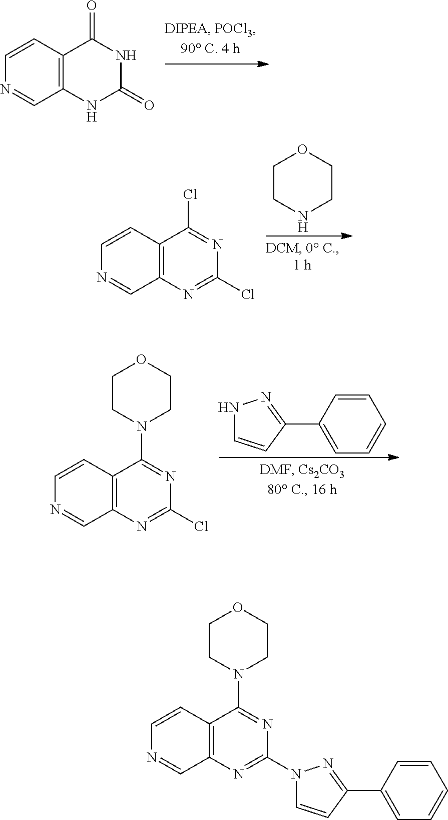 [SMILES: O=c1[nH]c(=O)c2ccncc2[nH]1 ; Clc1nc(Cl)c2ccncc2n1 ; C1COCCN1 ; Clc1nc(N2CCOCC2)c2ccncc2n1 ; c1ccc(-c2cc[nH]n2)cc1 ; c1ccc(-c2ccn(-c3nc(N4CCOCC4)c4ccncc4n3)n2)cc1]

### Step 1: Synthesis of 2,4-dichloropyrido[3,4-d]pyrimidine

To a mixture of pyrido[3,4-d]pyrimidine-2,4(1H,3H)-dione (250 mg, 1.53 mmol) in phosphoryl trichloride (5 mL) was added N,N-diisopropylethylamine (3.9 g, 30.6 mmol). The reaction mixture was refluxed for 4 h and concentrated. The residue was subjected to flash chromatography (eluted with methanol/dichloromethane=1:10 to 1:3) to afford 2,4-dichloropyrido[3,4-d]pyrimidine (39 mg, 13%) as yellow solid. LCMS (ESI) m/z: 199.9 [M+H]⁺.

### Step 2: Synthesis of 4-(2-chloropyrido[3,4-d]pyrimidin-4-yl)morpholine

To a solution of 2,4-dichloropyrido[3,4-d]pyrimidine (39 mg, 0.2 mmol) in 1,4-dioxane (4 mL) was added morpholine (26 mg, 0.3 mmol) and the mixture was stirred at 0° C. for 1 h. The reaction mixture was concentrated, and the residue was diluted with water (3 mL), and extracted with ethyl acetate (10 mL*2). The combined organic layer was dried over anhydrous sodium sulfate, filtered and concentrated. The resulting solid was washed with diethyl ether to afford 4-(2-chloropyrido[3,4-d]pyrimidin-4-yl)morpholine (31 mg, 62%) as yellow solid. LCMS (ESI) m/z: 251.1 [M+H]⁺.

### Step 3: Synthesis of 4-(2-(3-phenyl-1H-pyrazol-1-yl)pyrido[3,4-d]pyrimidin-4-yl)morpholine

To a solution of 4-(2-chloropyrido[3,4-d]pyrimidin-4-yl)morpholine (31 mg, 0.12 mmol) in N,N-dimethylformamide (4 mL) were added 3-phenyl-1H-pyrazole (27 mg, 0.18 mmol) and cesium carbonate (117 mg, 0.36 mmol). The reaction mixture was stirred at 80° C. 16 h and it was extracted with ethyl acetate (20 mL*2), washed with brine (10 mL*3), dried over sodium sulfate, filtered and concentrated. The residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to afford 4-(2-(3-phenyl-1H-pyrazol-1-yl)pyrido[3,4-d]pyrimidin-4-yl)morpholine (4.0 mg, 9%) as white solid. ¹H NMR (400 MHz, CDCl₃) δ 9.22 (s, 1H), 8.82 (d, J=2.8 Hz, 1H), 8.53 (d, J=6.0 Hz, 1H), 8.02-7.96 (m, 3H), 7.52-7.40 (m, 3H), 7.11 (d, J=2.8 Hz, 1H), 4.05 (t, J=4.4 Hz, 4H), 3.83 (t, J=4.4 Hz, 4H); LCMS (ESI) m/z: 359.1 [M+H]⁺.

### Synthesis of 4-(2-(3-phenyl-1H-pyrazol-1-yl)-7-(tetrahydrofuran-2-yl)pyrido[3,2-d]pyrimidin-4-yl)morpholine (Compound 58)

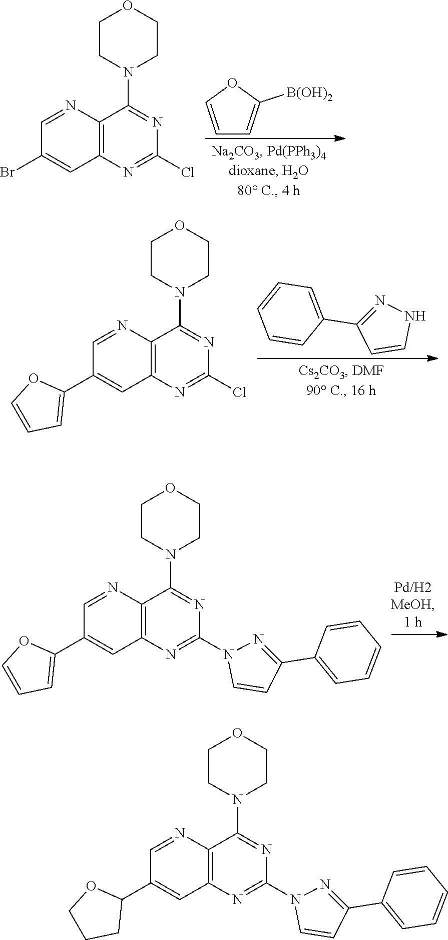

### Step 1: Synthesis of 4-(2-chloro-7-(furan-2-yl)pyrido[3,2-d]pyrimidin-4-yl)morpholine

To a solution of 4-(7-bromo-2-chloropyrido[3,2-d]pyrimidin-4-yl)morpholine (1 g, 3 mmol) in dioxane (30 mL)/H₂O (6 mL) were added furan-2-ylboronic acid (376 mg, 3.35 mmol), Na₂CO₃ (646 mg, 6.1 mmol) and Pd(PPh₃)₄ (351 mg, 0.3 mmol). The resultant reaction mixture was stirred at 80° C. for 4 h under nitrogen atmosphere. It was then concentrated and the residue was subjected to silica gel chromatography (PE/EA=4:1 to 1:1) to afford 4-(2-chloro-7-(furan-2-yl)pyrido[3,2-d]pyrimidin-4-yl)morpholine (620 mg, 64%) as a yellow solid. LCMS (ESI) m/z: 317.1 [M+H]⁺.

### Step 2: Synthesis of 4-(7-(furan-2-yl)-2-(3-phenyl-1H-pyrazol-1-yl)pyrido[3,2-d]pyrimidin-4-yl)morpholine

To a solution of 4-(2-chloro-7-(furan-2-yl)pyrido[3,2-d]pyrimidin-4-yl)morpholine (70 mg, 0.22 mmol), 3-phenyl-1H-pyrazole (35 mg, 0.24 mmol) in DMF (5 mL) was added Cs₂CO₃ (216 mg, 0.66 mmol). The resultant reaction mixture was stirred at 90° C. for 6 h. Then the reaction was quenched with water (5 mL) and the mixture was extracted with EtOAc (20*3 mL). The organic layer was combined, washed with brine (30 mL), dried over Na₂SO₄, filtered and concentrated. The residue was purified by prep-HPLC (0.05% formic acid/H₂O:CH₃CN=5%˜95%) to afford 4-(7-(furan-2-yl)-2-(3-phenyl-1H-pyrazol-1-yl)pyrido[3,2-d]pyrimidin-4-yl)morpholine (33.4 mg, 35%) as yellow solid.

¹H NMR (500 MHz, DMSO-d₆) δ 9.13 (d, J=2.5 Hz, 1H), 8.80 (d, J=3.0 Hz, 1H), 8.36 (d, J=2.5 Hz, 1H), 8.00-7.99 (m, 3H), 7.52-7.46 (m, 3H), 7.40 (t, J=7.5 Hz, 1H), 7.10 (d, J=3.0 Hz, 1H), 6.76 (dd, J=3.5, 2.0 Hz, 1H), 4.57 (bs, 4H), 3.85 (t, J=4.5 Hz, 4H). LCMS (ESI) m/z: 425.3 [M+H]⁺.

### Step 3: Synthesis of 4-(2-(3-phenyl-1H-pyrazol-1-yl)-7-(tetrahydrofuran-2-yl)pyrido[3,2-d]pyrimidin-4-yl)morpholine

To a solution of 4-(7-(furan-2-yl)-2-(3-phenyl-1H-pyrazol-1-yl)pyrido[3,2-d]pyrimidin-4-yl)morpholine (65 mg, 0.15 mmol) in MeOH (20 mL) was added 10% Pd/C (7 mg) and the reaction mixture was stirred at room temperature for 1 h under hydrogen atmosphere. The mixture was filtered and the filtrate was concentrated. The residue was purified by Prep-HPLC (0.05% FA/H₂O:CH₃CN=5%˜95%) to afford 4-(2-(3-phenyl-1H-pyrazol-1-yl)-7-(tetrahydrofuran-2-yl)pyrido[3,2-d]pyrimidin-4-yl)morpholine (10.6 mg, 16%) as yellow solid. ¹H NMR (400 MHz, DMSO-d₆) δ 8.79 (d, J=2.8 Hz, 1H), 8.72 (d, J=2.0 Hz, 1H), 8.45 (s, 1H), 8.05 (d, J=1.6 Hz, 1H), 7.99 (d, J=6.8 Hz, 2H), 7.49 (t, J=8.4 Hz, 2H), 7.40 (t, J=8.0 Hz, 1H), 7.09 (d, J=2.4 Hz, 1H), 5.11 (t, J=7.2 Hz, 1H), 4.60 (bs, 4H), 4.13-4.06 (m, 1H), 3.92 (dd, J=14.4, 6.8 Hz, 2H), 3.85-3.83 (m, 4H), 2.47-2.43 (m, 1H), 2.01 (pent, J=7.2 Hz, 2H), 1.89-1.82 (m, 1H). LCMS (ESI) m/z: 429.1 [M+H]⁺.

### Synthesis of 4-(2-(3-phenyl-1H-pyrazol-1-yl)pyrido[4,3-d]pyrimidin-4-yl)morpholine (Compound 59)

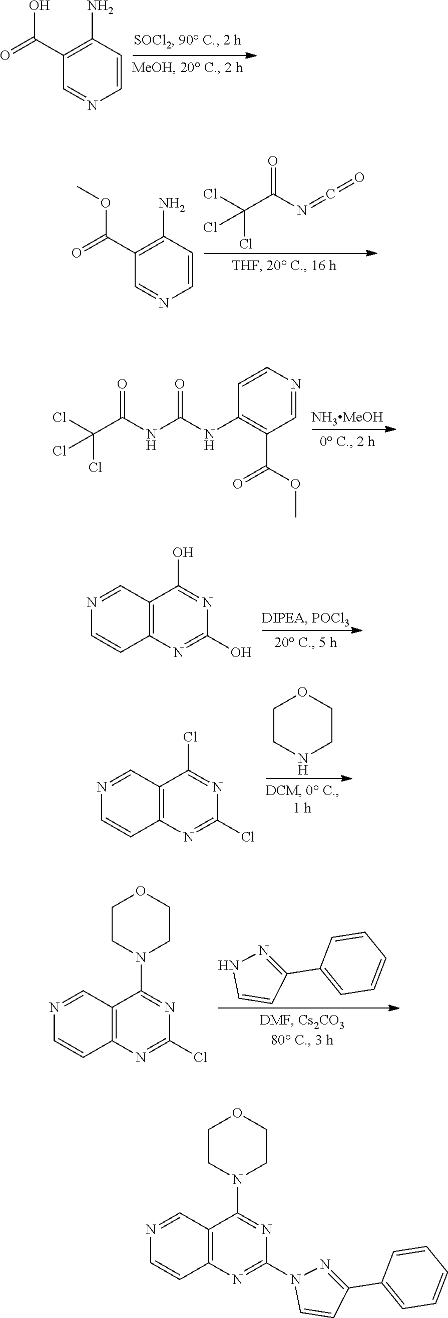

### Step 1: Synthesis of methyl 4-aminonicotinate

A suspension of 4-aminonicotinic acid (5.0 g, 36.2 mmol) in thionyl chloride (10 ml) was stirred at 90° C. for 1 h. The reaction mixture was concentrated to afford 4-aminonicotinoyl chloride (5.1 g, 90%) a as yellow solid. This was dissolved in dry methanol (50 mL) and stirred at 20° C. for 2 h, The reaction mixture was concentrated, aqueous sodium carbonate solution (200 mL) was added and the mixture was stirred further at 20° C. for 0.5 h and extracted with dichloromethane (150 mL*2), The combined organic layer was dried over anhydrous sodium sulfate, filtered and concentrated under reduced pressure to give methyl 4-aminonicotinate (5.1 g, 93%) as yellow solid. LCMS (ESI) m/z: 153.1 [M+H]⁺.

### Step 2: Synthesis of methyl 4-(3-(2,2,2-trichloroacetyl)ureido)nicotinate

To a solution of methyl 4-aminonicotinate (1.0 g, 6.57 mmol) in tetrahydrofuran (20 mL) at 0° C., 2,2,2-trichloroacetyl isocyanate (2.6 g, 13.8 mmol) was added dropwise and then stirred at 20° C. for 20 h. The resultant precipitate was collected by filtration and washed with methanol to give methyl 4-(3-(2,2,2-trichloroacetyl)ureido)nicotinate (1.5 g, 67%) as yellow solid. LCMS (ESI) m/z: 341.9 [M+H]⁺.

### Step 3: Synthesis of pyrido[4,3-d]pyrimidine-2,4-diol

To an ice cold solution of methyl 4-(3-(2,2,2-trichloroacetyl)ureido)nicotinate (750 mg, 2.2 mmol) in anhydrous methanol (7 mL) was added ammonia/methanol (2 mL). The suspension was stirred at 0° C. for 2 h at which time a yellow solid precipitated. The solid was collected by filtration and washed with methanol to give pyrido[4,3-d]pyrimidine-2,4-diol (300 mg, 84%) as yellow solid. ¹H NMR (500 MHz, DMSO-d₆) δ 11.43 (s, 2H), 8.92 (s, 1H), 8.58 (d, J=5.7 Hz, 1H), 7.08 (d, J=5.9 Hz, 1H). LCMS (ESI) m/z: 164.1 [M+H]⁺.

### Step 4: Synthesis of 2,4-dichloropyrido[4,3-d]pyrimidine

To a suspension of pyrido[4,3-d]pyrimidine-2,4-diol (300 mg, 1.84 mmol) in phosphorus oxychloride (6 mL) was added N,N-diisopropylethylamine (3 mL) and then the mixture was stirred at 20° C. for 5 h. It was then concentrated to afford 2,4-dichloropyrido[4,3-d]pyrimidine (368 mg, 99%) as red solid. LCMS (ESI) m/z: 202.2 [M+H]⁺.

### Step 5: Synthesis of 4-(2-chloropyrido[4,3-d]pyrimidin-4-yl)morpholine

A mixture of 2,4-dichloropyrido[4,3-d]pyrimidine (367 mg, 0.76 mmol) and morpholine (662 mg, 183 mmol) in dichloromethane (20 mL) was stirred at 0° C. for 1 h. It was then diluted with dichloromethane (50 mL) and washed with water (50 mL). The organic layer was concentrated and purified by Combi-Flash (Biotage, 40 g silica gel, methanol in dichloromethane from 1% to 6%) to obtain 4-(2-chloropyrido[4,3-d]pyrimidin-4-yl)morpholine (300 mg) as yellow solid. LCMS (ESI) m/z: 251.1 [M+H]⁺.

### Step 6: Synthesis of 4-(2-(3-phenyl-1H-pyrazol-1-yl)pyrido[4,3-d]pyrimidin-4-yl)morpholine

To a solution of 4-(2-chloropyrido[4,3-d]pyrimidin-4-yl)morpholine (250 mg, 1.0 mmol) in DMF (2 mL) were added 3-phenyl-1H-pyrazole (35 mg, 0.24 mmol) and cesium carbonate (130 mg, 0.4 mmol) and the resultant mixture was stirred at 80° C. under nitrogen for 3 h. The resultant mixture was filtered and the filtrate was subjected to prep-HPLC (BOSTON pHlex ODS 10 μm 21.2×250 mm 120 A. The mobile phase was acetonitrile/0.1% Ammonium bicarbonate) to obtain 4-(2-(3-phenyl-1H-pyrazol-1-yl)pyrido[4,3-d]pyrimidin-4-yl)morpholine (8.5 mg, 2.4%) as white solid. ¹H NMR (500 MHz, Chloroform-d) δ 9.26 (d, J=0.8 Hz, 1H), 8.70 (d, J=5.8 Hz, 1H), 8.66 (d, J=2.7 Hz, 1H), 8.04 (d, J=4.8 Hz, 2H), 7.81 (d, J=5.8, 0.8 Hz, 1H), 7.48-7.42 (m, 2H), 7.40-7.36 (m, 1H), 6.84 (d, J=2.7 Hz, 1H), 4.13 (t, J=4.0 Hz, 4H), 3.98 (t, J=4.0 Hz, 4H). LCMS (ESI) m/z: 359.3 [M+H]⁺.

### Synthesis of 4-(7-Ethyl-2-(3-(pyridin-4-yl)-1H-pyrazol-1-yl)pyrido[3,2-d]pyrimidin-4-yl)morpholine (Compound 60)

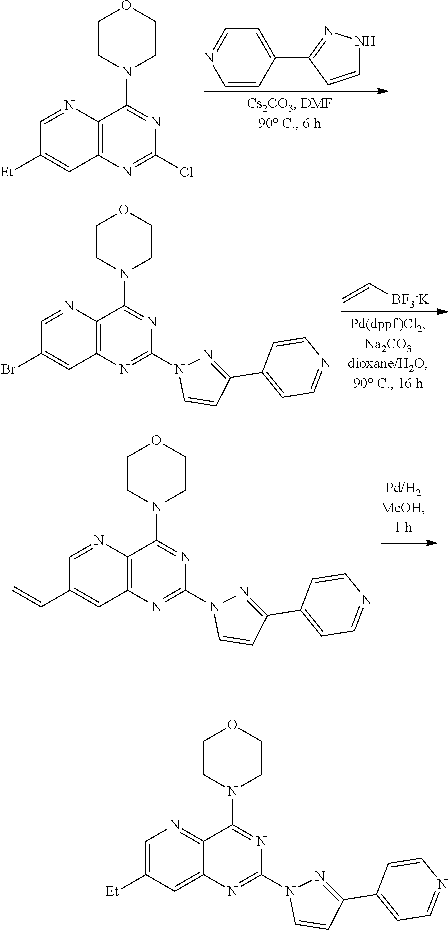

### Step 1: Synthesis of 4-(7-Bromo-2-(3-(pyridin-4-yl)-1H-pyrazol-1-yl)pyrido[3,2-d]pyrimidin-4-yl)morpholine

A mixture of 4-(7-bromo-2-chloropyrido[3,2-d]pyrimidin-4-yl)morpholine (160 mg, 0.49 mmol), 4-(1H-pyrazol-3-yl)pyridine (92 mg, 0.63 mmol) and cesium carbonate (319 mg, 0.98 mmol) in N,N-dimethylformamide (5 mL) was heated to 80° C. and stirred for 6 h. Then the reaction was quenched by the addition water (25 mL) and was extracted with dichloromethane (25 ml*3). The organic layer was dried over Na₂SO₄, filtered and concentrated. The obtained residue was purified by flash chromatography on silica gel (petroleum ether:ethyl acetate=4:1) to obtain 4-(7-bromo-2-(3-(pyridin-4-yl)-1H-pyrazol-1-yl)pyrido[3,2-d]pyrimidin-4-yl)morpholine (120 mg, 56%) as yellow solid. LCMS (ESI) m/z: 438.0[M+H]⁺.

### Step 2: Synthesis of 4-(2-(3-(Pyridin-4-yl)-1H-pyrazol-1-yl)-7-vinylpyrido[3,2-d]pyrimidin-4-yl)morpholine

A solution of 4-(7-bromo-2-(3-(pyridin-4-yl)-1H-pyrazol-1-yl)pyrido[3,2-d]pyrimidin-4-yl)morpholine (120 mg, 0.27 mmol), potassium vinyltrifluoroborate (72 mg, 0.54 mmol), 1,1′-bis(diphenylphosphino)ferrocene-palladium(II)dichloride (20 mg, 0.027 mmol) and sodium carbonate (67 mg, 0.54 mmol) in water (2.0 mL) and dioxane (4.0 mL) was stirred at 90° C. for 1 h under argon atmosphere. The mixture was then diluted with ethyl acetate (50 mL) and washed with water (25 mL). The organic layer was concentrated and purified by purified by flash chromatography (dichloromethane:methanol=20:1) to obtain 4-(2-(3-(pyridin-4-yl)-1H-pyrazol-1-yl)-7-vinylpyrido[3,2-d]pyrimidin-4-yl)morpholine (80 mg, 77%) as white solid. LCMS (ESI) m/z: 386.3 [M+H]⁺.

### Step 3: Synthesis of 4-(7-Ethyl-2-(3-(pyridin-4-yl)-1H-pyrazol-1-yl)pyrido[3,2-d]pyrimidin-4-yl)morpholine

Palladium on carbon (10 mg, 10% loading) was added to a solution of 4-(2-(3-(pyridin-4-yl)-1H-pyrazol-1-yl)-7-vinylpyrido[3,2-d]pyrimidin-4-yl)morpholine (80 mg, 0.21 mmol) in methanol (5 ml) and the resultant mixture was stirred at 20° C. for 1 h under hydrogen atmosphere. The mixture was then filtered, concentrated and the obtained residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate) to obtain 4-(7-ethyl-2-(3-(pyridin-4-yl)-1H-pyrazol-1-yl)pyrido[3,2-d]pyrimidin-4-yl)morpholine (24.3 mg, 30%) as white solid. ¹H NMR (400 MHz, DMSO-d₆) δ 8.85 (d, J=2.0 Hz, 1H), 8.69-8.67 (m, 3H), 8.02 (d, J=2.0 Hz, 1H), 7.94-7.92 (m, 2H), 7.24 (d, J=2.0 Hz, 1H), 4.59 (bs, 4H), 3.82 (t, J=4.2 Hz, 4H), 2.86 (q, J=6.0 Hz, 2H), 1.32 (t, J=6.0 Hz, 3H); LCMS (ESI) m/z: 388.0 [M+H]⁺.

### Synthesis of (tert-butyl 2,2-dimethyl-4-[1-(4-morpholinopyrido[3,2-d]pyrimidin-2-yl)pyrazol-3-yl]piperidine-1-carboxylate (Compound 61) and 4-[2-[3-(2,2-dimethyl-4-piperidyl)pyrazol-1-yl]pyrido[3,2-d]pyrimidin-4-yl]morpholine (Compound 62)

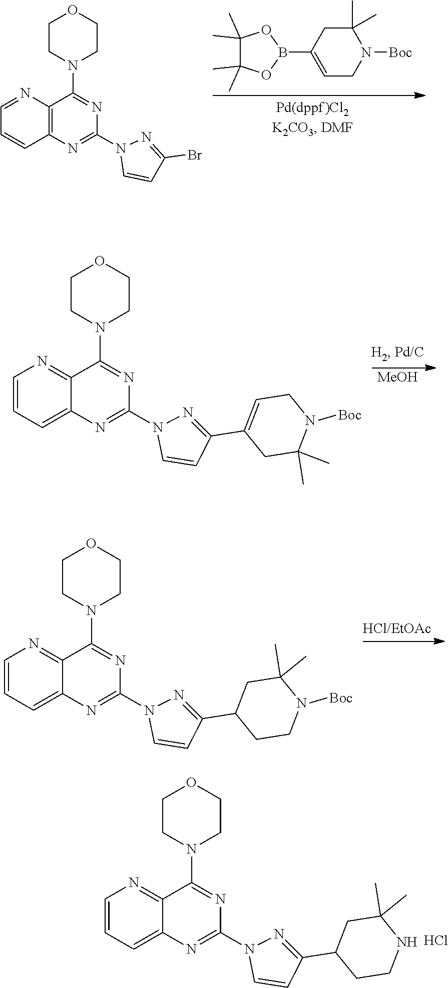

### Step 1: Synthesis of tert-butyl 6,6-dimethyl-4-[1-(4-morpholinopyrido[3,2-d]pyrimidin-2-yl)pyrazol-3-yl]-2,5-dihydropyridine-1-carboxylate

To a solution of 4-[2-(3-bromopyrazol-1-yl)pyrido[3,2-d]pyrimidin-4-yl]morpholine (150 mg, 415.11 umol) in dioxane (1 mL) and H₂O (0.2 mL), were added tert-butyl 6,6-dimethyl-4-(4,4,5,5-tetramethyl-1,3,2-dioxaborolan-2-yl)-2,5-dihydropyridine-1-carboxylate (140 mg, 415 umol), Pd(dppf)Cl₂ (32 mg, 42 umol), and K₂CO₃ (143 mg, 1.04 mol). The resultant mixture was stirred at 60° C. for 6 h under nitrogen. The mixture was then filtered and the filtrated was concentrated to obtain tert-butyl 6,6-dimethyl-4-[1-(4-morpholinopyrido[3,2-d]pyrimidin-2-yl)pyrazol-3-yl]-2,5-dihydropyridine-1-carboxylate (80 mg, 39%) as pale yellow solid. 1H NMR (400 MHz, CHLOROFORM-d) δ=8.62 (d, J=3.0 Hz, 1H), 8.54 (d, J=2.6 Hz, 1H), 8.24-8.18 (m, 1H), 7.59 (dd, J=4.1, 8.5 Hz, 1H), 6.59 (d, J=2.6 Hz, 1H), 6.44 (t, J=3.9 Hz, 1H), 4.62 (bs, 4H), 4.11 (bs, 2H), 3.96-3.91 (m, 4H), 2.04 (s, 2H), 1.52-1.47 (m, 15H); LCMS (ESI) m/z: 492.2 [M+H]⁺.

### Step 2: Synthesis of tert-butyl 2,2-dimethyl-4-[1-(4-morpholinopyrido[3,2-d]pyrimidin-2-yl)pyrazol-3-yl]piperidine-1-carboxylate

To a solution of tert-butyl 6,6-dimethyl-4-[1-(4-morpholinopyrido[3,2-d]pyrimidin-2-yl)pyrazol-3-yl]-2,5-dihydropyridine-1-carboxylate (55 mg, 112 umol) in MeOH (0.5 mL), was added Pd/C (100 mg, 10% purity), and the mixture was stirred at 20° C. for 40 min under hydrogen atmosphere (15 Psi). The mixture was filtered and the filtrate was evaporated to obtain tert-butyl 2,2-dimethyl-4-[1-(4-morpholinopyrido[3,2-d]pyrimidin-2-yl)pyrazol-3-yl]piperidine-1-carboxylate (80 mg) as white solid. ¹H NMR (400 MHz, METHANOL-d₄) δ 8.69 (dd, J=4.1, 1.6 Hz, 1H), 8.61 (d, J=2.6 Hz, 1H), 8.16 (dd, J=8.6, 1.5 Hz, 1H), 7.72 (dd, J=8.6, 4.2 Hz, 1H), 6.45 (d, J=2.6 Hz, 1H), 4.65 (bs, 4H), 3.97-3.85 (m, 5H), 3.27-3.11 (m, 2H), 2.17-2.05 (m, 1H), 1.97-1.88 (m, 1H), 1.86-1.71 (m, 2H), 1.57 (s, 3H), 1.48 (s, 9H), 1.44 (s, 3H). LCMS (ESI) for (C₂₆H₃₅N₇O₃) [M+H]⁺: 494.3.

### Step 3: Synthesis of 4-[2-[3-(2,2-dimethyl-4-piperidyl)pyrazol-1-yl]pyrido[3,2-d]pyrimidin-4-yl]morpholine

To a solution of tert-butyl 2,2-dimethyl-4-[1-(4-morpholinopyrido[3,2-d]pyrimidin-2-yl)pyrazol-3-yl]piperidine-1-carboxylate (40 mg, 81 umol) in MeOH (1 mL), was added HCl/MeOH (4 M, 2.40 mL) and the mixture was stirred at 20° C. for 1 h. The mixture was filtered and the filtrate was purified by prep-HPLC (Phenomenex luna C18 80*40 mm*3 um column, 10%-30% acetonitrile in a 0.04% hydrochloric acid solution in water, 7 min gradient) to obtain 4-[2-[3-(2,2-dimethyl-4-piperidyl)pyrazol-1-yl]pyrido[3,2-d]pyrimidin-4-yl]morpholine (5 mg, 15%) as white solid. ¹H NMR (400 MHz, METHANOL-d₄) δ 8.81 (d, J=4.0 Hz, 1H), 8.73 (bs, 1H), 8.33 (d, J=7.6 Hz, 1H), 7.87 (dd, J=8.0, 4.1 Hz, 1H), 6.63 (bs, 1H), 4.75 (bs, 4H), 3.93 (t, J=4.4 Hz, 4H), 3.45-3.35 (m, 3H), 2.31 (d, J=14.5 Hz, 1H), 2.24-2.15 (m, 1H), 2.02-1.87 (m, 2H), 1.53 (s, 3H), 1.50 (s, 3H). LCMS (ESI) for (C₂₁H₂₇N₇O) [M+H]⁺: 394.2.

### Synthesis of tert-butyl 3-[[1-(4-morpholinopyrido[3,2-d]pyrimidin-2-yl)pyrazol-3-yl]methyl]pyrrolidine-1-carboxylate (Compound 63) and 4-[2-[3-(pyrrolidin-3-ylmethyl)pyrazol-1-yl]pyrido[3,2-d]pyrimidin-4-yl]morpholine (Compound 64)

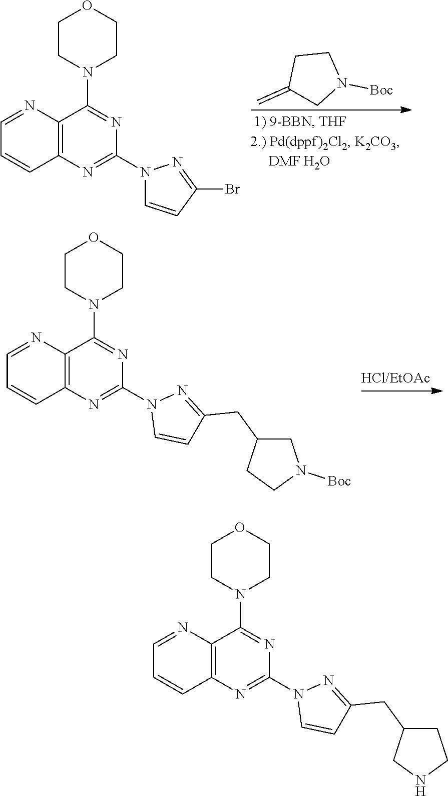 [SMILES: Brc1ccn(-c2nc(N3CCOCC3)c3ncccc3n2)n1 ; C=C1CCN(C(=O)OC(C)(C)C)C1 ; CC(C)(C)OC(=O)N1CCC(Cc2ccn(-c3nc(N4CCOCC4)c4ncccc4n3)n2)C1 ; c1cnc2c(N3CCOCC3)nc(-n3ccc(CC4CCNC4)n3)nc2c1]

### Step 1: Synthesis of tert-butyl 3-[[1-(4-morpholinopyrido[3,2-d]pyrimidin-2-yl)pyrazol-3-yl]methyl]pyrrolidine-1-carboxylate

A mixture of tert-butyl 3-methylenepyrrolidine-1-carboxylate (507 mg, 2.77 mmol) and 9-BBN (0.5M in THF, 5.54 mL) was stirred at 80° C. for 1 h. It was then cooled to 20° C. and to the resultant solution were added 4-[2-(3-bromopyrazol-1-yl)pyrido[3,2-d]pyrimidin-4-yl]morpholine (0.5 g, 1.38 mmol), Pd(dppf)Cl₂·CH₂Cl₂ (57 mg, 69 umol), K₂CO₃ (287 mg, 2.08 mmol), DMF (5 mL) and water (0.5 mL). The resulting mixture was heated at 80° C. for 15 h. 15 mL of water was then added to the reaction mixture and it was extracted with ethyl acetate (30 mL*2). The combined organic layers were washed with brine (15 mL), dried over Na₂SO₄ and concentrated. The crude product was purified by prep-HPLC (Phenomenex Gemini-NX 150*30 5u column; 20-50% acetonitrile in an a 10 mM ammonium bicarbonate solution in water, 8 min gradient) to obtain tert-butyl 3-[[1-(4-morpholinopyrido[3,2-d]pyrimidin-2-yl)pyrazol-3-yl]methyl]pyrrolidine-1-carboxylate (150 mg, 23%) as white solid. ¹H NMR (400 MHz, CHLOROFORM-d) δ=8.63 (d, J=2.6 Hz, 1H), 8.50 (d, J=7.1 Hz, 1H), 8.27 (dd, J=8.6, 1.6 Hz, 1H), 7.59 (dd, J=8.6, 4.1 Hz, 1H), 6.30 (d, J=2.5 Hz, 1H), 4.61 (bs, 4H), 3.93 (t, J=4.8 Hz, 4H), 3.68-3.39 (m, 2H), 3.36-3.20 (m, 1H), 3.14-2.97 (m, 1H), 2.95-2.80 (m, 2H), 2.70-2.48 (m, 1H), 2.09-1.97 (m, 1H), 1.72-1.63 (m, 1H), 1.46 (s, 9H). LCMS (ESI) for C₂₄H₃₁N₇O₃ [M+H]⁺: 466.3.

### Step 5: Synthesis of 4-[2-[3-(pyrrolidin-3-ylmethyl)pyrazol-1-yl]pyrido[3,2-d]pyrimidin-4-yl]morpholine

A mixture of tert-butyl 3-[[1-(4-morpholinopyrido[3,2-d]pyrimidin-2-yl)pyrazol-3-yl]methyl]pyrrolidine-1-carboxylate (120 mg, 258 umol) in 4M HCl/EtOAc (10 mL) was stirred at 25° C. for 1 h. The reaction mixture was concentrated and the crude product was purified by prep-HPLC (Phenomenex luna C18 80*40 3u column; 8-48% acetonitrile in an a 0.04% hydrochloric acid solution in water, 7 min gradient) to obtain 4-[2-[3-(pyrrolidin-3-ylmethyl)pyrazol-1-yl]pyrido[3,2-d]pyrimidin-4-yl]morpholine·HCl (82 mg, 79%) as white solid. ¹H NMR (400 MHz, METHANOL-d₄) δ=8.87 (d, J=3.3 Hz, 1H), 8.77 (d, J=2.4 Hz, 1H), 8.42 (d, J=8.5 Hz, 1H), 7.93 (dd, J=8.6, 4.3 Hz, 1H), 6.67 (d, J=2.4 Hz, 1H), 5.52-4.90 (m, 2H), 4.82-4.24 (m, 2H), 3.96 (t, J=4.4 Hz, 4H), 3.54 (dd, J=11.5, 7.6 Hz, 1H), 3.49-3.41 (m, 1H), 3.36-3.3 (m, 1H), 3.11-2.96 (m, 3H), 2.93-2.77 (m, 1H), 2.33-2.15 (m, 1H), 1.83 (qd, J=13.1, 8.7 Hz, 1H). LCMS (ESI) for C₁₉H₂₃N₇O [M+H]⁺: 366.2.

### Synthesis of 4-(7-(5-methoxypyridin-3-yl)-2-(1-(2,2,2-trifluoroethyl)-1H-pyrazol-3-yl)pyrido[3,2-d]pyrimidin-4-yl)morpholine (Compound 65)

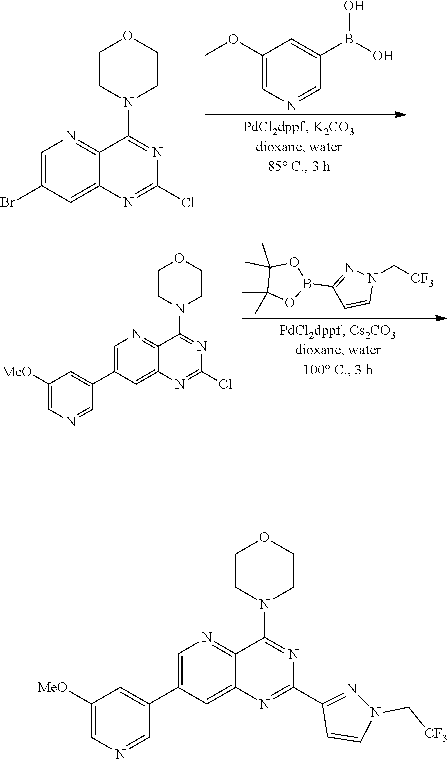

### Step 1: Synthesis of 4-(2-chloro-7-(5-methoxypyridin-3-yl)pyrido[3,2-d]pyrimidin-4-yl)morpholine

To a solution of 4-(7-bromo-2-chloropyrido[3,2-d]pyrimidin-4-yl)morpholine (328 mg, 1 mmol) in dioxane (9 mL) and water (1 mL) were added (5-methoxypyridin-3-yl)boronic acid (310 mg, 2 mmol), [1,1′-bis(diphenylphosphino)ferrocene]dichloropalladium(II) (73.1 mg, 0.1 mmol) and potassium carbonate (414 mg, 3 mmol) at 25° C. and the resultant mixture was stirred at 85° C. for 3 h under nitrogen atmosphere. It was cooled and then extracted with ethyl acetate (20 mL*2), washed with water (10 mL*2), dried over sodium sulfate, and concentrated. The crude product was purified by flash chromatography on silica gel (petroleum ether/ethyl acetate=1:1) to obtain 4-(2-chloro-7-(5-methoxypyridin-3-yl)pyrido[3,2-d]pyrimidin-4-yl)morpholine as white solid. (120 mg, 33.6%). LCMS (ESI) m/z: 358.0 [M+H]⁺.

### Step 2: Synthesis of 4-(7-(5-methoxypyridin-3-yl)-2-(1-(2,2,2-trifluoroethyl)-1H-pyrazol-3-yl)pyrido[3,2-d]pyrimidin-4-yl)morpholine

To a solution of 4-(2-chloro-7-(5-methoxypyridin-3-yl)pyrido[3,2-d]pyrimidin-4-yl)morpholine (120 mg, 0.3 mmol) in dioxane (8 mL) and water (1 mL) were added 3-(4,4,5,5-tetramethyl-1,3,2-dioxaborolan-2-yl)-1-(2,2,2-trifluoroethyl)-1H-pyrazole (165 mg, 0.6 mmol), [1,1′-bis(diphenylphosphino)ferrocene]dichloropalladium(II) (22 mg, 0.03 mmol) and cesium carbonate (292 mg, 0.9 mmol) at 25° C. The resultant mixture was stirred at 100° C. for 3 h under nitrogen atmosphere. The mixture was then extracted with ethyl acetate (20 mL*2), washed with water (10 mL*2), the organic layer was dried over sodium sulfate and concentrated. The residue was purified with prep-HPLC (BOSTON pHlex ODS 10 μm 21.2ᵢÁ250 mm 120 A. The mobile phase was acetonitrile/0.1% Ammonium bicarbonate) to obtain 4-(7-(5-methoxypyridin-3-yl)-2-(1-(2,2,2-trifluoroethyl)-1H-pyrazol-3-yl)pyrido[3,2-d]pyrimidin-4-yl)morpholine as white solid. (23.1 mg, 16.3%). ¹H NMR (400 MHz, DMSO-d₆) δ 9.20 (d, J=2.3 Hz, 1H), 8.76 (d, J=1.7 Hz, 1H), 8.62 (d, J=2.3 Hz, 1H), 8.41 (d, J=2.7 Hz, 1H), 8.00-7.94 (m, 2H), 7.07 (d, J=2.3 Hz, 1H), 5.28 (q, J=9.1 Hz, 2H), 4.54 (bs, 4H), 3.96 (s, 3H), 3.86-3.81 (m, 4H); LCMS (ESI) m/z: 472.1 [M+H]⁺.

### Synthesis of 4-(2-(3-(tetrahydro-2H-pyran-4-yl)-1H-pyrazol-1-yl)pyrido[3,2-d]pyrimidin-4-yl)morpholine (Compound 66)

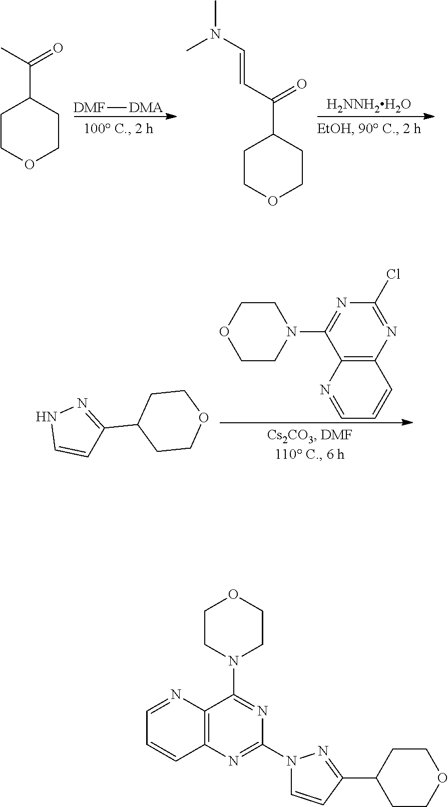

### Step 1: Synthesis of 3-(dimethylamino)-1-(tetrahydro-2H-pyran-4-yl)prop-2-en-1-one

To a solution of 1-(tetrahydro-2H-pyran-4-yl)ethan-1-one (1 g, 7.81 mmol) in toluene (10 ml) was added N,N-dimethylformamide dimethylacetal (2.79 g, 23.4 mmol). Then the reaction mixture was stirred at 100° C. for 2 h and concentrated to afford 3-(dimethylamino)-1-(tetrahydro-2H-pyran-4-yl)prop-2-en-1-one (1.2 g, 62%). LCMS (ESI) m/z: 184.2 [M+H]⁺.

### Step 2: Synthesis of 3-(tetrahydro-2H-pyran-4-yl)-1H-pyrazole

To a solution of 3-(dimethylamino)-1-(tetrahydro-2H-pyran-4-yl)prop-2-en-1-one (1.1 g, 6 mmol) in ethanol (10 mL) was added hydrazine hydrate (5 mL) and the mixture was stirred at 90° C. for 2 h. It was then concentrated and the residue was purified by prep-HPLC (Boston C18 21*250 mm 10 μm column. The mobile phase was acetonitrile/0.05% trifluoroacetic acid aqueous solution) to obtain 3-(tetrahydro-2H-pyran-4-yl)-1H-pyrazole (0.67 g, 64%) as yellow oil. LCMS (ESI) m/z: 153.3 [M+H]⁺.

### Step 3: Synthesis of 4-(2-(3-(tetrahydro-2H-pyran-4-yl)-1H-pyrazol-1-yl)pyrido[3,2-d]pyrimidin-4-yl)morpholine

To a solution of 4-(2-chloropyrido[3,2-d]pyrimidin-4-yl)morpholine (200 mg, 0.8 mmol) and 3-(tetrahydro-2H-pyran-4-yl)-1H-pyrazole (134 mg, 0.88 mmol) in N,N-dimethylformamide (10 mL) was added cesium carbonate (782 mg, 2.4 mmol). The reaction mixture was stirred at 110° C. for 6 h and concentrated. The residue obtained subjected to prep-HPLC (Boston C18 21*250 mm 10 μm column. The mobile phase was acetonitrile/10 mM formic acid aqueous solution) to obtain 4-(2-(3-(tetrahydro-2H-pyran-4-yl)-1H-pyrazol-1-yl)pyrido[3,2-d]pyrimidin-4-yl)morpholine (101.5 mg, 34.7%) as white solid. ¹H NMR (400 MHz, DMSO-d₆) δ 8.73 (dd, J=4.0, 1.6 Hz, 1H), 8.62 (d, J=2.8 Hz, 1H), 8.15 (dd, J=8.4, 1.6 Hz, 1H), 7.80 (dd, J=8.8, 4.0 Hz, 1H), 6.47 (d, J=2.8 Hz, 1H), 4.54 (bs, 4H), 3.95-3.93 (m, 2H), 3.82 (t, J=4.8, 4H), 3.50-3.46 (m, 2H), 3.00-2.94 (m, 1H), 1.87-1.84 (m, 2H), 1.77-1.68 (m, 2H). LCMS (ESI) m/z: 367.0 [M+H]⁺.

### Synthesis of 4-(2-(1-phenyl-1H-pyrazol-3-yl)pyrido[3,2-d]pyrimidin-4-yl)morpholine (Compound 67)

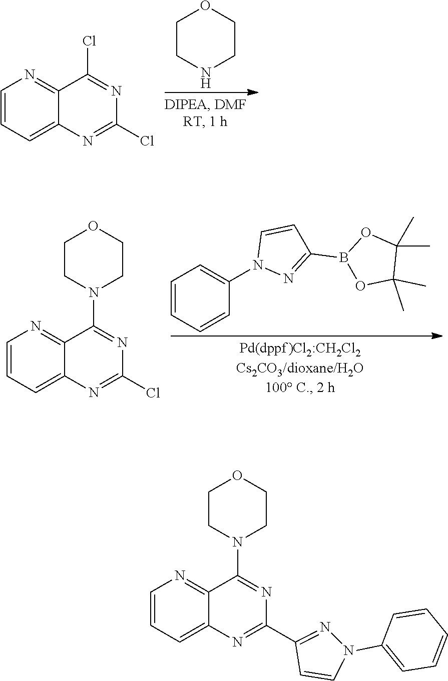 [SMILES: Clc1nc(Cl)c2ncccc2n1 ; C1COCCN1 ; Clc1nc(N2CCOCC2)c2ncccc2n1 ; CC1(C)OB(c2ccn(-c3ccccc3)n2)OC1(C)C ; c1ccc(-n2ccc(-c3nc(N4CCOCC4)c4ncccc4n3)n2)cc1]

### Step 1: Synthesis of 4-(2-chloropyrido[3,2-d]pyrimidin-4-yl)morpholine

To a solution of 2,4-dichloropyrido[3,2-d]pyrimidine (2.0 g, 10.0 mmol), N,N-diisopropylethylamine (2.58 g, 20.0 mmol) in N,N-dimethylformamide (15 mL) was added morpholine (870 mg, 10.0 mmol) at 28° C. After the addition, the mixture was stirred for another 1 h and poured into water (100 mL). The formed precipitate was collected by filtration and dried under vacuum to afford 4-(2-chloropyrido[3,2-d]pyrimidin-4-yl)morpholine (2.0 g, 80%) as yellow solid. LCMS (ESI) m/z: 251.1/253.1 [M+H]⁺.

### Step 2: Synthesis of 4-(2-(1-phenyl-1H-pyrazol-3-yl)pyrido[3,2-d]pyrimidin-4-yl)morpholine

A mixture of 4-(2-chloropyrido[3,2-d]pyrimidin-4-yl)morpholine (150 mg, 0.6 mmol), 1-phenyl-3-(4,4,5,5-tetramethyl-1,3,2-dioxaborolan-2-yl)-1H-pyrazole (162 mg, 0.6 mmol), 1,1′-bis(diphenylphosphino)ferrocene-palladium(II)dichloride dichloromethane complex (49 mg, 0.06 mmol) and cesium carbonate (390 mg, 1.2 mmol) in dioxane (8 mL) and water (1 mL) was stirred at 100° C. under nitrogen atmosphere for 2 h. The resultant mixture was poured into water, extracted with ethyl acetate (100 mL*2) and concentrated. The crude product was purified by prep-HPLC (Column Xbridge 21.2*250 mm C18, 10 um, Mobile Phase A: water (10 mmol/L ammonium bicarbonate) B: acetonitrile) to afford 4-(2-(1-phenyl-1H-pyrazol-3-yl)pyrido[3,2-d]pyrimidin-4-yl)morpholine (95.8 mg, 43.3%) as white solid. ¹H NMR (400 MHz, DMSO-d₆) δ 8.78 (dd, J=4.0, 2.0 Hz, 1H), 8.61 (d, J=2.4 Hz, 1H), 8.24 (dd, J=8.8, 1.6 Hz, 1H), 7.97-7.95 (m, 2H), 7.82 (dd, J=8.8, 4.0 Hz, 1H), 7.56 (t, J=7.6 Hz, 2H), 7.39 (t, J=7.2 Hz, 1H), 7.22 (d, J=2.8 Hz, 1H), 4.54 (bs, 4H), 3.83 (t, J=4.8 Hz, 4H); LCMS (ESI) m/z: 359.1 [M+H]⁺.

### Synthesis of 4-(2-(1-(2,2,2-trifluoroethyl)-1H-pyrazol-3-yl)pyrido[3,2-d]pyrimidin-4-yl)morpholine (Compound 68)

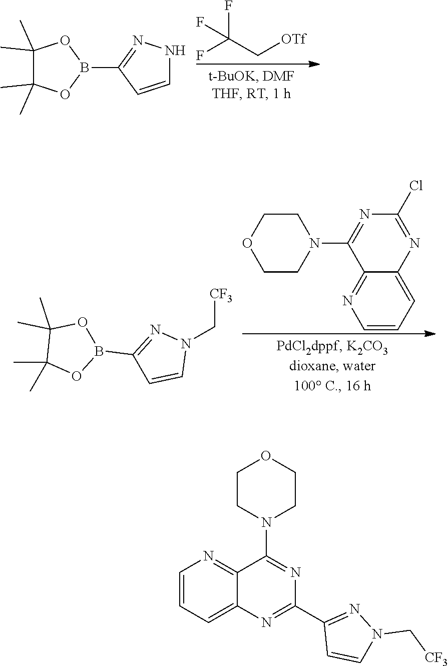

### Step 1: Synthesis of 3-(4,4,5,5-tetramethyl-1,3,2-dioxaborolan-2-yl)-1-(2,2,2-trifluoroethyl)-1H-pyrazole

To a solution of 3-(4,4,5,5-tetramethyl-1,3,2-dioxaborolan-2-yl)-1H-pyrazole (388 mg, 2 mmol) in N,N-dimethylformamide (10 mL) and tetrahydrofuran (10 mL) were added 2,2,2-trifluoroethyl trifluoromethanesulfonate (1.4 g, 6 mmol) and potassium t-butoxide (44 mg, 0.4 mmol) at 25° C. The resultant mixture was stirred at room temperature for 1 h. 30 mL of water was added to the mixture and it was extracted with ethyl acetate (20 mL*3). The organic layer was dried over sodium sulfate, and concentrated to afford 3-(4,4,5,5-tetramethyl-1,3,2-dioxaborolan-2-yl)-1-(2,2,2-trifluoroethyl)-1H-pyrazole (500 mg, 90.6%). LCMS (ESI) m/z: 277.1 [M+H]⁺.

### Step 2: Synthesis of 4-(2-(1-(2,2,2-trifluoroethyl)-1H-pyrazol-3-yl)pyrido[3,2-d]pyrimidin-4-yl)morpholine

To a solution of 3-(4,4,5,5-tetramethyl-1,3,2-dioxaborolan-2-yl)-1-(2,2,2-trifluoroethyl)-1H-pyrazole (138 mg, 0.5 mmol) in dioxane (9 mL) and water (1 mL) were added 4-(2-chloropyrido[3,2-d]pyrimidin-4-yl)morpholine (125 mg, 0.5 mmol), [1,1′-bis(diphenylphosphino)ferrocene]dichloropalladium(II) (37 mg, 0.05 mmol) and potassium carbonate (207 mg, 1.5 mmol) at 25° C. The reaction mixture was then stirred at 100° C. for 16 h under argon atmosphere. The mixture was then extracted with ethyl acetate (20 mL*2), washed with water (10 mL*2), the organic layer was dried and concentrated. The resultant residue was purified with prep-HPLC (BOSTON pHlex ODS 10 μm 21.2ᵢÁ250 mm 120 A. The mobile phase was acetonitrile/0.1% Ammonium bicarbonate) to obtain 4-(2-(1-(2,2,2-trifluoroethyl)-1H-pyrazol-3-yl)pyrido[3,2-d]pyrimidin-4-yl)morpholine as white solid (9.6 mg, 5.3%). ¹H NMR (400 MHz, DMSO-d₆) δ 8.77 (dd, J=4.1, 1.6 Hz, 1H), 8.19 (dd, J=8.5, 1.6 Hz, 1H), 7.94 (d, J=2.3 Hz, 1H), 7.80 (dd, J=8.5, 4.0 Hz, 1H), 7.05 (d, J=2.3 Hz, 1H), 5.27 (q, J=9.2 Hz, 2H), 4.51 (s, 4H), 3.80 (t, J=4.8 Hz, 4H); LCMS (ESI) m/z: 365.1 [M+H]⁺.

### Synthesis of 4-[2-(1H-indazol-3-yl)pyrido[3,2-d]pyrimidin-4-yl]morpholine (Compound 69)

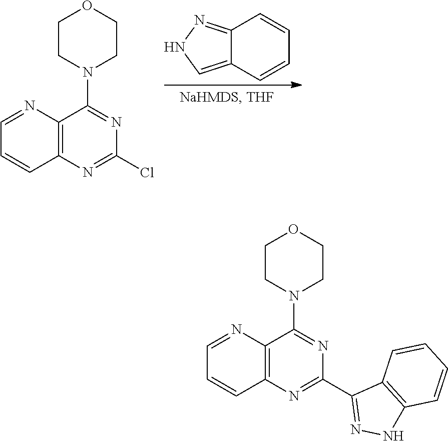 [SMILES: Clc1nc(N2CCOCC2)c2ncccc2n1 ; c1ccc2n[nH]cc2c1 ; c1cnc2c(N3CCOCC3)nc(-c3n[nH]c4ccccc34)nc2c1]

A solution of 2H-indazole (94 mg, 798 umol) and NaHMDS (1 M, 1.60 mL) in THF (2 mL) was stirred for 0.5 h at 0° C., followed by the addition of 4-(2-chloropyrido[3,2-d]pyrimidin-4-yl)morpholine (200 mg, 798 umol). The resultant mixture was further stirred at 15° C. for 14 h. The mixture was concentrated and the crude product obtained was purified by prep-HPLC (Waters Xbridge BEH C18 100*30 mm*10 um column; 35-60% acetonitrile in an 10 mM ammonium bicarbonate in water, 8 min gradient) to obtain 4-[2-(1H-indazol-3-yl)pyrido[3,2-d]pyrimidin-4-yl]morpholine (34 mg, 13%) as pale yellow solid. ¹H NMR (400 MHz, DMSO-d6) δ 9.25 (s, 1H), 8.50-8.49 (m, 1H), 7.80-7.75 (m, 3H), 7.63-7.61 (m, 2H), 7.25-7.22 (m, 1H), 4.36 (bs, 4H), 3.75 (t, J=4.8 Hz, 4H); LCMS (ESI for C₁₈H₁₆N₆O) [M+H]⁺: 333.2. (NOE experiments confirmed the regioselectivity and the product).

### Synthesis of 1-(7-(furan-3-yl)-4-morpholinopyrido[3,2-d]pyrimidin-2-yl)-3-m-tolyl-1H-pyrazol-5-ol (Compound 70)

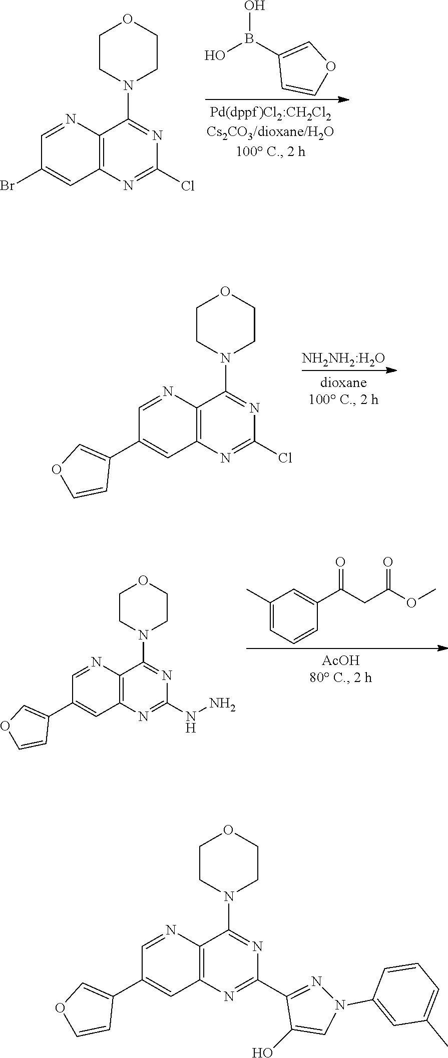

### Step 1: Synthesis of 4-(2-chloro-7-(furan-3-yl)pyrido[3,2-d]pyrimidin-4-yl)morpholine

A mixture of 4-(7-bromo-2-chloropyrido[3,2-d]pyrimidin-4-yl)morpholine (500 mg, 1.52 mmol), furan-3-ylboronic acid (170 mg, 1.52 mmol), 1,1′-bis(diphenylphosphino)ferrocene-palladium(II)dichloride dichloromethane complex (122 mg, 0.15 mmol) and potassium carbonate (420 mg, 3.04 mmol) in dioxane (20 mL) and water (2 mL) was stirred at 100° C. under nitrogen atmosphere for 2 h. The resultant mixture was poured into water, extracted with ethyl acetate (150 mL*2) and the combined organic phase was concentrated. The residue was purified by silica gel column chromatography (40% ethyl acetate in petroleum ether) to afford 4-(2-chloro-7-(furan-3-yl)pyrido[3,2-d]pyrimidin-4-yl)morpholine (360 mg, 75%) as grey solid. LCMS (ESI) m/z: 316.8/318.9 [M+H]⁺.

### Step 2: Synthesis of 4-(7-(furan-3-yl)-2-hydrazinylpyrido[3,2-d]pyrimidin-4-yl)morpholine

A mixture of 4-(2-chloro-7-(furan-3-yl)pyrido[3,2-d]pyrimidin-4-yl)morpholine (300 mg, 0.95 mmol) and hydrazine hydrate (98%, 2 mL) in dioxane 10 mL) was stirred at 100° C. for 2 h. The resultant precipitate was collected by filtration and dried under vacuum to afford 4-(7-(furan-3-yl)-2-hydrazinylpyrido[3,2-d]pyrimidin-4-yl)morpholine (180 mg, 60%) as yellow solid. LCMS (ESI) m/z: 312.9 [M]⁺.

### Step 3: Synthesis of 1-(7-(furan-3-yl)-4-morpholinopyrido[3,2-d]pyrimidin-2-yl)-3-m-tolyl-1H-pyrazol-5-ol

A mixture of 4-(7-(furan-3-yl)-2-hydrazinylpyrido[3,2-d]pyrimidin-4-yl)morpholine (150 mg, 0.48 mmol), ethyl 3-oxo-3-m-tolylpropanoate (117 mg, 0.57 mmol) and acetic acid (5 mL) was stirred at 80° C. for 2 h. The mixture was poured into water and the formed precipitate was collected by filtration and dried under vacuum to afford 150 mg of a grey solid, which was further purified by prep-HPLC (Column Xbridge 21.2*250 mm C18, 10 um, Mobile Phase A: water (10 mmol/L ammonium bicarbonate) B: acetonitrile) to afford 1-(7-(furan-3-yl)-4-morpholinopyrido[3,2-d]pyrimidin-2-yl)-3-m-tolyl-1H-pyrazol-5-ol (72.9 mg, 33.3%) as grey solid. ¹H NMR (400 MHz, CDCl₃) δ 13.50 (s, 1H), 8.80 (d, J=2.2 Hz, 1H), 8.03-7.94 (m, 2H), 7.78 (s, 1H), 7.70 (d, J=7.7 Hz, 1H), 7.60 (t, J=1.7 Hz, 1H), 7.31 (t, J=7.6 Hz, 1H), 7.18 (d, J=7.5 Hz, 1H), 6.84 (dd, J=1.8, 0.8 Hz, 1H), 5.99 (s, 1H), 4.72 (bs, 4H), 3.96 t, J=4.0 Hz, 4H), 2.42 (s, 3H); LCMS (ESI) m/z: 454.8 [M]⁺.

### Synthesis of 1-(4-morpholinopyrido[3,2-d]pyrimidin-2-yl)-3-phenyl-1H-pyrazol-5-ol (Compound 71)

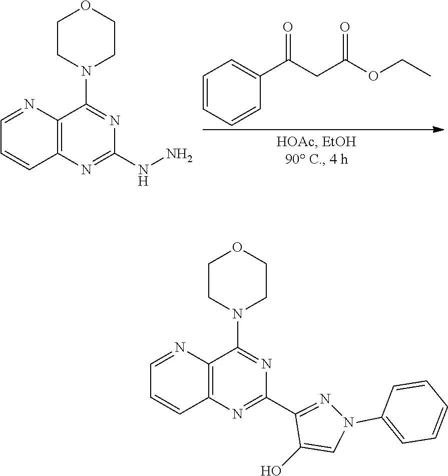 [SMILES: NNc1nc(N2CCOCC2)c2ncccc2n1 ; CCOC(=O)CC(=O)c1ccccc1 ; Oc1cn(-c2ccccc2)nc1-c1nc(N2CCOCC2)c2ncccc2n1]

A mixture of 4-(2-hydrazineylpyrido[3,2-d]pyrimidin-4-yl)morpholine (210 mg, 0.85 mmol), ethyl 3-oxo-3-phenylpropanoate (164 mg, 0.85 mmol) and acetic acid (0.1 mL) in ethanol (8 mL) was stirred at 90° C. for 4 h. The reaction mixture was concentrated and the residue was purified under prep-HPLC (Boston C18 21*250 mm 10 μm column. The mobile phase was acetonitrile/10 mM formic acid aqueous solution) to obtain 1-(4-morpholino pyrido[3,2-d]pyrimidin-2-yl)-3-phenyl-1H-pyrazol-5-ol (55.2 mg, 17%) as yellow solid. ¹H NMR (400 MHz, DMSO-d₆) δ 8.76 (dd, J=4.2, 1.7 Hz, 1H), 8.29 (dd, J=8.5, 1.6 Hz, 1H), 7.92-7.87 (m, 2H), 7.84 (dd, J=8.5, 4.2 Hz, 1H), 7.46 (t, J=7.4 Hz, 2H), 7.38 (t, J=7.2 Hz, 1H), 6.17 (s, 1H), 4.66 (bs, 4H), 3.91-3.82 (m, 4H). LCMS (ESI) m/z: 375.1 [M+H]⁺.

The following compound was synthesized according to the protocol described above:

| Name | Structure | NMR, MS | # |
|---|---|---|---|
| 1-(4-morpholinopyrido[3,2- d]pyrimidin-2-yl)-3-m-tolyl- 1H-pyrazol-5-ol | 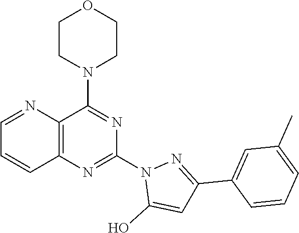 [SMILES: Cc1cccc(-c2cc(O)n(-c3nc(N4CCOCC4)c4ncccc4n3)n2)c1] | ¹H NMR (400 MHz, DMSO) δ 13.25 (bs, 1H), 8.75 (dd, J = 4.0, 1.6 Hz, 1H), 8.28 (dd, J = 8.4, 1.2 Hz, 1H), 7.84 (dd, J = 8.4 Hz, 4Hz, 1H), 7.74-7.64 (m, 2H), 7.34 (t, J = 8.0 Hz, 1H), 7.20 (d, J = 8 Hz, 1H), 6.16 (s, 1H), 4.50 (bs, 4H), 3.85 (t, J = 4.4Hz, 4H), 2.38 (s, 3H); LCMS (ESI) m/z: 389.1 [M + H]⁺. | 72 |

### Synthesis of 2-(4-morpholinopyrido[2,3-d]pyrimidin-2-yl)-5-phenyl-2,4-dihydro-3H-pyrazol-3-one (Compound 73)

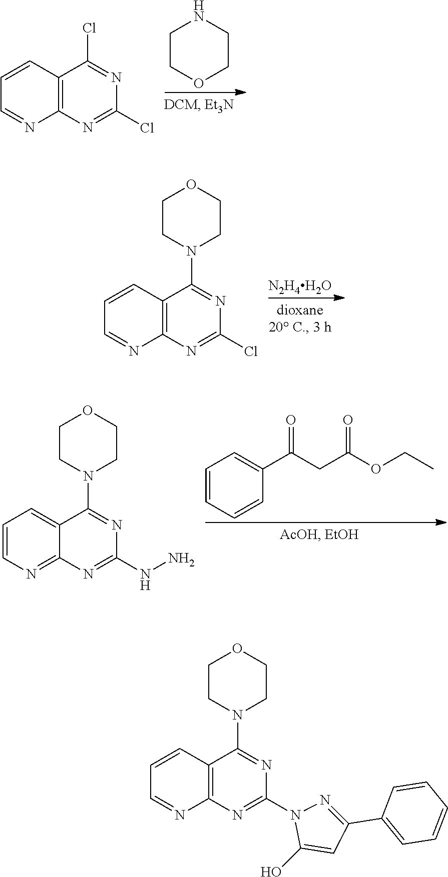

### Step 1: Synthesis of 4-(2-chloropyrido[2,3-d]pyrimidin-4-yl)morpholine

To a solution of 2,4-dichloropyrido[2,3-d]pyrimidine (0.6 g, 3 mmol) and triethylamine (600 mg, 6 mmol) in dichloromethane (10.0 mL) was added morpholine (0.27 g, 3.15 mmol) at −20° C. and the resulting solution was stirred at −20˜−10° C. under nitrogen for 30 min. The reaction was quenched with water (2 mL), dried over sodium sulfate, filtered and concentrated. The crude product was crystallized using petroleum ether and ethyl acetate (4:1) to obtain 4-(2-chloropyrido[2,3-d]pyrimidin-4-yl)morpholine (0.5 g, 45%) as off-white solid. LCMS (ESI) m/z: 251.1 [M+H]⁺.

### Step 2: Synthesis of 4-(2-hydrazineylpyrido[2,3-d]pyrimidin-4-yl)morpholine

To a suspension of 4-(2-chloropyrido[2,3-d]pyrimidin-4-yl)morpholine (0.25 g, 1 mmol) in 1,4-dioxane (4 mL) was added hydrazine monohydrate (0.25 g, 5 mmol), and the reaction was stirred for 0.5 h at 25° C. The mixture was filtered and concentrated to give 4-(2-hydrazineylpyrido[2,3-d]pyrimidin-4-yl)morpholine (0.18 g, 73%) as yellow solid. LCMS (ESI) m/z: 247.1 [M+H]⁺.

### Step 3: Synthesis of 2-(4-morpholinopyrido[2,3-d]pyrimidin-2-yl)-5-phenyl-2,4-dihydro-3H-pyrazol-3-one

A mixture of 4-(2-hydrazineylpyrido[2,3-d]pyrimidin-4-yl)morpholine (210 mg, 0.85 mmol), ethyl 3-oxo-3-phenylpropanoate (164 mg, 0.85 mmol) and acetic acid (0.1 mL) in ethanol (8 mL) was stirred at 90° C. for 2 h. The mixture was concentrated and the crude product was purified by prep-HPLC (Boston C18 21*250 mm 10 μm column. The mobile phase was acetonitrile/10 mM formic acid aqueous solution) to obtain 2-(4-morpholinopyrido[2,3-d]pyrimidin-2-yl)-5-phenyl-2,4-dihydro-3H-pyrazol-3-one as brown solid (15 mg, 5%). ¹H NMR (400 MHz, DMSO-d6) δ 8.95 (d, J=2.9 Hz, 1H), 8.55 (d, J=8.2 Hz, 1H), 7.90 (d, J=7.3 Hz, 2H), 7.52-7.43 (m, 3H), 7.39 (t, J=7.1 Hz, 1H), 6.16 (s, 1H), 4.10 (t, J=3.6 Hz, 4H), 3.84 (t, J=4.0 Hz, 4H); LCMS (ESI) m/z: 375.1 [M+H]⁺.

### Synthesis of 4-(2-(4-phenyl-1H-1,2,3-triazol-1-yl)pyrido[3,2-d]pyrimidin-4-yl)morpholine (Compound 74)

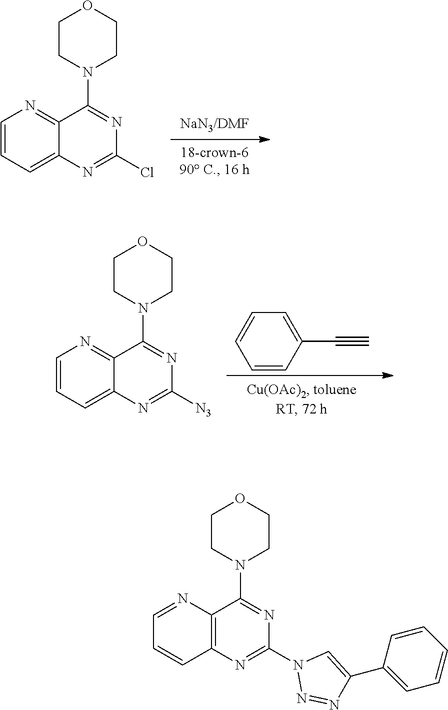

### Step 1: Synthesis of 4-(2-azidopyrido[3,2-d]pyrimidin-4-yl)morpholine

A mixture of 4-(2-chloropyrido[3,2-d]pyrimidin-4-yl)morpholine (500 mg, 2.0 mmol), sodium azide (161 mg, 2.4 mmol) and 18-crown-6 (53 mg, 0.2 mmol) in N,N-dimethylformamide (10 mL) was stirred at 90° C. for 16 h. The resultant mixture was poured into water and the formed precipitate was collected by filtration and dried under vacuum to afford 4-(2-azidopyrido[3,2-d]pyrimidin-4-yl)morpholine (400 mg, 75%) as yellow solid. LCMS (ESI) m/z: 258.1 [M+H]⁺.

### Step 2: Synthesis of 4-(2-(4-phenyl-1H-1,2,3-triazol-1-yl)pyrido[3,2-d]pyrimidin-4-yl)morpholine

A mixture of 4-(2-azidopyrido[3,2-d]pyrimidin-4-yl)morpholine (260 mg, 0.97 mmol), ethynylbenzene (395 mg, 3.88 mmol) and cupric acetate (175 mg, 0.97 mmol) in toluene (15 mL) was stirred at 25° C. for 72 h under nitrogen atmosphere. The mixture was concentrated and the residue was purified by silica gel column chromatography (25% methanol in dichloromethane) to afford 4-(2-(4-phenyl-1H-1,2,3-triazol-1-yl)pyrido[3,2-d]pyrimidin-4-yl)morpholine (110 mg, 31%) as off-white solid. ¹H NMR (400 MHz, DMSO-d₆) δ 9.40 (s, 1H), 8.84 (dd, J=4.1, 1.4 Hz, 1H), 8.27 (d, J=8.4 Hz, 1H), 8.12-8.03 (m, 2H), 7.90 (dd, J=8.5, 4.2 Hz, 1H), 7.51 (t, J=7.6 Hz, 2H), 7.44-7.36 (m, 1H), 4.28 (bs, 4H), 3.90-3.81 (m, 4H); LCMS (ESI) m/z: 359.9 [M]⁺.

### Synthesis of 4-(2-(4-phenyl-2H-1,2,3-triazol-2-yl)pyrido[3,2-d]pyrimidin-4-yl)morpholine hydrochloride (Compound 75)

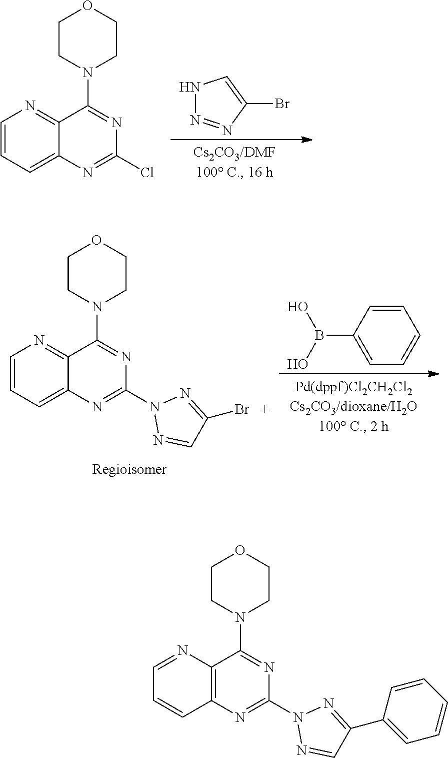

### Step 1: Synthesis of 4-(2-(4-bromo-2H-1,2,3-triazol-2-yl)pyrido[3,2-d]pyrimidin-4-yl)morpholine

A mixture of 4-(2-chloropyrido[3,2-d]pyrimidin-4-yl)morpholine (500 mg, 2.0 mmol), 4-bromo-1H-1,2,3-triazole (323 mg, 2.2 mmol) and cesium carbonate (1.3 g, 4.0 mmol) in N,N-dimethylformamide (15 mL) was stirred at 100° C. for 16 h. The mixture was poured into water and the formed precipitate was collected by filtration to afford 430 mg of yellow solid. This was further triturated with ethyl acetate (40 mL) and the resultant precipitate was collected by filtration to afford 4-(2-(4-bromo-1H-1,2,3-triazol-1-yl)pyrido[3,2-d]pyrimidin-4-yl)morpholine (230 mg, undesired regioisomer) as grey solid and the filtrate was concentrated to afford 4-(2-(4-bromo-2H-1,2,3-triazol-2-yl)pyrido[3,2-d]pyrimidin-4-yl)morpholine (180 mg) as yellow solid. LCMS (ESI) m/z: 361.8/363.8 [M+H]⁺

### Step 2: Synthesis of 4-(2-(4-phenyl-2H-1,2,3-triazol-2-yl)pyrido[3,2-d]pyrimidin-4-yl)morpholine hydrochloride

A mixture of 4-(2-(4-bromo-2H-1,2,3-triazol-2-yl)pyrido[3,2-d]pyrimidin-4-yl)morpholine (150 mg, 0.42 mmol), phenylboronic acid (61 mg, 0.5 mmol), 1,1′-bis(diphenylphosphino)ferrocene-palladium(II)dichloride dichloromethane complex (35 mg, 0.042 mmol) and cesium carbonate (150 mg, 0.46 mmol) in water (1 mL) and dioxane (10 mL) was stirred at 100° C. under nitrogen atmosphere for 2 h. The mixture was concentrated and the resultant residue was purified by silica gel column chromatography (20% dichloromethane in methanol) and further purified by prep-HPLC (Column Xbridge 21.2*250 mm C18, 10 um, Mobile Phase A: water (10 mmol/L ammonium bicarbonate) B: acetonitrile) to afford 4-(2-(4-phenyl-2H-1,2,3-triazol-2-yl)pyrido[3,2-d]pyrimidin-4-yl)morpholine (80 mg) and 4-(2-(4-phenyl-1H-1,2,3-triazol-1-yl)pyrido[3,2-d]pyrimidin-4-yl)morpholine (15 mg) as a white solid respectively. ¹H NMR (400 MHz, DMSO-d₆) δ 8.83 (d, J=3.0 Hz, 1H), 8.69 (s, 1H), 8.28 (d, J=7.6 Hz, 1H), 8.03 (d, J=7.6 Hz, 2H), 7.88 (dd, J=8.5, 4.1 Hz, 1H), 7.55 (t, J=7.6 Hz, 2H), 7.47 (t, J=7.1 Hz, 1H), 4.63 (bs, 4H), 3.85 (t, J=4.0 Hz, 4H); LCMS (ESI) m/z: 359.9 [M+H]⁺.

Note: The starting material in step-2 contained minor amounts of the regioisomer from the step-1.

### Synthesis of 4-(2-(5-methyl-1H-benzo[d][1,2,3]triazol-1-yl)pyrido[3,2-d]pyrimidin-4-yl)morpholine (Compound 76)

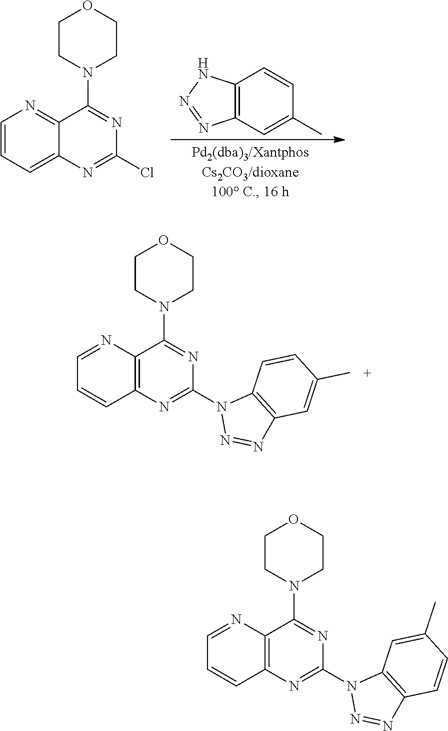

A mixture of 4-(2-chloropyrido[3,2-d]pyrimidin-4-yl)morpholine (250 mg, 1.0 mmol), 5-methyl-1H-benzo[d][1,2,3]triazole (133 mg, 1.0 mmol), tris(dibenzylideneacetone)dipalladium(0) (46 mg, 0.05 mmol), 4,5-bis(diphenylphosphino)-9,9-dimethylxanthene (58 mg, 0.1 mmol) and cesium carbonate (650 mg, 2.0 mmol) in dioxane (10 mL) was stirred at 100° C. under nitrogen atmosphere for 16 h. The mixture was poured into water, extracted with ethyl acetate (100 mL*2), the organic phase dried and concentrated. The obtained crude product was purified by silica gel column chromatography (20% ethyl acetate in petroleum ether) and further washed with methanol (30 mL) to afford 4-(2-(5-methyl-1H-benzo[d][1,2,3]triazol-1-yl)pyrido[3,2-d]pyrimidin-4-yl)morpholine (90 mg, 26%) as yellow solid. (Note: 1 HNMR showed it was a mixture of 2 isomers, but only one peak was observed on HPLC). ¹H NMR (400 MHz, CDCl₃) δ 8.72 (dt, J=3.9, 1.9 Hz, 1H), 8.44-8.34 (m, 1H), 8.28 (ddd, J=7.8, 6.0, 1.7 Hz, 1H), 8.05-7.90 (m, 1H), 7.68 (ddd, J=8.5, 4.1, 2.4 Hz, 1H), 7.46-7.27 (m, 1H), 4.72 (s, 4H), 4.02-3.91 (m, 4H), 2.60-2.56 (m, 3H); LCMS (ESI) m/z: 348.1 [M+H]⁺.

### Synthesis of 4-ethyl-2-(4-morpholinopyrido[3,2-d]pyrimidin-2-yl)-5-phenyl-1,2,4-triazol-3-one (Compound 77)

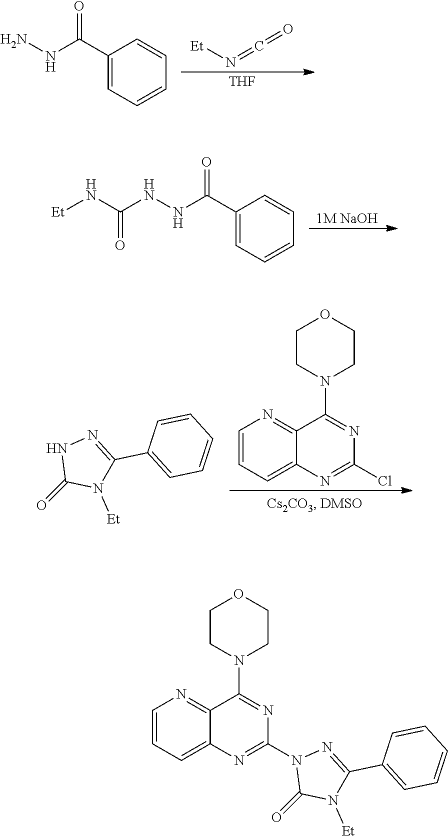

### Step 1: Synthesis of 1-benzamido-3-ethyl-urea

To a solution of benzohydrazide (1.00 g, 7.34 mmol) in THF (10 mL) was added isocyanatoethane (574 mg, 8.08 mmol) dropwise, then stirred for 14 h at 25° C. The resultant precipitate was filtered to give 1-benzamido-3-ethyl-urea (1.30 g, 85%) as white solid.

### Step 2: Synthesis of 4-ethyl-3-phenyl-1H-1,2,4-triazol-5-one

A solution of 1-benzamido-3-ethyl-urea (300 mg, 1.45 mmol) in 1M NaOH (4 mL) was stirred for 14 h at 100° C. The resultant mixture was acidified with 1 M HCl to pH 7 and the resultant precipitate was filtered to give 4-ethyl-3-phenyl-1H-1,2,4-triazol-5-one (190 mg, 69%) as pale solid.

### Step 3: Synthesis of 4-ethyl-2-(4-morpholinopyrido[3,2-d]pyrimidin-2-yl)-5-phenyl-1,2,4-triazol-3-one

A mixture of 4-(2-chloropyrido[3,2-d]pyrimidin-4-yl)morpholine (160 mg, 0.638 mmol), 4-ethyl-3-phenyl-1H-1,2,4-triazol-5-one (181 mg, 0.957 mmol) and Cs₂CO₃ (624 mg, 1.91 mmol) in DMSO (3 mL) was stirred for 14 h at 90° C. The mixture was filtered and the filtrate was concentrated in vacuo. The crude product was purified by prep-HPLC (Waters Xbridge Prep OBD C18 150*40 mm*10 um column; 30-50% acetonitrile in 10 mM ammonium bicarbonate in water, 8 min gradient) to obtain 4-ethyl-2-(4-morpholinopyrido[3,2-d]pyrimidin-2-yl)-5-phenyl-1,2,4-triazol-3-one (60 mg, 23%) as white solid. ¹H NMR (400 MHz, CHLOROFORM-d) δ 8.64 (d, J=2.4 Hz, 1H), 8.20 (d, J=8.8 Hz, 1H), 7.70 (d, J=1.6 Hz, 2H), 7.57-7.52 (m, 4H), 4.71 (bs, 4H), 3.90-3.88 (m, 6H), 1.31 (t, J=7.6 Hz, 3H). LCMS (ESI for C₂₁H₂₁N₇O₂ [M+H]⁺: 404.4.

### Synthesis of 1-cyclopentyl-3-(4-morpholinopyrido[3,2-d]pyrimidin-2-yl)imidazolidin-2-one (Compound 78)

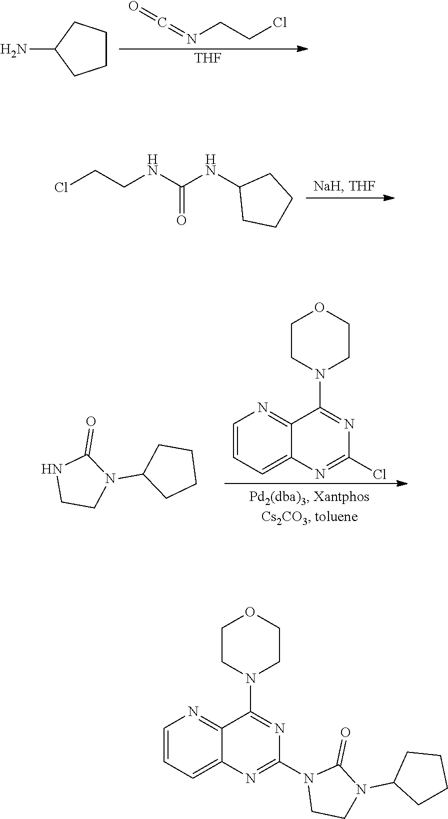

### Step 1: Synthesis of 1-(2-chloroethyl)-3-cyclopentyl-urea

To a solution of cyclopentanamine (0.6 g, 7.05 mmol) in THF (30 mL) was added dropwise 1-chloro-2-isocyanato-ethane (818 mg, 7.75 mmol) at 0° C. The mixture was stirred at 25° C. for 2 h. The resultant mixture was concentrated to obtain 1-(2-chloroethyl)-3-cyclopentyl-urea (1.3 g, 77%) as a white solid. LCMS (ESI) m/z: 191.0 [M+H]⁺

### Step 2: Synthesis of 1-cyclopentylimidazolidin-2-one

A solution of 1-(2-chloroethyl)-3-cyclopentyl-urea (0.7 g, 3.67 mmol) in THF (15 mL) was degassed and purged with nitrogen 3 times followed by the addition of NaH (367 mg, 9.18 mmol) at −20° C. The resultant mixture was stirred at −10° C. for 1 h and then at 0° C. for 1 h, and finally at 25° C. for 3 h. The mixture was then quenched with H₂O (2 mL), the aqueous phase was extracted with ethyl acetate (10 mL*3). The combined organic layers were dried over Na₂SO₄, filtered and concentrated under reduced pressure to obtain 1-cyclopentylimidazolidin-2-one (0.6 g, crude) as a pale yellow gum. LCMS (ESI) m/z: 155.3 [M+H]⁺

### Step 3: Synthesis of 1-cyclopentyl-3-(4-morpholinopyrido[3,2-d]pyrimidin-2-yl)imidazolidin-2-one

To a solution of 1-cyclopentylimidazolidin-2-one (138 mg, 896 umol) in toluene (3 mL) were added 4-(2-chloropyrido[3,2-d]pyrimidin-4-yl)morpholine (150 mg, 598 umol, Cs₂CO₃ (585 mg, 1.80 mmol), Pd₂(dba)₃ (55 mg, 60 umol) and Xantphos (35 mg, 60 umol) under nitrogen. The resultant mixture was stirred at 100° C. for 12 h. The mixture was concentrated and the residue was purified by prep-HPLC (Waters Xbridge Prep OBD C18 150*40 mm*10 um column; 25-55% acetonitrile in a 10 mM ammonium bicarbonate solution in water, 8 min gradient) to obtain 1-cyclopentyl-3-(4-morpholinopyrido[3,2-d]pyrimidin-2-yl)imidazolidin-2-one (102 mg, 274 umol, 46%) as a white solid. ¹H NMR (400 MHz, CHLOROFORM-d) δ=8.54-8.47 (m, 1H), 7.99-7.90 (m, 1H), 7.52-7.45 (m, 1H), 4.58 (bs, 4H), 4.45 (pent, J=8.4 Hz, 1H), 4.09 (t, J=8 Hz, 2H), 3.88 (t, J=4.8 Hz, 4H), 3.43 (t, J=4.4 Hz, 2H), 1.98-1.82 (m, 2H), 1.78-1.59 (m, 6H). LCMS (ESI) for C₁₉H₂₄N₆O₂ [M+H]⁺: 369.2.

### Synthesis of 1-(3-fluorophenyl)-3-(4-morpholinopyrido[3,2-d]pyrimidin-2-yl)imidazolidin-2-one (Compound 79)

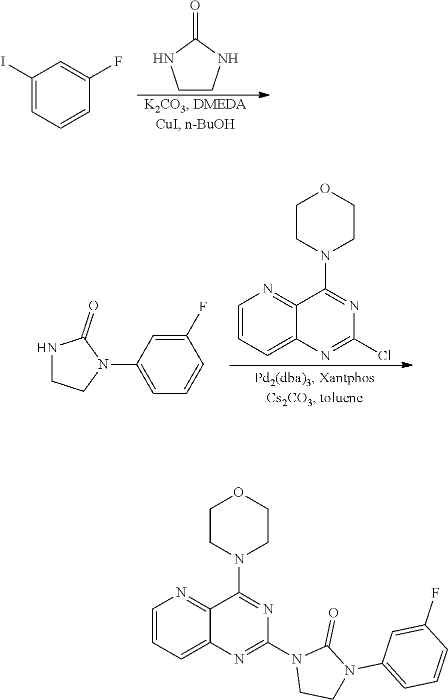

### Step 1: Synthesis of 1-(3-fluorophenyl)imidazolidin-2-one

To a solution of 1-fluoro-3-iodo-benzene (1 g, 4.50 mmol) in n-BuOH (50 mL) was added dropwise imidazolidin-2-one (1.94 g, 22.52 mmol) at 0° C. This was followed by the addition of CuI (86 mg, 451 umol), K₂CO₃ (1.87 g, 13.51 mmol) and DMEDA (119 mg, 1.35 mmol) to it and then the resultant mixture was stirred at 100° C. for 12 h. It was concentrated and the crude product was purified by flash column (ISCO 40 g silica, 60-70% ethyl acetate in petroleum ether, gradient over 20 min) to obtain 1-(3-fluorophenyl)imidazolidin-2-one (220 mg, 27%) as pale yellow gum. ¹H NMR (400 MHz, CHLOROFORM-d) Shift=7.44 (td, J=11.7, 2.2 Hz, 1H), 7.25 (s, 2H), 7.25-7.18 (m, 1H), 6.74 (ddt, J=8.2, 2.4, 1.2 Hz, 1H), 5.17 (bs, 1H), 3.91 (dd, J=8.8, 6.8 Hz, 2H), 3.67-3.50 (m, 2H)

### Step 2: Synthesis of 1-(3-fluorophenyl)-3-(4-morpholinopyrido[3,2-d]pyrimidin-2-yl)imidazolidin-2-one

To a solution of 1-(3-fluorophenyl)imidazolidin-2-one (200 mg, 1.11 mmol) and 4-(2-chloropyrido[3,2-d]pyrimidin-4-yl)morpholine (278 mg, 1.11 mmol) in toluene (5 mL) were added Cs₂CO₃ (1.08 g, 3.33 mmol), Pd₂(dba)₃ (102 mg, 111 umol) and Xantphos (64 mg, 111 umol, 0.1 eq) under nitrogen atmosphere. The mixture was stirred at 110° C. for 16 h and concentrated. The crude product was purified by prep-HPLC (Kromasil C18 (250*50 mm*10 um); column; 35-60% acetonitrile in a 10 mM ammonium bicarbonate solution in water, 10 min gradient) to obtain 1-(3-fluorophenyl)-3-(4-morpholinopyrido[3,2-d]pyrimidin-2-yl)imidazolidin-2-one (23 mg, 58 umol, 5%) as white solid. ¹H NMR (400 MHz, CHLOROFORM-d) δ=8.59-8.53 (m, 1H), 8.05-7.98 (m, 1H), 7.60-7.50 (m, 2H), 7.37-7.28 (m, 2H), 6.85-6.77 (m, 1H), 4.70 (bs, 4H), 4.31-4.21 (m, 2H), 3.99-3.86 (m, 6H). LCMS (ESI) for (C₂₀H₁₉FN₆O₂) [M+H]⁺: 395.1.

### Synthesis of 1-(4-morpholinopyrido[3,2-d]pyrimidin-2-yl)-3-phenyl-imidazolidin-2-one (Compound 80)

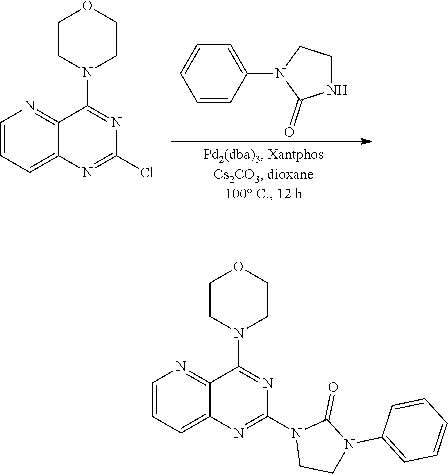 [SMILES: Clc1nc(N2CCOCC2)c2ncccc2n1 ; O=C1NCCN1c1ccccc1 ; O=C1N(c2ccccc2)CCN1c1nc(N2CCOCC2)c2ncccc2n1]

Compound 80 was synthesized according to the protocol described for the compound 79: ¹H NMR (400 MHz, CHLOROFORM-d) δ 9.08 (d, J=8.16 Hz, 1H), 8.72 (d, J=2.87 Hz, 1H), 7.75 (dd, J=8.60, 4.19 Hz, 1H), 7.58 (d, J=7.94 Hz, 2H), 7.38 (t, J=7.83 Hz, 2H), 7.20 (t, J=7.50 Hz, 1H), 5.22 (bs, 2H), 4.68 (bs, 2H), 4.40 (bs, 2H), 4.19 (bs, 2H), 3.96 (bs, 4H); LCMS: [M+H]⁺: 377.2

### Synthesis of 4-methyl-2-(4-morpholinopyrido[3,2-d]pyrimidin-2-yl)-5-phenyl-1,2,4-triazol-3-one (Compound 81)

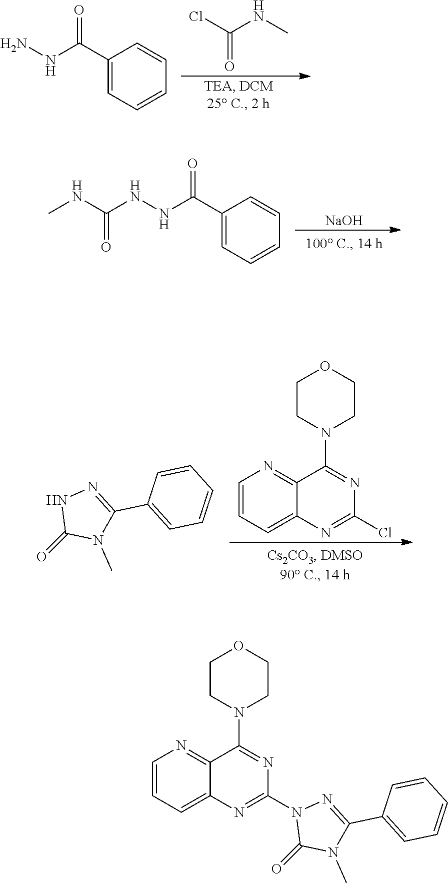

### Step 1: Synthesis of 1-benzamido-3-methyl-urea

A solution of benzohydrazide (1.00 g, 7.34 mmol), N-methylcarbamoyl chloride (2.06 g, 22.03 mmol) and TEA (2.23 g, 22.03 mmol) in DCM (10 mL) was stirred for 2 h at 15° C. The mixture was filtered, and the filtrate was concentrated in vacuo to obtain the crude product. It was purified by flash column (ISCO 20 g silica, 0˜60% ethyl acetate in petroleum ether, gradient over 30 min) to obtain 1-benzamido-3-methyl-urea (540 mg) as off-white solid.

### Step 2: Synthesis of 4-methyl-3-phenyl-1H-1,2,4-triazol-5-one

A solution of 1-benzamido-3-methyl-urea (300 mg, 1.55 mmol) in 1M NaOH (4 mL) was stirred for 14 h at 100° C. The mixture was then acidified with 1 M HCl to pH 7 and the resultant precipitate was filtered, washed with water and dried to obtain 4-methyl-3-phenyl-1H-1,2,4-triazol-5-one (130 mg, 47.79%) as off-white solid. ¹H NMR (400 MHz, DMSO-d₆) δ 11.89 (bs, 1H), 7.69 (dd, J=6.42, 2.87 Hz, 2H), 7.49-7.57 (m, 3H), 3.24 (s, 3H).

### Step 3: Synthesis of 4-methyl-2-(4-morpholinopyrido[3,2-d]pyrimidin-2-yl)-5-phenyl-1,2,4-triazol-3-one

To a solution of 4-(2-chloropyrido[3,2-d]pyrimidin-4-yl)morpholine (100 mg, 400 umol) in DMSO (1 mL), were added 4-methyl-3-phenyl-1H-1,2,4-triazol-5-one (70 mg, 400 umol) and Cs₂CO₃ (391 mg, 1.20 mmol) at 90° C. for 14 h. The mixture was filtered and the filtrate was concentrated. The crude was purified by prep-HPLC (Waters Xbridge 150*40 mm*10 um, column; 25%-50% acetonitrile in an a 10 mM ammonium bicarbonate solution in water, 8 min gradient) to obtain 4-methyl-2-(4-morpholinopyrido[3,2-d]pyrimidin-2-yl)-5-phenyl-1,2,4-triazol-3-one (22 mg, 14%) as white solid. ¹H NMR (400 MHz, CHLOROFORM-d) δ 8.65 (dd, J=4.05, 1.67 Hz, 1H), 8.21 (dd, J=8.58, 1.67 Hz, 1H), 7.74 (dd, J=7.63, 1.79 Hz, 2H), 7.62-7.50 (m, 4H), 5.13-4.25 (m, 4H), 4.01-3.89 (m, 4H), 3.45 (s, 3H), LCMS (ESI) for C₂₀H₁₉N₇O₂ [M+H]⁺: 390.2.

### Synthesis of 2-(4-morpholinopyrido[3,2-d]pyrimidin-2-yl)-5-phenyl-4H-1,2,4-triazol-3-one (Compound 82)

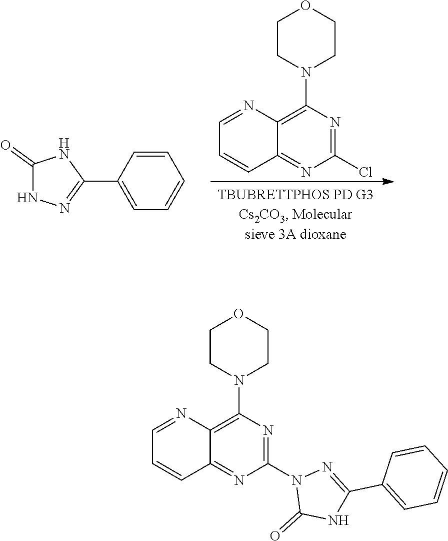

To a solution of 4-(2-chloropyrido[3,2-d]pyrimidin-4-yl)morpholine (200 mg, 798 umol), 3-phenyl-1,4-dihydro-1,2,4-triazol-5-one (154 mg, 957 umol), Cs₂CO₃ (520 mg, 1.60 mmol) and Molecular sieve 3A (20 mg, 1.00 eq) in dioxane (3 mL) was added TBUBRETTPHOS PD G3 (68 mg, 80 mol) and stirred for 16 h at 80° C. under nitrogen atmosphere. The resultant mixture was filtered and the filtrate was subjected to prep-HPLC (Waters Xbridge Prep OBD C18 150*40 mm*10 um column; 20%-50% acetonitrile in an 10 mM NH₄HCO₃ in water, 8 min gradient) to obtain 2-(4-morpholinopyrido[3,2-d]pyrimidin-2-yl)-5-phenyl-4H-1,2,4-triazol-3-one (76 mg, 25%) as white solid. ¹H NMR (400 MHz, DMSO-d₆+1 drop HCl) δ=8.74 (d, J=4 Hz, 1H), 8.57 (d, J=8.4 Hz, 1H), 8.00-7.98 (m, 2H), 7.89-7.86 (m, 1H), 7.52-7.45 (m, 3H), 5.05 (bs, 2H), 4.28 (bs, 2H), 3.79 (s, 4H). LCMS (ESI) for (C₁₉H₁₇N₇O₂) [M+H]⁺: 376.2

### Synthesis of tert-butyl 4-[4-(4-morpholinopyrido[3,2-d]pyrimidin-2-yl)pyrimidin-2-yl]piperidine-1-carboxylate (Compound 83), 4-[2-[2-(4-piperidyl)pyrimidin-4-yl]pyrido[3,2-d]pyrimidin-4-yl]morpholine (Compound 84) and 4-[2-[2-(1-methyl-4-piperidyl)pyrimidin-4-yl]pyrido[3,2-d]pyrimidin-4-yl]morpholine (Compound 85)

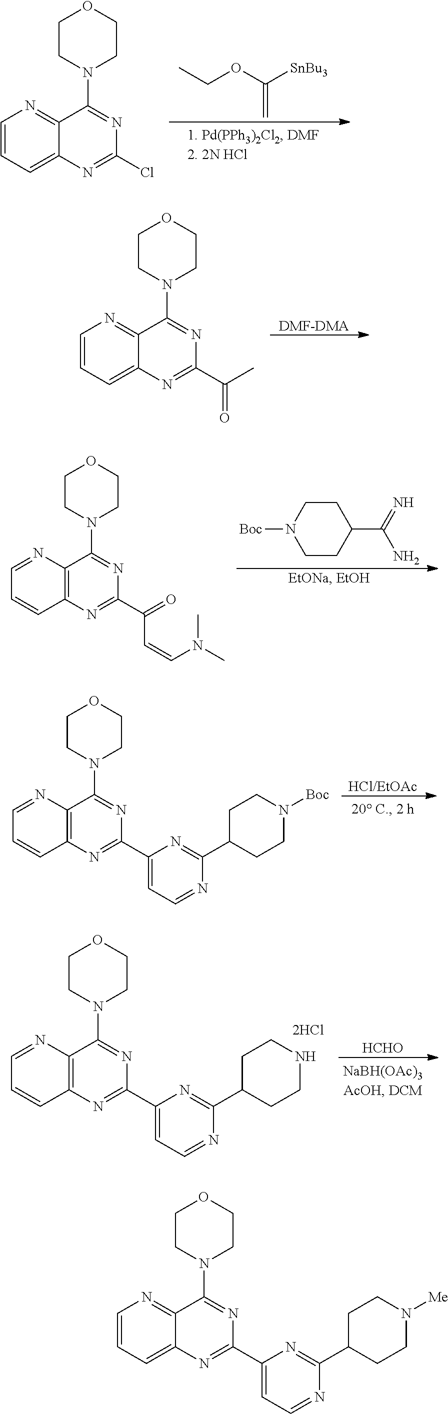

### Step 1: Synthesis of 1-(4-morpholinopyrido[3,2-d]pyrimidin-2-yl)ethanone

To a solution of 4-(2-chloropyrido[3,2-d]pyrimidin-4-yl)morpholine (5 g, 19.95 mmol) in DMF (100 mL) were added tributyl(1-ethoxyvinyl)stannane (8.64 g, 23.93 mmol) and Pd(dppf)Cl₂ (146 mg, 200 umol) under nitrogen atmosphere and the resultant mixture was stirred at 100° C. for 48 h. The mixture was cooled and then ethyl acetate (5 mL) and KF (4 g in 50 mL of water) were added and the resultant mixture was stirred at 25° C. for 3 h. The layers were separated, the aqueous phase was extracted with acetate (10 mL*3), the combined organic layers were washed with saturated NaHCO₃ (10 mL), brine (10 mL), dried over Na₂SO₄ and concentrated. The resultant crude product was dissolved in THF (2 mL), and HCl (2 M, 2 mL) was added and the mixture was stirred at 40° C. for 3 h. Water (30 mL) was added to the mixture and the aqueous solution was extracted with ethyl acetate (30 mL*8), The combined organic phase was washed with brine (20 mL*3), dried with anhydrous Na₂SO₄, filtered and concentrated. The crude product was purified by flash column (ISCO 40 g silica, 0-80% ethyl acetate in petroleum ether, gradient over 20 min) to obtain 1-(4-morpholinopyrido[3,2-d]pyrimidin-2-yl)ethanone (2.3 g, 8.91 mmol, 44.65%) as yellow solid; LCMS (ESI) m/z: 259.1 [M+H]⁺. ¹H NMR (400 MHz, CHLOROFORM-d) δ 8.78 (dd, J=4.1, 1.6 Hz, 1H), 8.29 (dd, J=8.5, 1.6 Hz, 1H), 7.67 (dd, J=8.5, 4.1 Hz, 1H), 4.52 (bs, 4H), 3.90 (t, J=4.8 Hz, 4H), 2.76 (s, 3H).

### Step 2: Synthesis of (Z)-3-(dimethylamino)-1-(4-morpholinopyrido[3,2-d]pyrimidin-2-yl)prop-2-en-1-one

A mixture of 1-(4-morpholinopyrido[3,2-d]pyrimidin-2-yl)ethanone (2.1 g, 8.13 mmol) in DMF-DMA (8.97 g, 75.28 mmol) was stirred at 100° C. for 16 h. It was cooled and the resultant precipitate was filtered, the solid was collected, washed with ethyl acetate (15 mL*3) and dried to obtain (Z)-3-(dimethylamino)-1-(4-morpholinopyrido[3,2-d]pyrimidin-2-yl)prop-2-en-1-one (1.7 g, 67%) as yellow solid. LCMS (ESI) m/z: 314.2 [M+H]⁺.

### Step 3: Synthesis of tert-butyl 4-[4-(4-morpholinopyrido[3,2-d]pyrimidin-2-yl)pyrimidin-2-yl]piperidine-1-carboxylate

To a mixture of tert-butyl 4-carbamimidoylpiperidine-1-carboxylate (130 mg, 574.43 umol) in EtOH (4 mL), were added (Z)-3-(dimethylamino)-1-(4-morpholinopyrido[3,2-d]pyrimidin-2-yl)prop-2-en-1-one (120 mg, 383 umol) and EtONa (52 mg, 766 umol). The resultant mixture was stirred at 80° C. for 16 h, then cooled, filtered and the filtrate was concentrated. The crude product was purified by prep-HPLC (Waters Xbridge Prep OBD C18 150*40 mm*10 um column; 30%-70% acetonitrile in an a 0.05% ammonium hydroxide and 10 mM ammonium bicarbonate solution, 8 min gradient) to obtain tert-butyl 4-[4-(4-morpholinopyrido[3,2-d]pyrimidin-2-yl)pyrimidin-2-yl]piperidine-1-carboxylate (50 mg, 27%) as pale yellow solid. ¹H NMR (400 MHz, CHLOROFORM-d) δ=8.87 (d, J=5.1 Hz, 1H), 8.76 (dd, J=4.2, 1.8 Hz, 1H), 8.37 (dd, J=8.5, 1.7 Hz, 1H), 8.19 (d, J=5.1 Hz, 1H), 7.67 (dd, J=8.6, 4.2 Hz, 1H), 4.64 (bs, 4H), 4.26 (bs, 2H), 3.94 (t, J=4.8 Hz, 4H), 3.30 (tt, J=11.7, 3.7 Hz, 1H), 2.90-2.80 (m, 2H), 2.13-2.03 (m, 2H), 1.94 (dq, J=8.4, 4.4 Hz, 2H), 1.48 (s, 9H). LCMS (ESI) for C₂₅H₃₁N₇O₃ [M+H]⁺:478.2.

### Step 4: Synthesis of 4-[2-[2-(4-piperidyl)pyrimidin-4-yl]pyrido[3,2-d]pyrimidin-4-yl]morpholine

To a mixture of tert-butyl 4-[4-(4-morpholinopyrido[3,2-d]pyrimidin-2-yl)pyrimidin-2-yl]piperidine-1-carboxylate (380 mg, 796 umol) in ethyl acetate (8 mL), was added HCl/EtOAc (20 mL) and the resulting mixture was stirred at 20° C. for 2 h. It was concentrated and the crude product was purified by prep-HPLC (Welch Xtimate C18 150*25 mm*5 um column; 10%-30% acetonitrile in a 0.04% hydrochloric acid solution in water, 10 min gradient) to obtain 4-[2-[2-(4-piperidyl)pyrimidin-4-yl]pyrido[3,2-d]pyrimidin-4-yl]morpholine (42 mg, 13%) as orange solid. ¹HNMR (400 MHz, METHANOL-d4) δ=9.17 (d, J=5.1 Hz, 1H), 9.02 (dd, J=4.1, 1.3 Hz, 1H), 8.75 (dd, J=8.6, 1.3 Hz, 1H), 8.49 (d, J=5.0 Hz, 1H), 8.06 (dd, J=8.6, 4.3 Hz, 1H), 5.32 (bs, 2H), 4.62 (bs, 2H), 4.01 (t, J=4.8 Hz, 4H), 3.63-3.47 (m, 3H), 3.29-3.22 (m, 2H), 2.49-2.40 (m, 2H), 2.39-2.25 (m, 2H). LCMS (ESI) for C₂₀H₂₅C₁₂N₇O [M+H]⁺:378.2

### Step 5: Synthesis of 4-[2-[2-(1-methyl-4-piperidyl)pyrimidin-4-yl]pyrido[3,2-d]pyrimidin-4-yl]morpholine

To a solution of 4-[2-[2-(4-piperidyl)pyrimidin-4-yl]pyrido[3,2-d]pyrimidin-4-yl]morpholine (200 mg, 530 umol) in DCM (10 mL), were added formaldehyde (129. mg, 1.59 mmol) and NaBH(OAc)₃ (225 mg, 1.06 mmol) and the resulting mixture was stirred at 20° C. for 16 h. The mixture was then filtered, and the filtrate was purified by prep-HPLC (Phenomenex Gemini-NX 150*30 mm*5 um column; 5%-35% acetonitrile in an a 10 mM ammonium bicarbonate solution, 8 min gradient) to obtain 4-[2-[2-(1-methyl-4-piperidyl)pyrimidin-4-yl]pyrido[3,2-d]pyrimidin-4-yl]morpholine (64 mg, 30%) as pale yellow solid. ¹H NMR (400 MHz, CHLOROFORM-d) δ=8.87 (bs, 1H), 8.75 (bs, 1H), 8.35 (d, J=8.8 Hz, 1H), 8.17 (bs, 1H), 7.70-7.61 (m, 1H), 4.63 (bs, 4H), 3.94 (bs, 4H), 3.11 (bs, 1H), 3.00 (bs, 2H), 2.33 (bs, 3H), 2.12 (bs, 6H). LCMS (ESI) for C₂₁H₂₅N₇O [M+H]⁺:392.2.

### Synthesis of tert-butyl 4-[4-(4-morpholinopyrido[3,2-d]pyrimidin-2-yl)pyrimidin-2-yl]piperazine-1-carboxylate (Compound 86) and 4-[2-(2-piperazin-1-ylpyrimidin-4-yl)pyrido[3,2-d]pyrimidin-4-yl]morpholine (Compound 87)

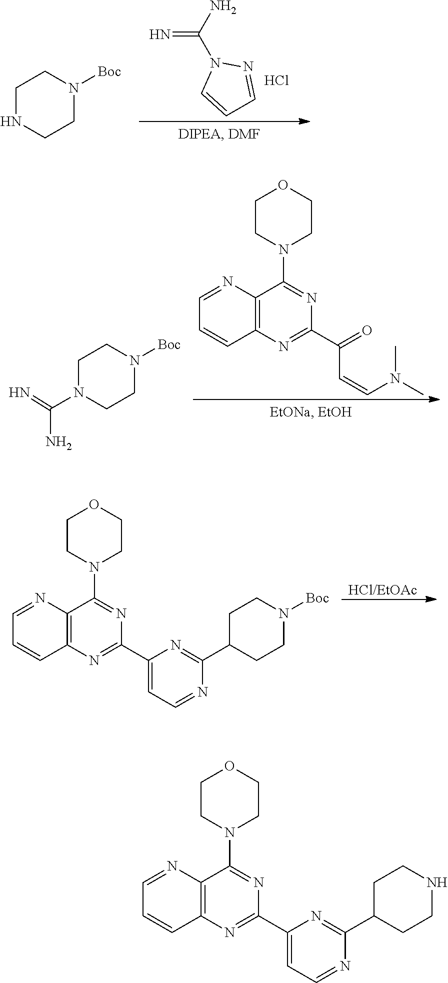

### Step 1: Synthesis of tert-butyl 4-carbamimidoylpiperazine-1-carboxylate

To a solution of tert-butyl piperazine-1-carboxylate (500 mg, 2.68 mmol) and pyrazole-1-carboxamidine;hydrochloride (394 mg, 2.68 mmol) in DMF (10 mL) was added DIPEA (35 mg, 269 umol) and the mixture was stirred at 20° C. for 16 h. It was concentrated under reduced pressure to give a crude product tert-butyl 4-carbamimidoylpiperazine-1-carboxylate (1.2 g, crude) as white solid; LCMS (ESI) m/z: 229.1 [M+H]⁺

### Step 2: Synthesis of tert-butyl 4-[4-(4-morpholinopyrido[3,2-d]pyrimidin-2-yl)pyrimidin-2-yl]piperazine-1-carboxylate

To a mixture of tert-butyl 4-carbamimidoylpiperazine-1-carboxylate (328 mg, 1.44 mmol) in EtOH (8 mL) was added (Z)-3-(dimethylamino)-1-(4-morpholinopyrido[3,2-d]pyrimidin-2-yl)prop-2-en-1-one (300 mg, 958 umol) and EtONa (130 mg, 1.91 mmol) and the resultant mixture was stirred at 80° C. for 16 h. The mixture was filtered and the filtrated was concentrated to give 700 mg crude product which was purified by prep-HPLC (Waters Xbridge Prep OBD C18 150*40 mm*10 um column; 40%-70% acetonitrile in an a 10 mM ammonium bicarbonate solution, 8 min gradient) to obtain tert-butyl 4-[4-(4-morpholinopyrido[3,2-d]pyrimidin-2-yl)pyrimidin-2-yl]piperazine-1-carboxylate (275 mg) as pale yellow solid. ¹H NMR (400 MHz, CHLOROFORM-d) δ=8.74 (dd, J=4.1, 1.7 Hz, 1H), 8.51 (d, J=5.1 Hz, 1H), 8.28 (dd, J=1.5, 8.4 Hz, 1H), 7.65 (dd, J=8.5, 4.1 Hz, 1H), 7.59 (d, J=4.9 Hz, 1H), 4.62 (bs, 4H), 4.00-3.90 (m, 8H), 3.61-3.51 (m, 4H), 1.50 (s, 9H). LCMS (ESI) for C₂₄H₃₀N₈O₃ [M+H]⁺: 479.3.

### Step 3: Synthesis of 4-[2-(2-piperazin-1-ylpyrimidin-4-yl)pyrido[3,2-d]pyrimidin-4-yl]morpholine

To a solution of tert-butyl 4-[4-(4-morpholinopyrido[3,2-d]pyrimidin-2-yl)pyrimidin-2-yl]piperazine-1-carboxylate (500 mg, 1.04 mmol) in EtOAc (5 mL) was added HCl/EtOAc (15 mL) and then the resultant mixture was stirred at 20° C. for 1 h. It was then filtered, and the filtrate was purified by prep-HPLC (Phenomenex Luna C8 250*50 mm*10 um column; 1%-20% acetonitrile in a 0.05% hydrochloric acid solution in water, 10 min gradient) to obtain 4-[2-(2-piperazin-1-ylpyrimidin-4-yl)pyrido[3,2-d]pyrimidin-4-yl]morpholine·HCl (282 mg, 65%) as a yellow solid. 1H NMR (400 MHz, METHANOL-d₄) δ=9.01 (d, J=4.2 Hz, 1H), 8.80-8.75 (m, 2H), 8.04 (dd, J=8.6, 4.2 Hz, 1H), 7.85 (d, J=4.9 Hz, 1H), 5.33-5.30 (m, 2H), 4.58 (bs, 2H), 4.38-4.36 (m, 4H), 4.01-3.99 (m, 4H), 3.40 (brd, J=4.4 Hz, 4H). LCMS (ESI) for C₁₉H₂₂N₈O [M+H]⁺:379.2.

The following compounds were synthesized according to the protocol described above.

| Name | Structure | NMR, MS | # |
|---|---|---|---|
| 4-[2-(2- tetrahydropyran-4- ylpyrimidin-4- yl)pyrido[3,2- d]pyrimidin-4- yl]morpholine | 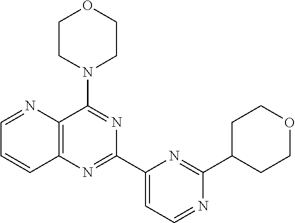 [SMILES: c1cnc2c(N3CCOCC3)nc(-c3ccnc(C4CCOCC4)n3)nc2c1] | ¹H NMR (400MHz, CHLOROFORM-d) δ = 8.89 (d, J = 5.1 Hz, 1H), 8.76 (dd, J = 4.2, 1.8Hz, 1H), 8.36 (dd, J = 8.4, 1.8Hz, 1H), 8.20 (d, J = 5.1 Hz, 1H), 7.67 (dd, J = 8.4, 4.2Hz, 1H), 4.64 (bs, 4H), 4.17-4.07 (m, 2H), 3.98-3.90 (m, 4H), 3.59 (dt, J = 11.6, 2.3Hz, 2H), 3.40 (tt, J = 11.4, 4.0Hz, 1H), 2.22-1.99 (m, 4H). LCMS (ESI) for C20H22N6O2 [M + H]+:379.2. | 88 |
| 4-[4-(4- morpholinopyrido[3,2- d]pyrimidin-2- yl)pyrimidin-2- yl]morpholine | 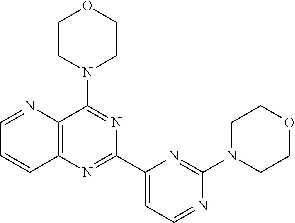 [SMILES: c1cnc2c(N3CCOCC3)nc(-c3ccnc(N4CCOCC4)n3)nc2c1] | ¹H NMR (400MHz, CHLOROFORM-d) δ 8.74 (dd, J = 4.1, 1.7Hz, 1H), 8.52 (d, J = 4.9Hz, 1H), 8.28 (dd, J = 8.4, 1.8Hz, 1H), 7.65 (dd, J = 8.4, 4.2Hz, 1H), 7.61 (d, J = 5.1 Hz, 1H), 4.62 (bs, 4H), 4.0-3.85 (m, 8H), 3.85-3.80 (m, 4H). LCMS (ESI) for C19H21N7O2 [M + H]+:380.2. | 89 |
| 4-(2-(2- phenylpyrimidin-4- yl)pyrido[3,2- d]pyrimidin-4- yl)morpholine | 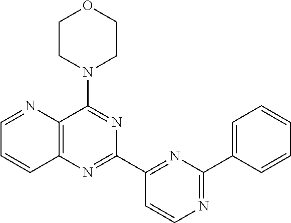 [SMILES: c1ccc(-c2nccc(-c3nc(N4CCOCC4)c4ncccc4n3)n2)cc1] | ¹H NMR (400 MHz, DMSO-d₆) δ 9.10 (d, J = 5.1 Hz, 1H), 8.89 (dd, J = 4.1, 1.7 Hz, 1H), 8.54 (dd, J = 7.3, 2.4 Hz, 2H), 8.37 (dd, J = 8.5, 1.7 Hz, 1H), 8.33 (d, J = 5.1 Hz, 1H), 7.90 (dd, J = 8.5, 4.1 Hz, 1H), 7.60-7.57 (m, 3H), 4.61 (bs, 4H), 3.85 (t, J = 4Hz, 4H). LCMS (ESI) m/z: 371.3 [M + H]⁺. | 90 |

### Synthesis of tert-butyl 4-[6-(4-morpholinopyrido[3,2-d]pyrimidin-2-yl)-2-pyridyl]piperidine-1-carboxylate (Compound 91) and 4-[2-[6-(4-piperidyl)-2-pyridyl]pyrido[3,2-d]pyrimidin-4-yl]morpholine (Compound 92)

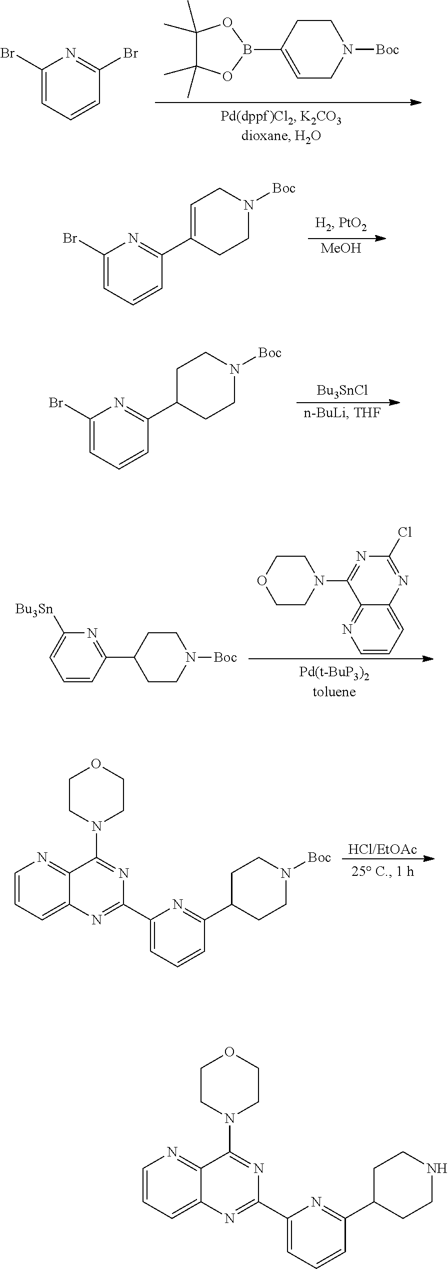

### Step 1: Synthesis of tert-butyl 4-(6-bromo-2-pyridyl)-3,6-dihydro-2H-pyridine-1-carboxylate

To a solution of 2,6-dibromopyridine (2 g, 8.44 mmol) and tert-butyl 4-(4,4,5,5-tetramethyl-1,3,2-dioxaborolan-2-yl)-3,6-dihydro-2H-pyridine-1-carboxylate (3.13 g, 10.13 mmol) in dioxane (20 mL) and H₂O (8 mL) were added Pd(dppf)Cl₂·CH₂Cl₂ (689 mg, 844 umol) and K₂CO₃ (2M in water, 8.44 mL). The resultant mixture was stirred at 90° C. under nitrogen for 12 h. Water (10 mL) was added to the mixture and it was extracted with EtOAc (20 mL*4). The organic layer was washed with brine (10 mL), dried over Na₂SO₄ and concentrated to give the crude product. It was purified by flash column (ISCO 40 g silica, 20-40% ethyl acetate in petroleum ether, over 20 min) to obtain tert-butyl 4-(6-bromo-2-pyridyl)-3,6-dihydro-2H-pyridine-1-carboxylate (1.5 g, 52%, Product-A) and tert-butyl 4-[6-(1-tert-butoxycarbonyl-3,6-dihydro-2H-pyridin-4-yl)-2-pyridyl]-3,6-dihydro-2H-pyridine-1-carboxylate (1.2 g, 32%, Product-B) as white solids. Product-A: ¹H NMR (400 MHz, CHLOROFORM-d) δ=7.42 (t, J=7.6 Hz, 1H), 7.30-7.15 (m, 2H), 6.62 (bs, 1H), 4.09-4.03 (m, 2H), 3.56 (bs, 2H), 2.52 (bs, 2H), 1.42 (s, 9H).

### Step 2: Synthesis of methyl tert-butyl 4-(6-bromo-2-pyridyl)piperidine-1-carboxylate

To a solution of tert-butyl 4-(6-bromo-2-pyridyl)-3,6-dihydro-2H-pyridine-1-carboxylate (1.5 g, 4.42 mmol) in EtOAc (20 mL) was added PtO₂ (100 mg, 442 umol) under nitrogen atmosphere. The suspension was degassed under vacuum and purged with hydrogen several times. The mixture was stirred under hydrogen (15 psi) at 15° C. for 6 h. The reaction mixture was filtered and the filtrate was concentrated. The crude product was purified by flash column (ISCO 20 g silica, 10-30% ethyl acetate in petroleum ether, gradient over 20 min) to obtain tert-butyl 4-(6-bromo-2-pyridyl)piperidine-1-carboxylate (0.8 g, 53%) as white solid.

### Step 3: Synthesis of tert-butyl 4-(6-tributylstannyl-2-pyridyl)piperidine-1-carboxylate

To a solution of tert-butyl 4-(6-bromo-2-pyridyl)piperidine-1-carboxylate (0.5 g, 1.47 mmol) in THF (10 mL) was added n-BuLi (2.5M, 762 uL) at −70° C. and the mixture was stirred at −70° C. for 1 h. Then tributyl(chloro)stannane (572 mg, 1.76 mmol) was added to the above solution at −70° C. and stirred for 1 h at that temperature and then at 20° C. for 12 h. 15 mL of water was added to the mixture and it was extracted with ethyl acetate (30 mL*2). The combined organic layers were washed with brine (15 mL) and dried over Na₂SO₄. Concentration and purification of the crude product by flash column (ISCO 10 g silica, 0-10% ethyl acetate in petroleum ether, gradient over 20 min) yielded tert-butyl 4-(6-tributylstannyl-2-pyridyl)piperidine-1-carboxylate (80 mg, 10%) as a colorless oil.

### Step 4: Synthesis of tert-butyl 4-[6-(4-morpholinopyrido[3,2-d]pyrimidin-2-yl)-2-pyridyl]piperidine-1-carboxylate

To a solution of tert-butyl 4-(6-tributylstannyl-2-pyridyl)piperidine-1-carboxylate (80 mg, 145 umol) in toluene (4 mL) were added 4-(2-chloropyrido[3,2-d]pyrimidin-4-yl)morpholine (36 mg, 145 umol) and Pd(t-Bu₃P)₂ (7 mg, 15 umol). Then the mixture was stirred at 100° C. for 12 h and concentrated. The crude product obtained was purified by prep-HPLC (Waters Xbridge Prep OBD C18 150*40 10u column; 40-70% acetonitrile in an a 10 mM ammonium bicarbonate solution in water, 8 min gradient) to obtain tert-butyl 4-[6-(4-morpholinopyrido[3,2-d]pyrimidin-2-yl)-2-pyridyl]piperidine-1-carboxylate (12 mg, 17%) as pale yellow solid. ¹H NMR (400 MHz, CHLOROFORM-d) δ=8.75-8.67 (m, 1H), 8.37 (dd, J=8, 5, 1.4 Hz, 1H), 8.32 (d, J=7.6 Hz, 1H), 7.81 (t, J=7.8 Hz, 1H), 7.64 (dd, J=8.5, 4.1 Hz, 1H), 7.30-7.28 (m, 1H), 4.63 (bs, 4H), 4.28 (bs, 2H), 4.04-3.90 (m, 4H), 3.28-3.11 (m, 1H), 2.88 (bt, J=12.6 Hz, 2H), 2.08 (br d, J=12.4 Hz, 2H), 1.82-1.66 (m, 2H), 1.50 (s, 9H). LCMS (ESI) for C₂₆H₃₂N₆O₃ [M+H]⁺: 477.3.

### Step 5: Synthesis of 4-[2-[6-(4-piperidyl)-2-pyridyl]pyrido[3,2-d]pyrimidin-4-yl]morpholine

A solution of tert-butyl 4-[6-(4-morpholinopyrido[3,2-d]pyrimidin-2-yl)-2-pyridyl]piperidine-1-carboxylate (9 mg, 19 umol) in 4M HCl/EtOAc (5 mL) was stirred at 25° C. for 30 min. The reaction mixture was concentrated and 10 mL deionized water was added to the residue. The resultant mixture was lyophilized to obtain 4-[2-[6-(4-piperidyl)-2-pyridyl]pyrido[3,2-d]pyrimidin-4-yl]morpholine·3HCl (8 mg, 89%) as pale yellow solid. ¹H NMR (400 MHz, METHANOL-d₄) δ=8.98 (dd, J=4.2, 1.4 Hz, 1H), 8.84-8.71 (m, 1H), 8.57 (d, J=7.6 Hz, 1H), 8.13 (t, J=7.8 Hz, 1H), 8.03 (dd, J=8.6, 4.3 Hz, 1H), 7.72 (d, J=7.8 Hz, 1H), 5.31 (bs, 2H), 4.60 (bs, 2H), 4.01 (bs, 4H), 3.60 (bd, J=12.8 Hz, 2H), 3.39-3.33 (m, 1H), 3.28-3.19 (m, 2H), 2.38-2.23 (m, 4H). LCMS (ESI) for C₂₁H₂₄N₆O [M+H]⁺: 377.2.

### Synthesis of 4-(2-(5-methoxy-2-phenylpyrimidin-4-yl)pyrido[3,2-d]pyrimidin-4-yl)morpholine (Compound 93) and 4-(4-morpholinopyrido[3,2-d]pyrimidin-2-yl)-2-phenylpyrimidin-5-ol (Compound 94)

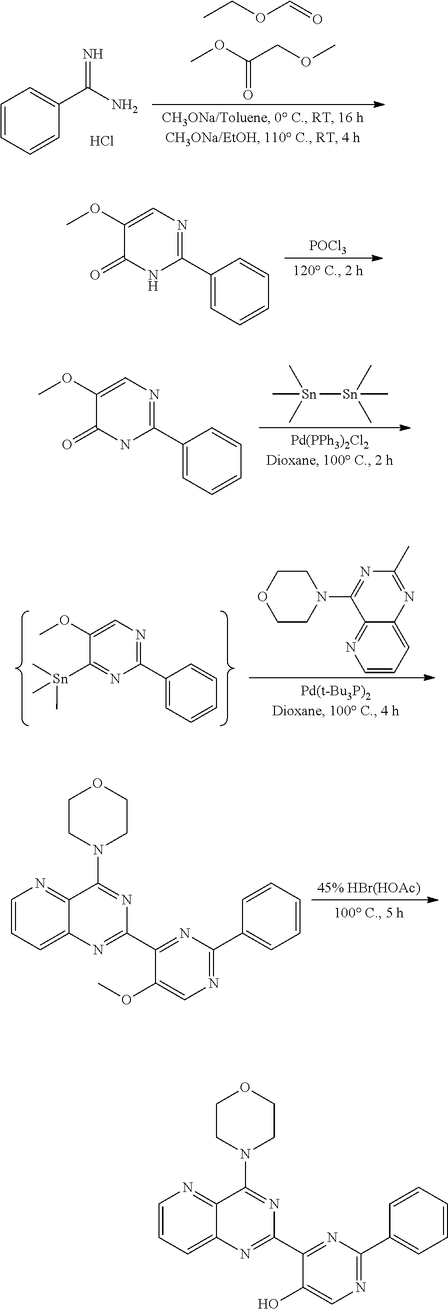

### Step 1: Synthesis of 5-methoxy-2-phenylpyrimidin-4(3H)-one

Methyl 2-methoxyacetate (6.24 g, 60.0 mmol) and ethylformate (4.44 g, 60.0 mmol) were added dropwise to a slurry of sodium methoxide (6.48 g, 120.0 mmol) in toluene (120 mL) in ice bath. After stirring at room temperature overnight, the resulting solution was concentrated in vacuo. The residue was mixed with benzimidamide hydrochloride (9.36 g, 60.0 mmol) and sodium methoxide (3.24 g, 60.0 mmol) in ethanol (200 mL) was refluxed at 110° C. for 6 h. The mixture was concentrated and the residue was acidified to pH 4˜5 with concentrated hydrochloric acid. The formed precipitate was collected by filtration and dried under vacuum to afford 5-methoxy-2-phenylpyrimidin-4(3H)-one (4.5 g, 37%) as grey solid. LCMS (ESI) m/z: 203.1 [M+H]⁺

### Step 2: Synthesis of 4-chloro-5-methoxy-2-phenylpyrimidine

A mixture of 5-methoxy-2-phenylpyrimidin-4-ol (2.0 g, 1.0 mmol) in phosphoryl trichloride (20 mL) was stirred at 120° C. for 2 h. The resultant mixture was poured into crushed ice. The resultant precipitate was collected by filtration and dried under vacuum to afford 4-chloro-5-methoxy-2-phenylpyrimidine (1.5 g, 68%) as grey solid. LCMS (ESI) m/z: 221.1/223.1 [M+H]⁺.

### Step 3: Synthesis of 4-(2-(5-methoxy-2-phenylpyrimidin-4-yl)pyrido[3,2-d]pyrimidin-4-yl)morpholine

A mixture of 4-chloro-5-methoxy-2-phenylpyrimidine (220 mg, 1.0 mmol), hexamethyldistannane (654 mg, 2.0 mmol) and bis(triphenylphosphine)palladium(II) chloride (71 mg, 0.1 mmol) in dioxane (10 mL) was stirred at 100° C. for 2 h under nitrogen atmosphere. The mixture was poured into dichloromethane (200 mL), the organic phase was washed successively with saturated aqueous potassium fluoride solution (100 mL), brine and concentrated to afford the 5-methoxy-2-phenyl-4-(trimethylstannyl)pyrimidine (400 mg) as brown oil. This product was mixed with 4-(2-chloropyrido[3,2-d]pyrimidin-4-yl)morpholine (200 mg, 0.8 mmol) and bis(tri-tert-butylphosphine)palladium (116 mg, 0.10 mmol) in dioxane (20 mL) and the resultant mixture was stirred at 100° C. for another 4 h. The resultant mixture was concentrated and crude product thus obtained was purified by silica gel column chromatography (25% methanol in dichloromethane) to afford 4-(2-(5-methoxy-2-phenylpyrimidin-4-yl)pyrido[3,2-d]pyrimidin-4-yl)morpholine (240 mg, 54% purity), which was further purified by prep-HPLC (Column Xbridge 21.2*250 mm C18, 10 um, Mobile Phase A: water (10 mmol/L ammonium bicarbonate) B: acetonitrile) to obtain the target compound (40 mg) as off-white solid. ¹H NMR (400 MHz, CDCl₃) δ 8.77 (dd, J=4.1, 1.7 Hz, 1H), 8.63 (s, 1H), 8.43-8.35 (m, 2H), 8.25 (dd, J=8.5, 1.7 Hz, 1H), 7.66 (dd, J=8.5, 4.2 Hz, 1H), 7.48-7.40 (m, 3H), 4.59 (bs, 4H), 3.98 (s, 3H), 3.90 (t, J=4.0 Hz, 4H); LCMS (ESI) m/z: 401.2 [M+H]⁺.

### Step 4: Synthesis of 4-(4-morpholinopyrido[3,2-d]pyrimidin-2-yl)-2-phenylpyrimidin-5-ol

A mixture of 4-(2-(5-methoxy-2-phenylpyrimidin-4-yl)pyrido[3,2-d]pyrimidin-4-yl)morpholine (200 mg) in hydrobromic acid (45% in acetic acid, 10 mL) was stirred at 100° C. for 4 h. The mixture was poured into water, extracted with ethyl acetate (100 mL*3) and the combined organic phase was concentrated. The residue was purified by prep-HPLC (Column Xbridge 21.2*250 mm C18, 10 um, Mobile Phase A: water (10 mmol/L ammonium bicarbonate) B: acetonitrile) to obtain 4-(4-morpholinopyrido[3,2-d]pyrimidin-2-yl)-2-phenylpyrimidin-5-ol (10.4 mg, 0.027 mmol) as yellow solid. ¹H NMR (400 MHz, DMSO-d₆) δ 14.02 (s, 1H), 8.90 (dd, J=4.1, 1.6 Hz, 1H), 8.76 (s, 1H), 8.51-8.38 (m, 3H), 7.92 (dd, J=8.5, 4.2 Hz, 1H), 7.54 (t, J=8 Hz, 2H), 7.48 (t, J=7.1 Hz, 1H), 4.69 (bs, 4H), 3.89 (d, J=4.3 Hz, 4H); LCMS (ESI) m/z: 386.9 [M+H]⁺.

### Synthesis of 4,4′-(2-(2-phenylpyrimidin-4-yl)pyrido[3,2-d]pyrimidine-4,7-diyl)dimorpholine (Compound 95)

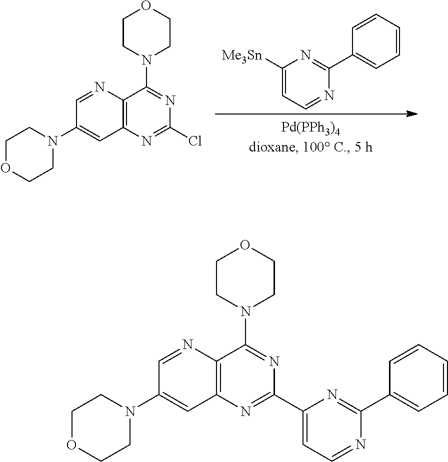

To a solution of 4,4′-(2-chloropyrido[3,2-d]pyrimidine-4,7-diyl)dimorpholine (0.06 g, 0.18 mmol) in dioxane (3 mL) were added 2-phenyl-4-(trimethylstannyl)pyrimidine (0.06 g, 0.18 mmol) and tetrakis(triphenylphosphine)palladium (0.02 g, 0.02 mmol) at 25° C. and the resulting mixture was stirred at 100° C. for 5 h under argon atmosphere. The reaction mixture was concentrated and crude product obtained was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product (0.0133 g, 16%) as off-white solid. ¹H NMR (400 MHz, DMSO-d₆) δ 9.07 (d, J=5.1 Hz, 1H), 8.80 (d, J=2.8 Hz, 1H), 8.60-8.49 (m, 2H), 8.28 (d, J=5.1 Hz, 1H), 7.58 (d, J=3.4 Hz, 3H), 7.47 (d, J=2.5 Hz, 1H), 4.53 (bs, 4H), 3.87-3.76 (m, 8H), 3.47 (s, 4H); LCMS (ESI) m/z: 456.2 [M+H]⁺.

### Synthesis of 4-(2-(5-methyl-2-phenylpyrimidin-4-yl)pyrido[3,2-d]pyrimidin-4-yl)morpholine (Compound 96)

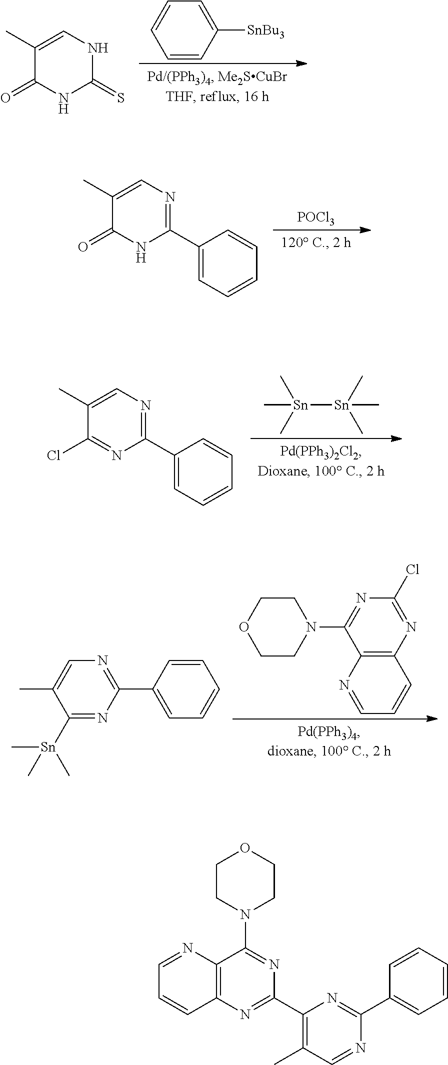

### Step 1: Synthesis of 5-Methyl-2-phenylpyrimidin-4(3H)-one

A mixture of 5-Methyl-2-thioxo-2,3-dihydropyrimidin-4(1H)-one (500 mg, 3.5 mmol), tributyl(phenyl)stannane (2.83 g, 7.7 mmol), copper(I) bromide-dimethyl Sulfide (1.59 g, 7.7 mmol) and tetrakis(triphenylphosphine)palladium (120 mg, 0.2 mmol) in THF (50 mL) was refluxed overnight under nitrogen atmosphere. The mixture was filtered, concentrated and purified by flash chromatography (dichloromethane/methanol=90/10) to obtain the target product as white solid (300 mg, 45.8%).

### Step 2: Synthesis of 4-Chloro-5-methyl-2-phenylpyrimidine

A mixture of 5-Methyl-2-phenylpyrimidin-4(3H)-one (290 mg, 1.56 mmol) in phosphorus oxychloride (5 mL) was stirred at 120° C. for 2 h under nitrogen atmosphere. The mixture was concentrated and the resultant crude product was purified by combined flash chromatography (petroleum ether/ethyl acetate=80/20) to obtain the target product as white solid (280 mg, 85.1%).

### Step 3: Synthesis of 5-Methyl-2-phenyl-4-(trimethylstannyl)pyrimidine

A mixture of 4-Chloro-5-methyl-2-phenylpyrimidine (280 mg, 1.37 mmol), hexamethyldistannane (900 mg, 1.37 mmol) and trans-dichlorobis(triphenyl-phosphine)palladium(II) (96 mg, 0.14 mmol) in dioxane (10 mL) was stirred at 100° C. for 1 h under nitrogen atmosphere. The resultant mixture was directly used in the next step without further purification.

### Step 4: Synthesis of 4-(2-(5-methyl-2-phenylpyrimidin-4-yl)pyrido[3,2-d]pyrimidin-4-yl)morpholine

4-(2-Chloropyrido[3,2-d]pyrimidin-4-yl)morpholine (340 mg, 1.37 mmol) and tetrakis(triphenylphosphine)palladium(158 mg, 0.14 mmol) were added to the mixture from step-3. The resultant mixture was stirred at 110° C. for 1 h under nitrogen atmosphere. It was filtered and the filtrated was concentrated and the residue was purified by prep-HPLC (Column Xbridge 21.2*250 mm C18, 10 um, Mobile Phase A: water (10 mmol/L ammonium bicarbonate) B: acetonitrile) to afford 4-(2-(5-methyl-2-phenylpyrimidin-4-yl)pyrido[3,2-d]pyrimidin-4-yl)morpholine as white solid (7.8 mg, 1.50% over two steps). ¹H NMR (400 MHz, CD₃OD) δ 8.88 (dd, J=4.2, 1.6 Hz, 1H), 8.86 (s, 1H), 8.46 (dd, J=6.7, 3.1 Hz, 2H), 8.26 (dd, J=8.6, 1.6 Hz, 1H), 7.84 (dd, J=8.5, 4.1 Hz, 1H), 7.51-7.46 (m, 3H), 4.66 (bs, 4H), 3.90 (t, J=4.2 Hz, 4H), 2.48 (s, 3H); LCMS (ESI) m/z: 385.1. [M+H]⁺.

### Synthesis of 4-(2-(6-phenylpyrazin-2-yl)pyrido[3,2-d]pyrimidin-4-yl)morpholine (Compound 97)

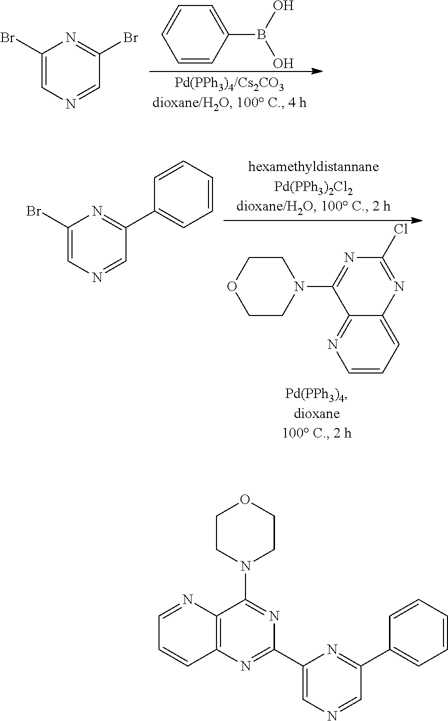

### Step 1: Synthesis of 2-bromo-6-phenylpyrazine

A mixture of 2,6-dibromopyrazine (2.36 g, 10.0 mmol), phenylboronic acid (1.22 g, 10.0 mmol), tetrakis(triphenylphosphine)-palladium (578 mg, 0.5 mmol) and cesium carbonate (6.5 g, 20.0 mmol) in dioxane (80 mL) and water (8 mL) was stirred at 100° C. under nitrogen atmosphere for 4 h. The resultant mixture was poured into water, extracted with ethyl acetate (200 mL*2) and the combined organic phase was concentrated. The residue was purified by silica gel column chromatography (20% ethyl acetate in petroleum ether) to afford 2-bromo-6-phenylpyrazine (700 mg, 30%) as white solid. LCMS (ESI) m/z: 235.1/237.0 [M+H]⁺.

### Step 2: Synthesis of 4-(2-(6-phenylpyrazin-2-yl)pyrido[3,2-d]pyrimidin-4-yl)morpholine

A mixture of 2-bromo-6-phenylpyrazine (234 mg, 1.0 mmol), hexamethyldistannane (654 mg, 2.0 mmol) and bis(triphenylphosphine)palladium(II) chloride (70 mg, 0.1 mmol) in dioxane (10 mL) was stirred at 100° C. for 2 h under nitrogen atmosphere. The mixture was poured into dichloromethane (200 mL), the organic phase was washed successively with saturated aqueous potassium fluoride solution (100 mL), brine and concentrated to afford the crude 2-phenyl-6-(trimethylstannyl)pyrazine (350 mg) as a brown oil. This product was mixed with 4-(2-chloropyrido[3,2-d]pyrimidin-4-yl)morpholine (200 mg, 0.8 mmol), tetrakis(triphenylphosphine)palladium (116 mg, 0.1 mmol) in dioxane (10 mL) and stirred at 100° C. for another 2 h. It was concentrated and the residue was subjected to silica gel column chromatography to afford 4-(2-(6-phenylpyrazin-2-yl)pyrido[3,2-d]pyrimidin-4-yl)morpholine (53.4 mg, 18.7%) as light yellow solid. ¹H NMR (400 MHz, DMSO-d₆) δ 9.54 (s, 1H), 9.37 (s, 1H), 8.86 (dd, J=4.1, 1.7 Hz, 1H), 8.34 (dd, J=8.5, 1.7 Hz, 1H), 8.31-8.24 (m, 2H), 7.88 (dd, J=8.5, 4.1 Hz, 1H), 7.63-7.54 (m, 3H), 4.59 (bs, 4H), 3.90-3.78 (m, 4H); LCMS (ESI) m/z: 371.1 [M+H]⁺.

### Synthesis of 4-(2-(2-(furan-3-yl)pyrimidin-4-yl)pyrido[3,2-d]pyrimidin-4-yl)morpholine (Compound 98)

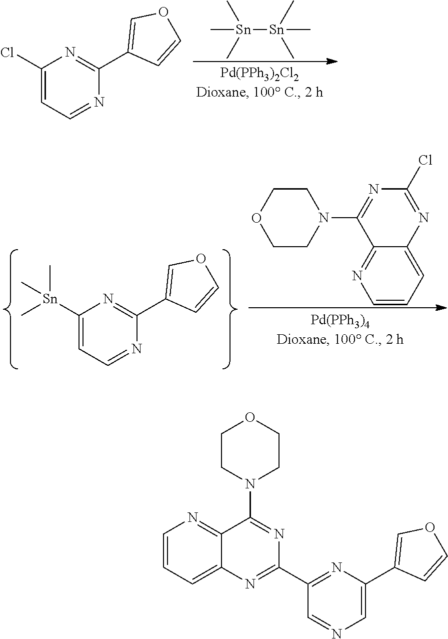

A mixture of 4-chloro-2-(furan-3-yl)pyrimidine (180 mg, 1.0 mmol), hexamethyldistannane (654 mg, 2.0 mmol) and bis(triphenylphosphine)palladium(II) chloride (70 mg, 0.1 mmol) in dioxane (10 mL) was stirred at 100° C. for 2 h under nitrogen atmosphere. The mixture was then poured into dichloromethane (200 mL). The organic phase was separated and washed successively with saturated aqueous potassium fluoride solution (100 mL), brine (100 mL) and concentrated to afford 2-(furan-3-yl)-4-(trimethylstannyl)pyrimidine (320 mg) as a brown oil. This product was mixed with 4-(2-chloropyrido[3,2-d]pyrimidin-4-yl)morpholine (200 mg, 0.8 mmol) and tetrakis(triphenylphosphine)palladium (116 mg, 0.1 mmol) in dioxane (10 mL) and stirred at 100° C. for another 2 h. The resultant mixture was concentrated and the residue was purified successively by silica gel column chromatography (25% methanol in dichloromethane) and prep-HPLC (Column Xbridge 21.2*250 mm C18, 10 um, Mobile Phase A: water (0.01% aqueous trifluoroacetic acid) B: acetonitrile) to afford 4-(2-(2-(furan-3-yl)pyrimidin-4-yl)pyrido[3,2-d]pyrimidin-4-yl)morpholine (53.4 mg, 18.7%) as grey solid. ¹H NMR (400 MHz, CDCl₃) δ 8.90 (d, J=5.1 Hz, 1H), 8.77 (dd, J=4.1, 1.7 Hz, 1H), 8.42 (d, J=0.8 Hz, 1H), 8.36 (dd, J=8.5, 1.7 Hz, 1H), 8.21 (d, J=5.1 Hz, 1H), 7.69 (dd, J=8.5, 4.1 Hz, 1H), 7.53 (t, J=1.7 Hz, 1H), 7.24-7.21 (m, 1H), 4.67 (bs, 4H), 3.96 (t, J=4.0 Hz, 4H); LCMS (ESI) m/z: 361.1 [M+H]⁺.

### Synthesis of 4-(2-(2-(furan-2-yl)pyrimidin-4-yl)pyrido[3,2-d]pyrimidin-4-yl)morpholine (Compound 99)

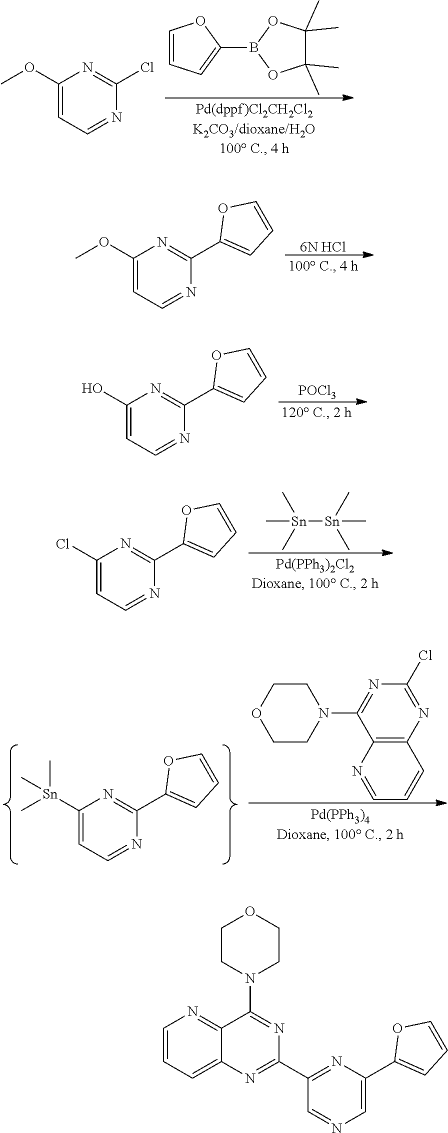

### Step 1: Synthesis of 2-(furan-2-yl)-4-methoxypyrimidine

A mixture of 2-chloro-4-methoxypyrimidine (1.44 g, 10.0 mmol), 2-(furan-2-yl)-4,4,5,5-tetramethyl-1,3,2-dioxaborolane (1.94 g, 1.0 mmol), 1,1′-bis(diphenylphosphino)ferrocene-palladium(II)dichloride dichloromethane complex (457 mg, 0.5 mmol) and potassium carbonate (2.76 g, 20.0 mmol) in water (4 mL) and dioxane (40 mL) was stirred at 100° C. under nitrogen atmosphere for 4 h. The resultant mixture was poured into water, extracted with ethyl acetate (200 mL*2) and the organic phase was concentrated. The residue was purified by silica gel column chromatography (30% ethyl acetate in petroleum ether) to afford 2-(furan-2-yl)-4-methoxypyrimidine (1.5 g, 85%) as brown oil. LCMS (ESI) m/z: 177.1 [M+H]⁺.

### Step 2: Synthesis of 2-(furan-2-yl)pyrimidin-4-ol

A mixture of 2-(furan-2-yl)-4-methoxypyrimidine (1.4 g, 7.9 mmol) and hydrochloric acid (6N, 15 mL) was stirred at 100° C. for 4 hours. The mixture was poured into water, basified with solid sodium bicarbonate, extracted with ethyl acetate (200 mL*2). The combined organic phase was dried and concentrated to afford 2-(furan-2-yl)pyrimidin-4-ol (1.1 g, 86%) as grey solid. LCMS (ESI) m/z: 163.1 [M+H]⁺.

### Step 3: Synthesis of 4-chloro-2-(furan-2-yl)pyrimidine

A mixture of 2-(furan-2-yl)pyrimidin-4-ol (1.0 g, 6.0 mmol) in phosphorus oxitrichloride (20 mL) was stirred at 120° C. for 2 h. The mixture was concentrated, the residue was dissolved in ethyl acetate (200 mL) and poured into crushed ice. The organic layer was separated, and the aqueous layer was extracted with ethyl acetate (100 mL*2). The combined organic phase was concentrated and the residue was purified by silica gel column chromatography (50% ethyl acetate in petroleum ether) to afford 4-chloro-2-(furan-2-yl)pyrimidine (600 mg, 54%) as grey solid. LCMS (ESI) m/z: 180.9/182.9 [M+H]⁺.

### Step 4: Synthesis of 4-(2-(2-(furan-2-yl)pyrimidin-4-yl)pyrido[3,2-d]pyrimidin-4-yl)morpholine

A mixture of 4-chloro-2-(furan-2-yl)pyrimidine (180 mg, 1.0 mmol), hexamethyldistannane (654 mg, 2.0 mmol) and bis(triphenylphosphine)palladium(II) chloride (70 mg, 0.1 mmol) in dioxane (10 mL) was stirred at 100° C. for 2 h under nitrogen atmosphere. The mixture was poured into dichloromethane (200 mL), the organic phase was washed successively with saturated aqueous potassium fluoride solution (100 mL), brine and concentrated to afford the 2-(furan-2-yl)-4-(trimethylstannyl)pyrimidine (280 mg) as a brown oil. This oily product was mixed with 4-(2-chloropyrido[3,2-d]pyrimidin-4-yl)morpholine (200 mg, 0.8 mmol) and tetrakis(triphenylphosphine)palladium (116 mg, 0.1 mmol) in dioxane (10 mL) and stirred at 100° C. for another 2 h. The resultant mixture was concentrated and the residue was purified by silica gel column chromatography (25% methanol in dichloromethane) and prep-HPLC (Column Xbridge 21.2*250 mm C18, 10 um, Mobile Phase A: water (10 mmol/L ammonium bicarbonate) B: acetonitrile) to afford 4-(2-(2-(furan-2-yl)pyrimidin-4-yl)pyrido[3,2-d]pyrimidin-4-yl)morpholine (7.8 mg, 2.1%) as off-white solid. ¹H NMR (400 MHz, CDCl₃) δ 8.94 (d, J=5.1 Hz, 1H), 8.77 (dd, J=4.1, 1.7 Hz, 1H), 8.39 (dd, J=8.5, 1.6 Hz, 1H), 8.22 (d, J=5.1 Hz, 1H), 7.69 (dd, J=8.6, 2.5 Hz, 2H), 7.50 (d, J=3.3 Hz, 1H), 6.61 (dd, J=3.4, 1.7 Hz, 1H), 4.67 (bs, 4H), 3.96 (t, J=4.0 Hz, 4H); LCMS (ESI) m/z: 361.8 [M+H]⁺.

### Synthesis of 4-(2-(2-(3-methoxyphenyl)pyrimidin-4-yl)pyrido[3,2-d]pyrimidin-4-yl)morpholine (Compound 100) and 3-(4-(4-morpholinopyrido[3,2-d]pyrimidin-2-yl)pyrimidin-2-yl)phenol (Compound 101)

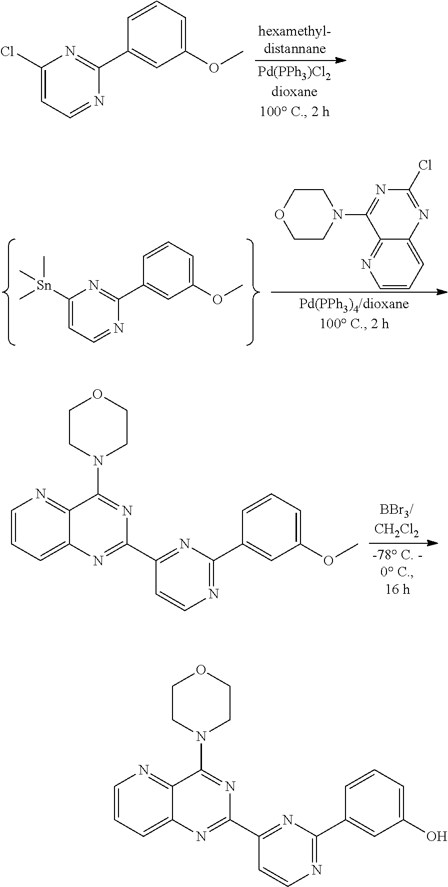

### Step 1: Synthesis of 4-(2-(2-(3-methoxyphenyl)pyrimidin-4-yl)pyrido[3,2-d]pyrimidin-4-yl)morpholine

A mixture of 4-chloro-2-(3-methoxyphenyl)pyrimidine (220 mg, 1.0 mmol), hexamethyldistannane (654 mg, 2.0 mmol) and bis(triphenylphosphine)palladium(II) chloride (70 mg, 0.1 mmol) in dioxane (10 mL) was stirred at 100° C. for 2 h under nitrogen atmosphere. The mixture was poured into dichloromethane (200 mL), the organic phase was washed successively with saturated aqueous potassium fluoride (100 mL), brine and concentrated to afford the 2-(3-methoxyphenyl)-4-(trimethylstannyl)pyrimidine (360 mg) as brown oil. This oil was mixed with 4-(2-chloropyrido[3,2-d]pyrimidin-4-yl)morpholine (200 mg, 0.8 mmol), and tetrakis(triphenylphosphine)palladium (116 mg, 0.1 mmol) in dioxane (10 mL) and stirred at 100° C. for another 2 h. The mixture was concentrated and the residue was purified by silica gel column chromatography (25% methanol in dichloromethane) to afford 4-(2-(2-(3-methoxyphenyl)pyrimidin-4-yl)pyrido[3,2-d]pyrimidin-4-yl)morpholine (190 mg, 0.48 mmol) as yellow solid. ¹H NMR (400 MHz, DMSO-d₆) δ 9.10 (d, J=5.1 Hz, 1H), 8.89 (dd, J=4.1, 1.7 Hz, 1H), 8.38-8.31 (m, 2H), 8.15-8.10 (m, 2H), 7.92-7.87 (m, 1H), 7.49 (d, J=7.9 Hz, 1H), 7.15 (dd, J=7.4, 2.6 Hz, 1H), 4.61 (bs, 4H), 3.88 (s, 3H), 3.87-3.83 (m, 4H); LCMS (ESI) m/z: 401.1 [M+H]⁺.

### Step 2: Synthesis of 3-(4-(4-morpholinopyrido[3,2-d]pyrimidin-2-yl)pyrimidin-2-yl)phenol

To a solution of 4-(2-(2-(3-methoxyphenyl)pyrimidin-4-yl)pyrido[3,2-d]pyrimidin-4-yl)morpholine (150 mg, 0.38 mmol) in dichloromethane (20 mL) was slowly added tribromoborane (17% in dichloromethane, 5 mL) at −78° C. under nitrogen atmosphere. The mixture was stirred for another 16 h and then poured into crushed ice and extracted with dichloromethane (100 mL*3). The combined organic phase was concentrated and the residue was purified by silica gel column chromatography (25% methanol in dichloromethane) to afford 100 mg of grey solid, which was further purified by prep-HPLC (Column Xbridge 21.2*250 mm C18, 10 um, Mobile Phase A: water (10 mmol/L ammonium bicarbonate) B: acetonitrile) to afford 3-(4-(4-morpholinopyrido[3,2-d]pyrimidin-2-yl)pyrimidin-2-yl)phenol (25.8 mg, 17.6%) as white solid. ¹H NMR (400 MHz, DMSO-d₆) δ 9.67 (s, 1H), 9.07 (d, J=5.1 Hz, 1H), 8.88 (dd, J=4.1, 1.6 Hz, 1H), 8.35 (dd, J=8.5, 1.6 Hz, 1H), 8.28 (d, J=5.1 Hz, 1H), 7.98 (dd, J=4.7, 2.4 Hz, 2H), 7.89 (dd, J=8.5, 4.1 Hz, 1H), 7.37 (t, J=8.1 Hz, 1H), 6.96 (dd, J=8.0, 1.4 Hz, 1H), 4.59 (bs, 4H), 3.85 (t, J=4.0 Hz, 4H); LCMS (ESI) m/z: 386.9 [M+H]⁺.

### Synthesis of 4-(2-(5-methoxy-2-m-tolylpyrimidin-4-yl)pyrido[3,2-d]pyrimidin-4-yl)morpholine (Compound 102) and 4-(4-morpholinopyrido[3,2-d]pyrimidin-2-yl)-2-m-tolylpyrimidin-5-ol (Compound 103)

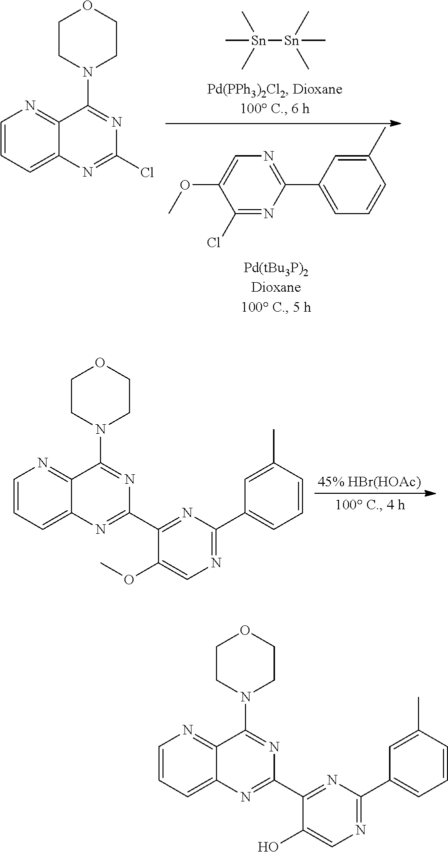

### Step 1: Synthesis of 4-(2-(5-methoxy-2-m-tolylpyrimidin-4-yl)pyrido[3,2-d]pyrimidin-4-yl)morpholine

A mixture of 4-(2-chloropyrido[3,2-d]pyrimidin-4-yl)morpholine (250 mg, 1.0 mmol), hexamethyldistannane (491 mg, 1.5 mmol) and bis(triphenylphosphine)palladium(II) chloride (70 mg, 0.02 mmol) in dioxane (5 mL) was stirred at 100° C. for 6 h under nitrogen atmosphere. The mixture was then poured into dichloromethane (300 mL) and the organic phase was washed successively with saturated aqueous potassium fluoride solution (150 mL), brine and concentrated to afford 4-(2-(trimethylstannyl)pyrido[3,2-d]pyrimidin-4-yl)morpholine (380 mg) as brown oil. This product was mixed with 4-chloro-5-methoxy-2-m-tolylpyrimidine (234 mg, 1.0 mmol) and bis(tri-tert-butylphosphine)palladium (51 mg, 0.1 mmol) in dioxane (10 mL) and stirred at 100° C. for another 6 h. The resultant mixture was purified first by silica gel column chromatography (25% methanol in dichloromethane) and then by prep-HPLC (Column Xbridge 21.2*250 mm C18, 10 um, Mobile Phase A: water (10 mmol/L ammonium bicarbonate) B: acetonitrile) to afford 4-(2-(5-methoxy-2-m-tolylpyrimidin-4-yl)pyrido[3,2-d]pyrimidin-4-yl)morpholine (100 mg) as white solid. ¹H NMR (400 MHz, CDCl₃) δ 8.77 (dd, J=4.1, 1.7 Hz, 1H), 8.62 (s, 1H), 8.29-8.16 (m, 3H), 7.66 (dd, J=8.5, 4.2 Hz, 1H), 7.34 (t, J=7.6 Hz, 1H), 7.24 (d, J=7.5 Hz, 1H), 4.60 (bs, 4H), 3.97 (s, 3H), 3.90 (t, J=4 Hz, 4H), 2.42 (s, 3H); LCMS (ESI) m/z: 415.0 [M+H]⁺.

### Step 2: Synthesis of 4-(4-morpholinopyrido[3,2-d]pyrimidin-2-yl)-2-m-tolylpyrimidin-5-ol

A mixture of 4-(2-(5-methoxy-2-m-tolylpyrimidin-4-yl)pyrido[3,2-d]pyrimidin-4-yl)morpholine (300 mg, crude) in hydrogen bromide (45% in acetic acid, 10 mL) was stirred at 100° C. for 5 h. The mixture was then poured into water, extracted with ethyl acetate (100 mL*3) and the combined organic phase was concentrated. The residue was purified by prep-HPLC (Column Xbridge 21.2*250 mm C18, 10 um, Mobile Phase A: water (10 mmol/L ammonium bicarbonate) B: acetonitrile) to afford 4-(4-morpholinopyrido[3,2-d]pyrimidin-2-yl)-2-m-tolylpyrimidin-5-ol (28.1 mg, 0.07 mmol) as grey solid. ¹H NMR (400 MHz, CDCl₃) δ 13.99 (s, 1H), 8.79 (dd, J=4.2, 1.7 Hz, 1H), 8.72 (s, 1H), 8.35-8.26 (m, 2H), 8.19 (dd, J=8.5, 1.7 Hz, 1H), 7.72 (dd, J=8.5, 4.2 Hz, 1H), 7.39 (t, J=7.6 Hz, 1H), 7.26-7.24 (m, 1H), 4.79 (bs, 4H), 4.00 (t, J=4.0 Hz, 4H), 2.46 (s, 3H); LCMS (ESI) m/z: 400.8 [M+H]⁺.

### Synthesis of 4-(2-(2-phenylpyrimidin-4-yl)-7-(pyridin-4-yl)pyrido[3,2-d]pyrimidin-4-yl)morpholine (Compound 104)

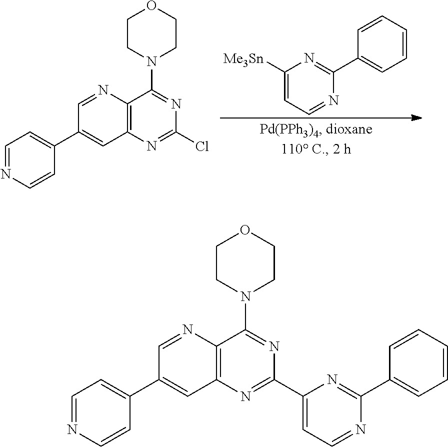

To a solution of 4-(2-chloro-7-(pyridin-4-yl)pyrido[3,2-d]pyrimidin-4-yl)morpholine (300 mg, 0.92 mmol) and 2-phenyl-4-(trimethylstannyl)pyrimidine (319 mg, 1.00 mmol) in dioxane was added tetrakis(triphenylphosphine)palladium (104 mg, 0.09 mmol). The reaction mixture was stirred at 110° C. under nitrogen atmosphere for 2 h. The mixture was then filtered and the filtrate was purified by prep-HPLC (Column Xbridge 21.2*250 mm C18, 10 um, Mobile Phase A: water (10 mmol/L ammonium bicarbonate) B: acetonitrile) to afford 4-(2-(2-phenylpyrimidin-4-yl)-7-(pyridin-4-yl)pyrido[3,2-d]pyrimidin-4-yl)morpholine as white solid (26.8 mg, 6.52%). ¹H NMR (400 MHz, DMSO-d₆) δ 9.34 (d, J=2.3 Hz, 1H), 9.13 (d, J=5.1 Hz, 1H), 8.79 (dd, J=6.3, 4.2 Hz, 3H), 8.59-8.53 (m, 2H), 8.36 (d, J=5.0 Hz, 1H), 8.07 (d, J=6.1 Hz, 2H), 7.63-7.57 (m, 3H), 4.64 (bs, 4H), 3.89 (d, J=5.1 Hz, 4H); LCMS (ESI) m/z: 447.8 [M]⁺.

### Synthesis of 4-(2-(2-phenylpyrimidin-4-yl)-7-(1H-pyrazol-3-yl)pyrido[3,2-d]pyrimidin-4-yl)morpholine (Compound 105)

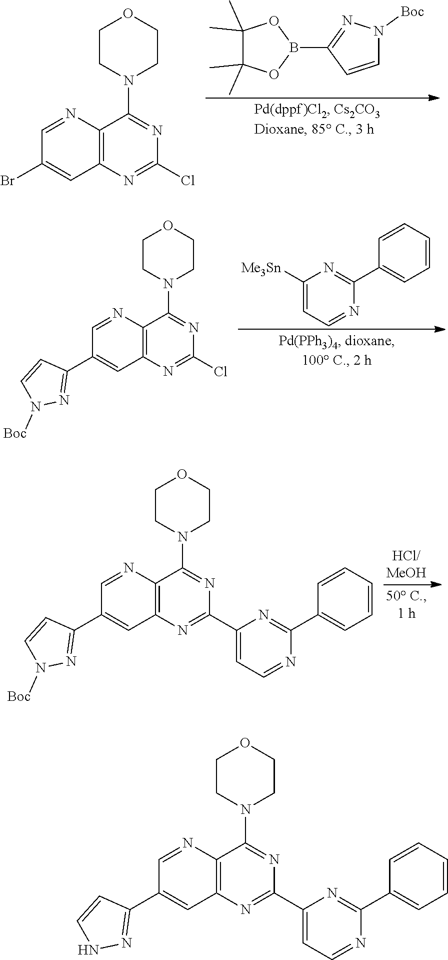

### Step 1: Synthesis of tert-butyl 3-(2-chloro-4-morpholinopyrido[3,2-d]pyrimidin-7-yl)-1H-pyrazole-1-carboxylate

To a solution of 4-(7-bromo-2-chloropyrido[3,2-d]pyrimidin-4-yl)morpholine (0.12 g, 0.36 mmol) in dioxane/water (2 mL/0.2 mL) were added tert-butyl 3-(4,4,5,5-tetramethyl-1,3,2-dioxaborolan-2-yl)-1H-pyrazole-1-carboxylate (0.11 g, 0.36 mmol), cesium carbonate (0.18 g, 0.55 mmol) and bis(diphenylphosphino)ferrocene-palladium(II)dichloride dichloromethane complex (0.026 g, 0.55 mmol) at 25° C. and the reaction mixture was heated and stirred at 85° C. for 3 h under argon. The mixture was then filtered and the filtrate was concentrated obtain the desired product (0.15 g, 100%) as white solid.

### Step 2: Synthesis of tert-butyl 3-(4-morpholino-2-(2-phenylpyrimidin-4-yl)pyrido[3,2-d]pyrimidin-7-yl)-1H-pyrazole-1-carboxylate

To a solution of tert-butyl 3-(2-chloro-4-morpholinopyrido[3,2-d]pyrimidin-7-yl)-1H-pyrazole-1-carboxylate (0.13 g, 0.31 mmol) in dioxane (3 mL) were added 2-phenyl-4-(trimethylstannyl)pyrimidine (0.11 g, 0.34 mmol) and tetrakis(triphenylphosphine) palladium (0.036 g, 0.031 mmol) at 25° C., and the resultant mixture was stirred at 100° C. for 2 h under argon. The reaction was quenched by the addition of aqueous potassium fluoride (5 mL) and the mixture was filtered. The filtrate was extracted with dichloromethane (10 mL*3), concentrated and purified by silica gel column (petroleum ether:acetic ester=3:1) to obtain the desired product (0.1 g, 60%) as yellow solid.

### Step 3: Synthesis of 4-(2-(2-phenylpyrimidin-4-yl)-7-(1H-pyrazol-3-yl)pyrido[3,2-d]pyrimidin-4-yl)morpholine

A solution of tert-butyl 3-(4-morpholino-2-(2-phenylpyrimidin-4-yl)pyrido[3,2-d]pyrimidin-7-yl)-1H-pyrazole-1-carboxylate (0.09 g, 0.17 mmol) in hydrochloric acid/methanol (2 mL) was stirred at 50° C. for 1 h. The resultant mixture was concentrated and the crude product thus obtained was purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain 4-(2-(2-phenylpyrimidin-4-yl)-7-(1H-pyrazol-3-yl)pyrido[3,2-d]pyrimidin-4-yl)morpholine (14.8 mg, 20%) as yellow solid. 1H NMR (400 MHz, DMSO) δ 13.33 (s, 1H), 9.38 (s, 1H), 9.11 (d, J=5.1 Hz, 1H), 8.66 (s, 1H), 8.56 (dd, J=6.6, 3.1 Hz, 2H), 8.34 (d, J=5.1 Hz, 1H), 7.94 (s, 1H), 7.59 (dd, J=5.1, 1.9 Hz, 3H), 7.17 (d, J=2.3 Hz, 1H), 4.61 (bs, 4H), 3.92-3.81 (m, 4H); LCMS (ESI) m/z: 437.1 [M+H]⁺.

### Synthesis of 3-methyl-4-(2-(2-phenylpyrimidin-4-yl)pyrido[3,2-d]pyrimidin-4-yl)morpholine (Compound 106)

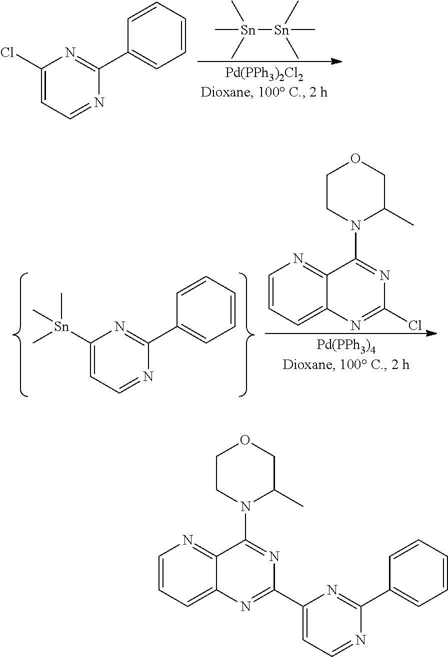

A mixture of 4-chloro-2-phenylpyrimidine (150 mg, 0.78 mmol), hexamethyldistannane (510 mg, 1.56 mmol), bis(triphenylphosphine)palladium(II) chloride (56 mg, 0.08 mmol) and dioxane (15 mL) was stirred at 100° C. for 2 h under nitrogen atmosphere. The mixture was then poured into dichloromethane (200 mL), the organic phase was washed successively with saturated aqueous potassium fluoride solution (100 mL), brine and concentrated to afford 2-phenyl-4-(trimethylstannyl)pyrimidine (210 mg) as a brown oil. This product was then mixed with 4-(2-chloropyrido[3,2-d]pyrimidin-4-yl)-3-methylmorpholine (200 mg, 0.75 mmol) and tetrakis(triphenylphosphine)palladium (93 mg, 0.08 mmol) in dioxane (15 mL) and the resultant mixture was stirred at 100° C. for another 2 h. The mixture was concentrated and the crude product obtained was purified by silica gel column chromatography (25% methanol in dichloromethane) and then further purified by prep-HPLC (Column Xbridge 21.2*250 mm C18, 10 um, Mobile Phase A: water (10 mmol/L ammonium bicarbonate) B: acetonitrile) to afford 3-methyl-4-(2-(2-phenylpyrimidin-4-yl)pyrido[3,2-d]pyrimidin-4-yl)morpholine (143.7 mg, 0.37 mmol) as white solid. ¹H NMR (500 MHz, CDCl₃) δ 9.01 (d, J=5.0 Hz, 1H), 8.77 (dd, J=4.1, 1.7 Hz, 1H), 8.63 (dd, J=7.8, 1.8 Hz, 2H), 8.39 (dd, J=8.5, 1.7 Hz, 1H), 8.29 (d, J=5.0 Hz, 1H), 7.71 (dd, J=8.5, 4.1 Hz, 1H), 7.56-7.50 (m, 3H), 6.50-4.50 (bs, 2H), 4.12 (d, J=10.9 Hz, 1H), 3.96 (dd, J=9.2, 2.4 Hz, 1H), 3.87 (d, J=9.2 Hz, 1H), 3.84-3.76 (m, 1H), 3.73 (d, J=13.2 Hz, 1H), 1.56 (d, J=6.8 Hz, 3H); LCMS (ESI) m/z: 385.2 [M+H]⁺.

### Synthesis of 4-(2-(3-methoxy-6-phenylpyridin-2-yl)pyrido[3,2-d]pyrimidin-4-yl)morpholine (Compound 107) and 2-(4-morpholinopyrido[3,2-d]pyrimidin-2-yl)-6-phenylpyridin-3-ol (Compound 108)

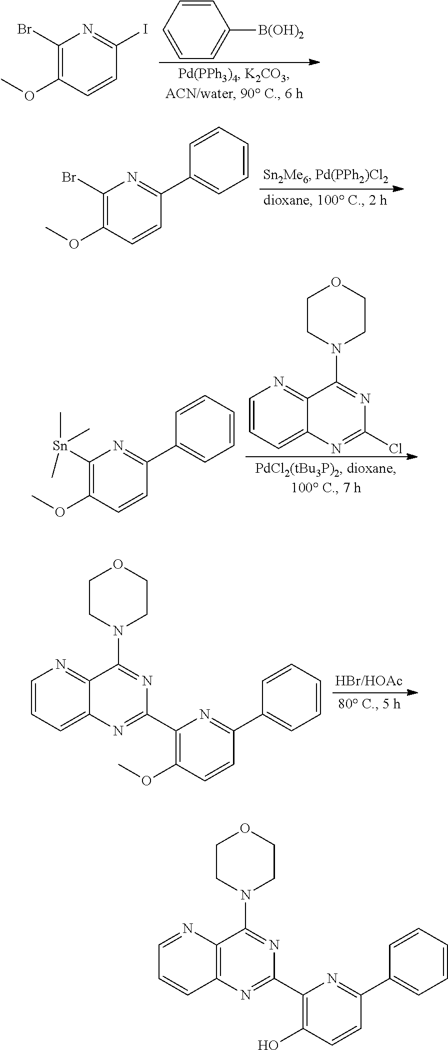

### Step 1: Preparation of 2-bromo-3-methoxy-6-phenylpyridine

A mixture of 2-bromo-6-iodo-3-methoxypyridine (1.56 g, 5.0 mmol), phenylboronic acid (610 mg, 5.0 mmol), tetrakis (triphenyl-phosphine)palladium (289 mg, 0.25 mmol) and potassium carbonate (1.38 g, 10.0 mmol) in acetonitrile (10 mL) and water (1 mL) was stirred at 90° C. under nitrogen atmosphere for 6 h. The mixture was then poured into water, extracted with ethyl acetate (100 mL*2) and the combined organic phase was concentrated. The residue was purified by silica gel column chromatography (15% ethyl acetate in petroleum ether) to afford 2-bromo-3-methoxy-6-phenylpyridine (1.5 g, 67%) as white solid.

### Step 2: Preparation of 3-methoxy-6-phenyl-2-(trimethylstannyl)pyridine

To a solution of 2-bromo-3-methoxy-6-phenylpyridine (0.3 g, 1.14 mmol) in dioxane (7 mL) were added 1,1,1,2,2,2-hexamethyldistannane (0.48 g, 1.48 mmol) and bis(triphenylphosphine)palladium(II) chloride (0.08 g, 0.11 mmol) at 25° C. and the resultant mixture was stirred at 100° C. for 0.5 h under argon protection. Aqueous potassium fluoride (5 mL) was added to the mixture and it was filtered and the filtrate was extracted with dichloromethane (10 mL*3). The combined organic layer was concentrated to obtain the target product as brown solid (0.34 g, 86%).

### Step 3: Preparation of 4-(2-(3-methoxy-6-phenylpyridin-2-yl)pyrido[3,2-d]pyrimidin-4-yl)morpholine

To a solution of 4-(2-chloropyrido[3,2-d]pyrimidin-4-yl)morpholine (0.35 g, 1.4 mmol) in dioxane (6 mL) were added 3-methoxy-6-phenyl-2-(trimethylstannyl)pyridine (0.34 g, 0.98 mmol) and bis(tri-tert-butylphosphine)palladium(0) (0.071 g, 0.031 mmol) at 25° C. and the resultant mixture was stirred at 100° C. for 5 h under argon protection. Aqueous potassium fluoride (5 mL) was added to the mixture and filtered. The filtrate was extracted with dichloromethane (10 mL*3) and the combined organic layer was dried and concentrated. The residue was purified by Prep. HPLC (SunFire C18, 4.6*50 mm, 3.5 um column Xbridge C18 3.5 μm 4.6×50 mm column. The mobile phase was acetonitrile/10 mM formic acid aqueous solution) to obtain the target product as yellow solid (0.04 g, 7%). ¹HNMR (400 MHz, DMSO) δ 8.86 (dd, J=4.1, 1.7 Hz, 1H), 8.20 (dd, J=8.5, 1.7 Hz, 1H), 8.07-7.97 (m, 3H), 7.85 (dd, J=8.5, 4.2 Hz, 1H), 7.68 (d, J=8.8 Hz, 1H), 7.45 (t, J=7.5 Hz, 2H), 7.37 (t, J=7.3 Hz, 1H), 4.44 (bs, 4H), 3.82 (s, 3H), 3.80-3.74 (m, 4H); LCMS (ESI) m/z: 399.9[M+]⁺.

### Step 4: Preparation of 2-(4-morpholinopyrido[3,2-d]pyrimidin-2-yl)-6-phenylpyridin-3-ol

A solution of 4-(2-(3-methoxy-6-phenylpyridin-2-yl)pyrido[3,2-d]pyrimidin-4-yl)morpholine (0.03 g, 0.075 mmol) in hydrobromic acid (33% in acetic acid (1.5 mL) was stirred at 80° C. for 5 h. The mixture was concentrated and purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the target product as off-white solid (6.7 mg, 23%). ¹H NMR (400 MHz, DMSO-d₆) δ 14.31 (s, 1H), 8.85 (dd, J=4.1, 1.6 Hz, 1H), 8.37 (dd, J=8.5, 1.6 Hz, 1H), 8.19 (d, J=7.3 Hz, 2H), 8.06 (d, J=8.6 Hz, 1H), 7.89 (dd, J=8.5, 4.2 Hz, 1H), 7.50 (t, J=7.7 Hz, 3H), 7.40 (t, J=7.3 Hz, 1H), 4.70 (bs, 4H), 3.88 (t, J=4 Hz, 4H); LCMS (ESI) m/z: 386.1 [M+H]⁺.

### Synthesis of 4-(2-indazol-1-ylpyrido[3,2-d]pyrimidin-4-yl)morpholine (Compound 109) and 4-(2-indazol-2-ylpyrido[3,2-d]pyrimidin-4-yl)morpholine (Compound 110)

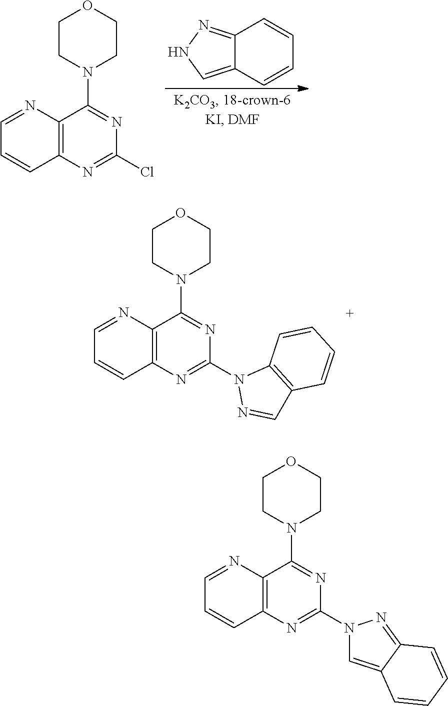

To a solution of 2H-indazole (80 mg, 677 umol) and 4-(2-chloropyrido[3,2-d]pyrimidin-4-yl)morpholine (221 mg, 880 umol) in DMF (1 mL) were added 18-CROWN-6 (1 mg, 5 umol), K₂CO₃ (133 mg, 965 mol) and KI (5 mg, 33 umol) and the resultant mixture was heated at 130° C. for 5 h. The mixture was filtered and the crude products from the filtrate were purified by prep-HPLC (Waters Xbridge BEH C18 100*25 mm*5 um column; 30-60% acetonitrile in an 10 mM ammonium bicarbonate in water, 10 min gradient) to obtain 4-(2-indazol-2-ylpyrido[3,2-d]pyrimidin-4-yl)morpholine (31 mg, 14%) and 4-(2-indazol-1-ylpyrido[3,2-d]pyrimidin-4-yl)morpholine (118 mg, 52%) as pale yellow solids.

Compound 109: ¹H NMR (400 MHz, DMSO-d6) δ 8.80 (d, J=8.4 Hz, 1H), 8.73 (d, J=3.6 Hz, 1H), 8.43 (s, 1H), 8.22 (d, J=8.4 Hz, 1H), 7.89 (d, J=8 Hz, 1H), 7.81 (dd, J=8.0, 3.6 Hz, 1H), 7.58 (t, J=8 Hz, 1H), 7.33 (t, J=7.6 Hz, 1H), 4.57 (bs, 4H), 3.85 (t, J=4.0 Hz, 4H). LCMS (ESI for C₁₈H₁₆N₆O [M+H]⁺: 333.2.

Compound 110: ¹H NMR (400 MHz, DMSO-d6) δ 9.33 (s, 1H), 8.80 (d, J=2.8 Hz, 1H), 8.24 (d, J=8.4 Hz, 1H), 7.86 (dd, J=8.4, 4.8 Hz, 1H), 7.78 (d, J=8.4 Hz, 1H), 7.72 (d, J=8.8 Hz, 1H), 7.33 (t, J=8.8 Hz, 1H), 7.11 (t, J=8.4 Hz, 1H), 4.57 (bs, 4H), 3.85 (t, J=4.8 hz, 4H). LCMS (ESI for C₁₈H₁₆N₆O [M+H]⁺: 333.2.

### Synthesis of 7-(furan-2-yl)-4-morpholino-N-phenylpyrido[3,2-d]pyrimidin-2-amine (Compound 111)

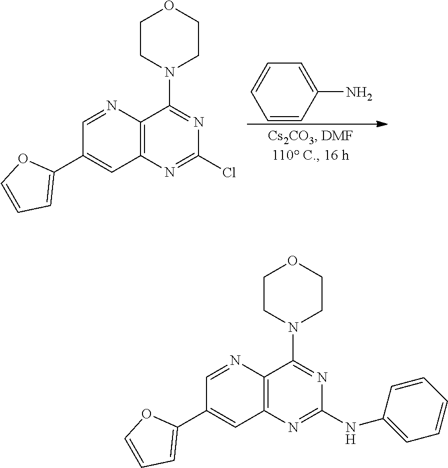 [SMILES: Clc1nc(N2CCOCC2)c2ncc(-c3ccco3)cc2n1 ; Nc1ccccc1 ; c1ccc(Nc2nc(N3CCOCC3)c3ncc(-c4ccco4)cc3n2)cc1]

To a solution of 4-(2-chloro-7-(furan-2-yl)pyrido[3,2-d]pyrimidin-4-yl)morpholine (200 mg, 0.63 mmol) and aniline (65 mg, 0.69 mmol) in DMF (7 mL) was added Cs₂CO₃ (619 mg, 1.90 mmol). The reaction mixture was stirred at 110° C. for 16 h and concentrated. The residue was subjected to prep-HPLC (0.05% FA/H₂O:CH₃CN=5%˜95%) to obtain 7-(furan-2-yl)-4-morpholino-N-phenylpyrido[3,2-d]pyrimidin-2-amine (35 mg, P: 100%, Y: 11%) as a yellow solid. ¹H NMR (400 MHz, DMSO-d₆) δ 9.28 (s, 1H), 8.86 (d, J=2.4 Hz, 1H), 7.98 (d, J=2.4 Hz, 1H), 7.93 (d, J=1.6 Hz, 1H), 7.89 (d, J=8.4 Hz, 2H), 7.37 (d, J=3.2 Hz, 1H), 7.29 (t, J=8.0 Hz, 2H), 6.94 (t, J=8.0 Hz, 1H), 6.74-6.72 (m, 1H), 4.40 (bs, 4H), 3.80 (t, J=4.4 Hz, 4H); LCMS (ESI) m/z: 374.3 [M+H]⁺.

### Synthesis of 4-(7-(furan-2-yl)-2-phenoxypyrido[3,2-d]pyrimidin-4-yl)morpholine (Compound 112)

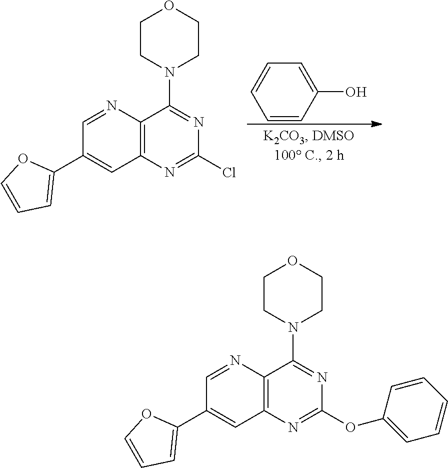 [SMILES: Clc1nc(N2CCOCC2)c2ncc(-c3ccco3)cc2n1 ; Oc1ccccc1 ; c1ccc(Oc2nc(N3CCOCC3)c3ncc(-c4ccco4)cc3n2)cc1]

To a solution of 4-(2-chloro-7-(furan-2-yl)pyrido[3,2-d]pyrimidin-4-yl)morpholine (200 mg, 0.63 mmol) and phenol (65 mg, 0.69 mmol) in DMF (8 mL) was added K₂CO₃ (262 mg, 1.90 mmol). The reaction mixture was stirred at 100° C. for 2 h and concentrated. The residue was purified by prep-HPLC (0.05% FA/H₂O:CH₃CN=5%˜95%) to afford 4-(7-(furan-2-yl)-2-phenoxypyrido[3,2-d]pyrimidin-4-yl)morpholine (40 mg, 17%) as yellow solid. ¹H NMR (400 MHz, DMSO-d₆) δ 9.02 (d, J=2.4 Hz, 1H), 8.01 (d, J=2.4 Hz, 1H), 7.93 (s, 1H), 7.47-7.41 (m, 3H), 7.25 (t, J=8.4 Hz, 3H), 6.72 (dd, J=3.2, 1.6 Hz, 1H), 4.37 (bs, 4H), 3.76 (t, J=4.8 Hz, 4H); LCMS (ESI) m/z: 375.1 [M+H]⁺.

### Synthesis of (E)-4-(2-(2-(3-methylbenzylidene)hydrazinyl)pyrido[3,2-d]pyrimidin-4-yl)morpholine (Compound 113)

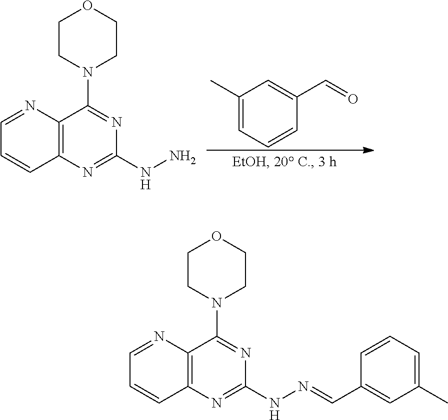

To a solution of 4-(2-hydrazinylpyrido[3,2-d]pyrimidin-4-yl)morpholine (80 mg, 0.32 mmol) and 3-methylbenzaldehyde (77 mg, 0.64 mmol) in ethanol (5.0 mL) was added acetic acid (19 mg, 0.32 mmol) and the resultant mixture was stirred at 20° C. under nitrogen for 2 h. It was concentrated and the residue was subjected to prep-HPLC (Boston C18 21*250 mm 10 μm column. The mobile phase was acetonitrile/0.01% aqueous trifluoroacetic acid) to obtain (E)-4-(2-(2-(3-methylbenzylidene)hydrazinyl)pyrido[3,2-d]pyrimidin-4-yl)morpholine (46.6 mg, 42%) as a yellow solid. ¹H NMR (400 MHz, DMSO) δ 10.93 (s, 1H), 8.46 (dd, J=4.0, 1.6 Hz, 1H), 8.11 (s, 1H), 7.87 (d, J=8.4 Hz, 1H), 7.61 (dd, J=8.5, 4.0 Hz, 1H), 7.47 (d, J=11.2 Hz, 2H), 7.32-7.28 (m, 1H), 7.17 (d, J=7.8 Hz, 1H), 4.42 (bs, 4H), 3.79 (t, J=4.2 Hz, 4H), 2.35 (s, 3H); LCMS (ESI) m/z: 349.2 [M+H]⁺.

The following compound was synthesized according to the protocol described above:

| Name | Structure | NMR, MS | # |
|---|---|---|---|
| (E)-3-((2-(4- morpholinopyrido[3,2- d]pyrimidin-2- yl)hydrazono)methyl)phenol | 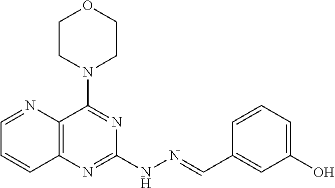 [SMILES: Oc1cccc(/C=N/Nc2nc(N3CCOCC3)c3ncccc3n2)c1] | ¹H NMR (400 MHz, DMSO-d₆) δ 10.91 (s, 1H), 9.55 (s, 1H), 8.47 (dd, J = 4.1, 1.6 Hz, 1H), 8.06 (s, 1H), 7.85 (dd, J = 8.5, 1.4 Hz, 1H), 7.61 (dd, J = 8.5, 4.1 Hz, 1H), 7.21 (t, J = 7.8 Hz, 1H), 7.15 (s, 1H), 7.04 (d, J = 7.6 Hz, 1H), 6.75 (dd, J = 8.0, 1.8 Hz, 1H), 4.43 (s, 4H), 3.79 (t, J = 4.4Hz, 4H); LCMS (ESI) m/z: 350.9 [M + H]⁺. | 114 |

### Synthesis of N-(3-methylphenethyl)-4-morpholinopyrido[3,2-d]pyrimidin-2-amine (Compound 115)

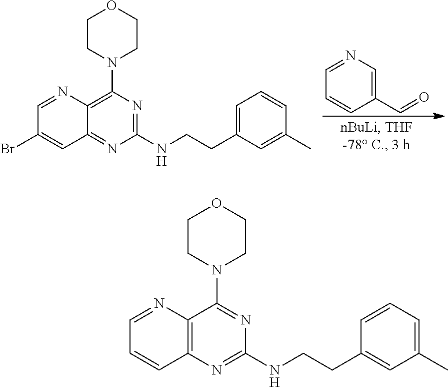 [SMILES: Cc1cccc(CCNc2nc(N3CCOCC3)c3ncc(Br)cc3n2)c1 ; O=Cc1cccnc1 ; Cc1cccc(CCNc2nc(N3CCOCC3)c3ncccc3n2)c1]

7-bromo-N-(3-methylphenethyl)-4-morpholinopyrido[3,2-d]pyrimidin-2-amine (854 mg, 2.0 mmol) was slowly added to a suspension of sodium hydride (80 mg, 2.0 mmol) in tetrahydrofuran (10 mL) at 0° C. After stirring the mixture for 10 min, it was cooled to −70° C., followed by the drop wise addition of n-butyllithium (0.8 mL 2.5 M in hexane) over a period of 15 min at −70° C. The mixture was further stirred for 20 minutes at −70° C. and nicotinaldehyde (320 mg, 3.0 mmol) in 2 mL tetrahydrofuran was added dropwise. After 2 h, the reaction was quenched with 4 mL concentrated hydrochloric acid in 5 mL water and 20 mL of diethyl ether. The organic layer was separated and washed with brine, dried over anhydrous sodium sulfate and concentrated. The resultant crude product was purified by prep-HPLC (Boston C18 21*250 mm 10 μm column. The mobile phase was acetonitrile/0.01% aqueous trifluoroacetic acid) to afford N-(3-methylphenethyl)-4-morpholinopyrido[3,2-d]pyrimidin-2-amine (25.7 mg, 7.0%) as white solid. ¹H NMR (400 MHz, DMSO) δ 8.33 (d, J=2.5 Hz, 1H), 7.69 (s, 1H), 7.50 (dd, J=8.3, 4.0 Hz, 1H), 7.19-7.15 (m, 1H), 7.14-6.97 (m, 3H), 6.88 (s, 1H), 4.30 (bs, 4H), 3.78-3.73 (m, 4H), 3.50 (dd, J=13.2, 6.4 Hz, 2H), 2.82 (t, J=6.8 Hz, 2H), 2.28 (s, 3H); LCMS (ESI) m/z: 350.3 [M+H]⁺.

### Synthesis of (E)-3-methylbenzaldehyde O-(4-morpholinopyrido[3,2-d]pyrimidin-2-yl) oxime (Compound 116)

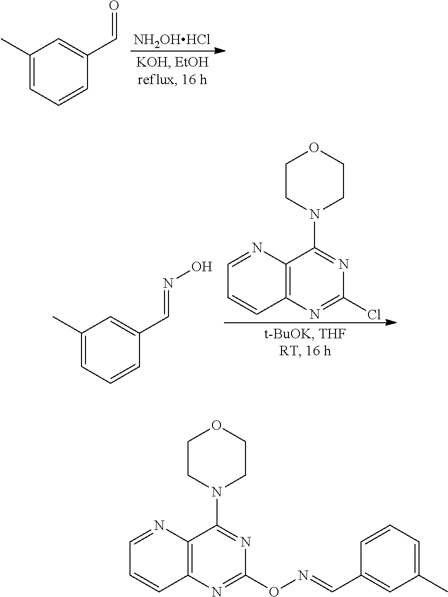

### Step 1: Synthesis of (E)-3-methylbenzaldehyde oxime

To a solution of 3-Methylbenzaldehyde (1.2 g, 10 mmol) and hydroxylamine hydrochloride (828 mg, 12 mmol) in ethanol (40 mL) was added a solution of potassium hydroxide (1.12 g, 20.0 mmol) in water (2 mL). The resultant mixture was heated to reflux for 16 h under nitrogen atmosphere. The mixture was further diluted with water and extracted with ethyl acetate (30 mL*3). The organic layer was combined, concentrated and purified by flash chromatography (dichloromethane/methanol=90/10) to obtain target compound as white solid (800 mg, 59.26%).

### Step 2: Synthesis of (E)-3-methylbenzaldehyde O-(4-morpholinopyrido[3,2-d]pyrimidin-2-yl) oxime

To a solution of (E)-3-methylbenzaldehyde oxime (100 mg, 0.74 mmol) in THF (5 mL) was added potassium-t-butoxide (83 mg, 0.74 mmol) and the mixture was stirred at 0° C. for 0.5 h. 4-(2-Chloropyrido[3,2-d]pyrimidin-4-yl)morpholine (185 mg, 0.74 mmol) was then added to the mixture and it was further stirred at room temperature for 16 h under nitrogen atmosphere. The mixture was filtered and the filtrate was concentrated. The residue was purified by prep-HPLC (Column Xbridge 21.2*250 mm C18, 10 um, Mobile Phase A: water (10 mmol/L ammonium bicarbonate) B: acetonitrile) to afford (E)-3-methylbenzaldehyde O-(4-morpholinopyrido[3,2-d]pyrimidin-2-yl) oxime as yellow solid. (8.7 mg, 3.37%). ¹H NMR (400 MHz, DMSO-d₆) δ 8.64 (d, J=4 Hz, 1H), 8.59 (s, 1H), 8.07 (dd, J=8.5, 1.7 Hz, 1H), 7.79-7.60 (m, 3H), 7.36 (dd, J=12.3, 4.8 Hz, 2H), 4.60 (bs, 4H), 3.87 (t, J=4 Hz, 4H), 2.42 (s, 3H); LCMS (ESI) m/z: 350.1. [M+H]⁺.

### Synthesis of 4-(2-(3-methoxy-4-phenyl-1H-pyrazol-1-yl)pyrido[3,2-d]pyrimidin-4-yl)morpholine (Compound 117)

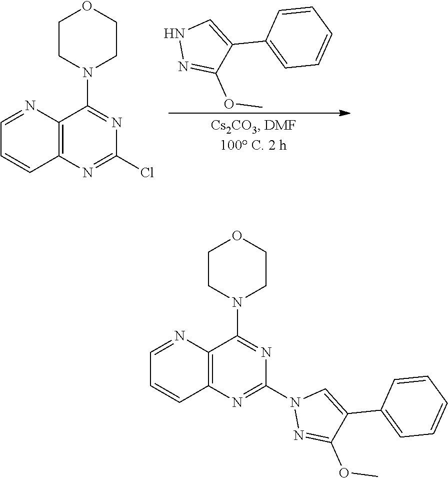 [SMILES: Clc1nc(N2CCOCC2)c2ncccc2n1 ; COc1n[nH]cc1-c1ccccc1 ; COc1nn(-c2nc(N3CCOCC3)c3ncccc3n2)cc1-c1ccccc1]

A mixture of 4-(2-chloropyrido[3,2-d]pyrimidin-4-yl)morpholine (100 mg, 0.4 mmol), 3-methoxy-4-phenyl-1H-pyrazole (70 mg, 0.4 mmol) and cesium carbonate (260 mg, 0.8 mmol) in N,N-dimethylformamide (5 mL) was stirred at 100° C. for 2 h. The mixture was cooled, and the resultant precipitate was filtered off. The filtrate was subjected to prep-HPLC (Column Xbridge 21.2*250 mm C18, 10 um, Mobile Phase A: water (10 mmol/L ammonium bicarbonate) B: acetonitrile) to obtain 4-(2-(3-methoxy-4-phenyl-1H-pyrazol-1-yl)pyrido[3,2-d]pyrimidin-4-yl)morpholine (68.0 mg, 42.5%) as white solid. ¹H NMR (400 MHz, CDCl₃) δ 8.65 (s, 1H), 8.60 (dd, J=4.1, 1.7 Hz, 1H), 8.20 (dd, J=8.5, 1.7 Hz, 1H), 7.76 (t, J=4.0 Hz, 2H), 7.58 (dd, J=8.5, 4.1 Hz, 1H), 7.40 (t, J=7.7 Hz, 2H), 7.29 (s, 1H), 4.62 (bs, 4H), 4.24 (s, 3H), 3.99-3.92 (m, 4H); LCMS (ESI) m/z: 388.8 [M+H]⁺.

### Synthesis of (2-((3-methylphenethyl)amino)-4-morpholinopyrido[3,2-d]pyrimidin-7-yl)(pyridin-3-yl)methanol (Compound 122), (2-((3-methylphenethyl)amino)-4-morpholinopyrido[3,2-d]pyrimidin-7-yl)(pyridin-3-yl)methanone (Compound 123) and N-(3-methylphenethyl)-4-morpholino-7-(pyridin-3-ylmethyl)pyrido[3,2-d]pyrimidin-2-amine (Compound 124)

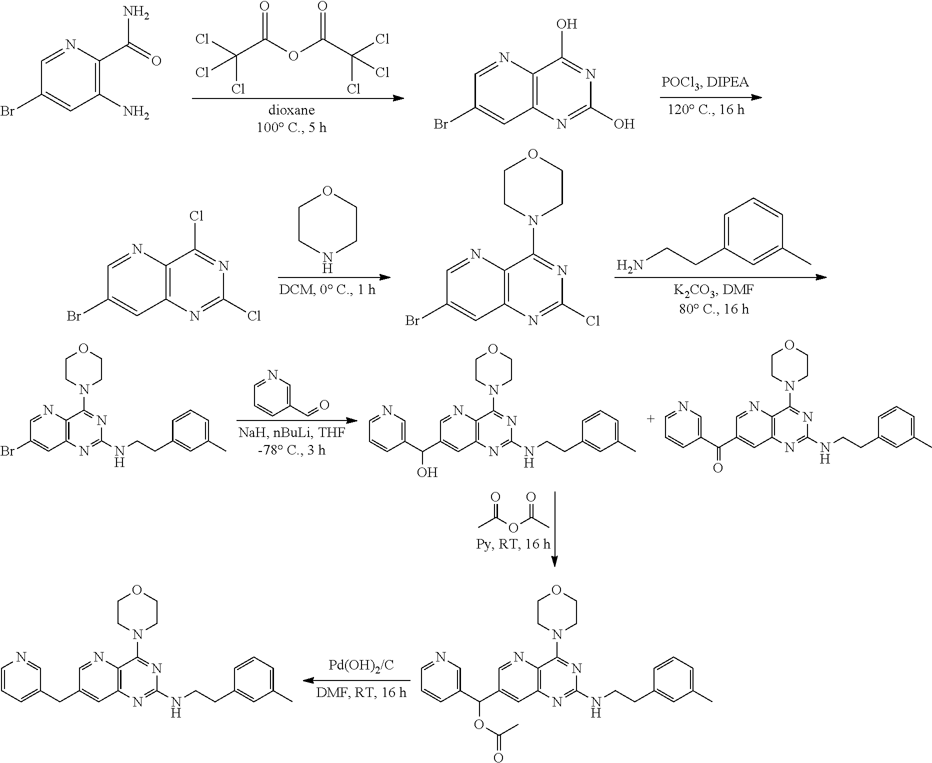

### Step 1: Synthesis of 7-bromopyrido[3,2-d]pyrimidine-2,4(1H,3H)-dione

To a stirred solution of 3-amino-5-bromopicolinamide (4.3 g, 20 mmol) in dry dioxane (50 mL) was added triphosgene (2.9 g, 10 mmol) under nitrogen atmosphere. The resulting dark orange reaction mixture was stirred under reflux for 30 minutes. Upon cooling, the solvent was removed under reduced pressure and the residue was subjected to silica gel flash chromatography (methanol/dichloromethane mixture gradient—5:95 to 15:95) to obtain 7-bromopyrido[3,2-d]pyrimidine-2,4(1H,3H)-dione (3.6 g, 75%) as white solid. ¹H NMR (400 MHz, DMSO) δ 11.60 (s, 1H), 11.30 (s, 1H), 8.54 (s, 1H), 7.74 (d, J=1.7 Hz, 1H); LCMS (ESI) m/z: 242.1 [M+H]⁺.

### Step 2: Synthesis of 7-bromo-2,4-dichloropyrido[3,2-d]pyrimidine

A mixture of 7-bromopyrido[3,2-d]pyrimidine-2,4(1H,3H)-dione (2.5 g, 10 mmol), phosphorus oxychloride (15 mL) and N,N-Diisopropylethylamine (1.0 mL) was stirred at 130° C. for 10 h. The volatiles were evaporated and azeotroped with toluene (2×100 mL). The residue was treated with ethyl acetate and filtered through a pad of celite. The filtrate was evaporated to obtain 7-bromo-2,4-dichloropyrido[3,2-d]pyrimidine (2.6 g, 94%). This material was taken to the next step without any further purification. LCMS (ESI) m/z: 277.9 [M+H]⁺.

### Step 3: Synthesis of 4-(7-bromo-2-chloropyrido[3,2-d]pyrimidin-4-yl)morpholine

A solution of 7-bromo-2,4-dichloropyrido[3,2-d]pyrimidine (2.0 g, 7.2 mmol) and morpholine (1.5 g, 18.0 mmol) in dichloromethane (20.0 mL) was stirred at 20° C. under nitrogen atmosphere for 2 h. The reaction mixture was concentrated, and the residue was subjected to flash column chromatography (ethyl acetate/petroleum ether 1:20) to obtain 4-(7-bromo-2-chloropyrido[3,2-d]pyrimidin-4-yl)morpholine (2.3 g, 97%) as white solid. ¹H NMR (400 MHz, DMSO) δ 8.87 (d, J=2.3 Hz, 1H), 8.40 (d, J=2.3 Hz, 1H), 5.30-4.04 (m, 4H), 3.35 (bs, 4H); LCMS (ESI) m/z: 329.0 [M+H]⁺.

### Step 4: Synthesis of 7-bromo-N-(3-methylphenethyl)-4-morpholinopyrido[3,2-d]pyrimidin-2-amine

A solution of 4-(7-bromo-2-chloropyrido[3,2-d]pyrimidin-4-yl)morpholine (2.0 g, 6.1 mmol), potassium carbonate (1.7 g, 12.2 mmol) and 2-m-tolylethanamine (1.6 g, 12.2 mmol) in N,N-dimethylformamide (10.0 mL) was stirred at 80° C. under nitrogen atmosphere for 16 h. The resultant mixture was poured into water, the precipitate was collected by filtration, washed with water and dried under vacuum to obtain 7-bromo-N-(3-methylphenethyl)-4-morpholinopyrido[3,2-d]pyrimidin-2-amine (1.7 g, 65%) as white solid. ¹H NMR (400 MHz, DMSO) δ 8.36 (s, 1H), 8.01-7.88 (m, 1H), 7.17-7.14 (m, 1H), 7.00 (d, J=7.3 Hz, 3H), 4.28 (bs, 4H), 3.74 (bs, 4H), 3.49 (s, 1H), 3.14-3.11 (m, 2H), 2.75 (bs, 2H), 2.28 (s, 3H); LCMS (ESI) m/z: 428.1 [M+H]⁺.

### Step 5: Synthesis of (2-(3-methylphenethylamino)-4-morpholinopyrido[3,2-d]pyrimidin-7-yl)(pyridin-3-yl)methanol and (2-((3-methylphenethyl)amino)-4-morpholinopyrido[3,2-d]pyrimidin-7-yl)(pyridin-3-yl)methanone

To a suspension of sodium hydride (80 mg, 2.0 mmol) in tetrahydrofuran (10 mL) at 0° C. was added 7-bromo-N-(3-methylphenethyl)-4-morpholinopyrido[3,2-d]pyrimidin-2-amine (854 mg, 2.0 mmol) slowly added portion wise. The resultant mixture was stirred for 10 min and cooled to −70° C. n-Butyllithium (0.8 mL 2.5 M in hexane) was added dropwise to the mixture over a period of 15 min and stirred for further 20 min at −70° C. Nicotinaldehyde (320 mg, 3.0 mmol) in 2 mL tetrahydrofuran was then added dropwise and stirred for further 2 h. The reaction was then quenched with 4 mL concentrated hydrochloric acid in 5 mL water and further diluted with 20 mL of diethyl ether. The organic layer was washed with brine, dried over anhydrous sodium sulfate and concentrated. The residue was subjected to prep-HPLC (Boston C18 21*250 mm 10 μm column. The mobile phase was acetonitrile/0.01% aqueous trifluoroacetic acid) to obtain (2-(3-methylphenethylamino)-4-morpholinopyrido[3,2-d]pyrimidin-7-yl)(pyridin-3-yl)methanol (260 mg, 29%) as white solid. ¹H NMR (400 MHz, DMSO) δ 8.67 (s, 1H), 8.47 (d, J=3.4 Hz, 1H), 8.33 (s, 1H), 7.81 (d, J=7.9 Hz, 1H), 7.65 (s, 1H), 7.36 (dd, J=7.8, 4.8 Hz, 1H), 7.17 (t, J=7.5 Hz, 1H), 7.19-7.042 (m, 3H), 6.87 (s, 1H), 6.34 (d, J=4.2 Hz, 1H), 5.93 (d, J=3.8 Hz, 1H), 4.27 (s, 4H), 3.71 (s, 4H), 3.49 (d, J=6.8 Hz, 2H), 2.80 (s, 2H), 2.27 (s, 3H); LCMS (ESI) m/z: 457.2 [M+H]⁺.

The byproduct compound 123 was also isolated from the prep-HPLC (5.1 mg, 1%) as white solid. ¹H NMR (400 MHz, DMSO) δ 8.97 (d, J=1.6 Hz, 1H), 8.88 (dd, J=4.8, 2.0 Hz, 1H), 8.59 (s, 1H), 8.21 (d, J=8.0 Hz, 1H), 7.88 (s, 1H), 7.64 (dd, J=8.0, 5.0 Hz, 1H), 7.18-7.23 (m, 2H), 7.10-6.93 (m, 3H), 4.32 (bs, 4H), 3.77 (bs, 4H), 3.51 (d, J=7.0 Hz, 2H), 2.82 (d, J=7.5 Hz, 2H), 2.26 (s, 3H); LCMS (ESI) m/z: 455.3 [M+H]⁺.

### Step 6: Synthesis of (2-(3-methylphenethylamino)-4-morpholinopyrido[3,2-d]pyrimidin-7-yl)(pyridin-3-yl)methyl acetate

To a solution of (2-(3-methylphenethylamino)-4-morpholinopyrido[3,2-d]pyrimidin-7-yl)(pyridin-3-yl)methanol (92 mg, 0.2 mmol) in pyridine (3 mL) was added acetic anhydride (204 mg, 2.0 mmol) dropwise at 0° C. After the addition, the reaction mixture was warmed up and stirred at room temperature overnight. It was concentrated, the residue was diluted with water (10 mL) and extracted with ethyl acetate (25 mL) twice. The combined organic phase was washed with brine (25 mL), dried over sodium sulfate and concentrated to obtain (2-(3-methylphenethylamino)-4-morpholinopyrido[3,2-d]pyrimidin-7-yl)(pyridin-3-yl)methyl acetate (60 mg, 60%) as white solid. LCMS (ESI) m/z: 499.2 [M+H]⁺.

### Step 7: Synthesis of N-(3-methylphenethyl)-4-morpholino-7-(pyridin-3-ylmethyl)pyrido[3,2-d]pyrimidin-2-amine

To a solution of (2-(3-methylphenethylamino)-4-morpholinopyrido[3,2-d]pyrimidin-7-yl)(pyridin-3-yl)methyl acetate (30 mg, 0.06 mmol), triethylamine (0.15 mL) in N,N-dimethylformamide (8 mL) was added 10% palladium hydroxide on activated carbon (6.0 mg) and the resultant mixture was stirred under hydrogen atmosphere at room temperature overnight. The reaction mixture was then filtered through a pad of celite and the filtrate was diluted with ethyl acetate/water (20 mL/20 mL). The organic layer was separated and the aqueous phase was extracted with ethyl acetate (25 mL) twice. The combined organic phase was washed with brine (25 mL), dried over sodium sulfate and concentrated. The residue was subjected to prep-HPLC (Boston C18 21*250 mm 10 μm column. The mobile phase was acetonitrile/0.01% aqueous trifluoroacetic acid) to obtain N-(3-methylphenethyl)-4-morpholino-7-(pyridin-3-ylmethyl)pyrido[3,2-d]pyrimidin-2-amine (9.9 mg, 37.5%) as white solid. ¹H NMR (400 MHz, DMSO) δ 8.59 (s, 1H), 8.44 (dd, J=4.8, 1.6 Hz, 1H), 8.29 (s, 1H), 7.72 (d, J=6.9 Hz, 1H), 7.54 (s, 1H), 7.34-7.30 (m, 1H), 7.17-7.14 (m, 1H), 7.10-6.96 (m, 3H), 6.88 (s, 1H), 4.27 (s, 4H), 4.07 (s, 2H), 3.72 (s, 4H), 3.47 (d, J=6.0 Hz, 2H), 2.79 (s, 2H), 2.27 (s, 3H); LCMS (ESI) m/z: 441.3 [M+H]⁺.

The following compound was synthesized according to the protocol described above:

| | | | Compd |
|---|---|---|---|
| Name | Structure | NMR, MS | # |
| (2-{[2-(3- methylphenyl) ethyl]amino}-4- (morpholin-4- yl)pyrido[3,2- d]pyrimidin-7- yl)(pyridin-3- yl)methanol | 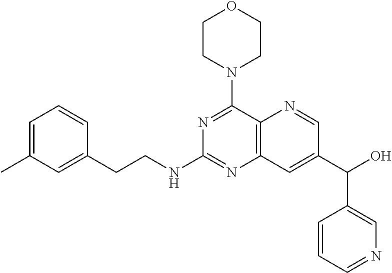 [SMILES: Cc1cccc(CCNc2nc(N3CCOCC3)c3ncc(C(O)c4cccnc4)cc3n2)c1] | 1H NMR (400 MHz, DMSO) δ 8.67 (s, 1H), 8.47 (d, J = 3.4 Hz, 1H), 8.33 (s, 1H), 7.81 (d, J = 7.9 Hz, 1H), 7.65 (s, 1H), 7.36 (dd, J = 7.8, 4.8 Hz, 1H), 7.17 (t, J = 7.5 Hz, 1H), 7.02 (dd, J = 21.0, 8.0 Hz, 3H), 6.87 (s, 1H), 6.34 (d, J = 4.2 Hz, 1H), 5.93 (d, J = 3.8 Hz, 1H), 4.27 (s, 4H), 3.71 (s, 4H), 3.49 (d, J = 6.8 Hz, 2H), 2.80 (s, 2H), 2.27 (s, 3H). LCMS (ESI) m/z: 457.2 [M + H]+. | 122 |
| N-[2-(3- methylphenyl) ethyl]-4- (morpholin-4- yl)-7-(pyridine- 3- carbonyl)pyrido [3,2-d] pyrimidin-2- amine | 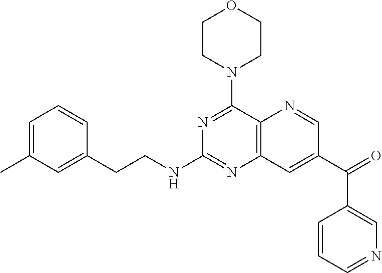 [SMILES: Cc1cccc(CCNc2nc(N3CCOCC3)c3ncc(C(=O)c4cccnc4)cc3n2)c1] | 1H NMR (400 MHz, DMSO) δ 8.97 (d, J = 1.6 Hz, 1H), 8.88 (dd, J = 4.8, 2.0 Hz, 1H), 8.59 (s, 1H), 8.21 (d, J = 8.0 Hz, 1H), 7.88 (s, 1H), 7.64 (dd, J = 8.0, 5.0 Hz, 1H), 7.18-7.23 (m, 2H), 7.10-6.93 (m, 3H), 4.35-4.31 (m, 4H), 3.79-3.72 (m, 4H), 3.51 (d, J = 7.0 Hz, 2H), 2.82 (d, J = 7.5 Hz, 2H), 2.26 (s, 3H). LCMS (ESI) m/z: 455.3 [M + H]+. | 123 |
| N-[2-(3- methylphenyl) ethyl]-4- (morpholin-4- yl)-7-[(pyridin- 3- yl)methyl] pyrido[3,2- d]pyrimidin-2- amine | 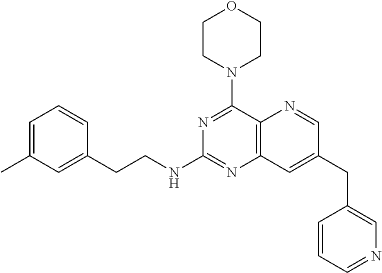 [SMILES: Cc1cccc(CCNc2nc(N3CCOCC3)c3ncc(Cc4cccnc4)cc3n2)c1] | 1H NMR (400 MHz, DMSO) δ 8.59 (s, 1H), 8.44 (dd, J = 4.8, 1.6 Hz, 1H), 8.29 (s, 1H), 7.72 (d, J = 6.9 Hz, 1H), 7.54 (s, 1H), 7.34-7.30 (m, 1H), 7.17- 7.14 (m, 1H), 7.10-6.96 (m, 3H), 6.88 (s, 1H), 4.27 (s, 4H), 4.07 (s, 2H), 3.72 (s, 4H), 3.47 (d, J = 6.0 Hz, 2H), 2.79 (s, 2H), 2.27 (s, 3H). LCMS (ESI) m/z: 441.3 [M + H]+. | 124 |

### Synthesis of morpholino-N-phenyl-7-(pyridin-3-ylmethyl)pyrido[3,2-d]pyrimidin-2-amine (Compound 125)

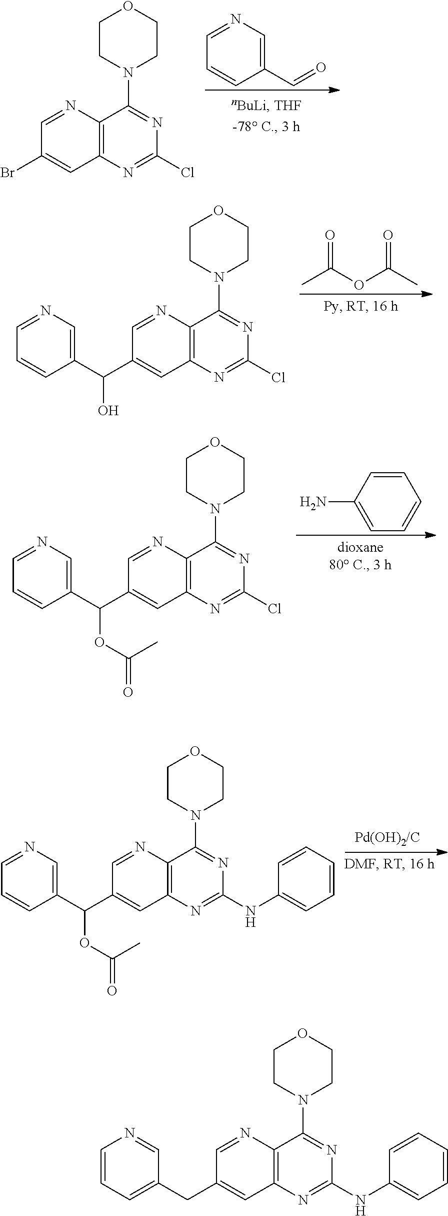

### Step 1: Synthesis of (2-chloro-4-morpholinopyrido[3,2-d]pyrimidin-7-yl)(pyridin-3-yl)methanol

To a suspension of sodium hydride (120 mg, 3.0 mmol) in tetrahydrofuran (18 mL) at 0° C. was added 4-(7-bromo-2-chloropyrido[3,2-d]pyrimidin-4-yl)morpholine (1.0 g, 3.0 mmol) portion wise. The resultant slurry was stirred for 10 min and then cooled to −70° C. Then, n-butyllithium (1.2 mL 2.5M in hexane) was added dropwise over a period of 15 min at −70° C. to the mixture and stirred further for 20 min at −70° C. To the resultant mixture, nicotinaldehyde (482 mg, 4.5 mmol) in 2 mL tetrahydrofuran was added dropwise and stirred for 2 h. The reaction was then quenched with 4 mL concentrated hydrochloric acid in 5 mL water and further diluted with 20 mL of diethyl ether. The organic layer was washed with brine and dried with anhydrous sodium sulfate and concentrated. The residue was subjected to flash column chromatography (ethyl acetate/petroleum ether 1:2) to obtain (2-chloro-4-morpholinopyrido[3,2-d]pyrimidin-7-yl)(pyridin-3-yl)methanol (600 mg, 56%). LCMS (ESI) m/z: 358.2 [M+H]⁺.

### Step 2: Synthesis of (2-chloro-4-morpholinopyrido[3,2-d]pyrimidin-7-yl)(pyridin-3-yl)methyl acetate

To a solution of (2-chloro-4-morpholinopyrido[3,2-d]pyrimidin-7-yl)(pyridin-3-yl)methanol (600 mg, 1.7 mmol) in pyridine (5 mL) was added acetic anhydride (1.7 g, 17 mmol) dropwise over an ice-bath condition. After the addition, the reaction mixture was stirred at room temperature overnight. It was concentrated, the residue was diluted with water (10 mL) and the mixture was extracted with ethyl acetate (25 mL) twice. The combined organic phase was washed with brine (25 mL), dried over sodium sulfate and concentrated to afford (2-chloro-4-morpholinopyrido[3,2-d]pyrimidin-7-yl)(pyridin-3-yl)methyl acetate (660 mg, 97%) as white solid. ¹H NMR (400 MHz, DMSO) δ 8.88 (d, J=2.0 Hz, 1H), 8.77 (d, J=2.0 Hz, 1H), 8.55 (d, J=4.6 Hz, 1H), 8.12 (d, J=2.1 Hz, 1H), 7.91 (d, J=8.0 Hz, 1H), 7.43 (dd, J=8.0, 4.8 Hz, 1H), 7.11 (s, 1H), 5.03-4.01 (m, 4H), 3.85-3.58 (m, 4H); LCMS (ESI) m/z: 400.2 [M+H]⁺.

### Step 3: Synthesis of (4-morpholino-2-(phenylamino)pyrido[3,2-d]pyrimidin-7-yl)(pyridin-3-yl)methyl acetate

To a solution of (2-chloro-4-morpholinopyrido[3,2-d]pyrimidin-7-yl)(pyridin-3-yl)methyl acetate (100 mg, 0.25 mmol) and aniline (47 mg, 0.5 mmol) in N,N-dimethylformamide (5 mL) was added potassium carbonate (69 mg, 0.5 mmol) portion-wise at an ice-bath temperature. After the addition, the reaction mixture was stirred at 80° C. overnight and concentrated. The residue was diluted with water (10 mL) and extracted with ethyl acetate (25 mL) twice. The combined organic phase was washed with brine (25 mL), dried over sodium sulfate and concentrated to afford (4-morpholino-2-(phenylamino)pyrido[3,2-d]pyrimidin-7-yl)(pyridin-3-yl)methyl acetate (100 mg, 88%) as white solid. LCMS (ESI) m/z: 457.2 [M+H]⁺.

### Step 4: Synthesis of 4-morpholino-N-phenyl-7-(pyridin-3-ylmethyl)pyrido[3,2-d]pyrimidin-2-amine

To a solution of (4-morpholino-2-(phenylamino)pyrido[3,2-d]pyrimidin-7-yl)(pyridin-3-yl)methyl acetate (100 mg, 0.22 mmol) and triethylamine (0.15 mL) in N,N-dimethylformamide (8 mL) was added 10% palladium hydroxide on activated carbon (20 mg) and the resultant mixture was stirred under hydrogen atmosphere at room temperature overnight. The mixture was filtered through a pad of celite and the filtrate was diluted with ethyl acetate/water (20 mL/20 mL). The organic layer was separated, and the aqueous layer was extracted with ethyl acetate (25 mL) twice. The combined organic phase was washed with brine (25 mL), dried over sodium sulfate, and concentrated. The residue was subjected to prep-HPLC (Boston C18 21*250 mm 10 μm column. The mobile phase was acetonitrile/0.01% aqueous trifluoroacetic acid) to obtain 4-morpholino-N-phenyl-7-(pyridin-3-ylmethyl)pyrido[3,2-d]pyrimidin-2-amine (59.4 mg, 68%) as white solid. ¹H NMR (400 MHz, DMSO) δ 9.23 (s, 1H), 8.62 (d, J=1.8 Hz, 1H), 8.56-8.35 (m, 2H), 7.85 (d, J=8.0 Hz, 2H), 7.75 (d, J=7.8 Hz, 1H), 7.69 (d, J=1.8 Hz, 1H), 7.34 (dd, J=8.0, 5.0 Hz, 1H), 7.28-7.23 (m, 2H), 6.93 (d, J=7.3 Hz, 1H), 4.37 (bs, 4H), 4.13 (s, 2H), 3.76 (t, J=4.8 Hz, 4H); LCMS (ESI) m/z: 399.1 [M+H]⁺.

### Syntheses of 4-(2-chloro-7-(pyridin-3-ylmethyl)pyrido[3,2-d]pyrimidin-4-yl)morpholine (Compound 128) and (E)-4-(2-(2-(3-methylbenzylidene)hydrazinyl)-7-(pyridin-3-ylmethyl)pyrido[3,2-d]pyrimidin-4-yl)morpholine (Compound 126)

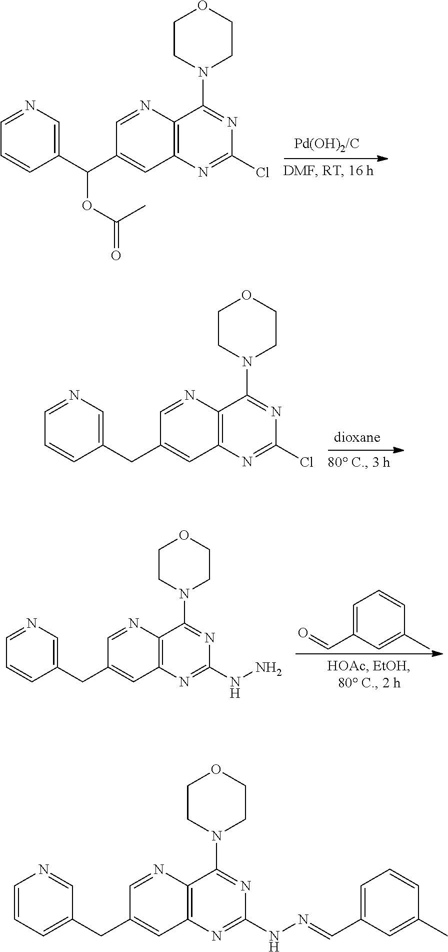

### Step 1: Synthesis of 4-(2-chloro-7-(pyridin-3-ylmethyl)pyrido[3,2-d]pyrimidin-4-yl)morpholine

A solution of (2-chloro-4-morpholinopyrido[3,2-d]pyrimidin-7-yl)(pyridin-3-yl)methyl acetate (300 mg, 0.75 mmol) and triethylamine (0.15 mL) in N,N-dimethylformamide (8 mL) was subjected to hydrogenation conditions over palladium hydroxide on activated carbon (10% load, 60.0 mg) and stirred under hydrogen atmosphere at room temperature overnight. The mixture was filtered through a pad of celite and the filtrate was diluted with ethyl acetate/water (20 mL/20 mL). The organic layer was separated and the aqueous layer was extracted with ethyl acetate (50 mL) twice. The combined organic phase was washed with brine (25 mL), dried over sodium sulfate, filtered and concentrated. The residue was subjected to prep-HPLC (Boston C18 21*250 mm 10 μm column. The mobile phase was acetonitrile/0.01% aqueous trifluoroacetic acid) to obtain 4-(2-chloro-7-(pyridin-3-ylmethyl)pyrido[3,2-d]pyrimidin-4-yl)morpholine (96 mg, 40%) as white solid. ¹H NMR (400 MHz, DMSO) δ 8.76 (d, J=2.2 Hz, 1H), 8.61 (d, J=1.9 Hz, 1H), 8.45 (dd, J=4.8, 1.6 Hz, 1H), 7.92 (d, J=2.1 Hz, 1H), 7.74 (d, J=7.7 Hz, 1H), 7.34 (dd, J=7.8, 4.8 Hz, 1H), 4.21 (s, 2H), 4.98-4.33 (m, 4H), 3.75 (t, J=4.4 Hz, 4H); LCMS (ESI) m/z: 342.1 [M+H]⁺.

### Step 2: Synthesis of (4-(2-hydrazinyl-7-(pyridin-3-ylmethyl)pyrido[3,2-d]pyrimidin-4-yl)morpholine

A solution of 4-(2-chloro-7-(pyridin-3-ylmethyl)pyrido[3,2-d]pyrimidin-4-yl)morpholine (95 mg, 0.28 mmol) and hydrazine hydrate (70 mg, 1.4 mmol) in dioxane (5.0 mL) was stirred at 90° C. under nitrogen atmosphere for 2 h. The reaction mixture was concentrated and precipitate formed was filtered to obtain 4-(2-hydrazinyl-7-(pyridin-3-ylmethyl)pyrido[3,2-d]pyrimidin-4-yl)morpholine (90 mg, 100%) as brown solid. LCMS (ESI) m/z: 338.3 [M+H]⁺.

### Step 3: Synthesis of (E)-4-(2-(2-(3-methylbenzylidene)hydrazinyl)-7-(pyridin-3-ylmethyl)pyrido[3,2-d]pyrimidin-4-yl)morpholine

To a solution of 4-(2-hydrazinyl-7-(pyridin-3-ylmethyl)pyrido[3,2-d]pyrimidin-4-yl)morpholine (95 mg, 0.28 mmol) and 3-methylbenzaldehyde (67 mg, 0.56 mmol) in ethanol (5.0 mL) was added acetic acid (19 mg, 0.32 mmol) and the resultant mixture was stirred at 80° C. under nitrogen for 2 h. It was concentrated and the residue was purified by HPLC (Boston C18 21*250 mm 10 μm column. The mobile phase was acetonitrile/0.01% aqueous trifluoroacetic acid) to obtain (E)-4-(2-(2-(3-methylbenzylidene)hydrazinyl)-7-(pyridin-3-ylmethyl)pyrido[3,2-d]pyrimidin-4-yl)morpholine (3.8 mg, 3.1%) as yellow solid. ¹H NMR (400 MHz, DMSO) δ 10.90 (s, 1H), 8.62 (s, 1H), 8.49-8.35 (m, 2H), 8.10 (s, 1H), 7.76 (d, J=7.7 Hz, 1H), 7.70 (s, 1H), 7.51-7.43 (m, 2H), 7.38-7.29 (m, 2H), 7.17 (d, J=7.5 Hz, 1H), 4.39 (bs, 4H), 4.13 (s, 2H), 3.78 (t, J=4.4 Hz, 4H), 2.35 (s, 3H); LCMS (ESI) m/z: 440.3 [M+H]⁺.

### Synthesis of 1-(4-morpholino-7-(pyridin-3-ylmethyl)pyrido[3,2-d]pyrimidin-2-yl)-3-m-tolyl-1H-pyrazol-5-ol (Compound 127)

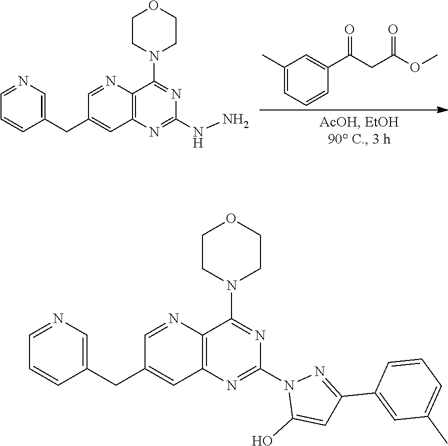

To a solution of 4-(2-hydrazinyl-7-(pyridin-3-ylmethyl)pyrido[3,2-d]pyrimidin-4-yl)morpholine (95 mg, 0.28 mmol) and methyl 3-oxo-3-m-tolylpropanoate (108 mg, 0.56 mmol) in ethanol (5.0 mL) was added glacial acetic acid (96 mg, 1.6 mmol) and the resultant mixture was stirred at 20° C. under nitrogen atmosphere for 2 h. The reaction mixture was concentrated, and the residue was subjected to prep-HPLC (Boston C18 21*250 mm 10 μm column. The mobile phase was acetonitrile/0.01% aqueous trifluoroacetic acid) to obtain 1-(4-morpholino-7-(pyridin-3-ylmethyl)pyrido[3,2-d]pyrimidin-2-yl)-3-m-tolyl-1H-pyrazol-5-ol (4.8 mg, 4.3%) as yellow solid. ¹H NMR (400 MHz, DMSO) δ 8.72 (d, J=2 Hz, 1H), 8.65 (s, 1H), 8.46 (d, J=4.4 Hz, 1H), 8.16 (s, 1H), 7.78 (d, J=8.0, 1H), 7.71-7.65 (m, 2H), 7.39-7.29 (m, 2H), 7.19 (d, J=7.2 Hz, 1H), 6.14 (s, 1H), 5.16-4.28 (m, 4H), 4.21 (s, 2H), 3.83 (bs, 4H), 2.38 (s, 3H); LCMS (ESI) m/z: 480.1 [M+H]⁺.

### Synthesis of N-(3-fluorophenyl)-4-morpholino-7-(pyridin-3-ylmethyl)pyrido[3,2-d]pyrimidin-2-amine (Compound 129)

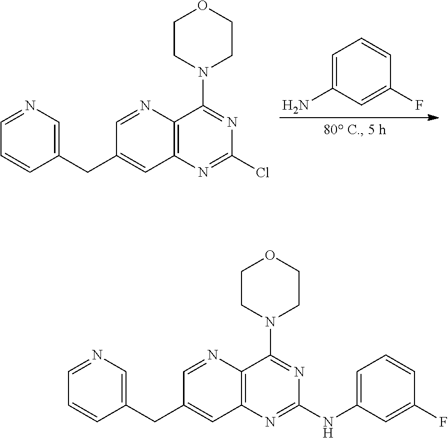 [SMILES: Clc1nc(N2CCOCC2)c2ncc(Cc3cccnc3)cc2n1 ; Nc1cccc(F)c1 ; Fc1cccc(Nc2nc(N3CCOCC3)c3ncc(Cc4cccnc4)cc3n2)c1]

A solution of 4-(2-chloro-7-(pyridin-3-ylmethyl)pyrido[3,2-d]pyrimidin-4-yl)morpholine (20 mg, 0.06 mmol) in 3-fluoroaniline (1.5 mL) was stirred at 80° C. for 5 h. The mixture was filtered, and the filtrate was subjected to prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain N-(3-fluorophenyl)-4-morpholino-7-(pyridin-3-ylmethyl)pyrido[3,2-d]pyrimidin-2-amine (9.6 mg, 41%) as white solid. ¹H NMR (400 MHz, DMSO) δ 9.46 (s, 1H), 8.62 (d, J=1.7 Hz, 1H), 8.47-8.39 (m, 2H), 7.97 (d, J=12.4 Hz, 1H), 7.76 (t, J=4.8 Hz, 2H), 7.54 (d, J=8.2 Hz, 1H), 7.34 (dd, J=7.8, 4.8 Hz, 1H), 7.27 (dd, J=15.4, 8.1 Hz, 1H), 6.71 (td, J=8.3, 2.4 Hz, 1H), 4.39 (bs, 4H), 4.14 (s, 2H), 3.76 (t, J=4.0 Hz, 4H); LCMS (ESI) m/z: 417.1 [M+H]⁺.

### Synthesis of 4-(2-phenoxy-7-(pyridin-3-ylmethyl)pyrido[3,2-d]pyrimidin-4-yl)morpholine (Compound 130)

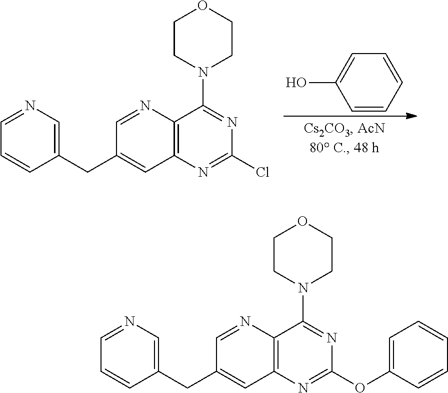 [SMILES: Clc1nc(N2CCOCC2)c2ncc(Cc3cccnc3)cc2n1 ; Oc1ccccc1 ; c1ccc(Oc2nc(N3CCOCC3)c3ncc(Cc4cccnc4)cc3n2)cc1]

A mixture of 4-(2-chloro-7-(pyridin-3-ylmethyl)pyrido[3,2-d]pyrimidin-4-yl)morpholine (20 mg, 0.06 mmol), cesium carbonate (38 mg, 0.12 mmol) and phenol (11 mg, 0.12 mmol) in acetonitrile (2 mL) was stirred at 80° C. for 48 h. The resultant mixture was filtered, and the filtrate was subjected to prep-HPLC (SunFire C18, 4.6*50 mm, 3.5 um column Xbridge C18 3.5 μm 4.6×50 mm column. The mobile phase was acetonitrile/10 mM formic acid aqueous solution) to obtain 4-(2-phenoxy-7-(pyridin-3-ylmethyl)pyrido[3,2-d]pyrimidin-4-yl)morpholine (6.8 mg, 29%) as white solid. ¹H NMR (400 MHz, DMSO) δ 8.60 (d, J=2.0 Hz, 1H), 8.58 (s, 1H), 8.43 (d, J=3.3 Hz, 2H), 7.72 (t, J=4.8 Hz, 2H), 7.41 (t, J=7.9 Hz, 2H), 7.32 (dd, J=7.7, 4.8 Hz, 1H), 7.23 (d, J=7.4 Hz, 1H), 7.19 (d, J=7.7 Hz, 2H), 4.36 (s, 4H), 4.13 (s, 2H), 3.2 (t, J=4.0 Hz, 4H), LCMS (ESI) m/z: 400.1 [M+H]⁺.

### Synthesis of 4-(2-(2-phenylpyrimidin-4-yl)-7-(pyridin-3-ylmethyl)pyrido[3,2-d]pyrimidin-4-yl)morpholine (Compound 131)

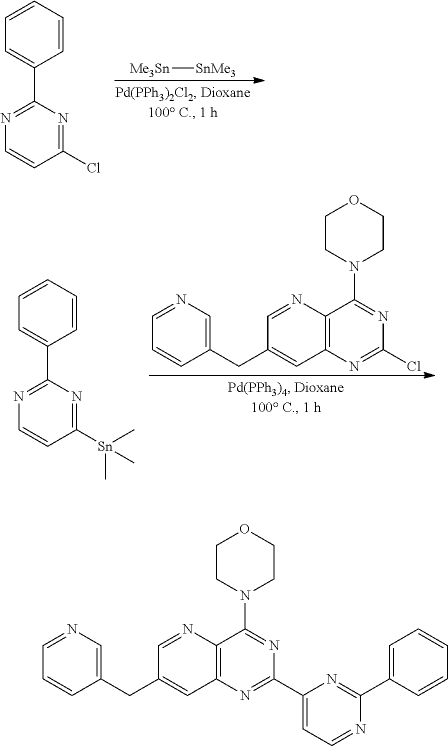

### Step 1: Synthesis of 2-phenyl-4-(trimethylstannyl)pyrimidine

To a solution of 4-chloro-2-phenylpyrimidine (0.03 g, 0.16 mmol) in dioxane (2 mL) were added 1,1,1,2,2,2-hexamethyldistannane (0.08 g, 0.25 mmol) and bis(triphenylphosphine)palladium(II) chloride (0.011 g, 0.02 mmol) at 25° C. and the reaction was stirred at 100° C. for 1 h under argon atmosphere. The resultant product was taken to the next step without any further purification. LCMS (ESI) m/z: 321.0 [M+H]⁺.

### Step 2: Synthesis of 4-(2-(2-phenylpyrimidin-4-yl)-7-(pyridin-3-ylmethyl)pyrido[3,2-d]pyrimidin-4-yl)morpholine

To a solution of 2-phenyl-4-(trimethylstannyl)pyrimidine (0.05 g, 0.15 mmol) in dioxane (2 mL) from step-1, were added 4-(2-chloro-7-(pyridin-3-ylmethyl)pyrido[3,2-d]pyrimidin-4-yl)morpholine (0.03 g, 0.09 mmol) and tetrakis(triphenylphosphine)palladium (0.01 g, 0.01 mmol) at 25° C. and the resultant mixture was stirred at 100° C. for 1 h under argon atmosphere. The mixture was filtered, and the filtrate was subjected to prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to obtain the title compound (6.5 mg, 16%) as white solid. ¹H NMR (400 MHz, DMSO) δ 9.09 (d, J=5.1 Hz, 1H), 8.86 (d, J=2.1 Hz, 1H), 8.67 (s, 1H), 8.53 (dd, J=6.6, 3.1 Hz, 2H), 8.47 (d, J=3.8 Hz, 1H), 8.31 (d, J=5.1 Hz, 1H), 8.20 (d, J=2.0 Hz, 1H), 7.81 (d, J=7.9 Hz, 1H), 7.63-7.53 (m, 3H), 7.37 (dd, J=7.8, 4.8 Hz, 1H), 4.57 (s, 4H), 4.27 (s, 2H), 3.94-3.74 (m, 4H); LCMS (ESI) m/z: 462.2 [M+H]⁺.

The following compound was synthesized according to the protocol described above:

| | | | Compd |
|---|---|---|---|
| Name | Structure | NMR, MS | # |
| 4-(2-(2-(3- fluorophenyl) pyrimidin-4- yl)-7-(pyridin- 3- ylmethyl) pyrido[3,2- d]pyrimidin-4- yl)morpholine | 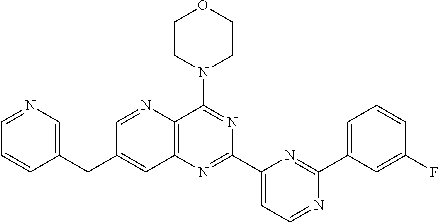 [SMILES: Fc1cccc(-c2nccc(-c3nc(N4CCOCC4)c4ncc(Cc5cccnc5)cc4n3)n2)c1] | ¹H NMR (400 MHz, DMSO) δ 9.11 (d, J = 5.1 Hz, 1H), 8.86 (d, J = 2.2 Hz, 1H), 8.67 (d, J = 1.8 Hz, 1H), 8.47 (dd, J = 4.8, 1.5 Hz, 1H), 8.38 (d, J = 7.9 Hz, 1H), 8.35 (d, J = 5.1 Hz, 1H), 8.23 (dd, J = 10.3, 2.4 Hz, 2H), 7.84-7.78 (m, 1H), 7.64 (td, J = 8.0, 6.1 Hz, 1H), 7.43 (td, J = 8.4, 1.9 Hz, 1H), 7.37 (dd, J = 7.7, 4.8 Hz, 1H), 4.57 (bs, 4H), 4.27 (s, 2H), 3.82 (t, J = 4.4Hz, 4H); LCMS (ESI) m/z: 480.2 [M + H]⁺. | 132 |

### Synthesis 4-morpholino-N-phenyl-7-(tetrahydrofuran-2-yl)pyrido[3,2-d]pyrimidin-2-amine (Compound 133)

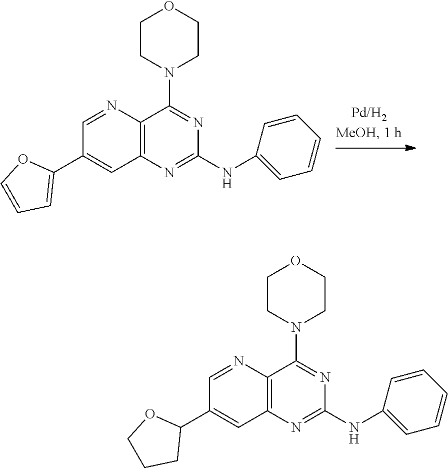 [SMILES: c1ccc(Nc2nc(N3CCOCC3)c3ncc(-c4ccco4)cc3n2)cc1 ; c1ccc(Nc2nc(N3CCOCC3)c3ncc(C4CCCO4)cc3n2)cc1]

To a solution of 7-(furan-2-yl)-4-morpholino-N-phenylpyrido[3,2-d]pyrimidin-2-amine (5 mg, 0.05 mmol) in MeOH (5 mL) was added Pd/C (10% loading, 2 mg). The reaction mixture was stirred at room temperature for 2 h under hydrogen atmosphere. Then the mixture was filtrated, and the filtrate was concentrated. The residue was subjected to prep-HPLC (0.05% FA/H₂O:CH₃CN=5%˜95%) to obtain 4-morpholino-N-phenyl-7-(tetrahydrofuran-2-yl)pyrido[3,2-d]pyrimidin-2-amine·formate (3 mg, 57%) as yellow solid. ¹H NMR (400 MHz, DMSO-d₆) δ 9.23 (s, 1H), 8.43 (d, J=2.0 Hz, 1H), 8.27 (s, 1H), 7.87 (d, J=7.6 Hz, 2H), 7.70 (s, 1H), 7.28 (t, J=8.0 Hz, 1H), 6.93 (d, J=7.2 Hz, 1H), 5.00 (t, J=7.2 Hz, 1H), 4.39 (s, 4H), 4.07-4.04 (m, 1H), 3.88-3.84 (m, 1H), 3.78 (t, J=4.8 Hz, 4H), 2.44-2.39 (m, 1H), 2.00-1.97 (m, 2H), 1.77 (dd, J=12.4, 8.0 Hz, 1H); LCMS (ESI) m/z: 378.3 [M+H]⁺.

The following compound was synthesized according to the protocol described above:

| | | | Compd |
|---|---|---|---|
| Name | Structure | NMR, MS | # |
| 4-(morpholin-4- yl)-7-(oxolan-2- yl)-N- phenylpyrido [3,2-d]pyrimidin- 2-amine | 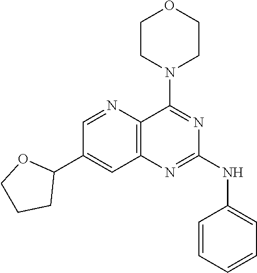 [SMILES: c1ccc(Nc2nc(N3CCOCC3)c3ncc(C4CCCO4)cc3n2)cc1] | 1H NMR (400 MHz, DMSO-d6) δ 9.23 (s, 1H), 8.43 (d, J = 2.0 Hz, 1H), 8.27 (s, 1H), 7.87 (d, J = 7.6 Hz, 2H), 7.70 (s, 1H), 7.28 (t, J = 8.0 Hz, 1H), 6.93 (d, J = 7.2 Hz, 1H), 5.00 (t, J = 7.2 Hz, 1H), 4.39 (s, 4H), 4.07-4.04 (m, 1H), 3.88-3.84 (m, 1H), 3.78 (t, J = 4.8 Hz, 4H), 2.44-2.39 (m, 1H), 2.00-1.97 (m, 2H), 1.77 (dd, J = 12.4, 8.0 Hz, 1H). LCMS (ESI) m/z: 378.3 [M + H]+. | 133 |

### Synthesis of 4-(2-phenoxy-7-(tetrahydrofuran-2-yl)pyrido[3,2-d]pyrimidin-4-yl)morpholine (Compound 134)

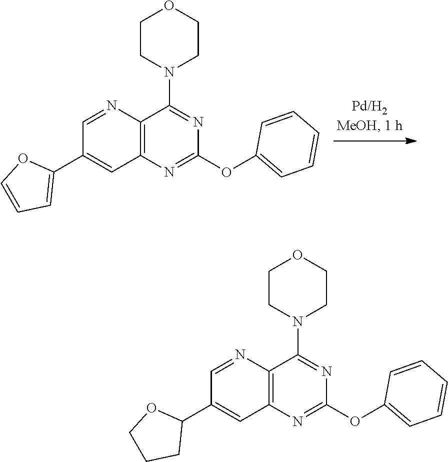 [SMILES: c1ccc(Oc2nc(N3CCOCC3)c3ncc(-c4ccco4)cc3n2)cc1 ; c1ccc(Oc2nc(N3CCOCC3)c3ncc(C4CCCO4)cc3n2)cc1]

To a solution of 4-(7-(furan-2-yl)-2-phenoxypyrido[3,2-d]pyrimidin-4-yl)morpholine (25 mg, 0.067 mmol) in MeOH (10 mL), was added 10% Pd/C (3 mg). The reaction mixture was stirred at room temperature for 1 h under hydrogen atmosphere, the filtered and filtrate was concentrated. The residue was subjected to prep-HPLC (0.05% FA/H₂O:CH₃CN=5%˜95%) to obtain 4-(2-phenoxy-7-(tetrahydrofuran-2-yl)pyrido[3,2-d]pyrimidin-4-yl)morpholine (12.1 mg, 48%) as yellow solid. ¹H NMR (400 MHz, DMSO-d₆) 8.61 (d, J=2.0 Hz, 1H), 7.70 (dd, J=2.0, 0.8 Hz, 1H), 7.52-7.41 (m, 2H), 7.26-7.20 (m, 3H), 5.00 (t, J=7.2 Hz, 17H), 4.38 (bs, 4H), 4.02 (dd, J=14.8, 6.8 Hz, 1H), 3.85 (dd, J=14.8, 7.2 Hz, 1H), 3.74 (t, J=7.2 Hz, 4H), 2.40 (dd, J=12.4, 6.4 Hz, 1H), 1.98-1.94 (m, 2H), 1.75 (dd, J=12.0, 8.0 Hz, 11H); LCMS (ESI) m/z: 379.2 [M+H]⁺.

The following compound was synthesized according to the protocol described above:

| | | | Compd |
|---|---|---|---|
| Name | Structure | NMR, MS | # |
| 4-[7-(oxolan-2- yl)-2- phenoxypyrido [3,2-d]pyrimidin- 4-yl]morpholine | 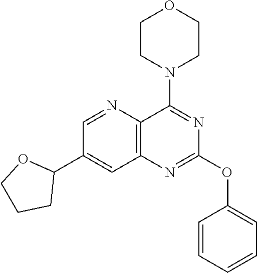 [SMILES: c1ccc(Oc2nc(N3CCOCC3)c3ncc(C4CCCO4)cc3n2)cc1] | 1H NMR (400 MHz, DMSO-d6) δ 8.61 (d, J = 2.0 Hz, 1H), 7.70 (dd, J = 2.0, 0.8 Hz, 1H), 7.52- 7.41 (m, 2H), 7.26-7.20 (m, 3H), 5.00 (t, J = 7.2 Hz, 1H), 4.38 (s, 4H), 4.02 (dd, J = 14.8, 6.8 Hz, 1H), 3.85 (dd, J = 14.8, 7.2 Hz, 1H), 3.74 (t, J = 7.2 Hz, 4H), 2.40 (dd, J = 12.4, 6.4 Hz, 1H), 1.98-1.94 (m, 2H), 1.75 (dd, J = 12.0, 8.0 Hz, 1H). LCMS (ESI) m/z: 379.2 [M + H]+ | 134 |

### Synthesis of 4-(2-(3-(1-methyl-1H-pyrazol-3-yl)phenyl)pyrido[3,2-d]pyrimidin-4-yl)morpholine (Compound 135)

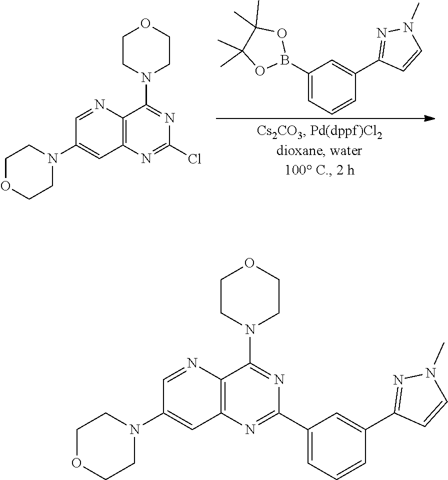 [SMILES: Clc1nc(N2CCOCC2)c2ncc(N3CCOCC3)cc2n1 ; Cn1ccc(-c2cccc(B3OC(C)(C)C(C)(C)O3)c2)n1 ; Cn1ccc(-c2cccc(-c3nc(N4CCOCC4)c4ncc(N5CCOCC5)cc4n3)c2)n1]

To a solution of 1-methyl-3-(3-(4,4,5,5-tetramethyl-1,3,2-dioxaborolan-2-yl)phenyl)-1H-pyrazole (0.05 g, 0.15 mmol) and 4,4′-(2-chloropyrido[3,2-d]pyrimidine-4,7-diyl)dimorpholine (0.068 g, 0.24 mmol) in dioxane/water (3 mL/0.5 mL) were added cesium carbonate (0.12 g, 1.97 mmol) and [1,1′-bis(diphenylphosphino)ferrocene]dichloropalladium(II) (0.011 g, 0.015 mmol) and the reaction mixture was stirred at 90° C. for 2 h. It was then poured into ice water and extracted with ethyl acetate (15 mL*3). The combined organic layer was washed with brine, dried and concentrated. The crude product obtained was purified by prep-HPLC (SunFire C18, 4.6*50 mm, 3.5 um column. The elution system used was a gradient of 5%-95% over 1.5 min at 2 ml/min and the solvent was acetonitrile/0.01% ammonium bicarbonate aqueous solution) to obtain 4,4′-(2-(3-(1-methyl-1H-pyrazol-3-yl)phenyl)pyrido[3,2-d]pyrimidine-4,7-diyl)dimorpholine (35 mg, 51%) as yellow solid. ¹H NMR (400 MHz, DMSO-d₆) δ 8.85 (s, 1H), 8.70 (d, J=2.7 Hz, 1H), 8.34 (d, J=7.8 Hz, 1H), 7.88 (d, J=7.6 Hz, 1H), 7.78 (d, J=2.1 Hz, 1H), 7.51 (t, J=7.8 Hz, 1H), 7.38 (d, J=2.8 Hz, 1H), 6.74 (d, J=2.2 Hz, 1H), 4.48 (s, 4H), 3.92 (s, 3H), 3.86-3.74 (m, 8H), 3.49-3.41 (m, 4H). LCMS (ESI) m/z: 458.1 [M+H]⁺.

The following compound was synthesized similarly to the protocol described above:

| Name | Structure | NMR, MS | # |
|---|---|---|---|
| 4-(2-(3-(1-methyl-1H- pyrazol-3- yl)phenyl)pyrido[3,2- d]pyrimidin-4- yl)morpholine | 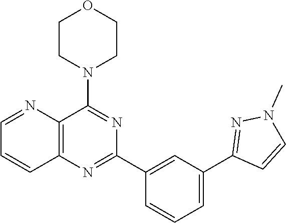 [SMILES: Cn1ccc(-c2cccc(-c3nc(N4CCOCC4)c4ncccc4n3)c2)n1] | ¹H NMR (400 MHz, CDCl₃) δ 8.88 (s, 1H), 8.68 (dd, J = 4.1, 1.7 Hz, 1H), 8.43 (d, J = 7.8 Hz, 1H), 8.24 (d, J = 8.3 Hz, 1H), 7.96 (d, J = 7.8 Hz, 1H), 7.62 (dd, J = 8.5, 4.1 Hz, 1H), 7.52 (t, J = 7.7 Hz, 1H), 7.42 (d, J = 2.2 Hz, 1H), 6.69 (d, J = 2.2 Hz, 1H), 4.63 (s, 4H), 3.99 (s, 3H), 3.97- 3.91 (m, 4H); LCMS (ESI) m/z: 372.9 [M]⁺. | 136 |

### Synthesis of 1-methyl-4-(2-(3-(1-methyl-1H-pyrazol-3-yl)phenyl)-4-morpholinopyrido[3,2-d]pyrimidin-7-yl)piperazin-2-one (Compound 137)

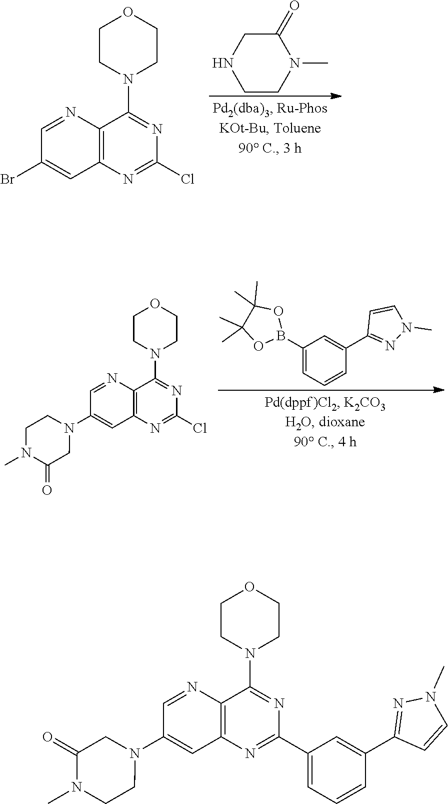

### Step 1: Synthesis of 4-(2-chloro-4-morpholinopyrido[3,2-d]pyrimidin-7-yl)-1-methylpiperazin-2-one

A mixture of 4-(7-bromo-2-chloropyrido[3,2-d]pyrimidin-4-yl)morpholine (100 mg, 0.304 mmol), 1-methylpiperazin-2-one (35 mg, 0.304 mmol), tris(dibenzylideneacetone)dipalladium (30 mg, 0.05 mmol), 2-dicyclohexylphosphino-2′,6′-diisopropoxy-1,1′-biphenyl (56 mg, 0.06 mmol) and sodium tert-butoxide (60 mg, 0.620 mmol) in toluene (10 mL) was stirred at 85° C. for 16 h. The reaction mixture was quenched with water (15 mL) and extracted with ethyl acetate (20 mL*3). The organic layer was combined, washed with brine (30 mL), dried over sodium sulfate, filtered and concentrated. The residue was subjected to flash chromatography [on silica gel using petroleum ether:ethyl acetate=75:25] to obtain 4-(2-chloro-4-morpholinopyrido[3,2-d]pyrimidin-7-yl)-1-methylpiperazin-2-one as yellow solid (45 mg, 40.9%). LCMS (ESI) m/z: 363.0[M+H]⁺.

### Step 2: Synthesis of 1-methyl-4-(2-(3-(1-methyl-1H-pyrazol-3-yl)phenyl)-4-morpholinopyrido[3,2-d]pyrimidin-7-yl)piperazin-2-one

To a solution of 4-(2-chloro-4-morpholinopyrido[3,2-d]pyrimidin-7-yl)-1-methylpiperazin-2-one (45 mg, 0.124 mmol) in dioxane (5 mL) and water (5 mL) were added 1-methyl-3-(3-(4,4,5,5-tetramethyl-1,3,2-dioxaborolan-2-yl)phenyl)-1H-pyrazole (29 mg, 0.248 mmol), potassium carbonate (37 mg, 0.266 mmol) and dichloro[1,1′-bis(diphenylphosphino)ferrocene]palladium(II) dichloromethane adduct (50 mg, 0.196 mmol). The resultant mixture was stirred at 90° C. for 2 h and cooled. It was then diluted with 10 mL of water and extracted with ethyl acetate (10 mL*3). The organic layer was combined, washed with brine (30 mL), dried over sodium sulfate, filtered and concentrated. The residue was subjected to prep-HPLC (0.05% ammonium bicarbonate:acetonitrile=5%˜95%) to obtain 1-methyl-4-(2-(3-(1-methyl-1H-pyrazol-3-yl)phenyl)-4-morpholino pyrido[3,2-d]pyrimidin-7-yl)piperazin-2-one (15.7 mg, 26.2%) as white solid. ¹H NMR (400 MHz, DMSO-d₆) δ 8.85 (s, 1H), 8.68 (d, J=2.9 Hz, 1H), 8.34 (d, J=7.8 Hz, 1H), 7.89 (d, J=7.7 Hz, 1H), 7.78 (d, J=2.1 Hz, 1H), 7.52 (t, J=7.7 Hz, 1H), 7.36 (d, J=2.8 Hz, 1H), 6.75 (d, J=2.2 Hz, 1H), 4.49 (s, 4H), 4.09 (s, 2H), 3.93 (s, 3H), 3.82 (d, J=4.4 Hz, 6H), 3.55-3.46 (m, 2H), 2.93 (s, 3H). LCMS (ESI) m/z: 484.8 [M]⁺.

### Synthesis of Compound 134

Compound 134 was prepared by synthetic protocols known to one of skill in the art.

| Name | Structure | NMR, MS | # |
|---|---|---|---|
| 4-{2-chloro-7- [(pyridin-3- yl)methyl]pyrido [3,2-d]pyrimidin- 4-yl}morpholine | 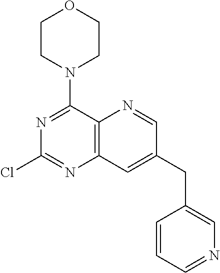 [SMILES: Clc1nc(N2CCOCC2)c2ncc(Cc3cccnc3)cc2n1] | 1H NMR (400 MHz, DMSO) δ 8.76 (d, J = 2.2 Hz, 1H), 8.61 (d, J = 1.9 Hz, 1H), 8.45 (dd, J = 4.8, 1.6 Hz, 1H), 7.92 (d, J = 2.1 Hz, 1H), 7.74 (d, J = 7.7 Hz, 1H), 7.34 (dd, J = 7.8, 4.8 Hz, 1H), 4.21 (s, 2H), 4.98- 4.33 (m, 4H), 3.79-3.72 (m, 4H). LCMS (ESI) m/z: 342.1 [M + H]+. | 128 |

### PIKfyve Inhibitory Activity, Metabolic Stability, Permeability, and Solubility

PIKfyve Biochemical Assay. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRET™ TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc® Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax® Discover Multimode Microplate Reader (Promega) using 0.3 sec/well integration time, 450BP donor filter and 600LP acceptor filters. Active test compounds that bound PIKfyve and displaced the tracer reduced BRET signal. IC50 values were then calculated by fitting the data to the normalized BRET ratio.

The results of the PIKfyve inhibition assays are summarized in the Table below.

| Compound | hPIKfyve | hPIKfyve BRET |
|---|---|---|
| No. | IC₅₀ (μM)^{a} | IC₅₀ (μM)^{a} |
| 1 | + | |
| 2 | NA | |
| 3 | NA | |
| 4 | + | |
| 5 | − | |
| 6 | NA | |
| 7 | NA | |
| 8 | NA | |
| 9 | +++ | |
| 10 | ++++ | |
| 11 | +++ | |
| 12 | ++ | + |
| 13 | + | |
| 14 | + | |
| 15 | +++ | ++ |
| 16 | ++++ | |
| 17 | ++ | |
| 18 | − | |
| 19 | ++ | |
| 20 | +++ | |
| 21 | ++ | |
| 22 | +++ | |
| 23 | +++ | |
| 24 | +++ | |
| 25 | + | |
| 26 | − | |
| 27 | +++ | |
| 28 | ++++ | |
| 29 | ++ | |
| 30 | ++ | |
| 31 | ++ | |
| 32 | + | |
| 33 | ++ | |
| 34 | +++ | |
| 35 | ++ | |
| 36 | +++ | |
| 37 | +++ | |
| 38 | +++ | |
| 39 | − | |
| 40 | ++++ | |
| 41 | +++ | |
| 42 | +++ | |
| 43 | ++++ | |
| 44 | +++ | |
| 45 | ++++ | |
| 46 | ++++ | |
| 47 | − | |
| 48 | +++ | |
| 49 | + | |
| 50 | − | |
| 51 | + | |
| 52 | + | |
| 53 | − | |
| 54 | ++++ | |
| 55 | +++ | |
| 56 | +++ | |
| 57 | ++ | |
| 58 | +++ | |
| 59 | + | |
| 60 | + | |
| 61 | NA | |
| 62 | NA | |
| 63 | + | |
| 64 | + | |
| 65 | + | |
| 66 | + | |
| 67 | ++ | |
| 68 | + | |
| 69 | + | |
| 70 | ++++ | |
| 71 | +++ | − |
| 72 | +++ | ++ |
| 73 | + | |
| 74 | ++ | |
| 75 | +++ | |
| 76 | ++ | |
| 77 | + | |
| 78 | − | |
| 79 | NA | |
| 80 | NA | |
| 81 | + | |
| 82 | ++ | |
| 83 | + | |
| 84 | − | |
| 85 | − | |
| 86 | + | |
| 87 | + | |
| 88 | + | |
| 89 | + | |
| 90 | +++ | ++ |
| 91 | NA | |
| 92 | + | |
| 93 | ++ | |
| 94 | +++ | |
| 95 | +++ | |
| 96 | + | |
| 97 | ++ | |
| 98 | + | |
| 99 | + | |
| 100 | ++ | |
| 101 | ++ | |
| 102 | ++ | |
| 103 | +++ | |
| 104 | ++++ | |
| 105 | +++ | |
| 106 | ++ | |
| 107 | + | |
| 108 | +++ | |
| 109 | ++ | |
| 110 | ++ | |
| 111 | ++ | |
| 112 | − | |
| 113 | +++ | +++ |
| 114 | ++ | |
| 115 | NA | |
| 116 | +++ | |
| 117 | NA | NA |
| 122 | | |
| 123 | | |
| 124 | ++ | |
| 125 | ++ | |
| 126 | +++ | |
| 127 | +++ | |
| 128 | − | |
| 129 | ++ | |
| 130 | + | |
| 131 | ++ | |
| 132 | ++ | |
| 133 | ++ | |
| 134 | + | |
| 135 | | |
| 136 | | |
| 137 | | |

^{a}++++ stands for <10 nM, +++ stands for 10-100 nM, ++ stands for 100-1000 nM, + stands for 1-10 μM, and − stands for >10 μM.

### Viability Assay to Assess TDP-43 Toxicity in FAB1 TDP-43 and PIKfyve TDP-43 Yeast Cells.

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3;pdr1::NATMX; fab1::G418^{R}, his3;leu2;ura3;met15;LYS2+) (FIG. 2).

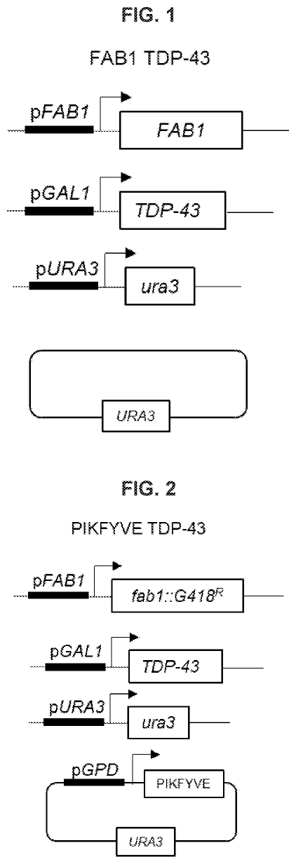

Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418^{R} pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 μM. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 μL/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

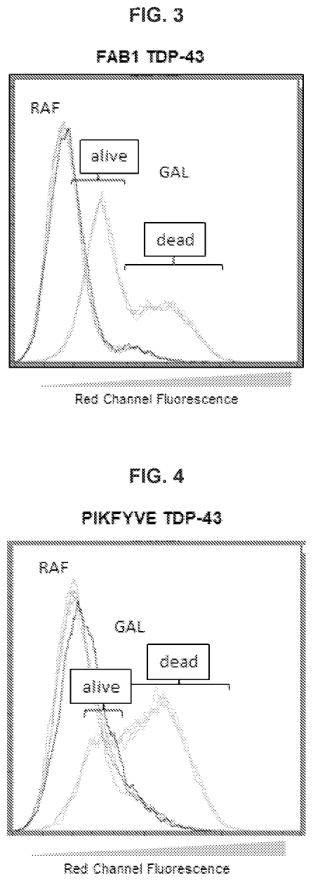

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

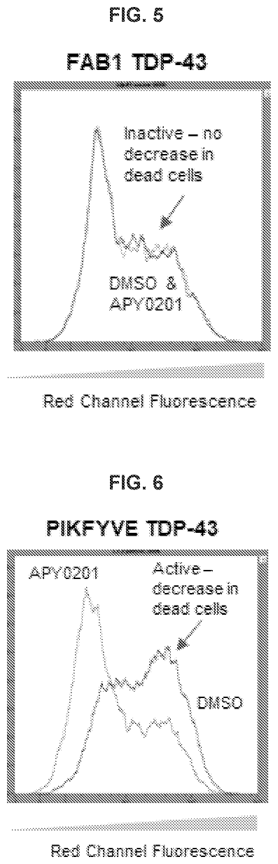

Activity of APY0201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

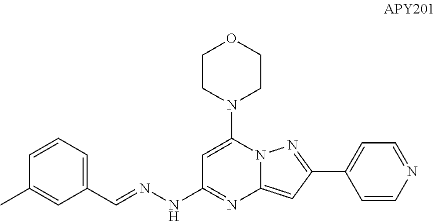 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | | FAB1 TDP-43 | PIKfyve TDP-43 |
|---|---|---|---|
| Structure | PIKfyve IC₅₀ (nM) | (active/inactive) | (active/inactive) |
| 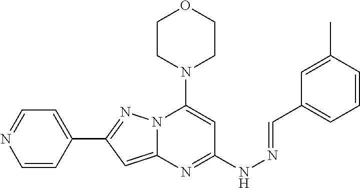 [SMILES: Cc1cccc(/C=N/Nc2cc(N3CCOCC3)n3nc(-c4ccncc4)cc3n2)c1] | 7.5 | Inactive | Active |
| 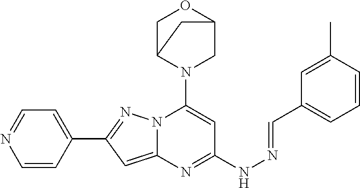 [SMILES: Cc1cccc(/C=N/Nc2cc(N3CC4CC3CO4)n3nc(-c4ccncc4)cc3n2)c1] | 12 | Inactive | Active |
| 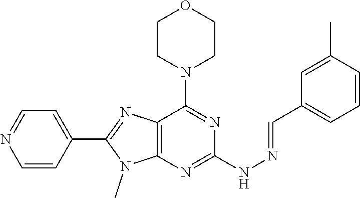 [SMILES: Cc1cccc(/C=N/Nc2nc(N3CCOCC3)c3nc(-c4ccncc4)n(C)c3n2)c1] | 4.9 | Inactive | Active |
| 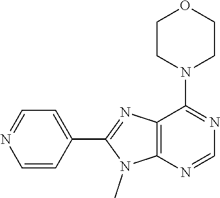 [SMILES: Cn1c(-c2ccncc2)nc2c(N3CCOCC3)ncnc21] | 640 | Inactive | Inactive |
| 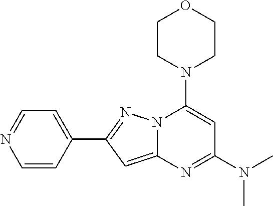 [SMILES: CN(C)c1cc(N2CCOCC2)n2nc(-c3ccncc3)cc2n1] | 2007 | Inactive | Inactive |
| 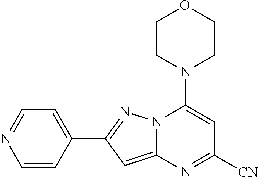 [SMILES: N#Cc1cc(N2CCOCC2)n2nc(-c3ccncc3)cc2n1] | >10000 | Inactive | Inactive |

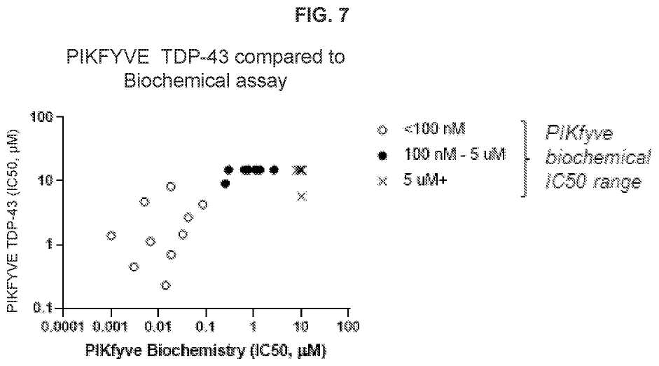

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 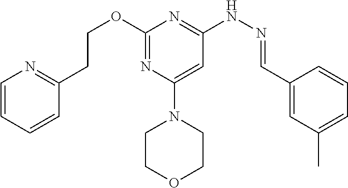 [SMILES: Cc1cccc(/C=N/Nc2cc(N3CCOCC3)nc(OCCc3ccccn3)n2)c1] | 0.003 | 0.450 |
| 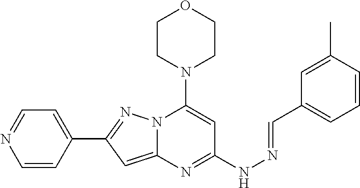 [SMILES: Cc1cccc(/C=N/Nc2cc(N3CCOCC3)n3nc(-c4ccncc4)cc3n2)c1] | 0.001 | 1.390 |
| 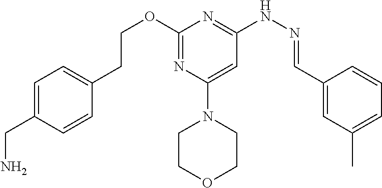 [SMILES: Cc1cccc(/C=N/Nc2cc(N3CCOCC3)nc(OCCc3ccc(CN)cc3)n2)c1] | 0.007 | 1.120 |
| 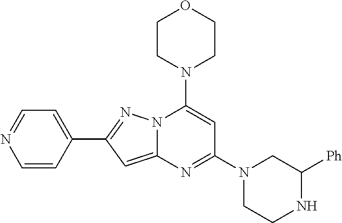 [SMILES: c1ccc(C2CN(c3cc(N4CCOCC4)n4nc(-c5ccncc5)cc4n3)CCN2)cc1] | 2.660 | >15 |
| 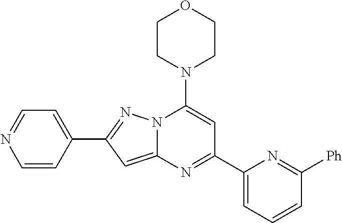 [SMILES: c1ccc(-c2cccc(-c3cc(N4CCOCC4)n4nc(-c5ccncc5)cc4n3)n2)cc1] | 0.014 | 0.230 |
| 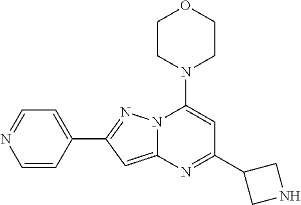 [SMILES: c1cc(-c2cc3nc(C4CNC4)cc(N4CCOCC4)n3n2)ccn1] | 8.020 | >15 |
| 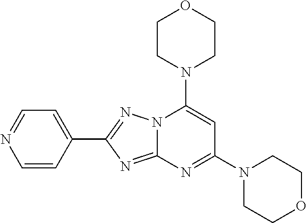 [SMILES: c1cc(-c2nc3nc(N4CCOCC4)cc(N4CCOCC4)n3n2)ccn1] | 9.200 | >15 |
| 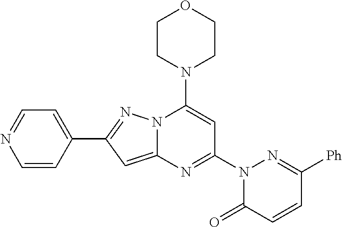 [SMILES: O=c1ccc(-c2ccccc2)nn1-c1cc(N2CCOCC2)n2nc(-c3ccncc3)cc2n1] | 0.295 | >15 |
| 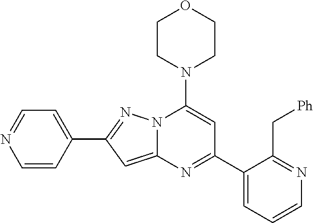 [SMILES: c1ccc(Cc2ncccc2-c2cc(N3CCOCC3)n3nc(-c4ccncc4)cc3n2)cc1] | 1.090 | >15 |
| 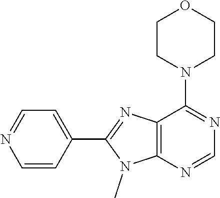 [SMILES: Cn1c(-c2ccncc2)nc2c(N3CCOCC3)ncnc21] | 0.640 | >15 |
| 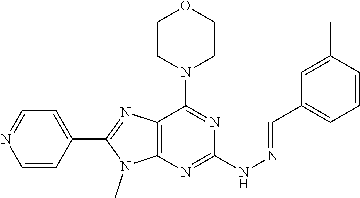 [SMILES: Cc1cccc(/C=N/Nc2nc(N3CCOCC3)c3nc(-c4ccncc4)n(C)c3n2)c1] | 0.005 | 4.720 |
| 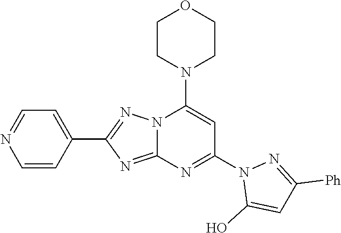 [SMILES: Oc1cc(-c2ccccc2)nn1-c1cc(N2CCOCC2)n2nc(-c3ccncc3)nc2n1] | 0.018 | 0.693 |
| 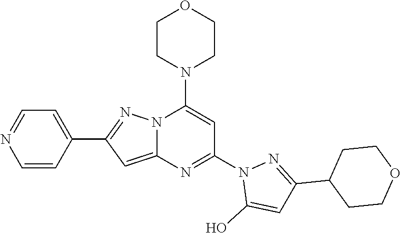 [SMILES: Oc1cc(C2CCOCC2)nn1-c1cc(N2CCOCC2)n2nc(-c3ccncc3)cc2n1] | 0.253 | 9.105 |
| 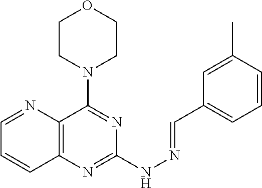 [SMILES: Cc1cccc(/C=N/Nc2nc(N3CCOCC3)c3ncccc3n2)c1] | 0.018 | 8.214 |
| 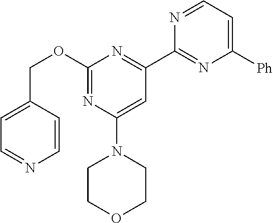 [SMILES: c1ccc(-c2ccnc(-c3cc(N4CCOCC4)nc(OCc4ccncc4)n3)n2)cc1] | 0.032 | 1.447 |
| 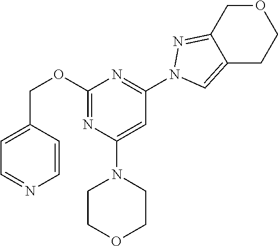 [SMILES: c1cc(COc2nc(N3CCOCC3)cc(-n3cc4c(n3)COCC4)n2)ccn1] | 1.343 | >15 |
| 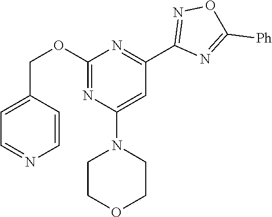 [SMILES: c1ccc(-c2nc(-c3cc(N4CCOCC4)nc(OCc4ccncc4)n3)no2)cc1] | >10 | >15 |
| 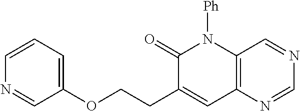 [SMILES: O=c1c(CCOc2cccnc2)cc2ncncc2n1-c1ccccc1] | >10 | >15 |
| 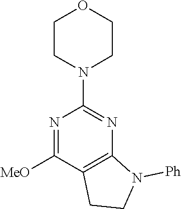 [SMILES: COc1nc(N2CCOCC2)nc2c1CCN2c1ccccc1] | 0.085 | 4.273 |
| 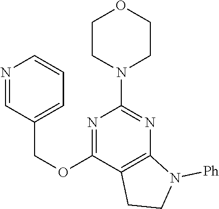 [SMILES: c1ccc(N2CCc3c(OCc4cccnc4)nc(N4CCOCC4)nc32)cc1] | 0.042 | 2.685 |
| 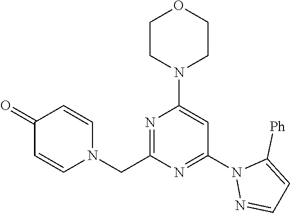 [SMILES: O=c1ccn(Cc2nc(N3CCOCC3)cc(-n3nccc3-c3ccccc3)n2)cc1] | >10 | >15 |
| 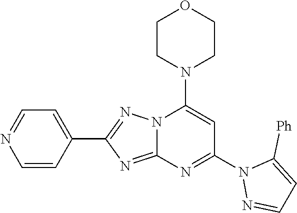 [SMILES: c1ccc(-c2ccnn2-c2cc(N3CCOCC3)n3nc(-c4ccncc4)nc3n2)cc1] | 0.767 | >15 |
| 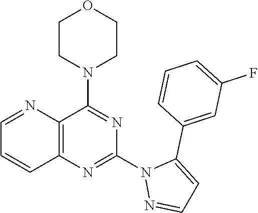 [SMILES: Fc1cccc(-c2ccnn2-c2nc(N3CCOCC3)c3ncccc3n2)c1] | >10 | 5.754 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of Formula I: 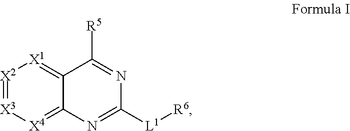
or a pharmaceutically acceptable salt thereof,
wherein
X¹ is N or CR¹;
X² is N or CR²;
X³ is N or CR³;
X⁴ is N or CR⁴;
R⁵ is 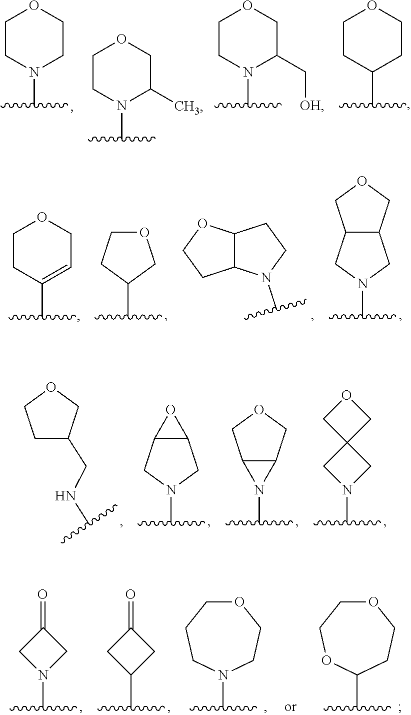
L¹ is 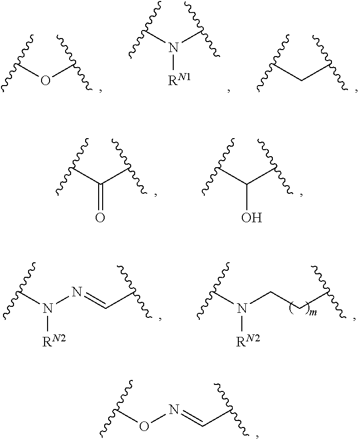
optionally substituted C₁₋₉ heteroarylene having at least one 5-membered ring, or optionally substituted C₂-C₉ heterocyclylene and R⁶ is optionally substituted C₁-C₆ alkyl, optionally substituted C₆-C₁₀ aryl, optionally substituted C₃-C₁₀ carbocyclyl, optionally substituted C₂-C₉ heteroaryl, optionally substituted C₂-C₉ heterocyclyl, optionally substituted C₆-C₁₀ aryl C₁-C₆ alkyl, or optionally substituted C₂-C₉ heterocyclyl C₁-C₆ alkyl; or L¹ and R⁶ combine to form an optionally substituted C₂-C₉ oxyheteroaryl, optionally substituted pyrimidin-4-yl, optionally substituted indazol-1-yl, optionally substituted indazol-2-yl, optionally substituted indazol-3-yl, optionally substituted benzotriazol-1-yl, optionally substituted pyrazin-2-yl, or optionally substituted pyrid-2-yl, or a C₆-C₁₀ aryl optionally substituted with an optionally substituted C₂-C₉ heteroaryl;
R¹ is hydrogen, halogen, or optionally substituted C₁₋₆ alkyl;
R² is hydrogen or optionally substituted C₂-C₉ heterocyclyl;
R³ is hydrogen, halogen, optionally substituted C₁-C₆ alkyl, optionally substituted C₁-C₆ heteroalkyl, or 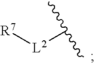
R⁴ is hydrogen, halogen, or optionally substituted C₁₋₆ alkyl;
L² is absent, 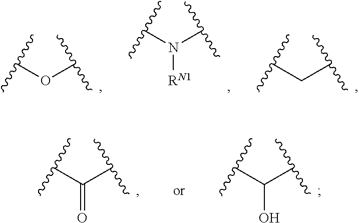
R⁷ is optionally substituted C₆₋₁₀ aryl, optionally substituted C₁-C₆ alkyl, optionally substituted C₃₋₁₀ carbocyclyl, optionally substituted C₁₋₉ heteroaryl, or optionally substituted C₁₋₉ heterocyclyl;
each of R^{N1}, R^{N2}, and R^{N3} is, independently, hydrogen or optionally substituted C₁₋₆ alkyl; and
m is 0, 1, 2, or 3;
wherein one and only one of X¹, X², X³, and X⁴ is N.

2. The compound of claim 1, wherein R⁵ is

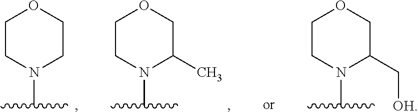

3. (canceled)

4. The compound of claim 1, wherein the compound has the structure of Formula Ta: 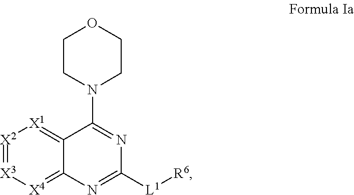
or a pharmaceutically acceptable salt thereof;
or the compound has the structure of Formula II: 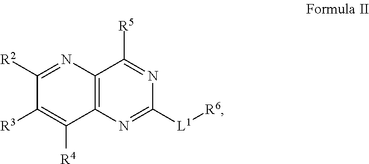
or a pharmaceutically acceptable salt thereof;
or the compound has the structure of Formula IIa: 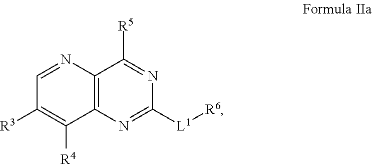
or a pharmaceutically acceptable salt thereof;
or the compound has the structure of Formula IIb: 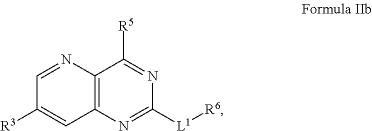
or a pharmaceutically acceptable salt thereof;
or the compound has the structure of Formula IIc: 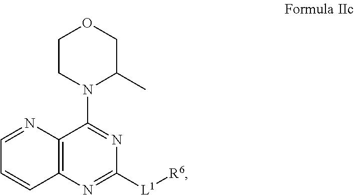
or a pharmaceutically acceptable salt thereof;
or the compound has the structure of Formula IId: 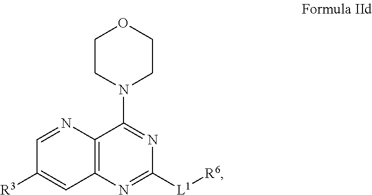
or a pharmaceutically acceptable salt thereof.

5-16. (canceled)

17. The compound of claim 1, wherein R³ is hydrogen, Br,

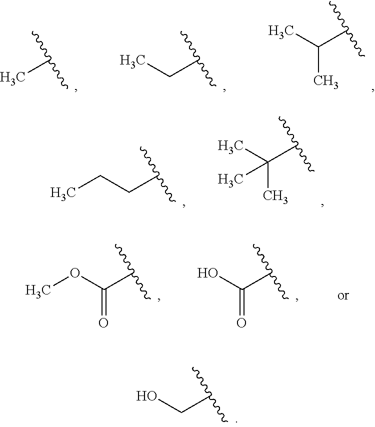

18-20. (canceled)

21. The compound of claim 1, wherein R³ is 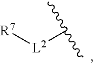
wherein L² is absent, or wherein L² is

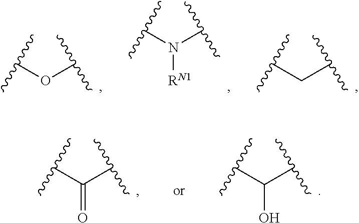

22-31. (canceled)

32. The compound of claim 1, wherein R⁷ is

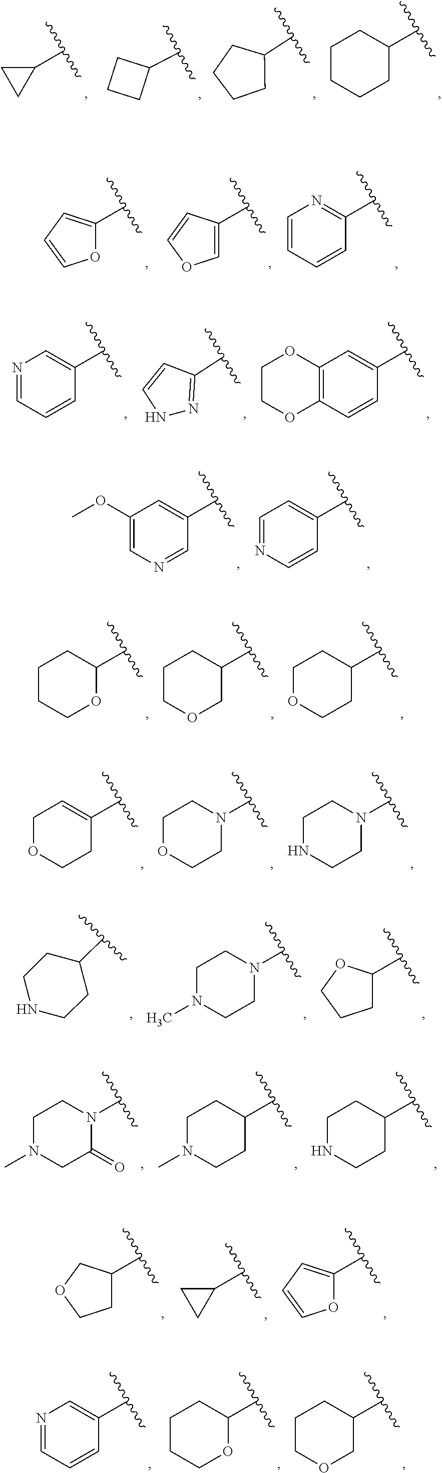

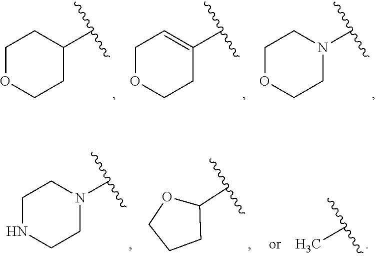

33-44. (canceled)

45. The compound of claim 1, wherein the compound has the structure of Formula IId: 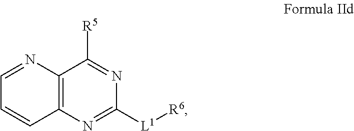
or a pharmaceutically acceptable salt thereof;
or the compound has the structure of Formula IIe: 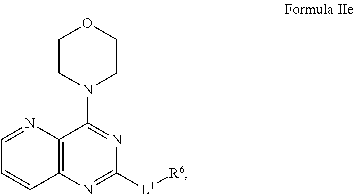
or a pharmaceutically acceptable salt thereof;
or the compound has the structure of Formula IIf: 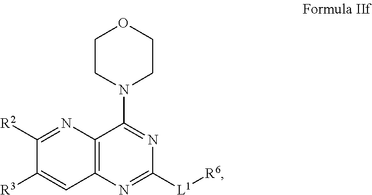
or a pharmaceutically acceptable salt thereof;
or the compound has the structure of Formula IIg: 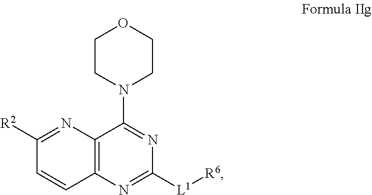
or a pharmaceutically acceptable salt thereof.

46-49. (canceled)

50. The compound of claim 1, wherein R² is

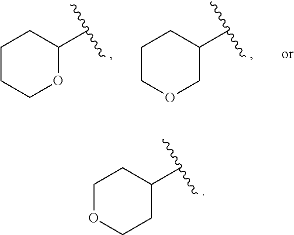 [SMILES: *C1CCCCO1 ; *C1CCCOC1 ; *C1CCOCC1]

51. (canceled)

52. The compound of claim 1, wherein the compound has the structure of Formula III: 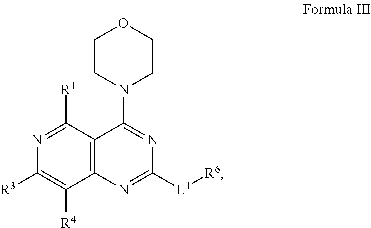
or a pharmaceutically acceptable salt thereof;
or the compound has the structure of Formula IIIa: 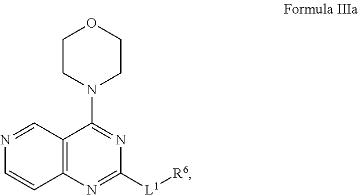
or a pharmaceutically acceptable salt thereof;
or the compound has the structure of Formula IV: 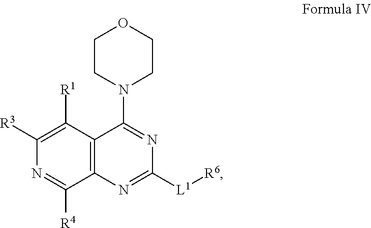
or a pharmaceutically acceptable salt thereof;
or the compound has the structure of Formula IVa: 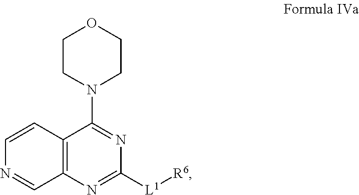
or a pharmaceutically acceptable salt thereof;
or the compound has the structure of Formula V: 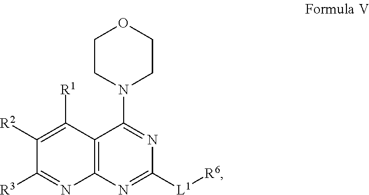
or a pharmaceutically acceptable salt thereof;
or the compound has the structure of Formula Va: 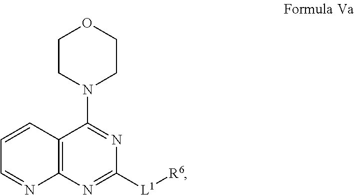
or a pharmaceutically acceptable salt thereof.

53-57. (canceled)

58. The compound of claim 1, wherein L¹ is 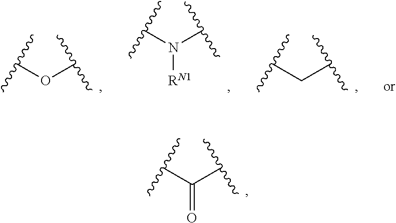
wherein R^{N1} is hydrogen or 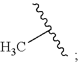
or L¹ is 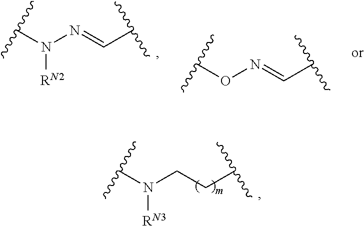
wherein R^{N2} is hydrogen or 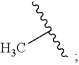
and wherein m is 1.

59-75. (canceled)

76. The compound of claim 1, wherein L¹ is 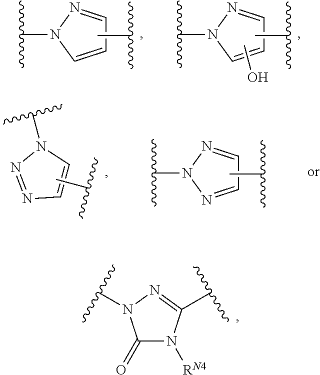
wherein R^{N4} is hydrogen or optionally substituted C₁₋₆ alkyl; or
L¹ is

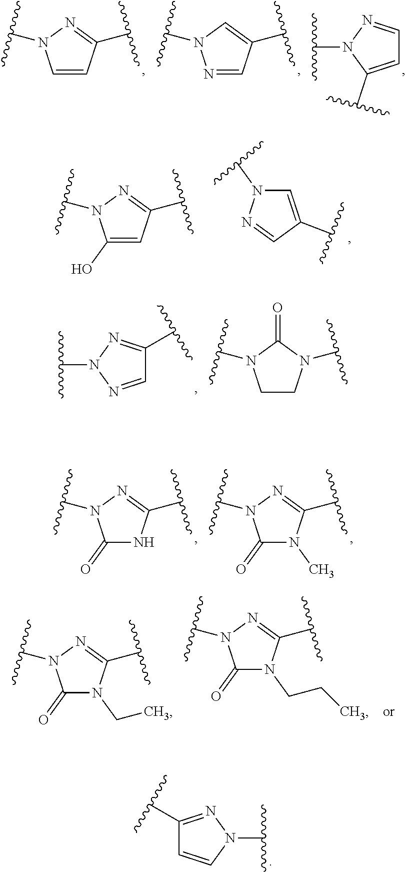

77-92. (canceled)

93. The compound of claim 1, wherein R⁶ is

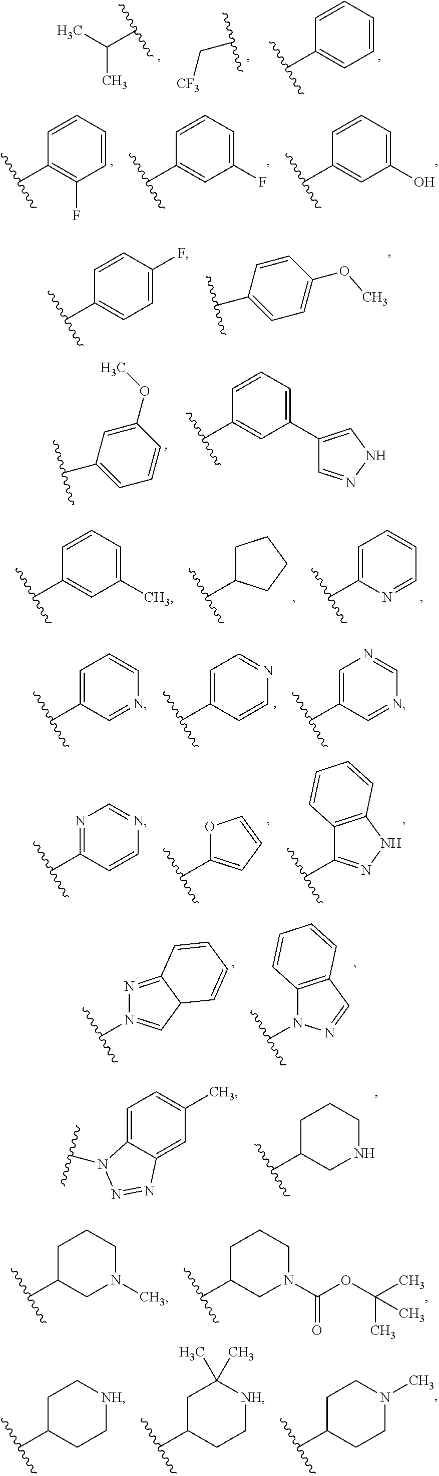

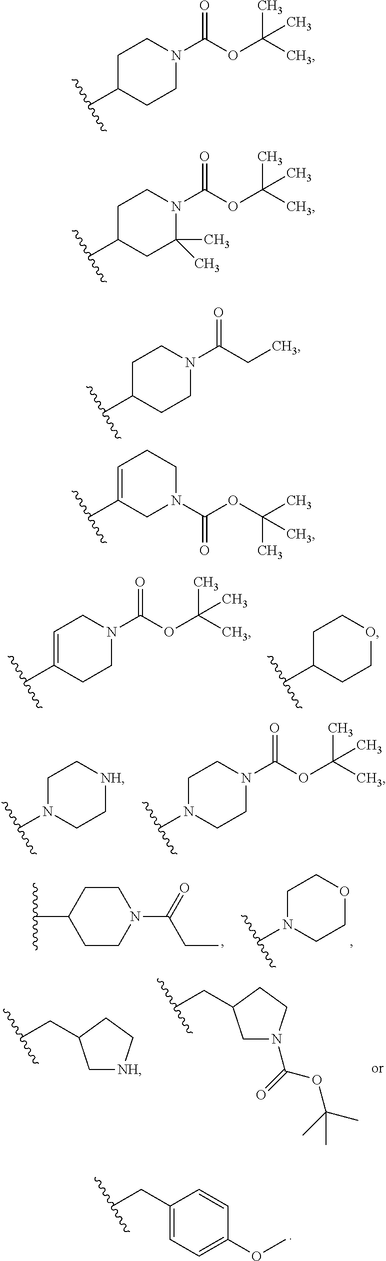

94-116. (canceled)

117. The compound of claim 1, wherein -L¹-R⁶ is

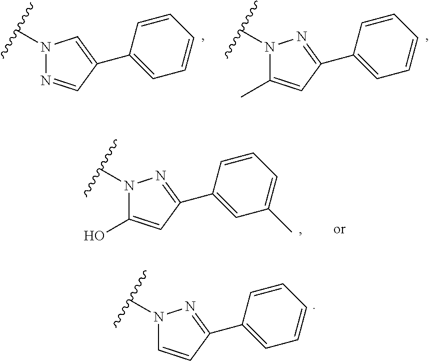 [SMILES: *n1cc(-c2ccccc2)cn1 ; *n1nc(-c2ccccc2)cc1C ; *n1nc(-c2cccc(C)c2)cc1O ; *n1ccc(-c2ccccc2)n1]

118-120. (canceled)

121. The compound of claim 1, wherein L¹ and R⁶ combine to form

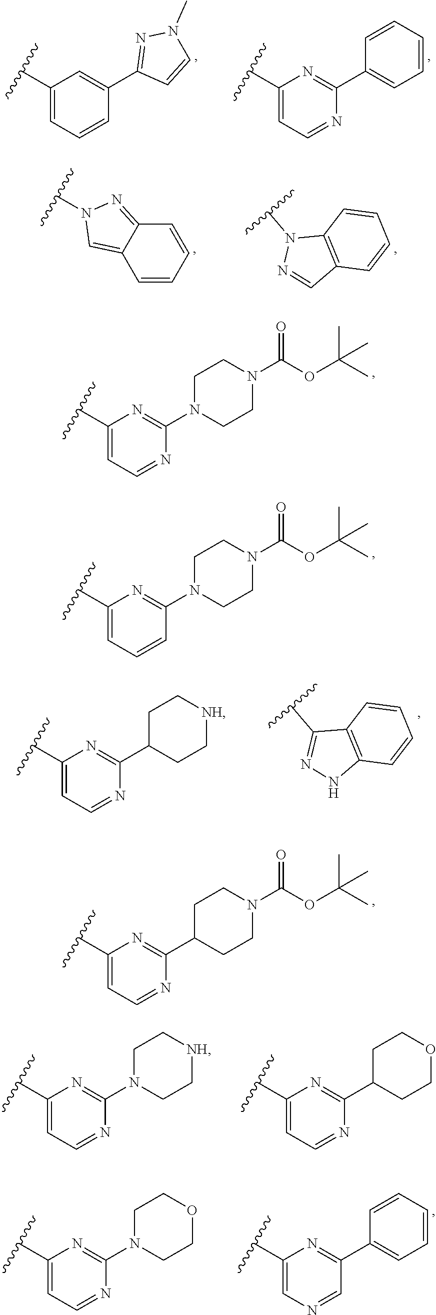

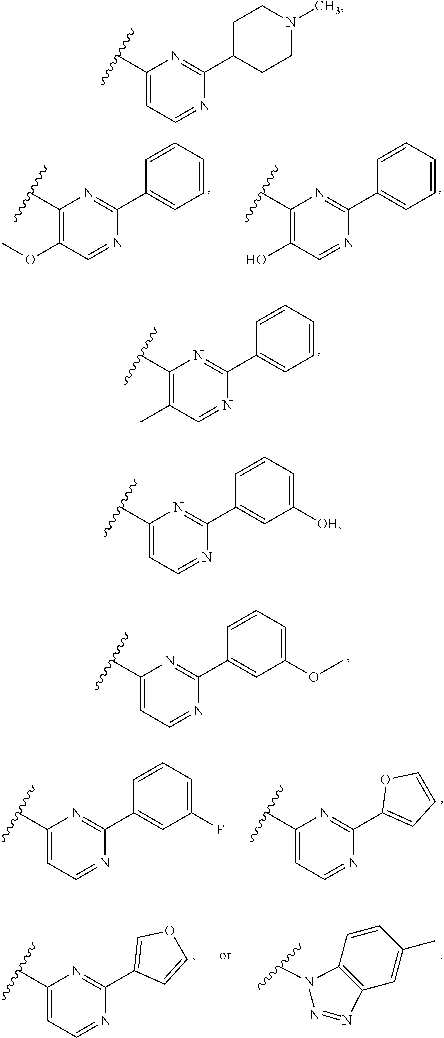

122-133. (canceled)

134. A compound of the following structure:

| # | Structure |
|---|---|
| 1 | 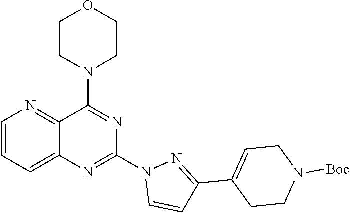 [SMILES: CC(C)(C)OC(=O)N1CC=C(c2ccn(-c3nc(N4CCOCC4)c4ncccc4n3)n2)CC1] |
| 2 | 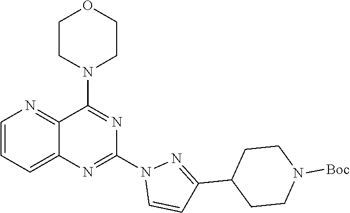 [SMILES: CC(C)(C)OC(=O)N1CCC(c2ccn(-c3nc(N4CCOCC4)c4ncccc4n3)n2)CC1] |
| 3 | 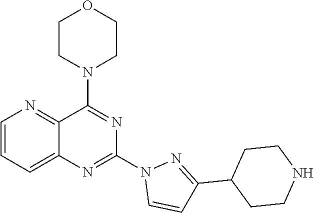 [SMILES: c1cnc2c(N3CCOCC3)nc(-n3ccc(C4CCNCC4)n3)nc2c1] |
| 4 | 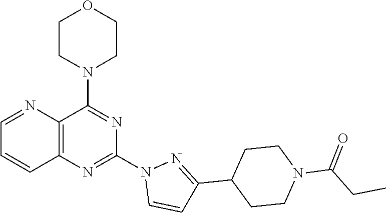 [SMILES: CCC(=O)N1CCC(c2ccn(-c3nc(N4CCOCC4)c4ncccc4n3)n2)CC1] |
| 5 | 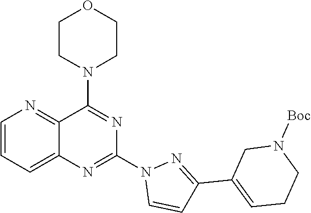 [SMILES: CC(C)(C)OC(=O)N1CCC=C(c2ccn(-c3nc(N4CCOCC4)c4ncccc4n3)n2)C1] |
| 6 | 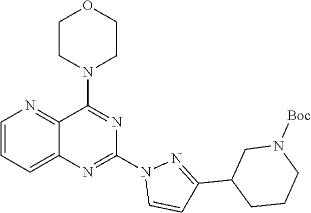 [SMILES: CC(C)(C)OC(=O)N1CCCC(c2ccn(-c3nc(N4CCOCC4)c4ncccc4n3)n2)C1] |
| 7 | 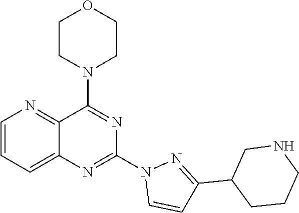 [SMILES: c1cnc2c(N3CCOCC3)nc(-n3ccc(C4CCCNC4)n3)nc2c1] |
| 8 | 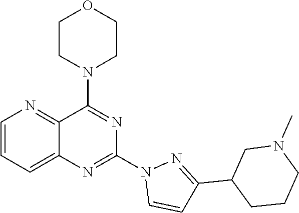 [SMILES: CN1CCCC(c2ccn(-c3nc(N4CCOCC4)c4ncccc4n3)n2)C1] |
| 9 | 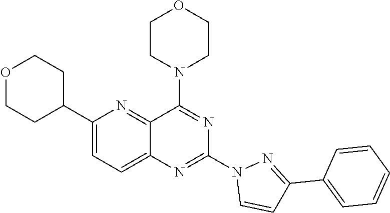 [SMILES: c1ccc(-c2ccn(-c3nc(N4CCOCC4)c4nc(C5CCOCC5)ccc4n3)n2)cc1] |
| 10 | 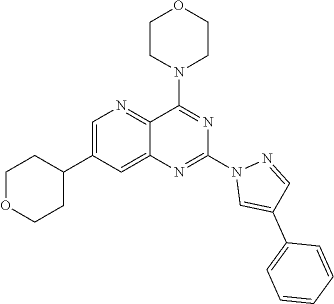 [SMILES: c1ccc(-c2cnn(-c3nc(N4CCOCC4)c4ncc(C5CCOCC5)cc4n3)c2)cc1] |
| 11 | 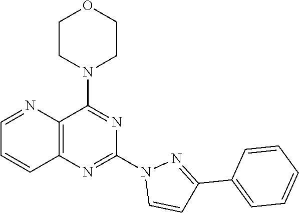 [SMILES: c1ccc(-c2ccn(-c3nc(N4CCOCC4)c4ncccc4n3)n2)cc1] |
| 12 | 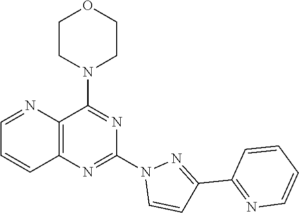 [SMILES: c1ccc(-c2ccn(-c3nc(N4CCOCC4)c4ncccc4n3)n2)nc1] |
| 13 | 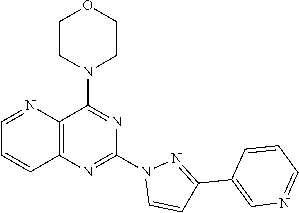 [SMILES: c1cncc(-c2ccn(-c3nc(N4CCOCC4)c4ncccc4n3)n2)c1] |
| 14 | 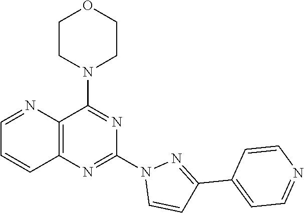 [SMILES: c1cnc2c(N3CCOCC3)nc(-n3ccc(-c4ccncc4)n3)nc2c1] |
| 15 | 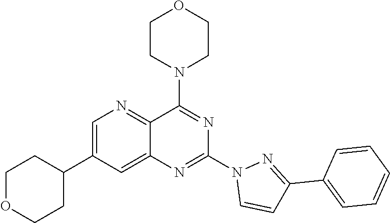 [SMILES: c1ccc(-c2ccn(-c3nc(N4CCOCC4)c4ncc(C5CCOCC5)cc4n3)n2)cc1] |
| 16 | 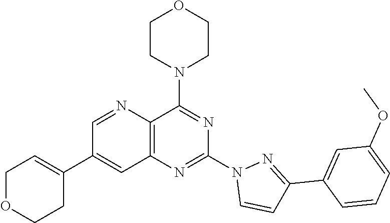 [SMILES: COc1cccc(-c2ccn(-c3nc(N4CCOCC4)c4ncc(C5=CCOCC5)cc4n3)n2)c1] |
| 17 | 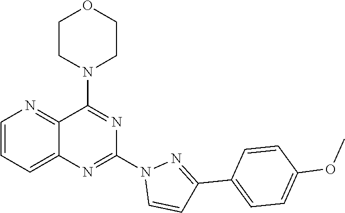 [SMILES: COc1ccc(-c2ccn(-c3nc(N4CCOCC4)c4ncccc4n3)n2)cc1] |
| 18 | 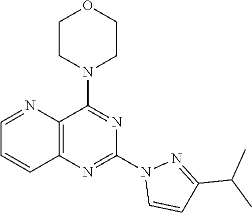 [SMILES: CC(C)c1ccn(-c2nc(N3CCOCC3)c3ncccc3n2)n1] |
| 19 | 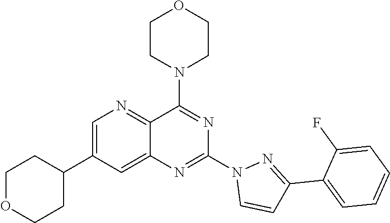 [SMILES: Fc1ccccc1-c1ccn(-c2nc(N3CCOCC3)c3ncc(C4CCOCC4)cc3n2)n1] |
| 20 | 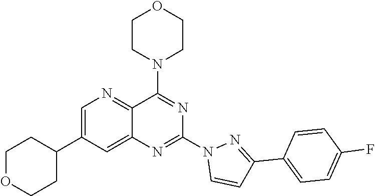 [SMILES: Fc1ccc(-c2ccn(-c3nc(N4CCOCC4)c4ncc(C5CCOCC5)cc4n3)n2)cc1] |
| 21 | 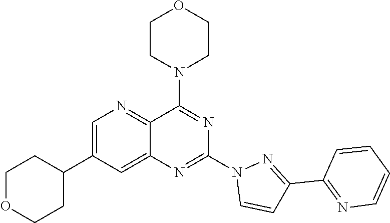 [SMILES: c1ccc(-c2ccn(-c3nc(N4CCOCC4)c4ncc(C5CCOCC5)cc4n3)n2)nc1] |
| 22 | 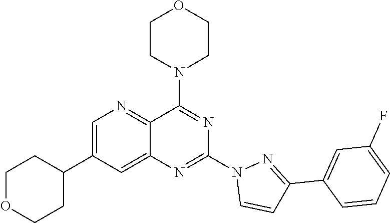 [SMILES: Fc1cccc(-c2ccn(-c3nc(N4CCOCC4)c4ncc(C5CCOCC5)cc4n3)n2)c1] |
| 23 | 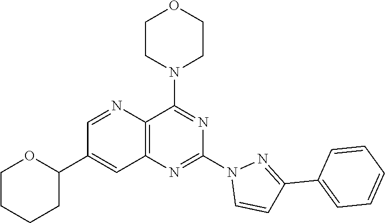 [SMILES: c1ccc(-c2ccn(-c3nc(N4CCOCC4)c4ncc(C5CCCCO5)cc4n3)n2)cc1] |
| 24 | 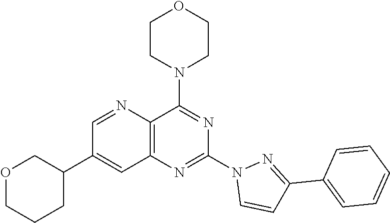 [SMILES: c1ccc(-c2ccn(-c3nc(N4CCOCC4)c4ncc(C5CCCOC5)cc4n3)n2)cc1] |
| 25 | 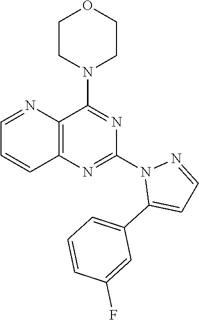 [SMILES: Fc1cccc(-c2ccnn2-c2nc(N3CCOCC3)c3ncccc3n2)c1] |
| 26 | 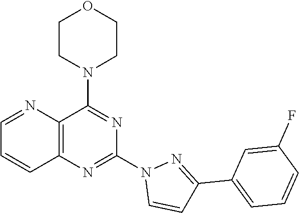 [SMILES: Fc1cccc(-c2ccn(-c3nc(N4CCOCC4)c4ncccc4n3)n2)c1] |
| 27 | 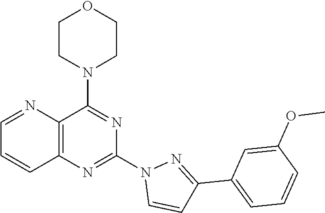 [SMILES: COc1cccc(-c2ccn(-c3nc(N4CCOCC4)c4ncccc4n3)n2)c1] |
| 28 | 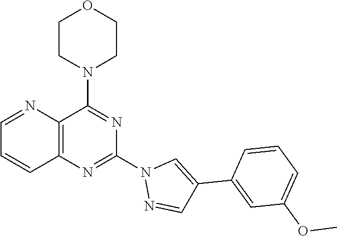 [SMILES: COc1cccc(-c2cnn(-c3nc(N4CCOCC4)c4ncccc4n3)c2)c1] |
| 29 | 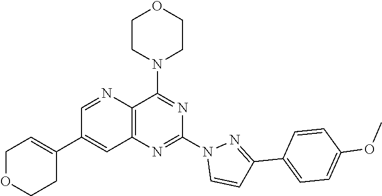 [SMILES: COc1ccc(-c2ccn(-c3nc(N4CCOCC4)c4ncc(C5=CCOCC5)cc4n3)n2)cc1] |
| 30 | 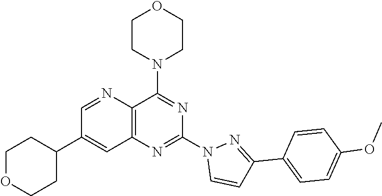 [SMILES: COc1ccc(-c2ccn(-c3nc(N4CCOCC4)c4ncc(C5CCOCC5)cc4n3)n2)cc1] |
| 31 | 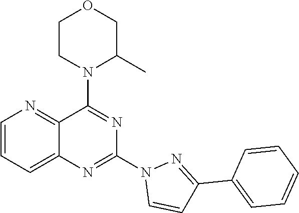 [SMILES: CC1COCCN1c1nc(-n2ccc(-c3ccccc3)n2)nc2cccnc12] |
| 32 | 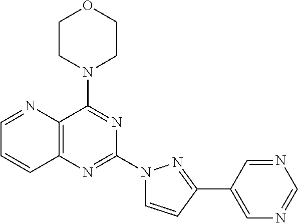 [SMILES: c1cnc2c(N3CCOCC3)nc(-n3ccc(-c4cncnc4)n3)nc2c1] |
| 33 | 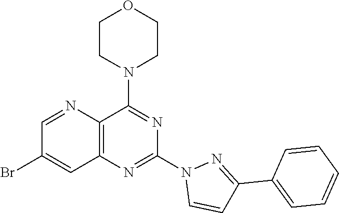 [SMILES: Brc1cnc2c(N3CCOCC3)nc(-n3ccc(-c4ccccc4)n3)nc2c1] |
| 34 | 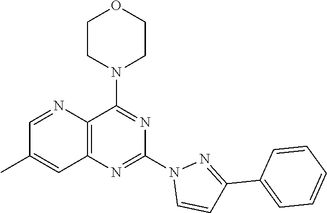 [SMILES: Cc1cnc2c(N3CCOCC3)nc(-n3ccc(-c4ccccc4)n3)nc2c1] |
| 35 | 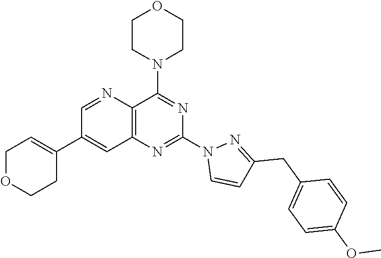 [SMILES: COc1ccc(Cc2ccn(-c3nc(N4CCOCC4)c4ncc(C5=CCOCC5)cc4n3)n2)cc1] |
| 36 | 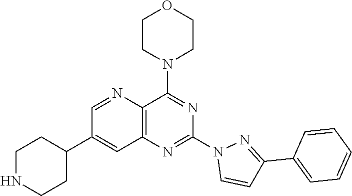 [SMILES: c1ccc(-c2ccn(-c3nc(N4CCOCC4)c4ncc(C5CCNCC5)cc4n3)n2)cc1] |
| 37 | 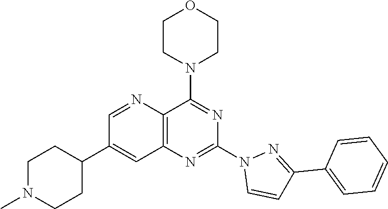 [SMILES: CN1CCC(c2cnc3c(N4CCOCC4)nc(-n4ccc(-c5ccccc5)n4)nc3c2)CC1] |
| 38 | 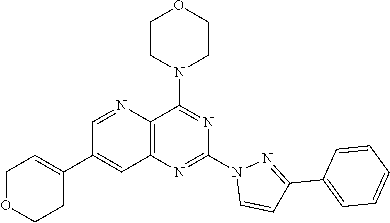 [SMILES: C1=C(c2cnc3c(N4CCOCC4)nc(-n4ccc(-c5ccccc5)n4)nc3c2)CCOC1] |
| 39 | 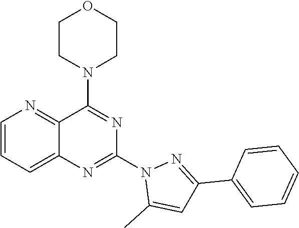 [SMILES: Cc1cc(-c2ccccc2)nn1-c1nc(N2CCOCC2)c2ncccc2n1] |
| 40 | 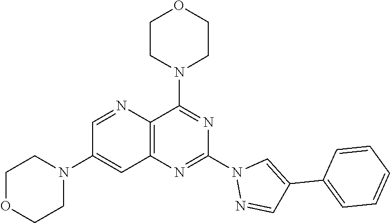 [SMILES: c1ccc(-c2cnn(-c3nc(N4CCOCC4)c4ncc(N5CCOCC5)cc4n3)c2)cc1] |
| 41 | 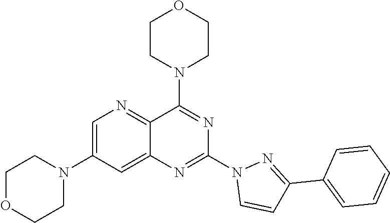 [SMILES: c1ccc(-c2ccn(-c3nc(N4CCOCC4)c4ncc(N5CCOCC5)cc4n3)n2)cc1] |
| 42 | 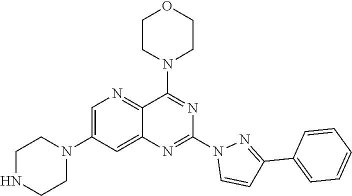 [SMILES: c1ccc(-c2ccn(-c3nc(N4CCOCC4)c4ncc(N5CCNCC5)cc4n3)n2)cc1] |
| 43 | 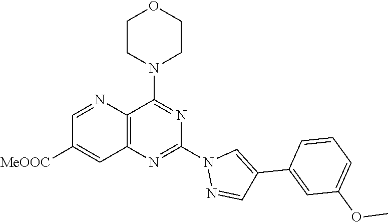 [SMILES: COC(=O)c1cnc2c(N3CCOCC3)nc(-n3cc(-c4cccc(OC)c4)cn3)nc2c1] |
| 44 | 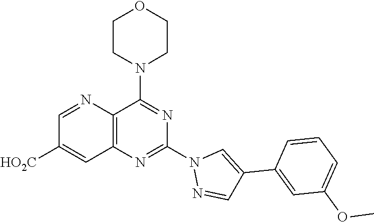 [SMILES: COc1cccc(-c2cnn(-c3nc(N4CCOCC4)c4ncc(C(=O)O)cc4n3)c2)c1] |
| 45 | 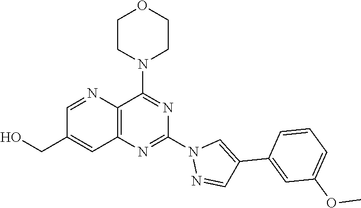 [SMILES: COc1cccc(-c2cnn(-c3nc(N4CCOCC4)c4ncc(CO)cc4n3)c2)c1] |
| 46 | 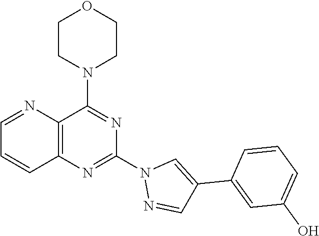 [SMILES: Oc1cccc(-c2cnn(-c3nc(N4CCOCC4)c4ncccc4n3)c2)c1] |
| 47 | 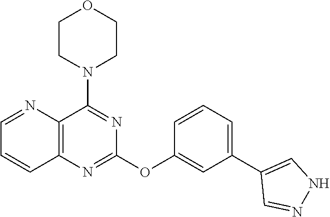 [SMILES: c1cc(Oc2nc(N3CCOCC3)c3ncccc3n2)cc(-c2cn[nH]c2)c1] |
| 48 | 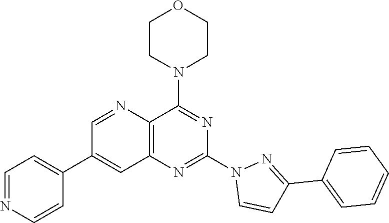 [SMILES: c1ccc(-c2ccn(-c3nc(N4CCOCC4)c4ncc(-c5ccncc5)cc4n3)n2)cc1] |
| 49 | 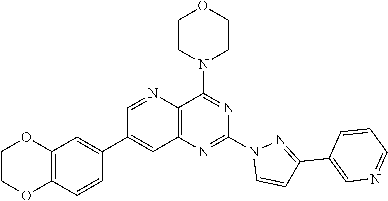 [SMILES: c1cncc(-c2ccn(-c3nc(N4CCOCC4)c4ncc(-c5ccc6c(c5)OCCO6)cc4n3)n2)c1] |
| 50 | 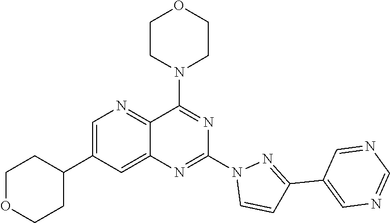 [SMILES: c1ncc(-c2ccn(-c3nc(N4CCOCC4)c4ncc(C5CCOCC5)cc4n3)n2)cn1] |
| 51 | 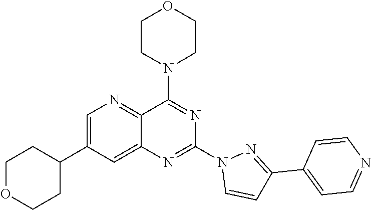 [SMILES: c1cc(-c2ccn(-c3nc(N4CCOCC4)c4ncc(C5CCOCC5)cc4n3)n2)ccn1] |
| 52 | 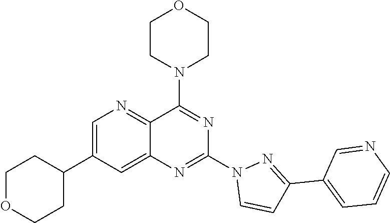 [SMILES: c1cncc(-c2ccn(-c3nc(N4CCOCC4)c4ncc(C5CCOCC5)cc4n3)n2)c1] |
| 53 | 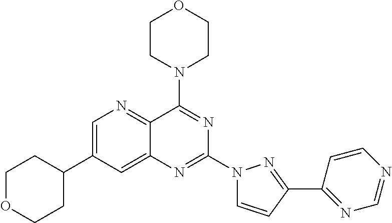 [SMILES: c1cc(-c2ccn(-c3nc(N4CCOCC4)c4ncc(C5CCOCC5)cc4n3)n2)ncn1] |
| 54 | 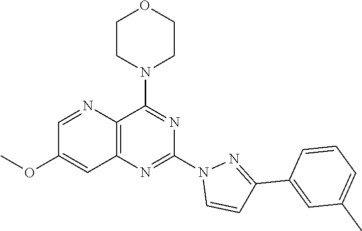 |
| 55 | 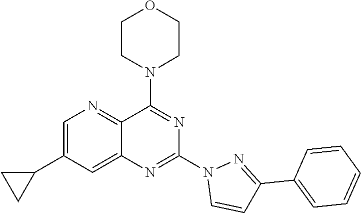 [SMILES: c1ccc(-c2ccn(-c3nc(N4CCOCC4)c4ncc(C5CC5)cc4n3)n2)cc1] |
| 56 | 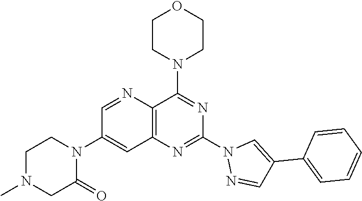 [SMILES: CN1CCN(c2cnc3c(N4CCOCC4)nc(-n4cc(-c5ccccc5)cn4)nc3c2)C(=O)C1] |
| 57 | 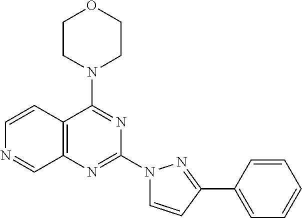 [SMILES: c1ccc(-c2ccn(-c3nc(N4CCOCC4)c4ccncc4n3)n2)cc1] |
| 58 | 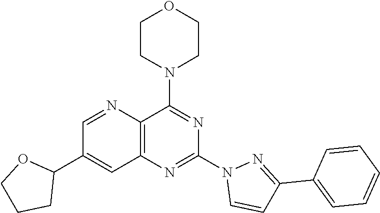 [SMILES: c1ccc(-c2ccn(-c3nc(N4CCOCC4)c4ncc(C5CCCO5)cc4n3)n2)cc1] |
| 59 | 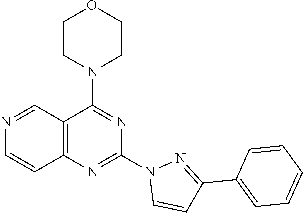 [SMILES: c1ccc(-c2ccn(-c3nc(N4CCOCC4)c4cnccc4n3)n2)cc1] |
| 60 | 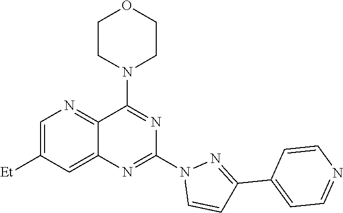 [SMILES: CCc1cnc2c(N3CCOCC3)nc(-n3ccc(-c4ccncc4)n3)nc2c1] |
| 61 | 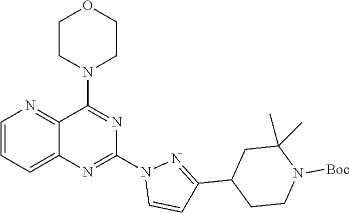 [SMILES: CC(C)(C)OC(=O)N1CCC(c2ccn(-c3nc(N4CCOCC4)c4ncccc4n3)n2)CC1(C)C] |
| 62 | 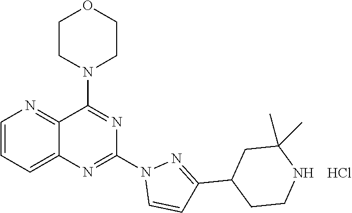 [SMILES: CC1(C)CC(c2ccn(-c3nc(N4CCOCC4)c4ncccc4n3)n2)CCN1 ; Cl] |
| 63 | 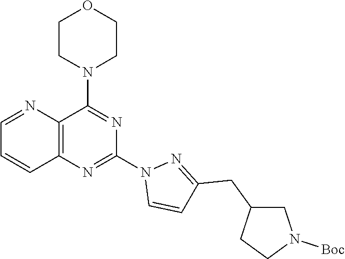 [SMILES: CC(C)(C)OC(=O)N1CCC(Cc2ccn(-c3nc(N4CCOCC4)c4ncccc4n3)n2)C1] |
| 64 | 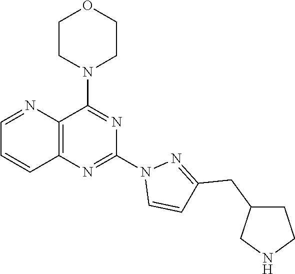 [SMILES: c1cnc2c(N3CCOCC3)nc(-n3ccc(CC4CCNC4)n3)nc2c1] |
| 65 | 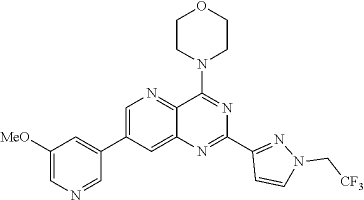 [SMILES: COc1cncc(-c2cnc3c(N4CCOCC4)nc(-c4ccn(CC(F)(F)F)n4)nc3c2)c1] |
| 66 | 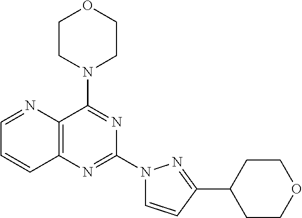 [SMILES: c1cnc2c(N3CCOCC3)nc(-n3ccc(C4CCOCC4)n3)nc2c1] |
| 67 | 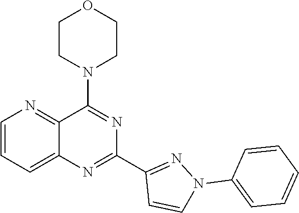 [SMILES: c1ccc(-n2ccc(-c3nc(N4CCOCC4)c4ncccc4n3)n2)cc1] |
| 68 | 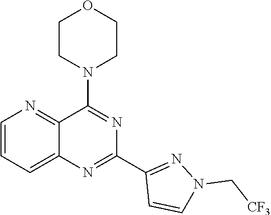 [SMILES: FC(F)(F)Cn1ccc(-c2nc(N3CCOCC3)c3ncccc3n2)n1] |
| 69 | 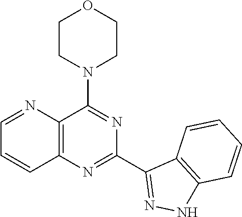 [SMILES: c1cnc2c(N3CCOCC3)nc(-c3n[nH]c4ccccc34)nc2c1] |
| 70 | 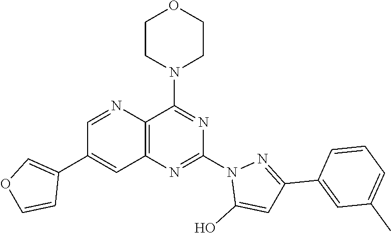 [SMILES: Cc1cccc(-c2cc(O)n(-c3nc(N4CCOCC4)c4ncc(-c5ccoc5)cc4n3)n2)c1] |
| 71 | 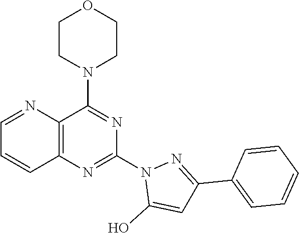 [SMILES: Oc1cc(-c2ccccc2)nn1-c1nc(N2CCOCC2)c2ncccc2n1] |
| 72 | 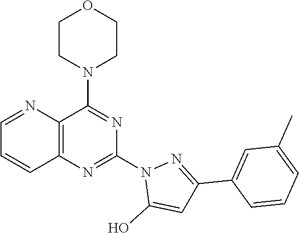 [SMILES: Cc1cccc(-c2cc(O)n(-c3nc(N4CCOCC4)c4ncccc4n3)n2)c1] |
| 73 | 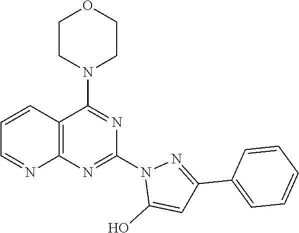 [SMILES: Oc1cc(-c2ccccc2)nn1-c1nc(N2CCOCC2)c2cccnc2n1] |
| 74 | 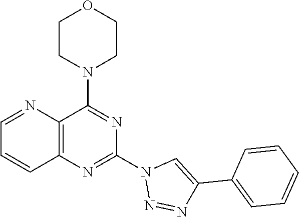 [SMILES: c1ccc(-c2cn(-c3nc(N4CCOCC4)c4ncccc4n3)nn2)cc1] |
| 75 | 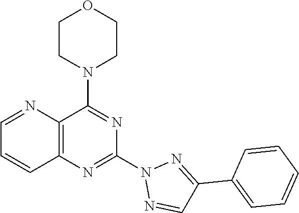 [SMILES: c1ccc(-c2cnn(-c3nc(N4CCOCC4)c4ncccc4n3)n2)cc1] |
| 76 | 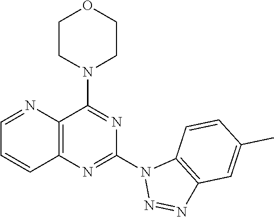 [SMILES: Cc1ccc2c(c1)nnn2-c1nc(N2CCOCC2)c2ncccc2n1] |
| 77 | 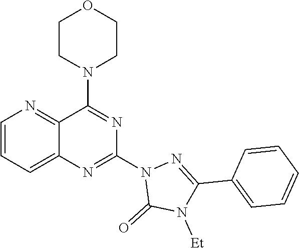 [SMILES: CCn1c(-c2ccccc2)nn(-c2nc(N3CCOCC3)c3ncccc3n2)c1=O] |
| 78 | 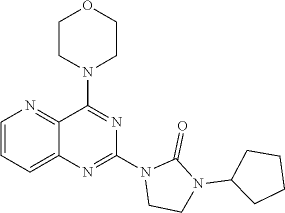 [SMILES: O=C1N(c2nc(N3CCOCC3)c3ncccc3n2)CCN1C1CCCC1] |
| 79 | 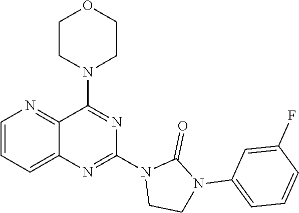 [SMILES: O=C1N(c2cccc(F)c2)CCN1c1nc(N2CCOCC2)c2ncccc2n1] |
| 80 | 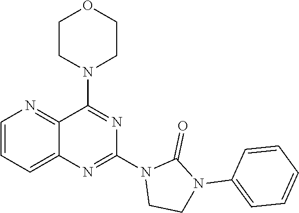 [SMILES: O=C1N(c2ccccc2)CCN1c1nc(N2CCOCC2)c2ncccc2n1] |
| 81 | 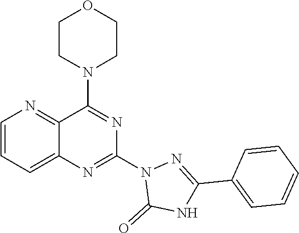 [SMILES: O=c1[nH]c(-c2ccccc2)nn1-c1nc(N2CCOCC2)c2ncccc2n1] |
| 82 | 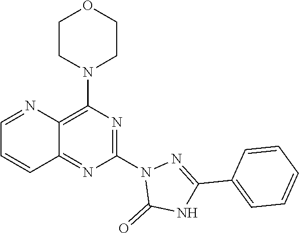 [SMILES: O=c1[nH]c(-c2ccccc2)nn1-c1nc(N2CCOCC2)c2ncccc2n1] |
| 83 | 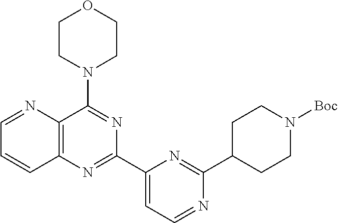 [SMILES: CC(C)(C)OC(=O)N1CCC(c2nccc(-c3nc(N4CCOCC4)c4ncccc4n3)n2)CC1] |
| 84 | 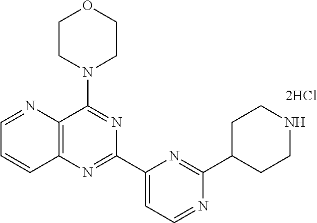 [SMILES: c1cnc2c(N3CCOCC3)nc(-c3ccnc(C4CCNCC4)n3)nc2c1] |
| 85 | 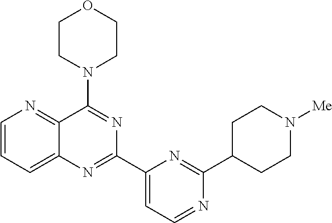 [SMILES: CN1CCC(c2nccc(-c3nc(N4CCOCC4)c4ncccc4n3)n2)CC1] |
| 86 | 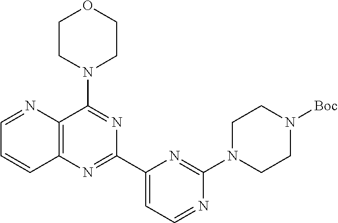 [SMILES: CC(C)(C)OC(=O)N1CCN(c2nccc(-c3nc(N4CCOCC4)c4ncccc4n3)n2)CC1] |
| 87 | 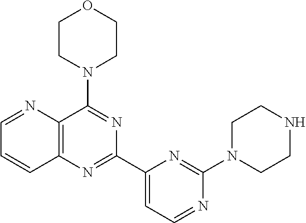 [SMILES: c1cnc2c(N3CCOCC3)nc(-c3ccnc(N4CCNCC4)n3)nc2c1] |
| 88 | 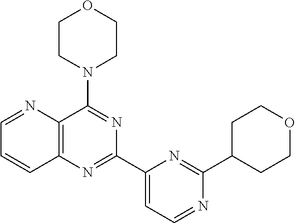 [SMILES: c1cnc2c(N3CCOCC3)nc(-c3ccnc(C4CCOCC4)n3)nc2c1] |
| 89 | 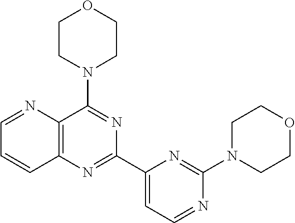 [SMILES: c1cnc2c(N3CCOCC3)nc(-c3ccnc(N4CCOCC4)n3)nc2c1] |
| 90 | 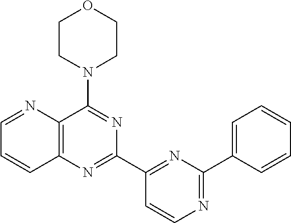 [SMILES: c1ccc(-c2nccc(-c3nc(N4CCOCC4)c4ncccc4n3)n2)cc1] |
| 91 | 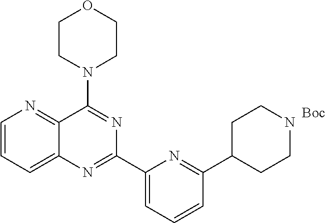 [SMILES: c1cc(-c2nc(N3CCOCC3)c3ncccc3n2)nc(C2CCNCC2)c1 ; "Boc"] |
| 92 | 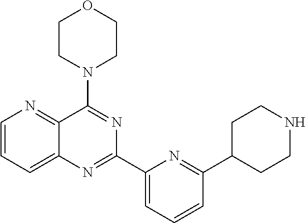 [SMILES: c1cc(-c2nc(N3CCOCC3)c3ncccc3n2)nc(C2CCNCC2)c1] |
| 93 | 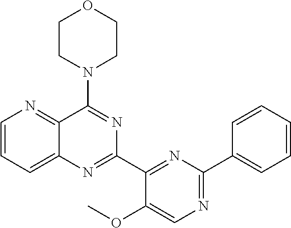 [SMILES: COc1cnc(-c2ccccc2)nc1-c1nc(N2CCOCC2)c2ncccc2n1] |
| 94 | 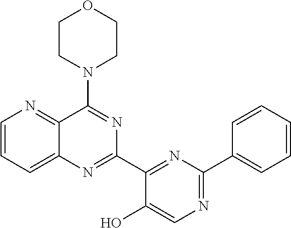 [SMILES: Oc1cnc(-c2ccccc2)nc1-c1nc(N2CCOCC2)c2ncccc2n1] |
| 95 | 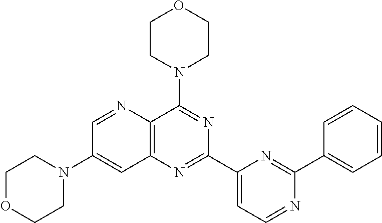 [SMILES: c1ccc(-c2nccc(-c3nc(N4CCOCC4)c4ncc(N5CCOCC5)cc4n3)n2)cc1] |
| [[92]] 96 | 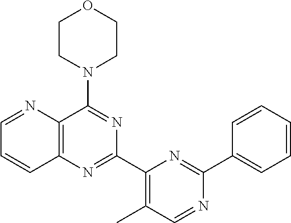 [SMILES: Cc1cnc(-c2ccccc2)nc1-c1nc(N2CCOCC2)c2ncccc2n1] |
| 97 | 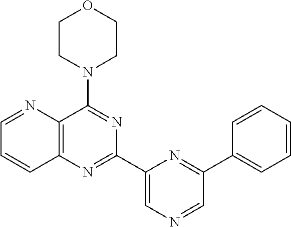 [SMILES: c1ccc(-c2cncc(-c3nc(N4CCOCC4)c4ncccc4n3)n2)cc1] |
| 98 | 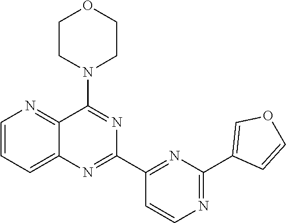 [SMILES: c1cnc2c(N3CCOCC3)nc(-c3ccnc(-c4ccoc4)n3)nc2c1] |
| 99 | 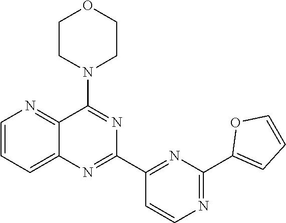 [SMILES: c1coc(-c2nccc(-c3nc(N4CCOCC4)c4ncccc4n3)n2)c1] |
| 100 | 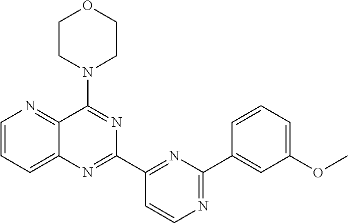 [SMILES: COc1cccc(-c2nccc(-c3nc(N4CCOCC4)c4ncccc4n3)n2)c1] |
| 101 | 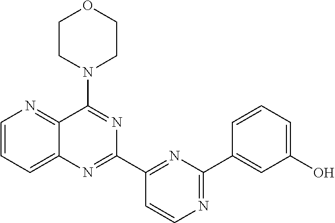 [SMILES: Oc1cccc(-c2nccc(-c3nc(N4CCOCC4)c4ncccc4n3)n2)c1] |
| 102 | 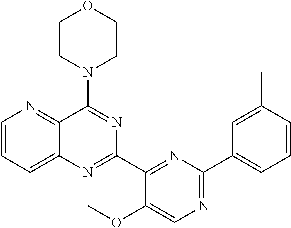 [SMILES: COc1cnc(-c2cccc(C)c2)nc1-c1nc(N2CCOCC2)c2ncccc2n1] |
| 103 | 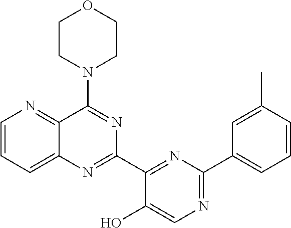 [SMILES: Cc1cccc(-c2ncc(O)c(-c3nc(N4CCOCC4)c4ncccc4n3)n2)c1] |
| 104 | 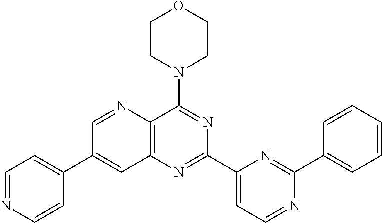 [SMILES: c1ccc(-c2nccc(-c3nc(N4CCOCC4)c4ncc(-c5ccncc5)cc4n3)n2)cc1] |
| 105 | 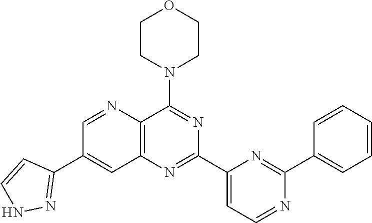 [SMILES: c1ccc(-c2nccc(-c3nc(N4CCOCC4)c4ncc(-c5cc[nH]n5)cc4n3)n2)cc1] |
| 106 | 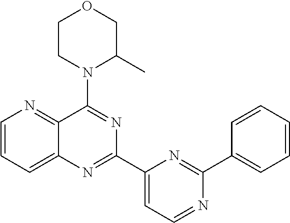 [SMILES: CC1COCCN1c1nc(-c2ccnc(-c3ccccc3)n2)nc2cccnc12] |
| 107 | 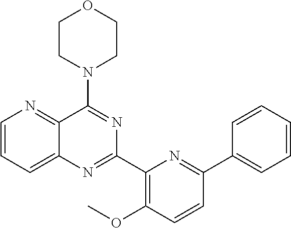 [SMILES: COc1ccc(-c2ccccc2)nc1-c1nc(N2CCOCC2)c2ncccc2n1] |
| 108 | 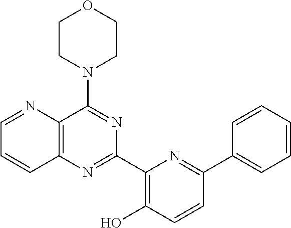 [SMILES: Oc1ccc(-c2ccccc2)nc1-c1nc(N2CCOCC2)c2ncccc2n1] |
| 109 | 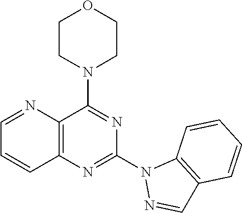 [SMILES: c1ccc2c(c1)cnn2-c1nc(N2CCOCC2)c2ncccc2n1] |
| 110 | 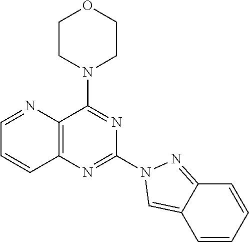 [SMILES: c1ccc2nn(-c3nc(N4CCOCC4)c4ncccc4n3)cc2c1] |
| 111 | 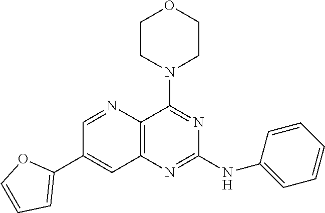 [SMILES: c1ccc(Nc2nc(N3CCOCC3)c3ncc(-c4ccco4)cc3n2)cc1] |
| 112 | 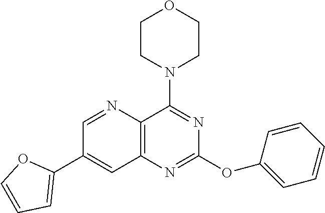 [SMILES: c1ccc(Oc2nc(N3CCOCC3)c3ncc(-c4ccco4)cc3n2)cc1] |
| 113 | 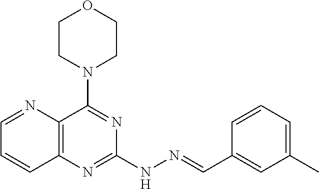 [SMILES: Cc1cccc(/C=N/Nc2nc(N3CCOCC3)c3ncccc3n2)c1] |
| 114 | 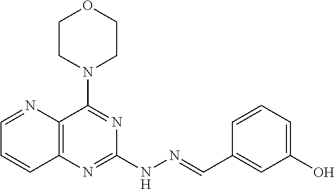 [SMILES: Oc1cccc(/C=N/Nc2nc(N3CCOCC3)c3ncccc3n2)c1] |
| 115 | 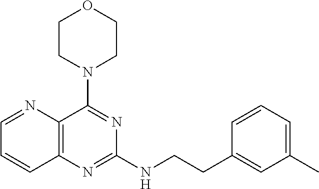 [SMILES: Cc1cccc(CCNc2nc(N3CCOCC3)c3ncccc3n2)c1] |
| 116 | 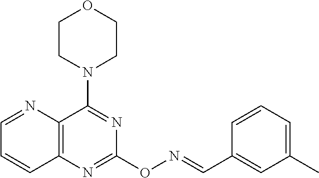 [SMILES: Cc1cccc(/C=N/Oc2nc(N3CCOCC3)c3ncccc3n2)c1] |
| 117 | 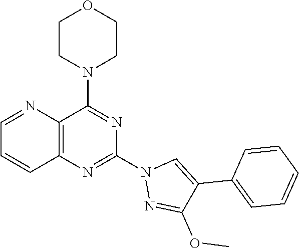 [SMILES: COc1nn(-c2nc(N3CCOCC3)c3ncccc3n2)cc1-c1ccccc1] |
| 122 | 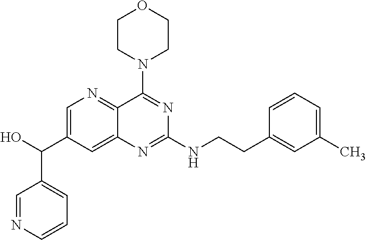 [SMILES: Cc1cccc(CCNc2nc(N3CCOCC3)c3ncc(C(O)c4cccnc4)cc3n2)c1] |
| 123 | 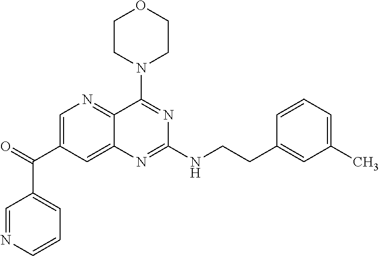 [SMILES: Cc1cccc(CCNc2nc(N3CCOCC3)c3ncc(C(=O)c4cccnc4)cc3n2)c1] |
| 124 | 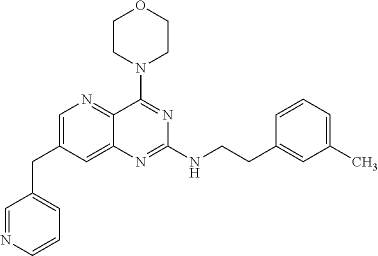 [SMILES: Cc1cccc(CCNc2nc(N3CCOCC3)c3ncc(Cc4cccnc4)cc3n2)c1] |
| 125 | 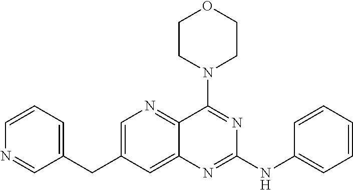 [SMILES: c1ccc(Nc2nc(N3CCOCC3)c3ncc(Cc4cccnc4)cc3n2)cc1] |
| 126 | 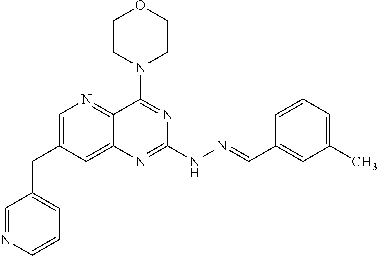 [SMILES: Cc1cccc(/C=N/Nc2nc(N3CCOCC3)c3ncc(Cc4cccnc4)cc3n2)c1] |
| 127 | 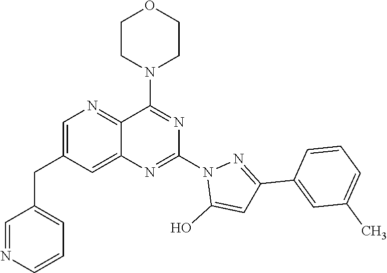 [SMILES: Cc1cccc(-c2cc(O)n(-c3nc(N4CCOCC4)c4ncc(Cc5cccnc5)cc4n3)n2)c1] |
| 128 | 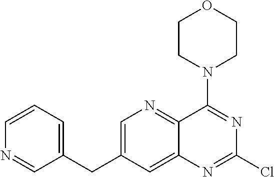 [SMILES: Clc1nc(N2CCOCC2)c2ncc(Cc3cccnc3)cc2n1] |
| 129 | 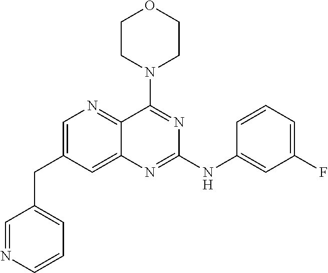 [SMILES: Fc1cccc(Nc2nc(N3CCOCC3)c3ncc(Cc4cccnc4)cc3n2)c1] |
| 130 | 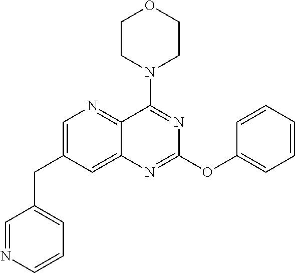 [SMILES: c1ccc(Oc2nc(N3CCOCC3)c3ncc(Cc4cccnc4)cc3n2)cc1] |
| 131 | 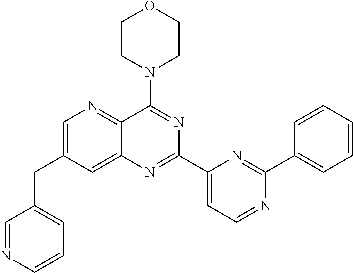 [SMILES: c1ccc(-c2nccc(-c3nc(N4CCOCC4)c4ncc(Cc5cccnc5)cc4n3)n2)cc1] |
| 132 | 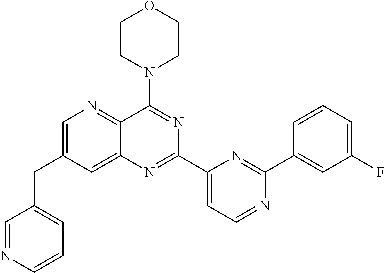 [SMILES: Fc1cccc(-c2nccc(-c3nc(N4CCOCC4)c4ncc(Cc5cccnc5)cc4n3)n2)c1] |
| 133 | 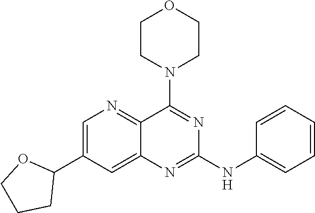 [SMILES: c1ccc(Nc2nc(N3CCOCC3)c3ncc(C4CCCO4)cc3n2)cc1] |
| 134 | 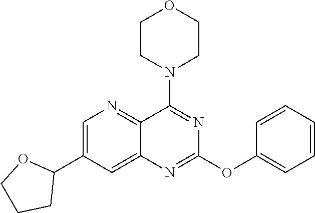 [SMILES: c1ccc(Oc2nc(N3CCOCC3)c3ncc(C4CCCO4)cc3n2)cc1] |
| 135 | 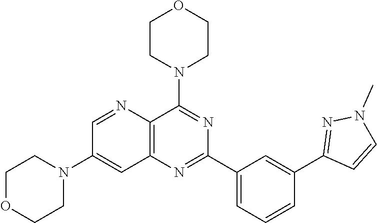 [SMILES: Cn1ccc(-c2cccc(-c3nc(N4CCOCC4)c4ncc(N5CCOCC5)cc4n3)c2)n1] |
| 136 | 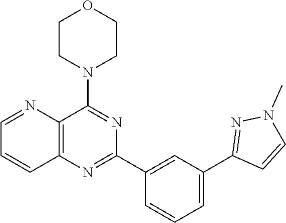 [SMILES: Cn1ccc(-c2cccc(-c3nc(N4CCOCC4)c4ncccc4n3)c2)n1] |
| 137 | 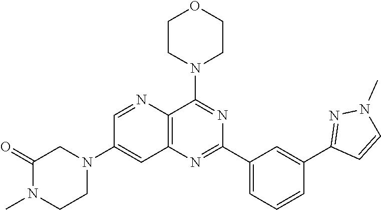 [SMILES: CN1CCN(c2cnc3c(N4CCOCC4)nc(-c4cccc(-c5ccn(C)n5)c4)nc3c2)CC1=O] |

135. (canceled)

136. A pharmaceutical composition comprising the compound of claim 1, or a pharmaceutically acceptable salt thereof, and a pharmaceutically acceptable excipient.

137. A method of treating a neurological disorder in a subject in need thereof, the method comprising administering to the subject an effective amount of the compound of claim 1, or a pharmaceutically acceptable salt thereof.

138. The method of claim 137, wherein the neurological disorder is FTLD-TDP, chronic traumatic encephalopathy, ALS, Alzheimer's disease, LATE, or frontotemporal lobar degeneration.

139. (canceled)

140. A method of inhibiting toxicity in a cell related to a protein, the method comprising contacting the cell with the compound of claim 1 or a pharmaceutically acceptable salt thereof.

141. The method of claim 140, wherein the toxicity is TDP-43-related toxicity, or C9orf72-related toxicity.

142-147. (canceled)
